(19) United States
(12) Patent Application Publication
(10) Pub. No.: US 2024/0271138 A1
(43) Pub. Date: Aug. 15, 2024

(54) METHODS AND COMPOSITIONS FOR MODULATING STING SIGNALING AND INNATE IMMUNE RESPONSES

(71) Applicants: The Broad Institute, Inc., Cambridge, MA (US); Massachusetts Institute of Technology, Cambridge, MA (US); The General Hospital Corporation, Boston, MA (US)

(72) Inventors: Nir Hacohen, Cambridge, MA (US); Matteo Gentile, Cambridge, MA (US); Bingxu Liu, Cambridge, MA (US)

(73) Assignees: The Broad Institute, Inc., Cambridge, MA (US); Massachusetts Institute of Technology, Cambridge, MA (US); The General Hospital Corporation, Boston, MA (US)

(21) Appl. No.: 18/568,797

(22) PCT Filed: Jun. 10, 2022
(86) PCT No.: PCT/US2022/033081

§ 371 Date: Dec. 8, 2023

Related U.S. Application Data
(62) Division of application No. 63/209,316, filed on Jun. 10, 2021.
(60) Provisional application No. 63/304,554, filed on Jan. 28, 2022.

(51) Int. Cl. C12N 15/113; A61K 39/00; A61P 37/02; C07K 16/18

(52) U.S. Cl. CPC C12N 15/113; A61P 37/02; C07K 16/18; A61K 2039/505; C12N 2310/11; C12N 2310/14

(57) ABSTRACT

Provided herein are compositions and methods for enhancing the activity of Stimulator of Interferon Genes (STING) with one or more agents for inhibiting the activity of one or more negative regulators of STING, including DNAJC13 and ESCRT.

### CROSS REFERENCE TO RELATED APPLICATIONS

This application is a national stage filing under 35 U.S.C. § 371 of International PCT Application PCT/US2022/033081, filed Jun. 10, 2022, which claims the benefit under 35 U.S.C. § 119(c) of U.S. Provisional Application No. 63/209,316, filed Jun. 10, 2021, entitled “METHODS AND COMPOSITIONS FOR MODULATING STING SIGNALING AND INNATE IMMUNE RESPONSES,” and U.S. Provisional Application No. 63/304,554, filed Jan. 28, 2022, entitled “METHODS AND COMPOSITIONS FOR MODULATING STING SIGNALING AND INNATE IMMUNE RESPONSES,” the entire disclosures of each of which are hereby incorporated by reference in their entireties.

### GOVERNMENT LICENSE RIGHTS

This invention was made with government support under Grant Nos. AI133524, AI158495, and EB025854 awarded by the National Institutes of Health. The government has certain rights in the invention.

### REFERENCE TO AN ELECTRONIC SEQUENCE LISTING

The contents of the electronic sequence listing (B119570133US02-SEQ-TNG.txt; Size: 88,602 bytes; and Date of Creation: Dec. 7, 2023) is herein incorporated by reference in its entirety.

### BACKGROUND

The Stimulator of Interferon Genes (STING) is an innate regulator of immune response against pathogen- or self-derived cytosolic DNA. The STING pathway consists of a cascade of molecular events that are essential for inducing protective immunity and can cause autoimmunity if dysregulated. STING acts as an intracellular sensor of cyclic di-nucleotides and as an adaptor for cyclic GMP-AMP (cGAMP) after its generation by the DNA sensor by cyclic GMP-AMP (cGAMP) Synthase (cGAS) (Ablasser et al., 2013; Gao et al., 2013; Sun et al., 2013; Wu et al., 2013). Homodimeric STING is localized at the endoplasmic reticulum (ER) (Ishikawa and Barber, 2008) and undergoes a cGAMP-dependent conformational switch that triggers its exit from the ER and trafficking to the Golgi. At the Golgi, STING palmitoylation is required for STING clustering and activation of type I IFN responses via Tank Binding Kinase 1 (TBK1) at the Trans-Golgi Network (TGN) (Mukai et al., 2016). After TBK1 phosphorylates STING at residue S366, phospho-STING forms a platform at the carboxy-terminal tail (CTT) for recruitment of IRF3 (Liu et al., 2015a). IRF3 is then phosphorylated by TBK1, homodimerizes and translocates to the nucleus resulting in activation of a type I IFN response. In addition to type I IFN induction, upon cDN ligation, STING induces autophagy independently of the classical macroautophagy machinery but dependent on ATG16L1, an ancestral and conserved function of the response (Fischer et al., 2020; Gui et al., 2019). Thus, STING intracellular trafficking and signaling activities are tightly connected. Recently, progress has been made in characterizing the pathways regulating STING trafficking and degradation. In particular, trafficking to the endolysosomal compartment is essential for STING degradation and signaling shutdown, but the proteins governing STING trafficking to the endosomal compartment and the signals triggering its degradation require further investigation (Ablasser and Hur, 2020; Gonugunta et al., 2017).

Further knowledge and identification of STING interacting genes as therapeutic points of intervention to modulate STING activation and innate immune responses would be of great use in the art to enhance STING signaling in cancer therapy, immunotherapy, pathogen defense, and the like.

### SUMMARY OF THE INVENTION

Provided herein are compositions and methods for enhancing the activity of Stimulator of Interferon Genes (STING) with one or more agents for inhibiting the activity of one or more negative regulators of STING, including DNAJC13 and ESCRT. It is contemplated that the modulation of STING through the inhibition of the negative STING regulator proteins of this disclosure (e.g., DNAJC13 and ESCRT) can provide a therapeutic approach for treatment of a variety of diseases such as cancers, pathogen infection, autoimmune diseases or cellular senescence, by augmenting the innate immune response associated with the activation of STING signaling.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein. For purposes of clarity, not every component may be labeled in every drawing. It is to be understood that the data illustrated in the drawings in no way limit the scope of the disclosure. In the drawings:

FIGS. 1A-1E show a time-resolved map of STING interactors during trafficking by proximity-ligation mass spectrometry. FIG. 1A shows schematics of STING-TurboID fusion. STING was fused to TurboID at the CTT. Addition of biotin allows labeling of neighboring proteins in a 10 nm radius. Labeled proteins can be then enriched after cell lysis with streptavidin pulldown. FIG. 1B shows an immunoblot of Streptavidin-HRP (Strept.), STING and TBK1 in input and post streptavidin pull-down (PD: strept.) after cGAMP stimulation for the indicated times. FIG. 1C shows a scheme of the time-course used for STING-TurboID proteomics. Reporter TMT labeling ions used for each condition are indicated. FIG. 1D shows STRING generated network of filtered STING interactors after statistical analysis (top) and relative enrichment at the different time points (bottom). Colors in the top represent annotation of cellular compartments. Proteins were filtered on adj. pᵥₐₗᵤₑ<0.07 to include TBK1 (adj. pᵥₐₗᵤₑ=0.0611). FIG. 1E shows a heat-map of the filtered proteins with enrichment at the different timepoints. n=2 per time-point.

FIGS. 2A-2E show a genome-wide CRISPR screen identifies the HGS and VPS37A ESCRT subunits as required for STING degradation. FIG. 2A shows mNeonGreen (mNG) signal intensity in 293T (light gray) or a 293T cell line stably expressing a STING-mNeonGreen reporter non-stimulated (NS) (gray) or stimulated (dark gray) with 1 μg/mL 2′3′-cGAMP(pS)2 for 24 hours. One representative experiment of n=3 experiments. FIG. 2B shows the strategy for genome-wide CRISPR screen to identify regulators of STING trafficking and degradation. FIG. 2C shows a volcano-plot of log₂ fold change (log₂FC) vs −log₁₀(pᵥₐₗᵤₑ) after sequencing and analysis of the genome-wide CRISPR screen as in FIG. 2B. Genes of interest (−log₁₀ (pᵥₐₗᵤₑ)>2; e.g., HGS, VPS37A) are highlighted, as well as control guides (e.g., CTR sgRNA). FIG. 2D shows the percentage of STING-mNG positive cells in cells stimulated or not with 1 μg/mL 2′3′-cGAMP(pS)2 for 24 hours. Showed is ratio % STING-mNG positive of each sgRNA over % STING-mNG positive cells of the control non-targeting sgRNA (ntgRNA). Two independent sgRNAs per gene. n=2 independent experiments with n=2 technical replicates per experiment. Each dot represents an individual replicate. One-way ANOVA with Dunnet's multiple comparisons test. **** p<0.0001, *** p<0.001, *p<0.05, ns: not significant. FIG. 2E shows the intersection of all proteins identified by STING-TurboID proteomics (TurboID all) and filtered proteins (TurboID filtered) with hits from the genome-wide CRISPR screen with log₂FC>0 and −log₁₀(pᵥₐₗᵤₑ)>2.

FIGS. 3A-3K show that the ESCRT complex containing HGS and VPS37A regulates STING degradation and signaling shutdown FIG. 3A shows a heat-map of enriched proteins of the ESCRT machinery and TBK1 identified in the filtered STING-TurboID dataset. FIG. 3B shows mNeonGreen levels in 293T STING-mNeonGreen cell lines KO for the indicated genes before (gray—NS) or after (dark gray) stimulation with 1 μg/mL 2′3′-cGAMP(PS)2 for 24 hours. Line represents gating strategy and numbers represent % STING-mNG positive cells post stimulation. 293T (light gray) are shown as a reference for mNG negative cells. One representative plot of n=2 independent experiments with n=2 technical replicates per experiment.

FIG. 3C shows the percentage of STING-mNeonGreen (mNG) positive cells in cells stimulated or not (NS) with 1 μg/mL 2′3′-cGAMP(pS)2 for 24 hours. Shown is the ratio % STING-mNG positive of each sgRNA over % STING-mNG positive cells of the control non-targeting sgRNA (ntgRNA). Two independent sgRNAs per gene. n=2 independent experiments with n=2 technical replicates per experiment. Each dot represents an individual replicate. One-way ANOVA with Dunnet's multiple comparison test. **** p<0.0001, *** p<0.001, ** p<0.01. FIG. 3D shows an immunoblot of the indicated proteins in 293T STING-TurboID stimulated with 2′3′-cGAMP for the indicated times, in the input and after streptavidin pulldown (PD: Strept.). One representative blot of n=3 independent experiments. FIG. 3E shows an immunoblot of the indicated proteins in the input and post HA co-immunoprecipitation in 293T cells stably transduced with either an empty vector (CTR) or the indicated STING mutants, after transfection with an mScarlet-HGS expressing vector. One representative blot of n=3 independent experiments. FIG. 3F shows an immunoblot of the indicated proteins in the input and post HA co-immunoprecipitation in 293T cells stably transduced with either an empty vector (CTR) or the indicated STING mutants, after transfection with an mScarlet-VPS37A expressing vector. One representative blot of n=3 independent experiments is shown. FIG. 3G shows an immunoblot of the indicated proteins in BJ1 fibroblasts KO for HGS with two independent guides per gene or transduced with a non-targeting sgRNA (ntgRNA). Cells were stimulated with 2′3′-cGAMP for the indicated times. One representative blot of n=3 independent experiments. FIG. 3H shows an immunoblot of the indicated proteins in BJ1 fibroblasts KO for VPS37A with two independent guides per gene or transduced with a non-targeting sgRNA (ntgRNA). Cells were stimulated with 2′3′-cGAMP for the indicated times. One representative blot of n=3 independent experiments. FIG. 3I shows the expression of mScarlet-VPS4ADN (VPS4A E228Q) after transfection in 293T stably expressing STING-HA and gating strategy for mScarlet- and mScarlet^{hi} cells. FIG. 3J STING-HA levels in cells as in FIG. 3E in mScarlet^{hi} (dark gray) and mScarlet^{hi} (light gray) populations that were either non-stimulated (NS) or treated with cGAMP for 6 hours. One representative plot of n=3 independent experiments with n=2 technical replicates per experiment. FIG. 3K shows the Median Fluorescence Intensity (MFI) of STING-HA signals shown as a ratio of MFI of the mScarlet^{hi} population over the MFI of the mScarlet^{hi} population. n=3 independent experiments with n=2 technical replicates per experiment. Each dot represents an individual replicate. One-way ANOVA with Dunnet's multiple comparisons test. *** p<0.001, ns: not significant

FIG. 4A-4D shows that ESCRT links STING degradation and autophagy resolution at the endosome. FIG. 4A shows immunofluorescence of CD63, HGS, and STING in 293T stably expressing STING-HA after stimulation with cGAMP for 2 hours (left panels). Dashed box represents the cropped region shown in the right panels. Dashed line represents the line used to plot normalized pixel intensity for each protein. FIG. 4B shows immunofluorescence of p62, HGS, and STING in 293T stably expressing STING-HA after stimulation with cGAMP for 2 hours (left panels). Dashed box represents the cropped region shown in the right panels. Dashed line represents the line used to plot normalized pixel intensity for each protein. FIG. 4C shows immunofluorescence of LC3B, HGS, and STING in 293T stably expressing STING-HA after stimulation with cGAMP for 2 hours (left panels). Dashed box represents the cropped region shown in the right panels. Dashed line represents the line used to plot normalized pixel intensity for each protein. FIG. 4D shows immunofluorescence of p62, CD63, and STING in 293T stably expressing STING-HA after stimulation with cGAMP for 2 hours (left panels). Dashed box represents the cropped region shown in the right panels. Dashed line represents the line used to plot normalized pixel intensity for each protein. FIGS. 4B-4D also show an enlargement of the structures indicated by the white arrow. One representative field of n≥3 fields in n=3 independent experiments. Scale bar is 10 μm. Control non-stimulated cells are shown in FIGS. 11A-11D.

FIGS. 5A-5H show that STING ubiquitination creates a platform at the endosome for autophagy resolution and STING degradation. FIG. 5A shows mNeonGreen levels in the 293T STING-mNeonGreen reporter cell line before (gray—NS) or after (dark gray) stimulation with 4 μg/mL 2′3′-cGAMP(pS)2 for 8 hours in presence or in absence of 0.5 μM of the UBA1 inhibitor MLN7243. Line represents gating strategy and numbers represent % STING-mNeonGreen positive cells post stimulation (dark gray). 293T (light gray) are shown as a reference for mNeonGreen negative cells. One representative plot of n=3 independent experiments with n=2 technical replicates per experiment. FIG. 5B shows the percentage of STING-mNeonGreen (mNG) positive cells in cells stimulated or not as in FIG. 5A. Shown is the ratio % STING-mNG positive post simulation over % STING-mNG positive cells of non-stimulated cells. n=3 independent experiments with n=2 technical replicates per experiment. Each dot represents an individual replicate. Paired t-test. **** p<0.0001 FIG. 5C shows an immunoblot of the indicated proteins in the input or after streptavidin pull-down in 293T STING-TurboID cells stimulated or not in presence or in absence of MLN7243 for 3 hours. One representative blot of n=3 independent experiments. FIG. 5D shows an immunoblot of the indicated proteins in BJ1 fibroblasts stimulated or not with cGAMP in presence or in absence of MLN7243 for 2 hours. One representative blot of n=3 independent experiments. FIGS. 5E and 5F show immunofluorescence of p62, HGS, and STING in the absence (FIG. 5E) or presence (FIG. 5F) of MLN7243. FIGS. 5G and 5H show the immunofluorescence of p62, CD63, and STING in the absence (FIG. 5G) or presence (FIG. 5H) of MLN7243 in 293T stably expressing STING-HA after stimulation with cGAMP for 2 hours. Dashed boxes represent the cropped regions shown in the right panels. FIGS. 5G-5H also show an enlargement of the structures indicated by the white arrow. One representative field of n≥5 fields in n=2 independent experiments. Scale bar is 10 μm. Control non-stimulated cells are shown in FIG. 30.

FIGS. 6A-6H show that a patient mutation in the ESCRT-I subunit UBAP1 induces a constitutive STING-dependent type I IFN response. FIG. 6A shows an immunoblot of the indicated proteins in the input or after streptavidin pulldown in 293T STING-TurboID stimulated with cGAMP for the indicated times. One representative blot of n=2 independent experiments. FIG. 6B shows a scheme of UBAP1 domains and of the mutation introduced (stop codon at position G98) to obtain UBAP1DN. UMA: UBAP1-MVB12-Associated domain; SOUBA: Solenoid of ubiquitin associated domains (UBA). FIG. 6C shows the expression of mScarlet-UBAP1DN after transfection in 293T stably expressing STING-HA and gating strategy for mScarlet and mScarlet^{hi} populations. FIG. 6D shows STING-HA levels in cells as in FIG. 6C in mScarlet (dark gray) and mScarlet^{hi} (light gray) that were either non-stimulated (NS) or treated with cGAMP for 6 hours. One representative plot of n=3 independent experiments with n=2 technical replicates per experiment. FIG. 6E shows the Median Fluorescence Intensity (MFI) of STING-HA signals shown as a ratio of MFI of the mScarlet^{hi} population over the MFI of the mScarlet population. n=3 independent experiments with n=2 technical replicates per experiment. Each dot represents an individual replicate. One-way ANOVA with Dunnet's multiple comparisons. ** p<0.01, ns: not significant. FIG. 6F shows an immunoblot of the indicated proteins in BJ1 expressing mScarlet (CTR) or mScarlet-UBAP1DN stimulated with 2′3′-cGAMP for the indicated times. FIG. 6G shows an immunoblot of the indicated proteins in BJ1 expressing mScarlet (CTR) or mScarlet-UBAP1DN transduced with spCas9 and a control sgRNA or a STING targeting sgRNA. One experiment representative of n=3 independent experiments. FIG. 6H shows RNAseq derived heat-map for expression of selected ISGs in the indicated samples. Each column represents a technical replicate.

FIGS. 7A-7E show functionality of the STING-TurboID construct and Gene Ontology (GO) clusters upon cGAMP stimulation. FIG. 7A shows the immunofluorescence of DAPI, STING-TurboID, and Streptavidin-Cy5, at the indicated time-points in 293T STING-TurboID cells post cGAMP stimulation or non-stimulated (NS). One representative field of n≥3 fields. Scale bar is 10 μm. FIG. 7B shows Cluster 1 from cluster analysis. Molecular Function, Cellular compartment and Biological process GO terms enrichment (left) and plot of change in enrichment of statistically significant proteins at the indicated time-points. FIG. 7C shows Cluster 2 from cluster analysis. Molecular Function, Cellular compartment and Biological process GO terms enrichment (left) and plot of change in enrichment of statistically significant proteins at the indicated time-points. FIG. 7D shows Cluster 3 from cluster analysis. Molecular Function, Cellular compartment and Biological process GO terms enrichment (left) and plot of change in enrichment of statistically significant proteins at the indicated time-points. FIG. 7E shows log 2FC of p62 at the indicated time-points.

FIGS. 8A-8D show Gene Ontology (GO) (left panels) and Reactome (right panels) enrichments calculated through STRING for the full TurboID dataset at 30 minutes (FIG. 8A), 1 hour (FIG. 8B), 2 hours (FIG. 8C) and 6 hours (FIG. 8D) post cGAMP stimulation. Terms that are positively enriched are in dark gray, terms that are negatively enriched are in light gray. Size of bubbles represent the number of genes mapped in each category. FDR: False Discovery Rate.

FIGS. 9A and 9B show sorting and subsequent confirmation of a CRISPR screen of cGAMP(pS)2-stimulated cells expressing spCas9 and STING-mNG. FIG. 9A shows the gating strategy for the genome-wide CRISPR screen. Gate-D in right-most graph not used. FIG. 9B shows mNeonGreen levels in reporter spCas9 expressing 293T STING-mNeonGreen cell lines transduced with the indicated sgRNAs before (gray-NS) or after (dark gray) stimulation with 1 μg/mL 2′3′-cGAMP(pS)2 for 24 hours. Line represents gating strategy, and numbers represent % STING-mNeonGreen positive cells post stimulation. 293T (light gray) are shown as a reference for mNeonGreen negative cells. One representative plot of n=2 independent experiments with n=2 technical replicates per experiment.

FIGS. 10A-10H show confirmation of genes regulating STING degradation identified by CRISPR screening. FIG. 10A shows HGS KO efficiency relative to FIGS. 3B-3C. FIG. 10B shows VPS37A KO efficiency relative to FIGS. 3B-3C. FIG. 10C shows an immunoblot of the indicated proteins in U937 KO for HGS with two independent guides per gene or transduced with a non-targeting sgRNA (CTR). 2′3′-cGAMP stimulation was for 6 hours. One representative blot of n=3 independent experiments. FIG. 10D shows an immunoblot of the indicated proteins in U937 KO for VPS37A with two independent guides per gene or transduced with a non-targeting sgRNA (CTR). 2′3′-cGAMP stimulation was for 6 hours. One representative blot of n=3 independent experiments. FIG. 10E shows U937 cells were treated with 20 μg/mL cGAMP for 24 hours and alive cells were quantified with Cell Titer Glo. Alive cells are shown as percentage ratio RLU in cGAMP treated samples over non-stimulated samples. n=4 independent experiments. One-Way ANOVA with Dunnet's multiple comparison test. FIG. 10F shows FSC/SSC and Annexin V plots for U937 treated as in FIG. 10E. One representative experiment of n=4 independent experiments. FIG. 10G shows the percentage of cells in FSC/SSC gate for cells treated as in FIG. 10E. n=4 independent experiments. One-Way ANOVA with Holm-Sidak multiple comparison test. FIG. 10H shows the percentage Annexin V positive cells in FSC/SSC gate for cells treated as in FIG. 10E. n=4 independent experiments. One-Way ANOVA with Holm-Sidak multiple comparison test. **** p<0.0001, *** p<0.001, ** p<0.01, *p<0.05.

FIGS. 11A-11H show interaction of ESCRT components with STING. FIGS. 11A-11D show immunofluorescence of CD63, HGS, and STING (FIG. 11A); p62, HGS, and STING (FIG. 11B); LC3B, HGS, and STING (FIG. 11C); p62, CD63, and STING (FIG. 11D) in 293T stably expressing STING-HA and non-stimulated, relative to FIGS. 4A-4D. FIG. 11E shows immunofluorescence of NBR1, HGS, and STING in 293T stably expressing STING-HA non-stimulated (top) or stimulated with cGAMP for 2 hours (bottom). Dashed box represents the cropped region enlarged in the right panel for the +cGAMP condition. Scale bar is 10 μm. One representative field of n≥3 independent fields in n=3 independent experiments. FIG. 11F shows an immunoblot of the indicated proteins in the input and post mNeonGreen (mNG) co-immunoprecipitation in U937 stably expressing mNG or STING-mNG stimulated or not with cGAMP. FIG. 11G shows HA intracellular staining in 293T stably expressing STING-HA non-stimulated (NS) or treated with cGAMP for 6 hours in presence or in absence of the drug U18666A. One representative experiment of n=3 independent experiments with n=2 technical replicates per experiment. FIG. 11H shows Median Fluorescence Intensity (MFI) of STING-HA shown as % MFI of stimulated over non-stimulated in presence or absence of U18666A, related to FIG. 11F. n=3 independent experiments with n=2 technical replicates per experiment. Each dot represents an individual replicate **** p<0.0001.

FIGS. 12A-12O show involvement of ESCRT components in ubiquitination and degradation of STING. FIG. 12A shows an immunoblot of the indicated proteins in 293T stably expressing STING-HA and FLAG-ubiquitin in the input or after FLAG pulldown (IP: Flag-Ubiquitin) for cells stimulated with 2′3′-cGAMP in presence or absence of MLN7243 for 2 hours. One representative blot of n=2 independent experiments. FIG. 12B shows HA intracellular staining in 293T stably expressing HA labeled STING WT or K289R. One representative plot of n=3 technical replicates. FIG. 12C shows Median Fluorescence intensity for the STING mutants as in FIG. 12B. Each dot represents one technical replicate. Paired t-test, *** p<0.001. FIG. 12D shows HA intracellular staining in 293T stably expressing HA labeled STING WT or STING 3KR (K20R/K150R/K236R) or STING 5KR mutants (K20R/K150R/K236R/K338R/K370R) stimulated (light gray) or not (dark gray) with cGAMP for 6 hours. One representative plot of n=3 independent experiments with n=2 technical replicates per experiment. FIG. 12E shows Median Fluorescence Intensity (MFI) of cells as in FIG. 12D shown as % MFI of cGAMP stimulated over non-stimulated (NS) for each mutant. n=3 independent experiments with n=2 technical replicates per experiment. Each dot represents an individual replicate. One-way ANOVA with Dunnet's multiple comparisons. **** p<0.0001, *** p<0.001. FIG. 12F shows immunofluorescence of DAPI, GM130, and STING WT or STING 5KR before or after stimulation with cGAMP for 2 hours. FIGS. 12G-12J show immunofluorescence in 293T stably expressing STING-HA after stimulation with cGAMP for 2 hours. FIG. 12G shows immunofluorescence of CD63, HGS, and STING in absence of MLN7243. FIG. 12H shows immunofluorescence of CD63, HGS, and STING in presence of MLN7243. FIG. 12I shows immunofluorescence of LC3B, HGS, and STING in absence of MLN7243. FIG. 12J shows immunofluorescence of LC3B, HGS, and STING in presence of MLN7243. Dashed boxes represent the cropped regions shown in the right panels. One representative field of n≥5 fields in n=2 independent experiments. Scale bar is 10 μm. Control non-stimulated cells are shown in FIGS. 30A-30B. FIG. 12K shows an immunoblot of the indicated proteins in CD14⁺ monocytes isolated from a healthy donor stimulated with 5 μg/mL of 2′3′-cGAMP in the medium for 6 hours in presence or in absence of MLN7243. One representative blot of n=3 independent donors in n=2 independent experiments. FIG. 12L shows STING-mNG levels in 293T STING-mNG cells treated with 4 μg/mL cGAMP(pS)2 for 6 hours in presence of the indicated drugs. One representative experiment of n=3 independent experiments with n=2 technical replicates. FIG. 12M shows the percentage MFI of STING-mNG signal expressed as ratio MFI of stimulated cells as in FIG. 12L over MFI of unstimulated cells. n=3 independent experiments with n=2 technical replicates. Friedman test with Dunn's multiple comparison test. **** p<0.0001, ** p<0.01, ns: not significant.

FIG. 13 shows the alignment of STING in different species. Indicated in red are the Lysine positions in human STING (SEQ ID NOs: 22-32).

FIGS. 14A-14D show immunofluorescence of p62, HGS and STING (FIG. 14A); p62, CD63 and STING (FIG. 14B); CD63, HGS and STING (FIG. 14C); LC3B, CD63 and STING (FIG. 14D) in 293T stably expressing STING-HA treated (bottom panels) or non-treated (DMSO—top panels) with MLN7243.

FIGS. 15A-15O show induction of a constitutive STING-dependent type I IFN response in cells with mutant UBAP1. FIG. 15A shows an immunoblot of the indicated proteins. FIG. 15B shows immunofluorescence of DAPI, GM130 or EEA1, and phospho-STING in BJ1 fibroblasts expressing either a control vector or UBAP1DN as in FIG. 6A. One representative field of n=5 field per condition for n=2 independent experiments. Scale bar is 10 μm. FIG. 15C shows an immunoblot of the indicated proteins. Related to FIG. 6G. FIG. 15D shows volcano-plots of the differentially expressed genes (DEGs) for the indicated comparisons. Dashed lines to indicate significant DEGs are drawn at log₂FC≥1 and log₂FC≤−1 and −log₁₀(adjusted p value)≥2. Significant downregulated genes are in gray, upregulated in dark gray. nt: non-targeting sgRNA.

FIG. 15E shows GO analysis of significantly enriched processes in the indicated conditions. Positively enriched terms are in dark gray, negatively enriched in light gray. Size of bubbles represents the number of mapped genes in each category. FDR: False Discovery Rate. FIG. 15F shows the expression of mScarlet-COPA K230N after transfection in 293T stably expressing STING-HA and gating strategy for mScarlet and mScarlet^{hi} populations. FIG. 15G shows STING-HA levels in cells as in FIG. 15E in mScarlet (dark gray) and mScarlet^{hi} (light gray) cells non-stimulated (NS) or stimulated with cGAMP for 6 hours. One representative plot of n=3 independent experiments with n=2 technical replicates per experiment. FIG. 15H shows Median Fluorescence Intensity (MFI) of STING-HA signals shown as a ratio of MFI of the mScarlet^{hi} population over the MFI of the mScarlet^{hi} population. n=3 independent experiments with n=2 technical replicates per experiment. Each dot represents an individual replicate. One-way ANOVA with Dunnet's multiple comparisons. ** p<0.01, ns: not significant. FIG. 15I shows an immunoblot of the indicated proteins in primary human MDDCs transduced with either a control vector (mScarlet) or mScarlet-UBAP1DN stimulated with cGAMP (top dose 10 μg/mL, 1:2 dilutions) or pI:C+Lipofectamine for 6 hours. One representative donor of n=4 donors in n=2 independent experiments. FIG. 15J shows CD86 and mScarlet expression in cells transduced as in FIG. 15I. One representative donor of n=4 donors in n=2 independent experiments. FIG. 15K shows the quantification of % CD86+ cells in mScarlet+ gate. n=4 donors in n=2 independent experiments. FIG. 15L shows an immunoblot of the indicated proteins in BJ1 fibroblasts non-stimulated (−) or stimulated with cGAMP for the indicated times. One representative experiment of n=3 experiments. FIG. 15M shows immunofluorescence of GM130, HA-STING, and CD63 in 293T stably expressing STING-HA (left panel) or TMEM192-STING-HA (right panel). DAPI is in gray in the merged image. Scale bar is 20 μm. One field representative of n≥5 fields per construct of n=2 technical replicates. FIG. 15N shows an immunoblot of the indicated proteins in 293T stably expressing WT STING-HA (WT) or TMEM192-STING-HA (T192) stimulated or not with 2 μg/mL cGAMP for 2 hours. One experiment representative of n=2 independent experiments with n=2 technical replicates. FIG. 15O shows an immunoblot of the indicated proteins in BJ1 expressing mScarlet (CTR) or mScarlet-UBAP1DN transduced with spCas9 and a control sgRNA or a cGAS targeting sgRNA. One experiment representative of n=3 independent experiments.

FIG. 16 shows a model depicting how ubiquitination of STING recruits ESCRT at the endosome for STING degradation and autophagy. Top. Activated STING traffics from the ER to the Golgi and then to the endosome. Ubiquitination of STING drives its association with ESCRT at the late endosome. Association of ESCRT to STING creates an organizing center for phagophore closure and maturation at the endosome, leading to amphisome formation and autophagy resolution. STING is then degraded in the lysosome, leading to signaling shutdown and autophagy resolution. Pathogenic mutants of the ESCRT-I subunit UBAP1 block this process and lead to exacerbated STING responses. Bottom. At steady state, cGAS primes tonic STING trafficking between the ER and the lysosomes through the endosomal compartment. A UBAP1 mutant blocks ESCRT function and leads to accumulation of phosphorylated STING at the endosome that leads to constitutive STING activation.

FIGS. 17A-17I show that STING activation depends on its density on the organelles. FIG. 17A shows a schematic view of the density dependent STING activation model. Upper, concentrated STING is able to mediate TBK1 for trans-phosphorylation. Lower, diluted STING is unable to mediate TBK1's transphosphorylation. FIG. 17B shows a schematic view of the STING LBD domain fusion protein for targeting to different organelles. FIG. 17C shows immunoblots of organelles targeting STING LBD or WT STING stimulated with DMSO or DiABZI. FIG. 17D shows mNG fluorescence of indicated fusion proteins stimulated with DMSO or 1 μM DiABZI for 2 hours. FIG. 17E shows mNG fluorescence of indicated fusion proteins stimulated with DMSO or 1 μM DiABZI for 2 hours. FIG. 17F shows immunoblots of indicated fusion proteins stimulated with DMSO or 1 μM DiABZI for 2 h. FIG. 17G shows a schematic view of the construct of WT STING, TMEM192 fuse with STING LBD, TMEM192 fuse with STING CTT. FIG. 17H shows immunoblots of indicated fusion proteins stimulated with DMSO or 1 μM DiABZI for 2 hours. FIG. 17I shows mNG fluorescence of indicated fusion proteins stimulated with DMSO or 1 μM DiABZI for 2 h.

FIGS. 18A-18F show STING oligomerization controls its translocation. FIG. 18A shows an alignment of human STING oligomerization motif. Left, cartoon model. Right, stick model with highlight of specific amino acid. FIG. 18B shows immunoblots of 293T cells transduced with WT or indicated STING mutants upon 1 μM DiABZI (upper) or 5 μg/mL digitonin together with 2 μg/mL cGAMP (lower) treatment for 0, 2 hours. FIG. 18C shows immunofluorescence of STING in 293T cells transduced with WT or indicated STING mutants upon DMSO or 1 μM DiABZI treatment for 2 hours. FIG. 18D shows a highlight of the SAVI mutant sites and mutagenesis scanning discovered constitutive active STING sites in cryo-EM Apo human STING structure (6NT5). FIG. 18E shows immunoblots of 293T cells transduced with WT or indicated STING mutants upon 1 μM DiABZI (upper) treatment for 0, 2 hours. FIG. 18F shows immunofluorescence of STING in 293T cells transduced with WT or indicated STING mutants upon DMSO or 1 μM DiABZI treatment for 2 hours.

FIGS. 19A-19E show genome-wide CRISPR screening to comprehensively identify regulators of STING-induced cell death. FIG. 19A shows the viability of U937 transduced with non-targeting guide (sgCTR) or STING-targeting guide (sgSTING) 0 days (day 1) or 1 day (day 2) after the indicated amount of cGAMP treatment. FIG. 19B shows a schematic view of CRISPR based genome wide screening design. FIG. 19C shows a plot of genes with Average Log Fold Change (Average LFC) as X-axis value and Average-Log (p-value) as Y-axis value in the 20 μg/mL cGAMP treated group. FIG. 19D shows a plot of genes Average Log Fold Change (Average LFC) in 4 μg/mL cGAMP group (X-axis) and 1 μg/mL 2′3′-cGAM (PS)2 (Y-axis). U937 and Thp1 viability titration curve upon cGAMP activation. FIG. 19E shows the viability of U937 viability with non-targeting sgRNA (sgCTRL) or sgRNA targeting DNAJC13 (sgDNAJC13) measured by Cell Titer Glo 1 day after indicated amount of cGAMP stimulation for 24 hours.

FIGS. 20A-20G show that DNAJC13 dampens STING oligomerization to regulate its trafficking and degradation. Immunoblots of U937 (FIG. 20A) or Thp1 (FIG. 20B) transduced with sgCTRL or sgDNAJC13 (DJ13-1, DJ13-2) are stimulated with 20 μg/mL cGAMP for indicated time. FIG. 20C shows immunoblots of sgCTRL or sgDNAJC13 U937 cells transduced with control ORF (blasticidin only) or with guide-resistant DNAJC13 (DJ13-HA) stimulated with 20 μg/mL cGAMP. FIG. 20D shows immunofluorescence of sgCTRL and sgDNAJC13 U937 cells treated with digitonin (5 μg/mL) only or digitonin (5 μg/mL) with 20 μg/mL cGAMP for 5 hours. FIG. 20E shows STING Palmitoylation level measurement in sgCTRL and sgDNAJC13 U937 cells stimulated with 5 μg/mL cGAMP overnight. Upper, Schematic view of click chemistry based palmitoylated protein labelling process. Lower, Immunoblot of STING response and palmitoylation level. FIG. 20F shows an immunoblot of SDS-PAGE and Native-PAGE of sgCTRL and sgDNAJC13 (DJ13-1, DJ13-2) U937 cells stimulated 2′3′-cGAM (PS)2 for 0, 4 hours. FIG. 20G shows an immunoblot of SDS-PAGE and Native-PAGE of sgCTRL and sgDNAJC13 (DJ13-1, DJ13-2) BJ1 cells stimulated digitonin (5 μg/mL) with 20 μg/mL cGAMP for 0, 2, 4 hours.

FIGS. 21A-21E show that DNAJC13 constantly binds with STING. FIG. 21A shows a plot of proteins identified from STING-HA IP-MS proteomics experiment with Log 2FoldChange (STING-HA/mock transfection) (X-axis) and −Log (p-Value) (Y-Axis). FIG. 21B shows co-immunoprecipitation of STING-HA with mCherry (mCh) or mCherry fused with DNAJC13 (mCh-DNAJC13) together with digitonin only or digitonin and cGAMP stimulation for 4 hours. FIG. 21C shows immunofluorescence of SNX2, DNAJC13, and STING in 293T cells transduced with STING-HA. Two representative fields were selected. FIG. 21D shows a plot of protein relative abundance by STING-TurboID labelling at different time points upon cGAMP stimulation in 293T cells transduced with STING-TurboID. FIG. 21E shows immunoblots of STING-TurboID labelling target proteins (TBK1, DNAJC13, SNX1, SNX2) at different time points upon cGAMP stimulation in 293T cells transduced with STING-TurboID.

FIGS. 22A-22F show endosome localization and J domain activity are essential for DNAJC13's function on STING. FIG. 22A shows an immunoblot of sgCTRL and sgDNAJC13 (g1, g2) SW1990 or HSC4 cells. FIG. 22B shows immunofluorescence of DNAJC13 in 293T cells treated with DMSO only or with 1 μm VPS34-IN1. FIG. 22C shows immunoblots of sgCTRL or sgDNAJC13 U937 cells transduced with control ORF (blasticidin only) or with guide-resistant WT or K17A DNAJC13 stimulated with 20 μg/mL cGAMP. FIG. 22D shows immunoblots of sgCTRL or sgDNAJC13 U937 cells treated with indicated VPS34-IN1 and cGAMP (20 μg/mL). FIG. 22E shows immunoblots of sgCTRL or sgDNAJC13 STING-HA transduced 293T cells treated with digitonin (5 μg/mL) and cGAMP (2 μg/mL) for 0, 3, 5 hours, together with DMSO only or with 1 μM VPS34-IN1. FIG. 22F shows an immunoblot of sgDNAJC13 BJ1 cells transduced with control ORF (blasticidin only) or with guide-resistant WT or HQ mutant DNAJC13 stimulated digitonin (5 μg/mL) with 0.5 μg/mL cGAMP for 0, 3, 5 hours.

FIGS. 23A-23D show structural analysis and model of STING conformational change. FIG. 22A shows a crystal structure alignment of apo human STING (4f5w) or human STING with cGAMP (4ksy). FIG. 23B shows cryo-EM structure alignment of apo chicken STING (6NT6) and chicken STING with cGAMP (6NT7), zoom in view of oligomerization motif is shown on the right. FIG. 23C shows immunofluorescence of STING in 293T transduced with STING (1-341)-HA treated with DMSO only or with 1 μM DiABZI for 2 hours. FIG. 23D shows a schematic model of STING oligomerization motif change in WT apo STING, STING with agonist, STING AQQA mutant with agonist, STING R284S mutant and STING S272A.

FIGS. 24A-24B show a CRISPR screening assay and hits analysis. FIG. 24A shows U937 was either titrated with different dose of 2′3′-cGAMP or 2′3′-cGAM (PS)2 for 1 day, and cell viability was measured by Cell Titer Glo right after treatment (“Day 1”), 1 days after treatment (“Day2”), 2 days after treatment (“Day3”). FIG. 24B shows a plot of gene LFC in 20 μg/mL 2′3′-cGAMP group (X axis value) and gene LFC in 4 μg/mL 2′3′-cGAM (PS)2 group (Y axis value). With annotation of genes that related to STING downstream effector (TBK1, IRF3, IKBKE), STING trafficking essential genes (TMED2, TMED10), ESCRT genes (CHMP1A, CHMP3), macroautophagy initiation genes (ATG101, ATG9A, RB1CC1, PIK3C3).

FIG. 25 shows Thp1 with non-targeting sgRNA (sgCTRL) or sgRNA targeting DNAJC13 (sgDNAJC13) measured by Cell Titer Glo 1 day after indicated amount of cGAMP stimulation for 24 hours.

FIGS. 26A-26C show STING response upon cGAMP with digitonin permeabilization or non-CDN (cyclic dinucleotide) STING agonists stimulation in U937 cells. FIG. 26A shows immunoblots U937 cells with non-targeting sgRNA (sgCTR) or DNAJC13 targeting sgRNA (sgDNAJC13) stimulated with 5 μg/mL Digitonin and 20 μg/mL cGAMP for 0, 3, 6 hours. FIG. 26B shows immunoblots of sgCTRL (C) or sgDNAJC13 (DJ13) U937 cells stimulated with cither 20 μg/mL cGAMP, 1 μM DiABZI or 10 mM MSA2 for 0, 3 hours. FIG. 26C shows immunoblots of sgCTRL (C) or sgDNAJC13 (DJ13) U937 cells stimulated with indicated dose of DiABZI or MSA2 for 0, 3 hours.

FIGS. 27A-27C show DNAJC13 effect on STING is independent of STING palmitoylation. FIG. 27A shows sgCTRL (C) or sgDNAJC13 (D) Thp1 cells are treated with 20 μg/mL cGAMP for 0, 4, 8 hours, together with DMSO only or 1 μM H151. FIG. 27B shows immunoblots of sgCTRL or sgDNAJC13 (sgDJ13-1) 293T cells expressing STING-HA stimulated with 5 μg/mL digitonin and 2 μg/mL cGAMP for 0, 3, 5 hours, together with DMSO only or 1 μM H151. FIG. 27C shows immunoblots of sgCTRL or sgDNAJC13 (sgDJ13-1) 293T cells expressing STING-HA stimulated with 5 μg/mL digitonin and 2 μg/mL cGAMP for 0, 3, 5 hours, together with STING (WT) or STING (C88/91S).

FIGS. 28A-28C show DNAJC13 knockout delays STING degradation upon high dose of STING agonists stimulation in BJ1 cells. Immunoblots of sgCTRL (sgCTR) or sgDNAJC13 (sgDNAJC13-1, sgDNAJC13-2) BJ1 cells are stimulated with 2 μg/mL (FIG. 28A) or 20 μg/mL cGAMP (FIG. 28B) with 5 μg/mL digitonin for 0, 3, or 6 hours. FIG. 28C shows immunoblots of sgCTRL (C) or sgDNAJC13 (D) BJ1 cells stimulated with DMSO only, 20 μM or 40 μM MSA for 2, 4, 6 hours.

FIGS. 29A-29F show that a targeted CRISPR screen identifies UBE2N as a regulator of STING degradation. FIG. 29A shows a correlation plot of Average Log 2 Fold-Change (AVGLFC) for the CRISPR screen in STING-mNG or STING-HA cell lines. Color and size represent average p-value (AVGp Val) for the screen in mNeonGreen cells. FIG. 29B shows log 2 fold-change (FC) enrichment of the indicated proteins in TurboID proteomics described in FIGS. 1A-1E. FIG. 29C shows the percentage of STING-mNG positive cells in cells stimulated or not with 1 μg/mL 2′3′-cGAMP(pS)2 for 6 hours transduced with the indicated sgRNAs. FIG. 29D shows the ratio % STING-mNG positive of each sgRNA over % STING-mNG positive cells of the control non-targeting sgRNA (ntgRNA). test. ** p<0.01. FIG. 29E shows an immunoblot of the indicated proteins in BJ1 fibroblasts stimulated with 0.5 μg/mL cGAMP for the indicated times. FIG. 29F shows an immunoblot of the indicated proteins in 293T STING-TurboID transduced with a control guide or knockout for UBE2N.

FIGS. 30A and 30B show STING-mNeonGreen and STING-TurboID screens. FIG. 30A shows volcano plots of log 2 fold change (log 2FC) vs −log 10(pvalue) after sequencing and analysis of the targeted screens in STING-mNeonGreen and STING-HA cell lines. FIG. 30B shows an immunoblot of the indicated proteins in 293T STING-TurboID stimulated for 1 hour with 2 μg/mL cGAMP.

### DETAILED DESCRIPTION

Activation of STING signaling is vital for mediating immune responses against a variety of intracellular pathogens, such as viruses and bacteria, in response to the accumulation of either cytosolic double stranded DNA or dinucleotides characteristic of these pathogens. STING is also highly implicated in immune responses to cancer, due to the recognition of cytosolic self-DNA. Due to its role in activating and recruiting a variety of immune cells, such as T-cells, to tumors, agents that enhance the activity of STING may be used to treat cancer or to augment the efficacy of current therapies. This disclosure provides compositions and methods for enhancing the activity of STING signaling, particularly through the inhibition of newly discovered STING interactors that negatively regulate STING signaling activity, including ESCRT and DNAJC13. The disclosure provides methods and compositions for targeted inhibition of negative regulators of STING signaling, including ESCRT and DNAJC13, such that STING signaling—and in turn, the innate immune response—becomes enhanced and/or augmented. Such methods and compositions can be used to enhance or otherwise augment STING activation and innate immune responses, thereby providing an approach to augmenting and/or enhancing cancer immunotherapies and the like.

### Definitions

Unless otherwise defined herein, scientific and technical terms used in connection with the present disclosure shall have the meanings that are commonly understood by those of ordinary skill in the art (e.g., the skilled artisan). The meaning and scope of the terms are clear; however, in the event of any latent ambiguity, definitions provided herein take precedent over any dictionary or extrinsic definition. Further, unless otherwise required by context, singular terms shall include pluralities and plural terms shall include the singular. In this disclosure, the use of “or” means “and/or” unless stated otherwise. Furthermore, the use of the term “including,” as well as other forms, such as “includes” and “included,” is not limiting. Also, terms such as “element” or “component” encompass both elements and components comprising one unit and elements and components that comprise more than one subunit unless specifically stated otherwise.

General terminology in cell and molecular biology can be found in “The Merck Manual of Diagnosis and Therapy”, 19th Edition, published by Merck Research Laboratories, 2006 (ISBN 0-911910-19-0); Robert S. Porter et al. (Eds.), The Encyclopedia of Molecular Biology, published by Blackwell Science Ltd., 1994 (ISBN 0-632-02182-9). General terminology definitions in molecular biology are also given in Benjamin Lewin, Genes X, published by Jones & Bartlett Publishing, 2009 (ISBN-10: 0763766321); Kendrew et al. (Eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Generally, nomenclatures used in connection with, and techniques of, cell and tissue culture, molecular biology, immunology, microbiology, genetics, and protein and nucleic acid chemistry and hybridization described herein are those well-known and commonly used in the art. The methods and techniques of the present disclosure are generally performed according to conventional methods well known in the art and as described in various general and more specific references that are cited and discussed throughout the present disclosure unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of subjects.

That the present disclosure may be more readily understood, select terms are defined below.

### A, an, and the

The singular forms “a”, “an”, and “the” include the plural unless the text clearly indicates otherwise. Similarly, the term “or” is intended to include “and” unless the text clearly indicates otherwise. Although methods and materials similar or equivalent to those described herein can be used in the practice or testing of the present disclosure, suitable methods and materials are described below. The abbreviation “e.g.” is derived from the Latin phrase “exempli gratia” and is used herein to give a non-limiting example. For this reason, the abbreviation “for example (e.g.)” is synonymous with the term “for example”.

### About

The terms “approximately” or “about,” as may be used interchangeably herein, and as applied to one or more values of interest, refer to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction of (i.e., percentage greater than or percentage less than) the stated reference value unless otherwise stated or otherwise evident from the context (for example, when such number would exceed 100% of a possible value).

### Cancer

The term “cancer”, as may be used herein, refers to a cell or population of cells characterized by uncontrolled proliferation. The term “tumor”, as may be used herein, refers to a contiguous population of cancer cells. A cancer may be benign, meaning that it is localized to a single tissue, or malignant, meaning that it spreads to other parts of the body through the circulatory and/or lymphatic system. A cell or population of cells may be “pre-cancerous”, meaning that they share some characteristics of a cancer and risk developing into a cancer. Cells may become cancerous as a result of accumulated mutations in their genome. Examples of cancer include but are not limited to colorectal cancer, lung cancer, breast cancer, pancreatic cancer, prostate cancer, bladder cancer, kidney cancer, thyroid cancer, uterine cancer, cervical cancer, ovarian cancer, testicular cancer, esophageal cancer, stomach cancer, liver cancer, brain cancer, peritoneal cancer, lymphoma, leukemia, multiple myeloma, neuroblastoma, osteosarcoma, and soft tissue sarcoma.

### Decrease

The terms “decrease”, “reduced”, “reduction”, or “inhibit” are all generally statistically significant herein. The terms are used to indicate a decrease in quantity, concentration, level, or the like. However, to prevent misunderstandings, “decreased”, “decreasing”, “decreasing”, or “inhibiting” is a reduction of at least 10% compared to the reference level, e.g., at least about 20% compared to the reference level. The % reduction may also be at least about 20%, or at least about 30%, or at least about 40%, or at least about 50%, or at least about 60%, or at least about 70%, or at least about 80%, or at least about 90%, or up to 100% reduction compared to the reference level. The reduction level may also be expressed in terms of fold-reduction, and includes at least a 2-fold reduction, or at least a 3-fold reduction, or at least a 3-fold reduction, or at least a 3-fold reduction, or at least a 3-fold reduction, or at least a 4-fold reduction, or at least a 5-fold reduction, or at least a 6-fold reduction, or at least a 7-fold reduction, or at least a 8-fold reduction, or at least a 9-fold reduction, or at least a 10-fold reduction, or at least a 11-fold reduction, or at least a 12-fold reduction, or at least a 13-fold reduction, or at least a 14-fold reduction, or at least a 15-fold reduction, or at least a 16-fold reduction, or at least a 17-fold reduction, or at least a 18-fold reduction, or at least a 19-fold reduction, or at least a 20-fold reduction, or at least a 25-fold reduction, or at least a 50-fold reduction, or more.

### Increase

The terms “increased”, “increase”, “enhance” or “activate” are all generally statistically significant herein. The terms are used to indicate an increase in quantity, concentration, level, or the like. To prevent misunderstanding, the terms “increased”, “increase”, “enhance”, or “activate” is increase of at least 10% compared to the reference level, e.g., at least about 20% compared to the reference level. The % increase may also be at least about 20%, or at least about 30%, or at least about 40%, or at least about 50%, or at least about 60%, or at least about 70%, or at least about 80%, or at least about 90%, or up to 100% increase compared to the reference level. The increase level may also be expressed in terms of fold-increase, and includes at least a 2-fold increase, or at least a 3-fold increase, or at least a 3-fold increase, or at least a 3-fold increase, or at least a 3-fold increase, or at least a 4-fold increase, or at least a 5-fold increase, or at least a 6-fold increase, or at least a 7-fold increase, or at least a 8-fold increase, or at least a 9-fold increase, or at least a 10-fold increase, or at least a 11-fold increase, or at least a 12-fold increase, or at least a 13-fold increase, or at least a 14-fold increase, or at least a 15-fold increase, or at least a 16-fold increase, or at least a 17-fold increase, or at least a 18-fold increase, or at least a 19-fold increase, or at least a 20-fold increase, or at least a 25-fold increase, or at least a 50-fold increase, or more.

### Substantially

The term “substantially,” as may be used herein, when used to describe the degree or abundance of an activity, generally refers to the value of the activity as being an amount which is achievable without undue effort. As can be appreciated, this amount may vary depending on the activity being performed, with simpler activities requiring a higher threshold and more complex activities requiring a lower threshold.

### Subject

As used herein, “subject” means a human or animal. Usually, the animal is a vertebrate such as a primate, rodent, livestock animal, or hunting animal. Primates include chimpanzees, cynomolgus monkeys, spider monkeys, and macaques such as rhesus monkeys. Rodents include mice, rats, hamsters, rabbits, guinea pigs, squirrels, woodchucks, ferrets. Livestock and game animals include cattle, horses, pigs, deer, bison, buffalo, cat species such as domesticated cats, dog species such as domesticated dogs, foxes, wolves, birds such as chickens, turkeys, ducks, geese, emus, ostriches, and fish such as trout, catfish, and salmon. In some embodiments, the subject is a mammal, such as a primate, such as a human. The terms “individual”, “patient” and “subject” are used interchangeably herein. Preferably, the subject is a mammal. The mammal can be a human, non-human primate, mouse, rat, dog, cat, horse, or cow, but is not limited to these examples. Mammals other than humans can be conveniently used, for example, as subjects that represent animal models of cancer, e.g., a particular type of cancer, such as, lung cancer. The subject can be male or female. In various embodiment, the subject is a patient that has or is at risk of having a disease state, such as cancer, and is in need to being evaluated, e.g., by a liquid biopsy, to test for the risk of having or developing a disease, e.g., cancer. In other embodiments, that subject is a patient that has already been diagnosed or identified as having or having a disease in need of treatment (e.g., cancer), or one or more complications associated with such diseases. In other embodiments, a subject is a patient that has already been treated for a disease (e.g., cancer) or one or more complications associated with a disease, such as cancer. Alternatively, a subject can also be a patient that has not been previously diagnosed as having a disease (e.g., cancer) or one or more complications associated with the disease. For example, a subject can be a patient that exhibits one or more risk factors for a disease, or one or more complications associated with the disease (e.g., cancer), or a patient that does not exhibit a risk factor.

### Subject in Need

A “subject in need” of a diagnosis and/or treatment for a particular condition (e.g., cancer) can be a subject who has a condition, has been diagnosed with a condition, or is at risk of developing the condition.

### Agent

As used herein, the term “agent” refers to any material which is capable of causing a specific outcome when applied to a biological setting. An agent can be a naturally occurring, semi-synthetic, or fully synthetic chemical compound. An agent can facilitate or interfere with one or more chemical reactions occurring in an organism, thereby having one or more biological effects. An agent having a biological effect is said to be bioactive. An agent may be administered to an organism with the intent of producing a desired biological effect. An agent may be administered to an organism in the presence of additional materials having no discernable biological effect, such as an excipient compound.

### Effective Amount

As used herein, the term “effective amount” refers to the amount of an administered agent that is sufficient to produce an intended biological effect in an organism that the agent is administered to. “Effective amount” is synonymous with the terms “effective dose” and “effective concentration”.

### Protein Degradation

The term “protein degradation” is used to refer to the various processes by which protein and peptide species are hydrolyzed into smaller peptide fragments and/or individual amino acids. Protein degradation can occur as a result of enzymatic processes, such as those that are catalyzed by proteases. Protein degradation in a cell can occur in a variety of membrane-enclosed compartments (organelles), such as in lysosomes, or in the cytoplasm, as occurs within the cytoplasmic proteasome complex.

### Small Molecule

The term “small molecule”, as used herein, refers to molecule having a relatively low molecular weight. A small molecule may have a molecular weight of less than 500 daltons, less than 600 daltons, less than 700 daltons, less than 800 daltons, less than 900 daltons, or less than 1 kilodalton. A small molecule may diffuse freely across a cell membrane. A small molecule may be an effector that modulates the function of a macromolecule, such as a protein. A small molecule may be an agonist or an inhibitor of an enzyme.

### Expression

The term “expression” is broadly used to refer to the processes by which one or more genes in a cell are transcribed by RNA polymerases to produce RNA transcripts which may be translated by ribosomes to produce one or more proteins. Expression may refer to either or both of the acts of transcribing genes to RNA and translating RNA into protein. Expression may be described in absolute terms, such as the number of a particular type of RNA transcript or protein present in a cell at a given time. Expression may also be described in terms that are relative, such as when cells are treated with one or more compounds that causes expression of one or more genes and/or proteins to increase or decrease.

### Activity

The term “activity”, as is used herein, refers to the rate at which an enzyme catalyzes a particular chemical reaction. Activity may be described in absolute terms, such as the number of reactions an enzyme catalyzes on average per second. Activity may also be described in terms that are relative, such as when cells are treated with one or more compounds that causes the activity of one or more enzymes to increase or decrease.

### Inhibition

The term “inhibit,” as is used herein, refers to the reduction of one or more particular chemical reactions in the presence of a particular compound, i.e., an inhibitor. Inhibition may be described in absolute terms, such as the number of reactions an enzyme catalyzes on average per second in the presence of the inhibitor, compared to the number of reactions the enzyme catalyzes on average per second in the absence of the inhibitor. Inhibition may also be described in terms that are relative, such as when cells are treated with one or more compounds that causes the activity of one or more enzymes to decrease. A compound that reduces the activity of an enzyme may interact the enzyme directly (e.g., through a physical interaction) or indirectly (e.g., by reducing the activity of one or more separate enzymes, the activity of which precede and are necessary for activity of the enzyme).

### Routes of Administration

An agent may be delivered to a subject by one or more routes of administration. An agent may be enterally administered through the gastrointestinal tract, parenterally administered through any non-enteral route of administration, or topically administered to an external surface of the subject. Parenteral administration can be performed by injection of an agent into a specific location, tissue, or organ of a subject (e.g., intradermal, intravenous, subcutaneous, or intramuscular injection). Alternatively, parenteral administration can be inhalational, wherein the agent is administered by oral and/or nasal inhalation for uptake in the respiratory tract.

### Pharmaceutical Composition

The term “pharmaceutical composition” refers to any composition or formulation that is suitable for administration to a subject. A pharmaceutical composition or a component thereof is said to be “pharmaceutically acceptable” if it is generally safe and non-toxic when administered to a subject.

The cGAS-STING Pathway

The methods and compositions described herein are based, at least in part, on the previously unrecognized utility of modulating the activity of specific negative regulators of STING for the purpose of modulating overall STING signaling in a cell or subject. These techniques substantially enhance current capabilities for treating subjects who are suspected of having, known to have, or known to have had a STING-related disease, such as a cancer, by specifically modifying the activity of STING in subjects' own cells.

The innate immune system encompasses a variety of pathways which detect and respond to the presence of intracellular and extracellular antigens, frequently through the upregulation of genes that encode one or more signaling proteins. Antigens may be either pathogen- or host-derived, and includes proteins, lipoproteins, and nucleic acids. One such innate immune signaling pathway is the cyclic GMP-AMP synthase-stimulator of interferon genes (cGAS-STING) pathway, referred to hereafter as the “STING pathway”. The STING pathway is an intracellular sensor of double-stranded DNA (dsDNA) which drives the expression of cytokines, particularly type 1 interferons, in response to cytosolic dsDNA.

Without wishing to be bound by theory, the STING pathway functions in four major steps, collectively referred to herein as “STING signaling”. First, cyclic GMP-AMP synthase (cGAS), a 522 amino acid protein localized to the cytosolic face of the plasma membrane, binds to dsDNA occurring in the cytoplasm irrespective of sequence, as a result of, for example, infection by a pathogen (e.g., a virus, bacteria, or intracellular parasite). In response to binding dsDNA, cGAS synthesizes 2′3′-cGAMP (cGAMP), a secondary messenger molecule that is released into the cytoplasm. Second, cGAMP is bound by the stimulator of interferon genes (STING), a 378-amino acid homodimeric protein localized to the endoplasmic reticulum membrane when inactive. Upon binding to cGAMP, STING undergoes a conformational change that triggers its movement (i.e., trafficking) from the endoplasmic reticulum to the Golgi network (referred to as “forward processing”). STING may also be activated by binding to a cyclic dinucleotide, such as c-di-GMP or c-di-AMP, that is secreted by an intracellular bacterial pathogen, thereby acting independently of cGAS activity. Third, Golgi-localized STING undergoes the covalent attachment of fatty acids, such as palmitic acid, to amino acid side chains (e.g., palmitoylation). Once palmitoylated, STING readily forms oligomers (complexes between multiple STING proteins) and subsequently recruits and activates type I IFN responses via tank binding kinase 1 (TBK1), which phosphorylates STING at serine 366. Finally, active phospho-STING recruits interferon regulatory factor 3 (IRF3), which is also phosphorylated by TBK1, homodimerizes, and traffics to the nucleus where it functions as a transcription factor to induce the expression of cytokines, namely type 1 interferons (IFNs, e.g., IFN-α, IFN-β, IFN-ε, IFN-κ, IFN-τ, and IFN-ω).

Type 1 IFNs, such as IFN-α and IFN-β, are secreted by many cell types as a result of STING signaling, including fibroblasts, macrophages, and lymphocytes (e.g., B-cells, T-cells, and natural killer (NK) cells) in order to mediate immune signaling in other cells. Type 1 IFNs (e.g., IFN-α, IFN-β, IFN-ε, IFN-κ, IFN-τ, and IFN-ω) bind to interferon receptors IFNAR1 and IFNAR2, which recruits Janus family kinase 1 (Jak1) and tyrosine kinase 2 (Tyk2). Jak1 and Tyk2 phosphorylate and activate IFNAR1 and IFNAR2, which in turn recruit and drive the phosphorylation of signal transducers and activators of transcription (STAT) proteins (collectively referred to as the Jak-STAT pathway). Phosphorylated STAT1 and STAT2, for example, associate with interferon regulatory factor 9 (IRF9) to form a complex termed the ISGF3 transcription factor, which binds to IFN stimulated response elements (ISREs) on interferon-stimulated genes (ISGs) to promote the expression of a milieu of proteins that collectively drive innate and adaptive immune responses.

STING signaling can also elicit the expression and secretion of pro-inflammatory cytokines, separately of IRF3, by for example stimulating the activity of other transcription factors, such as the nuclear factor kappa-light-chain-enhancer of activated B cells (NF-κB).

### STING Involvement in Cancer Immunity

In addition to the detection of pathogen-derived dsDNA, such as that originating from viruses, bacteria, and parasites, the STING pathway also plays a key role in mediating innate immune responses to tumor cells. Just as cGAS produces cGAMP to trigger the forward processing and activation of STING in response to pathogen-derived dsDNA (e.g., viral, bacterial, or parasite dsDNA), so too does it respond to the presence of cytosolic self-DNA, as may occur during cancer. Cytosolic self-DNA may originate in a cell during cancer from any of several, non-mutually exclusive sources. First, genomic instability during cancer may trigger the release of cytosolic genomic DNA (cgDNA) from the nucleus of a cell. Errors occurring during genomic replication may also lead to the accumulation of cgDNA. Second, cytosolic DNA may also originate from mitochondrial DNA (mtDNA), due to mitochondrial instability during cancer. Third, dsDNA may accumulate in the cytoplasm of certain cell types (e.g., phagocytes, such as macrophages and dendritic cells) due to phagocytosis of tumor cells in various stages of apoptosis, or due to phagocytosis of cell free DNA (cfDNA) that occurs from dead or dying cells. Similar to detection of pathogen-derived dsDNA, detection of cgDNA, mtDNA, and/or phagocytosed tumor-derived DNA by cGAS triggers forward processing and activation of STING, which in turn induces expression and secretion of cytokines, such as type 1 IFNs. Without wishing to be bound by theory, STING signaling has been implicated in various components of cancer immunity, including triggering cancer cell apoptosis and antigen release, cancer antigen presentation by antigen presenting cells (APCs, e.g., dendritic cells (DCs) and macrophages), T-cell priming and activation by APCs, promotion of T-cell trafficking to tumors, T-cell infiltration into tumors, and antigen recognition and killing of cancer cells by cytotoxic T-cells and natural killer (NK) cells (see, e.g., Zhu, Y. et al. “STING: a master regulator in the cancer-immunity cycle”. Mol Cancer 18, 152 (2019); and Jiang, M., et al. “cGAS-STING, an important pathway in cancer immunotherapy.” J Hematol Oncol 13, 81 (2020), which are herein incorporated by reference). STING signaling has further been implicated in the immune response to a wide variety of cancers including, but not limited to, breast cancer, colorectal cancer, lung cancer, lymphoma, melanoma, ovarian cancer, pancreatic cancer, prostate cancer, and squamous cell carcinoma.

Due to the involvement of the STING pathway in mediating cancer immunity, cancer treatments would benefit from techniques that modulate STING activity. Previous efforts to modulate STING activity have focused on the development of STING agonists which can be administered to a cancer patient to activate STING signaling in a patient's cells. One STING modulator to be developed for cancer therapy is the small molecule 6-dimethylxanthenone-4-acetic acid (DMXAA) (see, e.g., Prantner D., et al. “5,6-Dimethylxanthenone-4-acetic acid (DMXAA) activates stimulator of interferon gene (STING)-dependent innate immune pathways and is regulated by mitochondrial membrane potential.” J Biol Chem. 2012; 287:39776-88, which is herein incorporated by reference) Although treatment with DMXAA induces type 1 IFN expression, it was ultimately determined to have substantially weaker binding to human STING in comparison to murine STING and therefor abandoned in clinical trials. Most STING agonists that have been developed subsequently have been cyclic dinucleotide analogs of cGAMP which are either naturally occurring or synthetically designed to bind and activate STING (see, e.g., Ding, C., et al. “Small molecules targeting the innate immune cGAS-STING-TBK1 signaling pathway.” Acta Pharm Sin B. 2020 December; 10(12):2272-2298, which is herein incorporated by reference). Cyclic dinucleotide compounds such as these typically require delivery by a liposome or nanoparticle however, as they are generally unable to cross cell membranes on their own. Despite the potential benefit that modulators of STING signaling might have for stimulating immune responses against cancer, to date no therapeutic agent for enhancing STING signaling has been approved for clinical use in the United States (see, e.g., Le Naour, J., et al. “Trial watch: STING agonists in cancer therapy.” Oncoimmunology. 2020 June; 9(1), 1777624, which is herein incorporated by reference).

Paradoxically however, STING has also been reported to impair cancer immunity in certain contexts. An increase in STING expression during cancer has been correlated with increased infiltration of certain immune-suppressing cell types into tumors, such as myeloid-derived suppressor cells (MDSCs) and regulatory T cells (Tregs) (see, e.g., An X, et al. “An analysis of the expression and association with immune cell infiltration of the cGAS/STING pathway in pan-Cancer.” Mol Ther Nucleic Acids. 2018; 14:80-9, which is herein incorporated by reference). Moreover, agents that directly activate STING have been demonstrated to impair immunity, as treatment with DMXAA elicits stress pathways that induce apoptosis in T-cells (see, e.g., Larkin B, et al. “Cutting edge: activation of STING in T cells induces type I IFN responses and cell death.” J Immunol. 2017; 199:397-402, which is herein incorporated by reference). Similarly, treatment with exogenous cGAMP impairs T-cell proliferation and can cause T-cell death (see, e.g., Cerboni S, et al. “Intrinsic antiproliferative activity of the innate sensor STING in T lymphocytes.” J Exp Med. 2017; 214:1769-85, which is herein incorporated by reference). These discoveries indicate that, in spite of the potential benefit that modulation of STING signaling may have for cancer treatment, the use of agents that directly act upon STING risk limiting any potential benefit. Treatment with agents that modulate the activity of other proteins involved in STING signaling may be better suited to enhancing STING activity, without eliciting its immunosuppressive effects.

### Negative Regulators of STING

Cells must tightly regulate STING signaling in order to prevent improper activation of immune cells, as spontaneous activation of STING may lead to autoimmunity. The activity of factors (e.g., proteins) that promote STING activation, forward processing, and/or signaling (collectively referred to as “positive regulators”) is counterbalanced by factors (e.g., proteins) that impair STING activation, forward processing, and/or signaling (collectively referred to as “negative regulators”).

Negative regulators of STING include proteins involved in the turnover of active STING such as those involved in STING trafficking and degradation. Without wishing to be bound by theory, active STING is progressively trafficked to endosomes and modified with moieties of an 8.6 kDa protein called ubiquitin (i.e., ubiquitinated), marking it for protein degradation. In the late endosome, ubiquitinated STING recruits components of either the endosomal sorting complex required for transport-0 (ESCRT-0) complex or the ESCRT-1 complex, each of which contain ubiquitin-binding domains that bind to ubiquitinated proteins. Proteins, such as ubiquitinated STING, that are targeted by ESCRT-0 and/or ESCRT-1 are subsequently sorted into intralumenal vesicles (ILVs) in the endosomal lumen and degraded by proteases after fusion of the late endosome with lysosomes, which occurs, for example, through autophagy of late endosomes (endosomal microautophagy).

In some embodiments, a negative regulator of STING is hepatocyte growth factor-regulated tyrosine kinase substrate (HGS). Without wishing to be bound by theory, HGS (also referred to as “Hrs”) interacts with signal transducing adaptor molecule 1/2 (STAM1/2) to form the ESCRT-0 complex. HGS comprises a FYVE domain through which it interacts with the endosomal membrane, as well as a ubiquitin interacting motif through which it binds ubiquitinated proteins in the endosomal membrane (e.g., ubiquitinated STING). After binding to the endosomal membrane and ubiquitinated proteins (e.g., ubiquitinated STING), ESCRT-0 recruits the ESCRT-1 complex to initiate sorting of ubiquitinated proteins into ILVs for degradation.

The nucleotide sequence for an example human (Homo sapiens) gene encoding HGS is set forth as follows (Gene ID 9146; NM_004712.5):

(SEQ ID NO: 1)
GCGCCAGCTCGTAGCAGGGGAGCGCCCGCGGCGTCGGGTTTGGGCTGGAG
GTCGCCATGGGGCGAGGCAGCGGCACCTTCGAGCGTCTCCTAGACAAGGC
GACCAGCCAGCTCCTGTTGGAGACAGATTGGGAGTCCATTTTGCAGATCT
GCGACCTGATCCGCCAAGGGGACACACAAGCAAAATATGCTGTGAATTCC
ATCAAGAAGAAAGTCAACGACAAGAACCCACACGTCGCCTTGTATGCCCT
GGAGGTCATGGAATCTGTGGTAAAGAACTGTGGCCAGACAGTTCATGATG
AGGTGGCCAACAAGCAGACCATGGAGGAGCTGAAGGACCTGCTGAAGAGA
CAAGTGGAGGTAAACGTCCGTAACAAGATCCTGTACCTGATCCAGGCCTG
GGCGCATGCCTTCCGGAACGAGCCCAAGTACAAGGTGGTCCAGGACACCT
ACCAGATCATGAAGGTGGAGGGGCACGTCTTTCCAGAATTCAAAGAGAGC
GATGCCATGTTTGCTGCCGAGAGAGCCCCAGACTGGGTGGACGCTGAGGA
ATGCCACCGCTGCAGGGTGCAGTTCGGGGTGATGACCCGTAAGCACCACT
GCCGGGCGTGTGGGCAGATATTCTGTGGAAAGTGTTCTTCCAAGTACTCC
ACCATCCCCAAGTTTGGCATCGAGAAGGAGGTGCGCGTGTGTGAGCCCTG
CTACGAGCAGCTGAACAGGAAAGCGGAGGGAAAGGCCACTTCCACCACTG
AGCTGCCCCCCGAGTACCTGACCAGCCCCCTGTCTCAGCAGTCCCAGCTG
CCCCCCAAGAGGGACGAGACGGCCCTGCAGGAGGAGGAGGAGCTGCAGCT
GGCCCTGGCGCTGTCACAGTCAGAGGCGGAGGAGAAGGAGAGGCTGAGAC
AGAAGTCCACGTACACTTCGTACCCCAAGGCGGAGCCCATGCCCTCGGCC
TCCTCAGCGCCCCCCGCCAGCAGCCTGTACTCTTCACCTGTGAACTCGTC
GGCGCCTCTGGCTGAGGACATCGACCCTGAGCTCGCACGGTATCTCAACC
GGAACTACTGGGAGAAGAAGCAGGAGGAGGCTCGCAAGAGCCCCACGCCA
TCTGCGCCCGTGCCCCTGACGGAGCCGGCTGCACAGCCTGGGGAAGGGCA
CGCAGCCCCCACCAACGTGGTGGAGAACCCCCTCCCGGAGACAGACTCTC
AGCCCATTCCTCCCTCTGGTGGCCCCTTTAGTGAGCCACAGTTCCACAAT
GGCGAGTCTGAGGAGAGCCACGAGCAGTTCCTGAAGGCGCTGCAGAACGC
CGTCACCACCTTCGTGAACCGCATGAAGAGTAACCACATGCGGGGCCGCA
GCATCACCAATGACTCGGCCGTGCTCTCACTCTTCCAGTCCATCAACGGC
ATGCACCCGCAGCTGCTGGAGCTGCTCAACCAGCTGGACGAGCGCAGGCT
GTACTATGAGGGGCTGCAGGACAAGCTGGCACAGATCCGCGATGCCCGGG
GGGCGCTGAGTGCCCTGCGCGAAGAGCACCGGGAGAAGCTTCGCCGGGCA
GCCGAGGAGGCAGAGCGCCAGCGCCAGATCCAGCTGGCCCAGAAGCTGGA
GATAATGCGGCAGAAGAAGCAGGAGTACCTGGAGGTGCAGAGGCAGCTGG
CCATCCAGCGCCTGCAGGAGCAGGAGAAGGAGCGGCAGATGCGGCTGGAG
CAGCAGAAGCAGACGGTCCAGATGCGCGCGCAGATGCCCGCCTTCCCCCT
GCCCTACGCCCAGCTCCAGGCCATGCCCGCAGCCGGAGGTGTGCTCTACC
AGCCCTCGGGACCAGCCAGCTTCCCCAGCACCTTCAGCCCTGCCGGCTCG
GTGGAGGGCTCCCCAATGCACGGCGTGTACATGAGCCAGCCGGCCCCTGC
CGCTGGCCCCTACCCCAGCATGCCCAGCACTGCGGCTGATCCCAGCATGG
TGAGTGCCTACATGTACCCAGCAGGGGCCACTGGGGCGCAGGCGGCCCCC
CAGGCCCAGGCCGGACCCACCGCCAGCCCCGCTTACTCATCCTACCAGCC
TACTCCCACAGCGGGCTACCAGAACGTGGCCTCCCAGGCCCCACAGAGCC
TCCCGGCCATCTCTCAGCCTCCGCAGTCCAGCACCATGGGCTACATGGGG
AGCCAGTCAGTCTCCATGGGCTACCAGCCTTACAACATGCAGAATCTCAT
GACCACCCTCCCAAGCCAGGATGCGTCTCTGCCACCCCAGCAGCCCTACA
TCGCGGGGCAGCAGCCCATGTACCAGCAGATGGCACCCTCTGGCGGTCCC
CCCCAGCAGCAGCCCCCCGTGGCCCAGCAACCGCAGGCACAGGGGCCGCC
GGCACAGGGCAGCGAGGCCCAGCTCATTTCATTCGACTGACCCAGGCCAT
GCTCACGTCCGGAGTAACACTACATACAGTTCACCTGAAACGCCTCGTCT
CTAACTGCCGTCGTCCTGCCTCCCTGTCCTCTACTGCCGGTAGTGTCCCT
TCTCTGCGAGTGAGGGGGGGCCTTCACCCCAAGCCCACCTCCCTTGTCCT
CAGCCTACTGCAGTCCCTGAGTTAGTCTCTGCTTTCTTTCCCCAGGGCTG
GGCCATGGGGAGGGAAGGACTTTCTCCCAGGGGAAGCCCCCAGCCCTGTG
GGTCATGGTCTGTGAGAGGTGGCAGGAATGGGGACCCTCACCCCCCAAGC
AGCCTGTGCCCTCTGGCCGCACTGTGAGCTGGCTGTGGTGTCTGGGTGTG
GCCTGGGGCTCCCTCTGCAGGGGCCTCTCTCGGCAGCCACAGCCAAGGGT
GGAGGCTTCAGGTCTCCAGCTTCTCTGCTTCTCAGCTGCCATCTCCAGTG
CCCCAGAATGGTACAGCGATAATAAAATGTATTTCAGAAAGCA.

The amino acid sequence for an example human (Homo sapiens) HGS protein is set forth as follows (NP_004703.1):

(SEQ ID NO: 2)
MGRGSGTFERLLDKATSQLLLETDWESILQICDLIRQGDTQAKYAVNSIK
KKVNDKNPHVALYALEVMESVVKNCGQTVHDEVANKQTMEELKDLLKRQV
EVNVRNKILYLIQAWAHAFRNEPKYKVVQDTYQIMKVEGHVFPEFKESDA
MFAAERAPDWVDAEECHRCRVQFGVMTRKHHCRACGQIFCGKCSSKYSTI
PKFGIEKEVRVCEPCYEQLNRKAEGKATSTTELPPEYLTSPLSQQSQLPP
KRDETALQEEEELQLALALSQSEAEEKERLRQKSTYTSYPKAEPMPSASS
APPASSLYSSPVNSSAPLAEDIDPELARYLNRNYWEKKQEEARKSPTPSA
PVPLTEPAAQPGEGHAAPTNVVENPLPETDSQPIPPSGGPFSEPQFHNGE
SEESHEQFLKALQNAVTTFVNRMKSNHMRGRSITNDSAVLSLFQSINGMH
PQLLELLNQLDERRLYYEGLQDKLAQIRDARGALSALREEHREKLRRAAE
EAERQRQIQLAQKLEIMRQKKQEYLEVQRQLAIQRLQEQEKERQMRLEQQ
KQTVQMRAQMPAFPLPYAQLQAMPAAGGVLYQPSGPASFPSTFSPAGSVE
GSPMHGVYMSQPAPAAGPYPSMPSTAADPSMVSAYMYPAGATGAQAAPQA
QAGPTASPAYSSYQPTPTAGYQNVASQAPQSLPAISQPPQSSTMGYMGSQ
SVSMGYQPYNMQNLMTTLPSQDASLPPQQPYIAGQQPMYQQMAPSGGPPQ
QQPPVAQQPQAQGPPAQGSEAQLISFD.

In some embodiments, a negative regulator of STING is vacuolar protein sorting-associated protein 37A (VPS37A). Without wishing to be bound by theory, VPS37A (also referred to as “HCRP1”, “PQBP2”, and “SPG53”) interacts with tumor susceptibility gene 101 (Tsg101), vacuolar protein sorting-associated protein 28 (Vps28), and multivesicular body sorting factor (Mvb12) to form the ESCRT-1 complex. VPS37A further interacts with ubiquitin associated protein 1 (UBAP1) as part of ESCRT-1 for binding and sequestration of ubiquitinated proteins in the endosomal membrane (e.g., ubiquitinated STING). ESCRT-1 subsequently recruits the ESCRT-II complex to ubiquitinated proteins (e.g., ubiquitinated STING), followed by the ESCRT-III complex, which together facilitate the formation of ILVs.

The nucleotide sequence for an example human (Homo sapiens) gene encoding VPS37A is set forth as follows (Gene ID 137492; NM_152415.3):

(SEQ ID NO: 3)
GCTGGGCGGCCAGGCTCCCTGGCTGGCCGGTTTGGGCGTCTGGGCCGTGAAGGTGGGACCTCCTGTTCCGGGCCGCAAGTTTCC
CTCTCCAGCCGCCCGCCGTTCGTAGCATGTCCCCCAGAACTCGGGGAGCGCAGGCAGGACAGGCTTAGAGAAGACGCGGTCCCC
AGCGCTTGGGCCACGGACGTCCCACCCCGCTCCTCTGTCGCTGGAGAACCGCCGGGCCGAGCCACTGGGAGAAGCAGGCCAGAG
CCTTCCAGGGCCTCCGGCCCGTGGACCCGAGGAGGATGAGCTGGCTTTTTCCCCTGACCAAGAGCGCCTCCTCCTCCGCGGCTG
GGTCCCCCGGTGGCCTCACCAGCCTCCAGCAGCAGAAGCAGCGCCTGATCGAGTCCCTCCGGAACTCACACTCCAGTATAGCCG
AAATACAGAAAGATGTGGAATACAGATTGCCATTCACCATAAACAACCTGACAATTAACATTAATATATTGCTTCCTCCACAGT
TTCCTCAGGAAAAACCAGTGATCAGTGTTTATCCACCAATACGACATCACTTAATGGATAAACAAGGAGTGTATGTTACCTCTC
CATTAGTAAACAATTTTACAATGCACTCAGATCTTGGAAAAATTATTCAGAGTCTGTTGGATGAGTTTTGGAAGAATCCTCCAG
TTTTAGCTCCTACTTCAACAGCATTTCCTTATCTATACAGTAACCCAAGTGGGATGTCTCCTTATGCTTCTCAGGGTTTTCCAT
TTCTTCCTCCATATCCTCCACAAGAAGCAAACAGGAGTATCACTTCTTTATCTGTTGCTGACACTGTTTCTTCTTCAACAACAA
GTCATACCACAGCCAAGCCTGCCGCTCCTTCATTTGGTGTCCTTTCAAATCTGCCATTACCCATTCCCACAGTGGATGCTTCAA
TACCGACAAGCCAAAATGGTTTTGGGTACAAGATGCCAGATGTCCCTGATGCATTTCCAGAACTCTCAGAACTAAGTGTGTCAC
AACTCACAGATATGAATGAACAAGAGGAGGTATTACTAGAACAGTTTCTGACTTTGCCTCAACTAAAACAAATTATTACCGACA
AAGATGACTTAGTAAAAAGTATTGAGGAACTAGCAAGAAAAAATCTCCTTTTGGAGCCCAGCTTGGAAGCCAAAAGACAAACTG
TTTTAGATAAGTATGAATTACTTACACAGATGAAGTCCACTTTCGAAAAGAAGATGCAAAGGCAGCATGAACTTAGTGAGAGCT
GTAGTGCAAGTGCCCTTCAGGCAAGATTGAAAGTAGCTGCACATGAAGCTGAGGAAGAATCTGATAATATTGCAGAAGACTTCT
TGGAGGGAAAGATGGAAATAGATGATTTTCTCAGTAGCTTCATGGAAAAGAGAACAATTTGCCACTGTAGAAGAGCCAAGGAAG
AGAAACTTCAGCAGGCGATAGCAATGCACAGCCAATTTCATGCTCCACTATAGATTTTCCTGGAAACATGAACTGCCAAGAGAG
GAATGGGACACAAAACCAAACACTGTTTTATATTTATGGTTTGCAAACTGGCATTTCATCAGTGGCTAAATTCACAGATATCCT
ATATAGATTGTATACAGAACTGAGACTGATTTTGTACCGATTAGAATGATTGCTATGATCTTTGAGAAATTTTTCTGCACTATT
TGCACTGAAATGTTTATTTATTGTTGATAAATTGTATCATATTTAAGTTCCACTGCTGTTCCTCTTACCTTGATTAAATGCCTA
TGCATGTACTTTTAGCTAGTTTTTAATATTTTATAAAACTTCATTTAAATTTGTATTTTTAACTTGAAGTTCCATTTCTTTATC
AAGGATGGTATTTAGATTTTTTTCCTCTTAACCTTTTTTCAAAAACTATTTTCAACTGTGAGGAAACCCTTATTTTTCTTTCTT
TGTGGATAAAACTTTCAAAAGCAATTTAAGATATTCATAGTGTTAGGAAACACCAAACCTGCCTATGTGCCATCTCACAAAAGA
AACTTTTAATACCTACAATAAATCAAAAGAATAAACCAGCTGTTCTTATATATTGTTTCATTTTTAAAACTAAAGATGCATTTA
AGAAGCAATACAAGTAAATATTTTACCTAATAGGAAAAAAAAAAGTTGCCTTTCATTTAAACCATTCCAACAGAAATTCTTATG
CTAATTTAAAACATATATATATCTGGTAGGTTTGTGGTTGGATAGGTTTTCTAAATTCCTAATGTTAAAAACAATCTTTATGTT
AATATACACTAAATCTATACACAAAAAAAGTCAGTGAACTTTTCTGACCTTTACTGTGAGTTACCTTTTCCTAAGAGGAAAGCT
ATAGTAATAAGTAAAATTTAATTTTTAGGCAATCCTGATTTTTAATGAATTTAATTGAGTGTTCTTGTATACTACATTGAGCAG
TTTGCTTCTATACCGTGTCACAAAATTCATGTATTTCTTGAGAAGCCCTAAAAGCTCATAAAGGAAAATGCCGTGAACTATGTA
GCTCAGGCTTGGTAAGGTGCCATCTAAATTACAAAACAAACTAATGCATAATTTTGCTTAAATTTCATCCCAGTATGATTGTCT
TCCCAACACCAGCATATAGTATAGATTGTCTGTCTTTTTTATATTTTTTAGTTCTTCCTGTACATGTTTTTGGCAATAAAGTTA
TAGGAAGAACAAAATTATTTTGTTAGAATTAAAACATGCTTAATATTTAGTCTGTTTGTGGAGGGCAGGTATTCACGTGGACTG
AGATACAATGTTGGATACAGAAAATAACTTTCATTGTCTTCCTGACACTGTGCTAAGGACATGCTGTTAAAGCTTCAAAGTGAC
CAGATGAGGAAGGAATAATTAATTATTACTCCTGATTTGTAGATAACTGAGGTAAGAGTGTTTCAAATTTATGATAGTCTTTTG
GGTATTCAGAAACCTTTCCTTATACTGCACTGGCCACCAGAGCTTAATTTTCCCAGCAGTTACAGCAATGGGAGATAGAACAGT
CTCAATCTTTTGCCAACCATCAGGTTCCTAGAAACCAGGTAGGTGTATCCCATAACAAGGGAGGAGCATACCACAGCCCCTCAT
TTGATTAATTCATTTGATCTATCTATGTTATTAAGTACCTACTAGGAATAAGGCATTGTGGAAATACTATACAAAGATAAACAT
TGTTTAGATGCTTATCTACTTTCCTTTTCACCAGAAAAACAGAAAAAAAAGAAACATTTTCTTACAGAGTAAAAATGTTCTACA
TAATCACATGAGTAGTTCATCTCAGTGTTTTTTATTCTTTAAAGTTGAACTATCCCAGTTTCATTCTATACCATTCATTGGATA
ACCTTGTTACAACCCAGTCATGAAACAGAGCAGTGTGATCAGTTATCTGCATTTAACAAATAGACAAATCAGTTTACATAAAGG
TTATGTATGTCACCCACGATGAAAAGAATCTGCATTTGAATATGCCCGTATGAATGTGGGTTCTGTTTTTGCAACAGAGATTAA
GTGACCATTTTTTCTAATTTTATGGCTATATATTTTCTTCATAAAAATTGGTCACATCGGAGAAGCAGTGCCACAGGAAAAATG
AAATGCATGTGAAAGTTTGTATTCTGATTTTACAAGATGAGATAGAAATCAGAATTAAAGAGGAATACTTAGGAGTTACTAGGC
TAATCAGTGTACGAATTTGTCATAGGTAGAGATTTAAAGGTTAATATCTTAAAATAGAAGAAAATTCTAAATCAATCAATCAGT
GAGATATAAACTAAACAGACCCACTTCAAAGTTGAAAGAAATTTCTAGGCATAAATTGAGACTAGGAAATTTATATCAGAATAG
AGGGTGCTTGACACATATATATGCTTAAATTGAAGGACAGCTCAGATTCATTTTTAGGAGAAGAAAGTAAACTAATGTGCTCTT
AAAGAATAAAAATTTATTCTATGGTTTCTGTCTCTGATCATCACCTTCCATTCTATAAAAAGCTCAGTTACTGATTTGCTGGGT
CATGGTCAAAATTCTTACCTATTTATTTCATATCAACTTTAAAAAATAAATTACTTGCATTCTATATATTACTAATTGGGAAGT
AATATGCCTCAAATCAGTTTTATACTGGATTATTCCCTATGCTTTAAACCACTGCTCTCAATAAAACACTTCCTGATTAATGTT
TGATTATTAGATATTTTAGTCTTGTTGGGGATATTTTAGTCTTGTTGGGTTAGCCATGCTCTGAAGAATCTGTGAAAGTACAGT
AAAGTTTTAATAAGCAATAAATGTAACCTTTTATATAAATCTCAGTGCTAGGTTAACTTCTAATAAGCAGACGAACATGTTACA
TAAATTATAATGTCTGTCTTGTAAAAAAGTTGAGGGGACTAAAAGTTTATGACTCTGATATGGAAGTTGTCATATTAAAAAACT
ACATTTTAAAACATCAAATATTTATACTATTTGCTTTTCAAATAAAAGCATAGTGCTGTTTGGCATA.

The amino acid sequence for an example human (Homo sapiens) VPS37A protein is set forth as follows (NP_689628.2):

(SEQ ID NO: 4)
MSWLFPLTKSASSSAAGSPGGLTSLQQQKQRLIESLRNSHSSIAEIQKDV
EYRLPFTINNLTININILLPPQFPQEKPVISVYPPIRHHLMDKQGVYVTS
PLVNNFTMHSDLGKIIQSLLDEFWKNPPVLAPTSTAFPYLYSNPSGMSPY
ASQGFPFLPPYPPQEANRSITSLSVADTVSSSTTSHTTAKPAAPSFGVLS
NLPLPIPTVDASIPTSQNGFGYKMPDVPDAFPELSELSVSQLTDMNEQEE
VLLEQFLTLPQLKQIITDKDDLVKSIEELARKNLLLEPSLEAKRQTVLDK
YELLTQMKSTFEKKMQRQHELSESCSASALQARLKVAAHEAEEESDNIAE
DFLEGKMEIDDFLSSFMEKRTICHCRRAKEEKLQQAIAMHSQFHAPL.

Negative regulators of STING also include proteins involved in reducing the level of active STING in the Golgi and endosomes. Without wishing to be bound by theory, STING requires binding to cGAMP, modification with palmitoyl acid (i.e., palmitoylation), and phosphorylation to become active and induce expression of type 1 IFNs. Negative regulators of STING therefore include proteins which interfere any of these processes.

In some embodiments, a negative regulator of STING is DnaJ heat shock protein family member C13 (DNAJC13). DNAJC13 is contains a J-domain, through which it interacts with heat shock protein of 70 kDa (Hsp70) and heat shock cognate protein of 70 kDa (Hsc70), chaperone proteins that facilitate protein folding. Without wishing to be bound by theory, in the Golgi however, DNAJC13 interacts directly with STING, independently of Hsp70 and Hsc70. Through this interaction, DNAJC13 impedes palmitoylation of STING, preventing it from forming active oligomers.

The nucleotide sequence for an example human (Homo sapiens) gene encoding DNAJC13 is set forth as follows (Gene ID 23317; NM_001329126.2):

(SEQ ID NO: 5)
AGAGCGCGCTCGCTCAAAGCCTGAGCGAAGATGGCGGCCTCCAGAGTGAAACTCTGAGAGGCAGAGGGAGGAGGCGGAG
GGGGGGGGAGGCAGCGCCGCGGCGGCACCAGGAACCCGCGGCAGCGGAGCTGCAGGGCCCCGGCGGGGGAAAGGGGAAG
TGGTGAGGGTGAGGAGGCCGGAGGGCGCCTCATCCCACTCTGGAAGCTGAGCCGGCGGCGGGAGGAGCTAGGGCTGGAG
CCTCTCCAGCCTCCCGCGAAGGTTTGAGCACAAAATGAACATAATTAGGGAAAATAAGGATCTGGCATGTTTCTACACA
ACAAAACATTCATGGAGGGGGAAGTATAAGCGTGTCTTTTCAGTTGGAACTCATGCGATTACTACATATAATCCCAATA
CCTTAGAAGTAACAAATCAGTGGCCTTATGGAGACATTTGCAGCATCAGCCCTGTTGGAAAAGGACAAGGAACGGAGTT
CAACCTCACATTTCGTAAAGGCAGTGGAAAAAAGTCAGAAACTTTAAAATTTTCTACAGAGCACAGAACAGAACTTCTT
ACAGAAGCATTGAGATTTAGAACTGATTTTTCAGAGGGAAAAATCACAGGAAGGAGATACAACTGCTATAAGCATCACT
GGAGTGACTCAAGAAAACCTGTAATTTTGGAAGTAACTCCAGGAGGCTTTGACCAAATTAATCCTGCAACCAACAGAGT
ACTCTGTTCCTATGACTATAGAAATATTGAAGGATTTGTAGATCTCTCAGATTATCAAGGAGGATTTTGTATACTTTAT
GGAGGATTTAGTAGATTGCATTTATTTGCGTCAGAGCAAAGAGAAGAGATTATTAAAAGTGCAATAGACCATGCTGGTA
ACTACATAGGTATTTCATTGCGGATCAGGAAAGAGCCTTTAGAATTCGAGCAATATTTGAATCTTCGCTTTGGAAAATA
CAGCACTGATGAATCCATCACATCTTTAGCAGAGTTTGTAGTCCAAAAAATATCACCTAGACATTCGGAGCCTGTTAAA
AGAGTTCTAGCACTTACAGAAACATGTTTAGTAGAACGTGATCCGGCAACCTATAATATTGCAACATTGAAGCCTTTAG
GAGAAGTATTTGCGTTGGTCTGTGACTCAGAAAATCCACAACTTTTTACCATTGAATTTATAAAAGGGCAAGTACGGAA
ATATTCTTCAACAGAGAGAGATTCCTTATTAGCAAGTTTGCTGGATGGAGTAAGAGCCTCTGGTAATAGAGATGTTTGT
GTAAAAATGACACCAACCCATAAAGGTCAGCGATGGGGGTTACTCAGCATGCCTGTTGATGAGGAAGTAGAGAGCCTTC
ACCTCAGGTTCTTAGCTACGCCTCCAAATGGCAACTTTGCAGATGCTGTATTCAGGTTCAATGCTAATATTTCATACAG
TGGAGTCCTACATGCAGTAACACAAGATGGTCTCTTCTCAGAAAACAAAGAAAAACTGATCAATAATGCCATAACAGCA
TTACTGTCCCAAGAAGGGGATGTCGTTGCTTCAAATGCGGAACTTGAGAGTCAGTTCCAGGCTGTGAGGAGGCTTGTGG
CATCCAAAGCTGGTTTCCTGGCTTTCACTCAGCTTCCAAAGTTTCGCGAGCGTCTAGGGGTGAAGGTAGTAAAAGCACT
CAAAAGAAGCAACAACGGAATAATCCATGCAGCAGTTGATATGCTTTGTGCCCTTATGTGTCCCATGCATGATGACTAT
GACTTAAGACAAGAACAGTTGAACAAAGCTTCTCTTCTCTCGTCAAAGAAGTTTCTGGAAAACTTACTGGAGAAATTTA
ATTCCCATGTGGATCATGGGACTGGTGCCCTAGTTATTAGTTCGCTCTTGGACTTCCTTACCTTTGCCCTCTGTGCTCC
ATATAGTGAGACAACTGAAGGGCAGCAGTTTGATATGCTCTTGGAGATGGTAGCATCCAATGGAAGAACCCTTTTTAAA
CTTTTTCAGCATCCTTCCATGGCAATAATAAAAGGAGCTGGGTTGGTTATGAAGGCAATAATAGAGGAAGGTGATAAAG
AAATTGCTACAAAAATGCAGGAGCTTGCCCTAAGTGAAGGTGCCTTACCTCGACACTTGCATACTGCGATGTTTACAAT
AAGCTCAGATCAAAGGATGCTTACAAATAGACAGCTAAGTAGACATTTAGTGGGACTCTGGACAGCTGATAATGCAACT
GCAACAAACTTGTTGAAACGCATTTTGCCGCCAGGCTTGCTGGCATACTTGGAAAGCTCAGATCTCGTACCTGAGAAGG
ATGCTGATCGGATGCATGTTAGAGACAATGTGAAAATAGCAATGGATCAGTATGGAAAATTTAATAAAGTTCCAGAGTG
GCAAAGACTAGCTGGAAAAGCTGCTAAAGAAGTTGAAAAATTTGCCAAAGAAAAAGTGGATCTTGTATTGATGCACTGG
AGGGATAGGATGGGCATTGCTCAAAAAGAGGACAAAAACAATATGAATATAAATCAGAAGCCAGTGGTTCTTCGAAAGA
GAAGACAAAGAATAAAAATAGAAGCAAATTGGGATCTCTTCTATTATAGGTTTGGTCAAGACCATGCCAGGTCAAACCT
TATTTGGAATTTCAAAACACGAGAAGAACTGAAAGATACTCTTGAATCTGAAATGAGAGCATTTAATATTGACAGAGAA
CTTGGAAGTGCAAATGTGATCTCCTGGAACCACCATGAGTTTGAGGTTAAATATGAGTGCCTGGCAGAGGAAATTAAAA
TAGGAGACTATTACCTGAGATTACTATTGGAGGAAGATGAGAATGAAGAAAGTGGATCAATTAAGAGATCGTATGAATT
TTTCAATGAGCTTTATCATCGCTTCTTGCTCACCCCAAAAGTAAACATGAAGTGTTTATGTTTACAAGCCCTTGCTATT
GTTTATGGCAGATGTCACGAAGAAATAGGACCTTTTACAGATACCAGATATATCATTGGAATGTTAGAGAGGTGCACAG
ATAAACTTGAACGAGATAGGTTGATTCTCTTCCTTAACAAGTTGATCCTTAATAAGAAAAATGTTAAGGATCTCATGGA
TTCAAATGGAATAAGAATCCTTGTGGACTTGCTTACCCTTGCACATCTCCATGTAAGCCGAGCTACAGTACCACTGCAA
AGCAATGTAATTGAAGCTGCTCCAGATATGAAAAGAGAGAGTGAAAAGGAATGGTATTTTGGCAACGCAGACAAAGAAA
GGAGTGGCCCGTATGGATTTCATGAGATGCAAGAATTGTGGACCAAAGGAATGTTAAATGCAAAAACCAGATGCTGGGC
TCAAGGCATGGATGGATGGCGACCACTTCAGTCCATACCCCAGCTTAAGTGGTGTCTCTTAGCCAGTGGACAGGCTGTC
CTGAATGAAACTGACCTTGCTACCCTTATATTGAACATGTTGATCACAATGTGTGGATATTTTCCAAGCAGGGATCAAG
ACAATGCCATCATTCGGCCTCTACCCAAAGTGAAAAGACTGCTGTCAGATAGCACTTGCCTTCCCCATATTATTCAGCT
ACTGCTGACCTTTGACCCTATCCTTGTTGAGAAGGTTGCTATTTTGTTATACCATATCATGCAAGATAACCCACAGTTA
CCCCGCCTTTATCTGAGTGGAGTATTTTTCTTTATCATGATGTACACAGGTTCCAATGTGCTTCCTGTTGCTCGATTTT
TGAAATACACACATACCAAACAGGCTTTCAAGTCAGAAGAGACAAAAGGACAAGATATTTTTCAGAGAAGTATACTTGG
GCACATTCTACCTGAAGCAATGGTTTGTTACTTAGAAAATTATGAACCTGAAAAGTTTTCTGAGATTTTTCTAGGAGAA
TTTGATACTCCAGAAGCAATCTGGAGCAGTGAAATGAGGCGCCTGATGATAGAGAAGATTGCTGCCCATCTCGCGGATT
TCACACCTCGTCTTCAGAGTAACACAAGAGCACTTTATCAGTATTGCCCCATTCCTATAATCAACTATCCACAACTCGA
AAATGAACTATTTTGTAATATTTATTACCTCAAACAACTGTGTGATACACTCCGGTTTCCAGATTGGCCAATTAAAGAC
CCGGTTAAGCTTCTAAAAGATACCCTTGATGCCTGGAAGAAAGAAGTAGAAAAGAAGCCACCTATGATGTCAATAGATG
ATGCTTATGAAGTGCTTAATCTGCCTCAAGGACAGGGACCGCATGATGAGAGCAAGATTAGGAAAGCTTACTTCAGACT
TGCACAAAAGTACCACCCTGATAAGAATCCAGAAGGGAGGGACATGTTTGAAAAAGTAAATAAAGCATATGAATTTTTA
TGTACCAAATCAGCAAAAATAGTGGATGGGCCAGATCCAGAGAATATAATTTTAATTCTAAAAACACAGAGCATCCTCT
TCAACCGTCATAAAGAAGATTTACAGCCTTATAAATATGCAGGATACCCCATGCTTATTCGGACTATAACAATGGAAAC
TTCAGATGACCTCCTTTTCTCAAAAGAATCACCATTGTTGCCTGCGGCTACAGAGCTAGCTTTCCATACTGTCAACTGT
TCAGCCCTCAATGCTGAAGAGCTCAGAAGAGAGAATGGACTAGAGGTGTTACAAGAGGCATTTAGTCGCTGTGTGGCTG
TCTTGACTCGTGCTAGTAAACCAAGTGACATGTCAGTACAGGTGTGTGGATACATAAGTAAATGCTACAGTGTGGCTGC
TCAGTTTGAGGAATGCCGAGAGAAGATCACGGAAATGCCTAGCATCATCAAGGATCTCTGTCGGGTACTATATTTTGGC
AAGAGTATTCCCCGCGTAGCTGCTCTTGGGGTAGAATGTGTCAGTTCTTTTGCTGTGGATTTCTGGCTACAGACACACC
TATTTCAGGCTGGAATTTTGTGGTATCTCCTTGGTTTTCTGTTTAATTATGACTACACACTAGAAGAGAGTGGCATTCA
GAAAAGTGAAGAAACAAACCAGCAGGAGGTAGCAAACAGCCTTGCCAAACTGAGTGTCCATGCTCTGAGTCGCCTTGGA
GGGTATTTGGCTGAAGAACAAGCAACTCCAGAAAATCCAACCATAAGGAAAAGCTTAGCTGGCATGCTGACACCCTATG
TTGCTAGAAAACTTGCTGTGGCTAGTGTGACTGAGATTTTGAAGATGCTTAACAGCAACACAGAAAGTCCATATTTGAT
ATGGAACAATTCTACAAGAGCAGAATTACTTGAATTTCTTGAATCCCAACAAGAAAACATGATTAAAAAAGGTGATTGT
GACAAAACTTATGGATCAGAATTTGTCTACAGTGATCATGCCAAAGAACTTATTGTAGGGGAGATTTTTGTTAGGGTGT
ATAATGAAGTTCCTACTTTCCAACTGGAGGTTCCAAAAGCATTTGCTGCAAGTCTCTTGGATTATATAGGCTCGCAGGC
CCAATACTTGCACACATTCATGGCCATCACACACGCGGCAAAAGTGGAGTCAGAGCAACATGGAGATCGCTTACCGAGA
GTAGAAATGGCTTTGGAGGCTCTGAGAAATGTCATAAAATACAATCCAGGTTCTGAGAGTGAATGCATTGGGCACTTTA
AGTTGATATTTTCTCTTCTCCGAGTTCATGGAGCTGGTCAAGTGCAGCAGTTGGCTTTAGAGGTTGTGAATATAGTGAC
ATCTAACCAAGACTGTGTCAACAATATTGCTGAATCAATGGTTTTGTCCAGTTTATTGGCTCTTCTACATTCATTGCCA
TCAAGTCGTCAGCTTGTTCTGGAAACTCTTTATGCTTTGACATCGAGTACAAAAATAATCAAAGAAGCAATGGCAAAGG
GTGCTTTGATCTATTTACTGGATATGTTCTGCAATTCAACACATCCACAGGTTCGAGCCCAAACAGCAGAACTTTTTGC
CAAAATGACAGCAGATAAACTGATAGGTCCAAAGGTTCGAATTACGTTAATGAAATTTCTACCAAGCGTTTTCATGGAT
GCTATGAGAGACAATCCTGAAGCTGCTGTACATATTTTTGAAGGAACTCATGAAAATCCTGAGTTAATTTGGAATGATA
ATTCCAGAGATAAAGTGTCCACAACAGTTAGGGAAATGATGCTAGAGCACTTTAAAAATCAGCAGGACAACCCTGAGGC
AAACTGGAAGTTGCCTGAAGATTTTGCTGTGGTGTTTGGAGAAGCAGAGGGTGAACTTGCTGTTGGAGGAGTCTTCTTG
AGGATCTTTATTGCACAACCAGCCTGGGTTCTAAGAAAGCCTAGAGAATTTCTTATTGCCCTGTTAGAAAAATTAACTG
AGCTCCTAGAGAAGAACAATCCTCATGGAGAAACTCTGGAAACCTTGACAATGGCAACAGTGTGTCTCTTCAGCGCACA
ACCTCAGCTGGCAGATCAGGTCCCGCCATTGGGCCATCTTCCCAAAGTTATCCAGGCAATGAATCATAGGAACAATGCC
ATTCCTAAGAGTGCCATTCGGGTTATCCATGCCTTGTCTGAAAATGAGCTGTGTGTTCGAGCCATGGCATCTTTAGAGA
CCATTGGCCCACTGATGAATGGAATGAAAAAGCGAGCAGATACTGTTGGTCTAGCCTGTGAAGCAATTAATCGAATGTT
TCAGAAGGAGCAGAGTGAATTAGTAGCACAAGCCCTGAAAGCAGATTTGGTTCCATACCTCTTAAAATTACTCGAAGGC
ATTGGCCTTGAAAACCTGGACAGCCCAGCAGCCACTAAGGCTCAGATTGTTAAAGCTCTCAAGGCAATGACTCGAAGTT
TGCAGTATGGAGAACAGGTGAATGAAATCCTGTGCCGTTCTTCAGTCTGGAGTGCCTTCAAAGATCAGAAACATGATTT
GTTCATTTCTGAGTCACAAACAGCAGGATACCTCACAGGACCTGGAGTTGCTGGCTACCTTACCGCAGGTACATCTACA
TCAGTCATGTCTAACCTGCCACCTCCTGTAGACCATGAGGCAGGCGACCTTGGCTATCAGACTTGAAATATTCACGAGA
GACAATAAACGCTGAAAGGCCAGTGCCAAGTCCACATTCCTCCAGCTGATACGTTGAAGCAAACTCTTACTGCCTTTCT
CCTGGTTTCATGACAGTGTTATTCCTTTTTCTATAAATATATTTTTAGGAAAAAAAGTCAGTGATCCTAATTGTATCAC
ATTATAAGAAAGCACTCTGTGGATCAACATAAGTGGGTACACAAGAATTTTTTTTTTCTTGGTGTATGTAAGCACATTT
GTTCCTTTATATCTGTTTACAAAACTGTGAATCAAAAAGACAAAACTTTCTTCCTAGTTTTTGTAATTTTTTTTTTGAA
CTAGCATGACTGTAGGGTTGAGCTACAGTCAACAAAAATTGGGCTAAGTCACTTTTCCCCAGGAAAGAATATTTCCCTC
TCCTGCATCAAGTCTGCGTGGCCATCCTCCCCCCACCATCCAAGACTATTAGGTTTTGTCCCTGCACCCTTCACTGGCA
TCCTCAATCATTAACCTTCTGAAAGCTCACAGTACACATTAGTATGTATAACTGGCTTTACCAAATTGAATGAAAAGGA
GCTTGTGCAAAAAAATTTAAAAATGGATGTCAAGATGTTATGTAAAAGATGAGTGTAATTGTGAAATGTTCTATACACT
ATCAAATATATAAAGCTTTCTATATTGAATGTACATTATACAGATCATTCATATGTGTACATAAAATTTTAAAAATAAA
GGGAATTGACTGCTTTGTTAATGAGA.

The amino acid sequence for an example human (Homo sapiens) DNAJC13 protein is set forth as follows (NP_001316055.1):

(SEQ ID NO: 6)
MNIIRENKDLACFYTTKHSWRGKYKRVFSVGTHAITTYNPNTLEVTNQWP
YGDICSISPVGKGQGTEFNLTFRKGSGKKSETLKFSTEHRTELLTEALRF
RTDFSEGKITGRRYNCYKHHWSDSRKPVILEVTPGGFDQINPATNRVLCS
YDYRNIEGFVDLSDYQGGFCILYGGFSRLHLFASEQREEIIKSAIDHAGN
YIGISLRIRKEPLEFEQYLNLRFGKYSTDESITSLAEFVVQKISPRHSEP
VKRVLALTETCLVERDPATYNIATLKPLGEVFALVCDSENPQLFTIEFIK
GQVRKYSSTERDSLLASLLDGVRASGNRDVCVKMTPTHKGQRWGLLSMPV
DEEVESLHLRFLATPPNGNFADAVFRFNANISYSGVLHAVTQDGLFSENK
EKLINNAITALLSQEGDVVASNAELESQFQAVRRLVASKAGFLAFTQLPK
FRERLGVKVVKALKRSNNGIIHAAVDMLCALMCPMHDDYDLRQEQLNKAS
LLSSKKFLENLLEKFNSHVDHGTGALVISSLLDFLTFALCAPYSETTEGQ
QFDMLLEMVASNGRTLFKLFQHPSMAIIKGAGLVMKAIIEEGDKEIATKM
QELALSEGALPRHLHTAMFTISSDQRMLTNRQLSRHLVGLWTADNATATN
LLKRILPPGLLAYLESSDLVPEKDADRMHVRDNVKIAMDQYGKFNKVPEW
QRLAGKAAKEVEKFAKEKVDLVLMHWRDRMGIAQKEDKNNMNINQKPVVL
RKRRQRIKIEANWDLFYYRFGQDHARSNLIWNFKTREELKDTLESEMRAF
NIDRELGSANVISWNHHEFEVKYECLAEEIKIGDYYLRLLLEEDENEESG
SIKRSYEFFNELYHRFLLTPKVNMKCLCLQALAIVYGRCHEEIGPFTDTR
YIIGMLERCTDKLERDRLILFLNKLILNKKNVKDLMDSNGIRILVDLLTL
AHLHVSRATVPLQSNVIEAAPDMKRESEKEWYFGNADKERSGPYGFHEMQ
ELWTKGMLNAKTRCWAQGMDGWRPLQSIPQLKWCLLASGQAVLNETDLAT
LILNMLITMCGYFPSRDQDNAIIRPLPKVKRLLSDSTCLPHIIQLLLTFD
PILVEKVAILLYHIMQDNPQLPRLYLSGVFFFIMMYTGSNVLPVARFLKY
THTKQAFKSEETKGQDIFQRSILGHILPEAMVCYLENYEPEKFSEIFLGE
FDTPEAIWSSEMRRLMIEKIAAHLADFTPRLQSNTRALYQYCPIPIINYP
QLENELFCNIYYLKQLCDTLRFPDWPIKDPVKLLKDTLDAWKKEVEKKPP
MMSIDDAYEVLNLPQGQGPHDESKIRKAYFRLAQKYHPDKNPEGRDMFEK
VNKAYEFLCTKSAKIVDGPDPENIILILKTQSILFNRHKEDLQPYKYAGY
PMLIRTITMETSDDLLFSKESPLLPAATELAFHTVNCSALNAEELRRENG
LEVLQEAFSRCVAVLTRASKPSDMSVQVCGYISKCYSVAAQFEECREKIT
EMPSIIKDLCRVLYFGKSIPRVAALGVECVSSFAVDFWLQTHLFQAGILW
YLLGFLFNYDYTLEESGIQKSEETNQQEVANSLAKLSVHALSRLGGYLAE
EQATPENPTIRKSLAGMLTPYVARKLAVASVTEILKMLNSNTESPYLIWN
NSTRAELLEFLESQQENMIKKGDCDKTYGSEFVYSDHAKELIVGEIFVRV
YNEVPTFQLEVPKAFAASLLDYIGSQAQYLHTFMAITHAAKVESEQHGDR
LPRVEMALEALRNVIKYNPGSESECIGHFKLIFSLLRVHGAGQVQQLALE
VVNIVTSNQDCVNNIAESMVLSSLLALLHSLPSSRQLVLETLYALTSSTK
IIKEAMAKGALIYLLDMFCNSTHPQVRAQTAELFAKMTADKLIGPKVRIT
LMKFLPSVFMDAMRDNPEAAVHIFEGTHENPELIWNDNSRDKVSTTVREM
MLEHFKNQQDNPEANWKLPEDFAVVFGEAEGELAVGGVFLRIFIAQPAWV
LRKPREFLIALLEKLTELLEKNNPHGETLETLTMATVCLFSAQPQLADQV
PPLGHLPKVIQAMNHRNNAIPKSAIRVIHALSENELCVRAMASLETIGPL
MNGMKKRADTVGLACEAINRMFQKEQSELVAQALKADLVPYLLKLLEGIG
LENLDSPAATKAQIVKALKAMTRSLQYGEQVNEILCRSSVWSAFKDQKHD
LFISESQTAGYLTGPGVAGYLTAGTSTSVMSNLPPPVDHEAGDLGYQT.

### Agents for Inhibiting the Activity of Negative Regulators of STING

In one aspect, the present disclosure relates to the administration to a subject of an effective dose of an agent capable of inhibiting a negative regulator of STING. In some embodiments, an agent that is capable of inhibiting the activity of a negative regulator of STING inhibits the activity of the negative regulator of STING by up to 10%, up to 20%, up to 30%, up to 40%, up to 50%, up to 60%, up to 70%, up to 80%, up to 90%, up to 95%, up to 99%, or up to 100%. In some embodiments, an agent that is capable of inhibiting the activity of a negative regulator of STING enhances the level of STING signaling by up to 10%, up to 20%, up to 30%, up to 40%, up to 50%, up to 60%, up to 70%, up to 80%, up to 90%, up to 2-fold, up to 3-fold, up to 4-fold, up to 5-fold, up to 10-fold, up to 25-fold, up to 50-fold, up to 100-fold, up to 200-fold, up to 300-fold, up to 400-fold, up to 500-fold, up to 600-fold, up to 700-fold, up to 800-fold, up to 900-fold, or up to 1000-fold, as may be measured, for example, by the expression of STING, by the level of palmitoylated STING, the level of phosphorylated STING, or by the expression of one or more type 1 interferons (e.g. IFN-α, IFN-β, IFN-ε, IFN-κ, IFN-τ, and IFN-ω).

In some embodiments, an agent that is capable of inhibiting the activity of a negative regulator of STING acts systemically. In other embodiments, an agent that is capable of inhibiting the activity of a negative regulator of STING acts only within a localized region of a subject, such as within a specific organ, tissue, or population of cells (e.g., a tumor).

In some embodiments, an agent that is capable of in inhibiting the activity of a negative regulator of STING is a small molecule inhibitor. In some embodiments, an agent that is capable of inhibiting the activity of a negative regulator of STING is a peptide inhibitor. In some embodiments, an agent that is capable of inhibiting the activity of a negative regulator of STING is a small molecule or peptide inhibitor of a component of ESCRT-1. In some embodiments, an agent that is capable of inhibiting the activity of a negative regulator of STING is a small molecule or peptide inhibitor of a component of HGS or VPS37A. In some embodiments, an agent that is capable of inhibiting the activity of a negative regulator of STING is a small molecule or peptide inhibitor of a J-domain protein. In some embodiments, an agent that is capable of inhibiting the activity of a negative regulator of STING is a small molecule or peptide inhibitor of DNAJC13.

In some embodiments, an agent capable of inhibiting the activity of a negative regulator of STING is an oligonucleotide capable of interfering with expression of one or more negative regulators of STING. In some embodiments, an agent capable of interfering with expression of one or more negative regulators of STING is a small interfering RNA (siRNA), short hairpin RNA (shRNA), micro RNA (miRNA), or antisense oligonucleotide (ASO). In some embodiments, an agent capable of interfering with expression of one or more negative regulators of STING is an oligonucleotide capable of interfering with expression of a component of ESCRT-1. In some embodiments, an agent capable of interfering with expression of one or more negative regulators of STING is an oligonucleotide capable of interfering with expression of VPS37A, examples of which are commercially available or are otherwise well known within the art, each of which are incorporated by reference herein). In some embodiments, an agent capable of interfering with expression of one or more negative regulators of STING is an oligonucleotide capable of interfering with expression of HGS, examples of which are commercially available or are otherwise well known within the art, each of which are incorporated by reference herein). In some embodiments, an agent capable of interfering with expression of one or more negative regulators of STING is an oligonucleotide capable of interfering with expression of a J-domain protein. In some embodiments, an agent capable of interfering with expression of one or more negative regulators of STING is an oligonucleotide capable of interfering with expression of DNAJC13, examples of which are commercially available or are otherwise well known within the art, each of which are incorporated by reference herein).

In some embodiments, the agent is an oligonucleotide (e.g., siRNA, shRNA, miRNA, ASO) capable of interfering with expression of HGS that has a region of complementarity to the nucleotide sequence of SEQ ID NO: 1. In some embodiments, the agent is an oligonucleotide (e.g., siRNA, shRNA, miRNA, ASO) capable of interfering with expression of VPS37A that has a region of complementarity to the nucleotide sequence of SEQ ID NO: 3. In some embodiments, the agent is an oligonucleotide (e.g., siRNA, shRNA, miRNA, ASO) capable of interfering with expression of DNAJC13 that has a region of complementarity to the nucleotide sequence of SEQ ID NO: 5.

### Methods for Use

In one aspect, the present disclosure relates to the administration to a subject of an effective dose of an agent that results in the inhibition of one or more negative regulators of STING (e.g., VPS37A, HGS, DNAJC13). As used herein, the term “effective dose” refers to a dose that is sufficient to bring about one or more desired biological effects within a particular subject. In some embodiments, the subject is a human.

In some embodiments, administration of an effective dose of an agent that is capable of inhibiting the activity of a negative regulator of STING inhibits the activity of the negative regulator of STING by up to 10%, up to 20%, up to 30%, up to 40%, up to 50%, up to 60%, up to 70%, up to 80%, up to 90%, up to 95%, up to 99%, or up to 100%, relative to the activity of the negative regulator of STING before the agent is administered. In some embodiments, administration of an effective dose of an agent that is capable of inhibiting the activity of a negative regulator of STING enhances the level of STING signaling by up to 10%, up to 20%, up to 30%, up to 40%, up to 50%, up to 60%, up to 70%, up to 80%, up to 90%, up to 2-fold, up to 3-fold, up to 4-fold, up to 5-fold, up to 10-fold, up to 25-fold, up to 50-fold, up to 100-fold, up to 200-fold, up to 300-fold, up to 400-fold, up to 500-fold, up to 600-fold, up to 700-fold, up to 800-fold, up to 900-fold, or up to 1000-fold, relative to the level of STING signaling before the agent is administered, as may be measured, for example, by the expression of STING, by the level of palmitoylated STING, the level of phosphorylated STING, or by the expression of one or more type 1 interferons (e.g. IFN-α, IFN-β, IFN-ε, IFN-κ, IFN-τ, and IFN-ω)

In some embodiments, the administered agent is a small molecule or a peptide that inhibits the activity of VPS37A, HGS, and/or DNAJC13 by up to 10%, up to 20%, up to 30%, up to 40%, up to 50%, up to 60%, up to 70%, up to 80%, up to 90%, up to 95%, up to 99%, or up to 100%, relative to the activity before the agent is administered. In some embodiments, the administered agent is an oligonucleotide that inhibits the expression of VPS37A, HGS, and/or DNAJC13 by up to 10%, up to 20%, up to 30%, up to 40%, up to 50%, up to 60%, up to 70%, up to 80%, up to 90%, up to 95%, up to 99%, or up to 100%, relative to the activity before the agent is administered.

In some embodiments, the administered agent circulates in the bloodstream of the subject with a circulatory half-life of at least 10 minutes. In some embodiments, the administered agent circulates in the bloodstream of the subject with a circulatory half-life of up to 1 hour, up to 2 hours, up to 3 hours, up to 4 hours, up to 5 hours, up to 6 hours, up to 7 hours, up to 8 hours, up to 9 hours, up to 12 hours, up to 24 hours, up to 36 hours, up to 48 hours, up to 72 hours, or more than 72 hours.

In some embodiments, the agent is administered intravenously.

In some embodiments, the subject is a human patient. In some embodiments, the subject is a human patient that has, is suspected of having, or is at risk of having a disease which may be treated by eliciting or enhancing an immune response (e.g., an innate immune response). In some embodiments, the subject is a human patient that has, is suspected of having, or is at risk of having an infection caused by a pathogen (e.g., a virus, a bacterium, or a parasite). In some embodiments, the subject is a human patient that has, is suspected of having, or is at risk of having cancer.

In some embodiments, the administered agent is administered in the presence of one or more carriers or excipients. In some embodiments, the administered agent is administered in the presence of one or more agents for the treatment of cancer (e.g., a chemotherapeutic, an immunotherapeutic). In some embodiments, the administered agent is administered prior to the administration of one or more agents for the treatment of cancer (e.g., a chemotherapeutic, an immunotherapeutic). In some embodiments, the administered agent is administered following the administration of one or more agents for the treatment of cancer (e.g., a chemotherapeutic, an immunotherapeutic). In some embodiments, the administered agent is administered prior to, concurrently with, or following another therapy for cancer (e.g., radiotherapy, surgical intervention).

The agents and compositions of the disclosure may be administered to patients by any number of different routes, including enteral or parenteral routes. Enteral administration includes administration by the following routes: oral, sublingual, and rectal routes. Parenteral administration includes administration by the following routes: intravenous, cutaneous or subcutaneous, nasal, intramuscular, intraocular, transepithelial, intraperitoneal, topical (including dermal, ocular, rectal, nasal, inhalation and aerosol (i.e., pulmonary)), and rectal systemic routes.

Administration can be performed e.g., by injection, or ballistically using a delivery gun to accelerate their transdermal passage through the outer layer of the epidermis. The nanoparticles may also be delivered in aerosols. This is made possible by the small size of the nanoparticles.

### Pharmaceutical Compositions

In another aspect, the present disclosure relates to pharmaceutical compositions for inhibiting the activity of one or more negative regulators of STING in cells of a subject. In some embodiments, the contemplated pharmaceutical compositions comprise one or more agents capable of inhibiting the activity of one or more negative regulators of STING, e.g., HGS, VPS38A, and/or DNAJC13.

The contemplated pharmaceutical compositions may be in any form suitable for administration to a subject, e.g., a liquid composition, a solid composition, a gel composition, or aerosolized compositions thereof. Other compositions are also contemplated, including those that may delivered by transdermal patches, emulsions, foams, granules, implants, pellets, pills, sprays, suppositories, suspensions, tablets, and the like, so long as agent(s) may be delivered and increase the concentration of phagocytosed DNA in phagocytes of one or more biological tissues or liquids. Such compositions will generally comprise a carrier of some sort, for example a solid carrier or a liquid carrier such as water, petroleum, animal or vegetable oils, mineral oil, or synthetic oil. Physiological saline solution, or glycols such as ethylene glycol, propylene glycol or polyethylene glycol may also be included. Such compositions and preparations generally contain at least 0.1 wt % of the active agent. Such formulations are well known in the art.

In some embodiments, the contemplated pharmaceutical compositions may comprise one or more of a pharmaceutically acceptable excipients, carriers, buffers, stabilizers, delivery agents, isotonicizing agents, preservatives or antioxidants, or other materials well known to those skilled in the art, in addition to one or more agents for increasing the concentration of tumor-derived DNA in a phagocyte, e.g., a macrophage. Such materials should be non-toxic and should not interfere with the efficacy of the active ingredient. The precise nature of the excipients, carriers, buffers, stabilizers, delivery agents, isotonicizing agents, preservatives or antioxidants, or other materials may depend on the route of administration, e.g., intravenously, orally, topically, or through inhalation.

For administration through inhalation, the contemplated pharmaceutical composition will be in the form of a liquid or dry powder that is capable of being aerosolized, e.g., by an inhalation device for oral and/or nasal inhalation for delivery to the lungs. In embodiments where the pharmaceutical composition is a liquid that can be aerosolized, it will be in the form of an acceptable aqueous solution which is pyrogen-free and has suitable pH, isotonicity, and stability.

For intravenous, cutaneous, or subcutaneous injection, the contemplated pharmaceutical composition will be in the form of a parenterally acceptable aqueous solution which is pyrogen-free and has suitable pH, isotonicity, and stability. Those of relevant skill in the art are knowledgeable in means of preparing suitable solutions using, for example, solutions of containing the active agent(s) in, e.g., physiological saline, a dispersion prepared with glycerol, liquid polyethylene glycol, or oils.

In embodiments where the contemplated pharmaceutical composition is a liquid, the composition may be formulated to have a pH between about 3.0 and 9.0, or preferably between about 4.5 and 8.5. Ideally, a liquid pharmaceutical composition has a pH between about 5.0 and 8.0. The pH of a composition can be maintained by the use of a buffer such as acetate, citrate, phosphate, succinate, Tris or histidine, typically employed in the range from about 1 mM to 50 mM. The pH of compositions can otherwise be adjusted by using physiologically acceptable acids or bases.

The pharmaceutical compositions contemplated herein may also comprise preservatives. Preservatives are generally included in pharmaceutical compositions to retard microbial growth, thereby extending the shelf life of the compositions and allowing multiple use packaging. Examples of preservatives include phenol, meta-cresol, benzyl alcohol, parahydroxybenzoic acid and its esters, methyl paraben, propyl paraben, benzalconium chloride and benzethonium chloride. Preservatives are typically employed in the range of about 0.1 to 1.0% (w/v).

Compositions containing one or more agents for inhibiting the activity of one or more negative regulators of STING may also comprise one or more agents for the treatment of cancer.

Compositions containing one or more agents for inhibiting the activity of one or more negative regulators of STING are preferably administered to a subject in a sufficiently effective amount (i.e., for achieving an increase the concentration of phagocytosed DNA within phagocytes of the subject). In some embodiments, compositions containing one or more agents for inhibiting the activity of one or more negative regulators of STING are administered to a subject in a sufficiently effective amount concurrently, after, or prior to the administration of a sufficiently effective amount of one or more agents for the treatment of cancer. Examples of the techniques and protocols relevant for establishing the effective amount of a pharmaceutical composition can be found in Handbook of Pharmaceutical Additives, 2nd Edition (eds. M. Ash and I. Ash), 2001 (Synapse Information Resources, Inc., Endicott, New York, USA); Remington's Pharmaceutical Sciences, 20th Edition, 2000, pub. Lippincott, Williams & Wilkins; and Handbook of Pharmaceutical Excipients, 2nd edition, 1994).

### EXAMPLES

### Example 1: ESCRT-Dependent STING Degradation Curtails Steady-State and cGAMP-Induced Signaling

Intracellular DNA is a potent activator of innate immune responses via Stimulator of Interferon Genes (STING) (Ishikawa et al., 2009), which acts as an adaptor for cyclic GMP-AMP (cGAMP) after its generation by the DNA sensor by cyclic GMP-AMP (cGAMP) Synthase (cGAS) (Ablasser et al., 2013; Gao et al., 2013; Sun et al., 2013; Wu et al., 2013). In addition to the eukaryotic 2′3′-linked cGAMP, STING is also a sensor for 3′3′-linked cyclic-dinucleotides (cdNs) of bacterial origin (Burdette et al., 2011; Woodward et al., 2010). STING binding to its ligands induces both type I interferon (IFN) responses and autophagy, an ancestral and conserved activity important for clearance of intracellular pathogens (Gui et al., 2019). Activation of innate immune pathways by STING is highly conserved from metazoans to bacteria (Kranzusch et al., 2015; Morehouse et al., 2020). In addition to pathogen responses, the cGAS/STING axis is essential for antitumor immune responses, immune checkpoint therapy, development of autoimmune diseases and induction of cellular senescence (Motwani et al., 2019).

Homodimeric STING is localized at the endoplasmic reticulum (ER) (Ishikawa and Barber, 2008) and undergoes a cGAMP-dependent conformational switch that triggers its exit from the ER and trafficking to the Golgi. STING palmitoylation is required for STING clustering and activation of type I IFN responses via Tank Binding Kinase 1 (TBK1) at the Trans-Golgi Network (TGN) (Mukai et al., 2016). After TBK1 phosphorylates STING at residue S366, phospho-STING forms a platform at the carboxy-terminal tail (CTT) for recruitment of IRF3 (Liu et al., 2015a). IRF3 is then phosphorylated by TBK1, homodimerizes and translocates to the nucleus resulting in activation of a type I IFN response. In addition to type I IFN induction, upon CDN ligation, STING induces autophagy independently of the classical macroautophagy machinery but dependent on ATG16L1, an ancestral and conserved function of the response (Fischer et al., 2020; Gui et al., 2019). Thus, STING intracellular trafficking and signaling activities are tightly connected.

Recently, progress has been made in characterizing the pathways regulating STING trafficking and degradation. ER-to-Golgi STING translocation is facilitated by coatomer protein complex II (COPII) vesicles that rely on STING association with SEC24C (Gui et al., 2019). Other proteins have been shown to regulate this trafficking step, such as STEEP, or to stabilize STING on the ER membrane, such as STIM1 or TOLLIP (Pokataycv et al., 2020; Srikanth et al., 2019; Zhang et al., 2020). STING can also traffic from the ER to LC3B positive vesicles, although the mechanisms of this step are less clear (Gui et al., 2019). Interestingly, while proteins of the autophagy machinery have been shown to regulate STING signaling, their impact on STING degradation is ambiguous. Knockout of ATG5, ATG9A, ATG16L1 or ULK1/2 do not seem to regulate STING degradation, while ATG3 has been both shown to regulate STING degradation in human monocytic cells or to be dispensable in mouse embryonic fibroblasts (Fischer et al., 2020; Gonugunta et al., 2017; Gui et al., 2019; Prabakaran et al., 2018; Saitoh et al., 2009). Post-Golgi trafficking to the endolysosomal compartment is essential for STING degradation and signaling shutdown, but the proteins governing these processes and the signals triggering STING degradation require further investigation (Ablasser and Hur, 2020; Gonugunta et al., 2017). Overall, the hierarchy of STING interactions with the intracellular trafficking and autophagy machineries and how these pathways are integrated to regulate STING degradation and signaling shutdown remain to be clarified. Finally, the physiological role of trafficking of this innate immune sensor to distinct intracellular compartments remains unknown.

Presented herein is a systems approach to systematically and unbiasedly identify genes involved in STING cell biology and to uncover the physiological significance of these intracellular trafficking pathways. Based on proximity-ligation proteomics, a time-resolved map of STING trafficking and potential interactors in distinct intracellular compartments was created. Combining proteomics with a genome-wide CRISPR screen, an endosomal sorting complex required for trafficking (ESCRT) complex was identified including Hepatocyte growth factor-regulated tyrosine kinase substrate (HGS), Vacuolar Protein Sorting 37A (VPS37A) and Ubiquitin-associated protein 1 (UBAP1) as required for STING degradation and as a signal coordinator between STING trafficking to the endosome and autophagy resolution at this organelle. Similar to the phosphorylation of STING that creates a platform for IRF3 recruitment and type I IFN signaling at the TGN, it is shown that ubiquitination of STING creates a platform for ESCRT recruitment at the late endosome, therefore identifying a role for this post-translational modification in regulating STING post-Golgi trafficking. By using a targeted library for ubiquitin and autophagy related proteins, a role for Ubiquitin-conjugating enzyme E2 N (UBE2N) was also identified in STING degradation and its association with ESCRT. Ubiquitination mediated association of STING with ESCRT at the endosome drives autophagy resolution through phagophore closure and amphisome formation and is responsible for STING degradation, therefore clarifying how autophagy and STING trafficking to endosomes are integrated. Finally, it is shown that disruption of ESCRT function through a human pathogenic Ubiquitin-Associated Protein 1 (UBAP1) mutant leads to constitutive STING dependent type I IFN signaling in fibroblasts and monocyte derived dendritic cells at steady-state with an intracellular post-Golgi accumulation of activated STING. Based on these findings, a revised model of STING trafficking was proposed, in which the sensor is subject to a homeostatic degradative flux. Physiologically, ESCRT-dependent STING removal at the endosome acts as a gatekeeper to curtail spontaneous induction of STING activation at steady state and control the magnitude of STING responses to ligands.

### Results

Identification of Subcellular Compartments and Proteins in Proximity to STING During a Time Course of cGAMP Stimulation

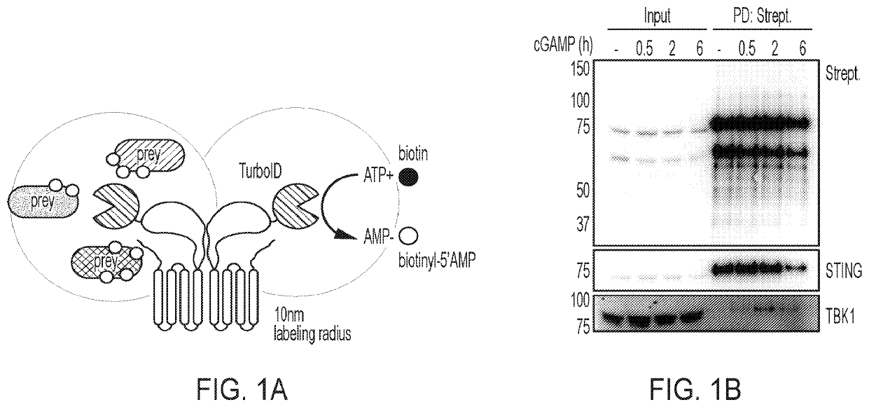

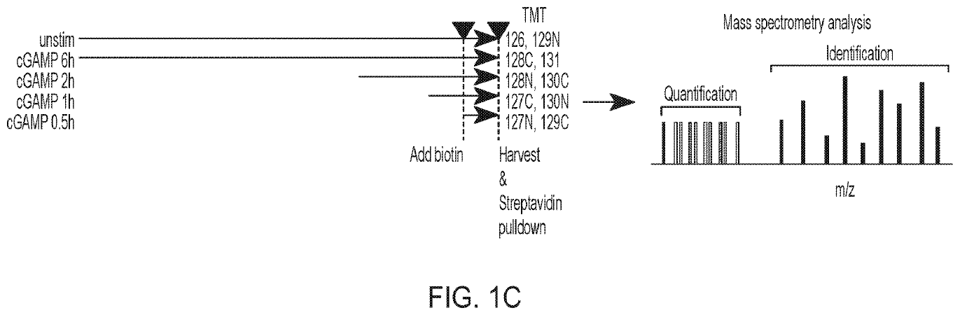

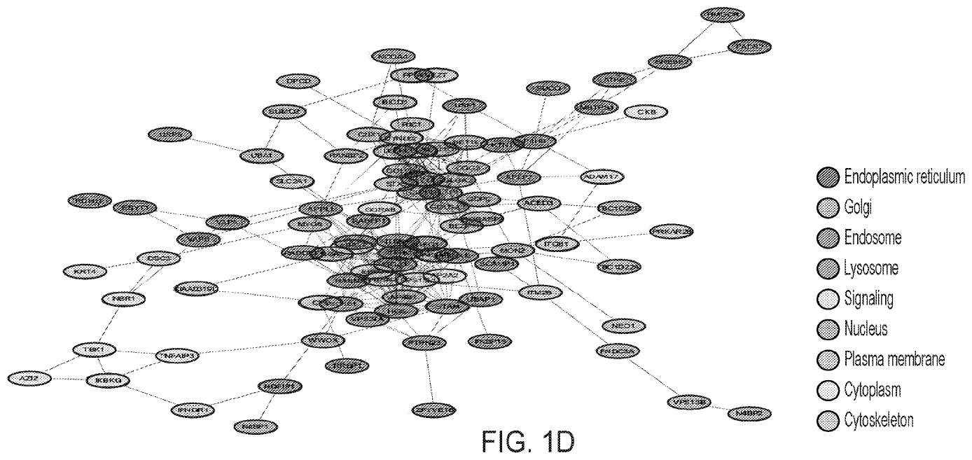

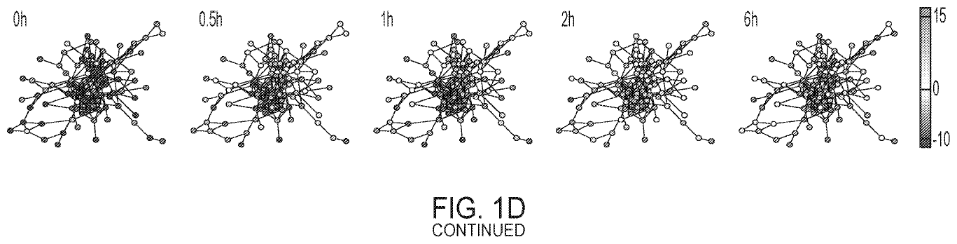

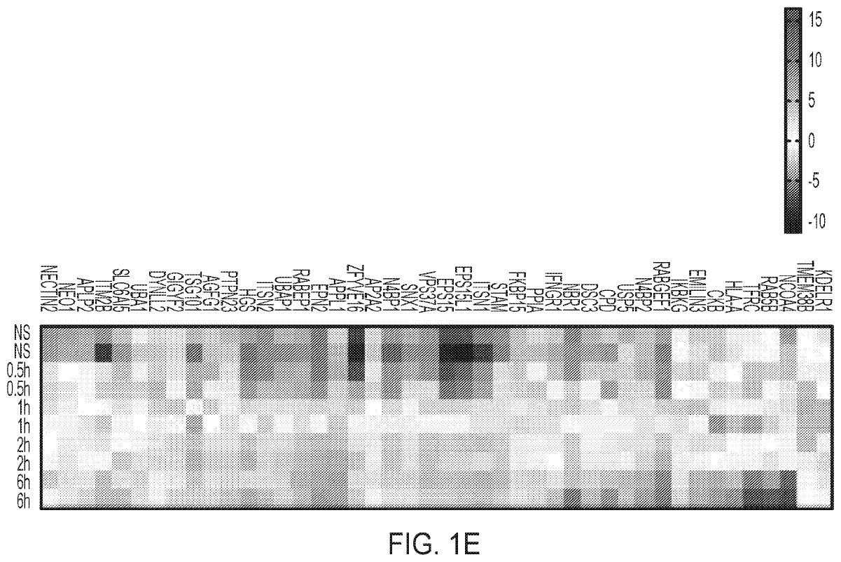

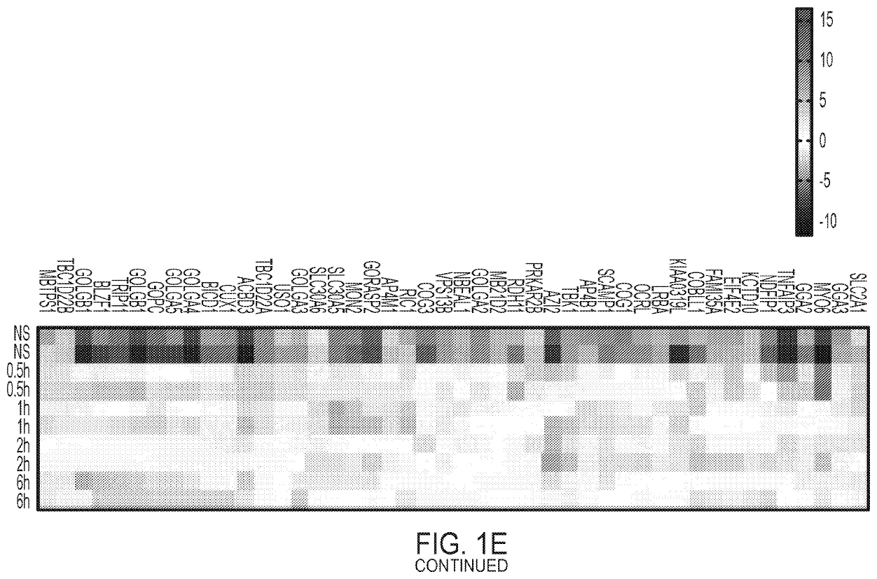

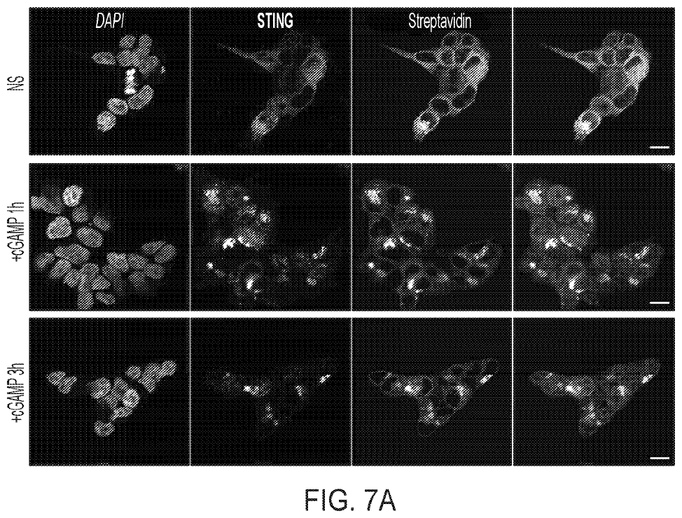

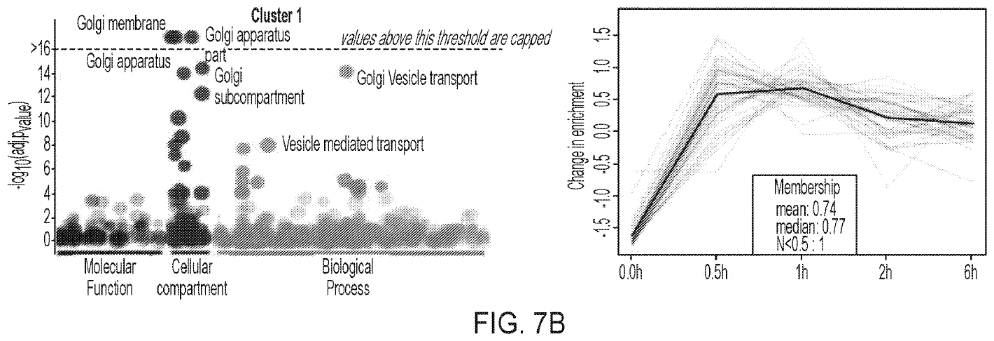

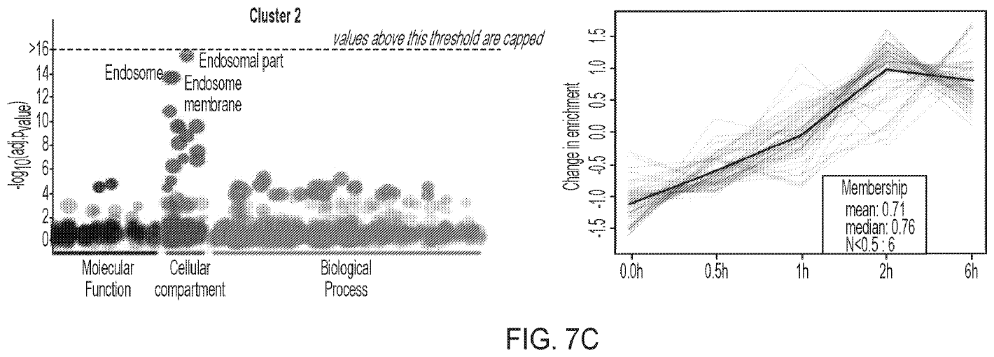

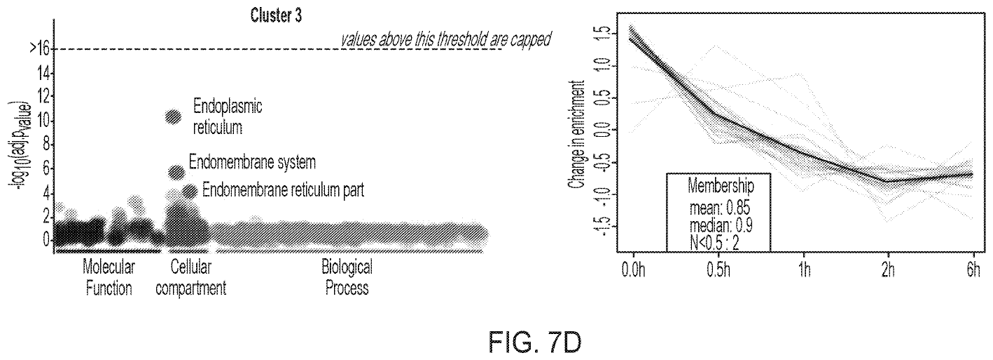

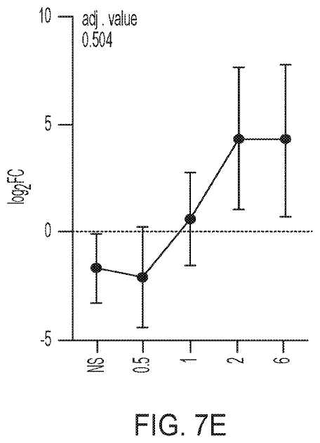

To establish the hierarchy of molecular mechanisms governing STING trafficking and to identify proteins that interact with STING during this process, recently developed proximity ligation technologies that overcome the limitations of classical co-immunoprecipitation in identifying native interactions were used. STING was fused to the biotin ligase TurboID (FIG. 1A) (Branon et al., 2018) for rapid (i.e. 10 minutes) ligation of biotin to proteins within a 10 nm radius, followed by enrichment of labeled proteins via streptavidin pull-down. To verify that the construct was functional, microscopy was performed in 293T cells stably expressing STING-TurboID and confirmed STING translocation upon cGAMP stimulation and co-localization with streptavidin staining (FIG. 7A). Moreover, streptavidin pulldown following a cGAMP stimulation time-course showed time-dependent TBK1 labeling with signal increasing at 30 minutes, peaking at 2 hours and decreasing at 6 hours (FIG. 1B). Finally, the decrease in STING levels at the 6 hour time point indicated that this time course would cover the full trafficking pathway from the ER to the lysosome.

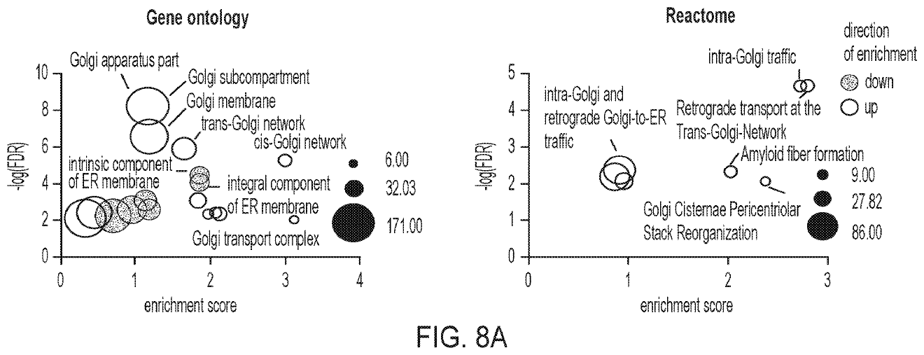

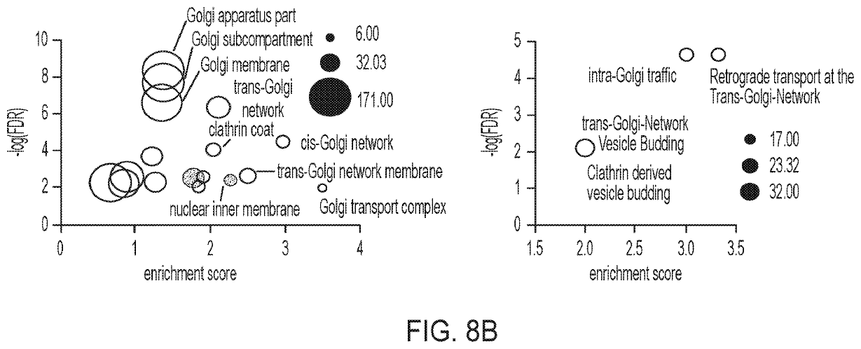

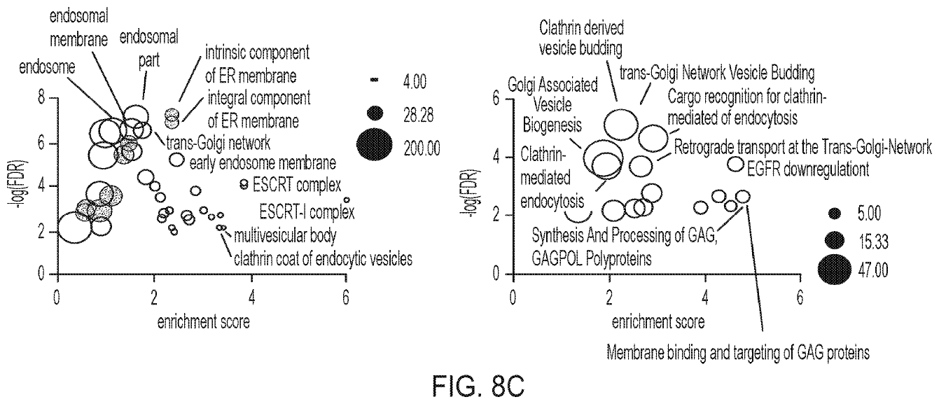

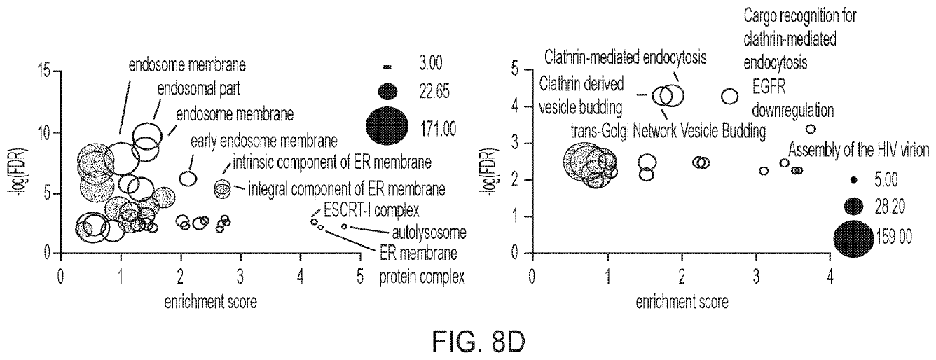

To map protein-protein interactions, proximity labeling was performed followed by quantitative mass spectrometry analysis. STING-TurboID expressing cells were stimulated with cGAMP, then were biotin-labeled for 30 minutes at different time points post-stimulation (FIG. 1C). Streptavidin enriched lysates were digested to peptides with trypsin and labeled with MS-differentiable tandem mass tags (TMT). After labeling, the samples were pooled and analyzed by liquid-chromatography tandem mass spectrometry (LC-MS/MS). Over 2000 proteins were identified in total across the different time-points. Fuzzy c-means clustering identified three protein clusters. Clusters and single timepoints were analyzed to identify enriched Gene Ontology (GO) and Reactome terms (FIGS. 7B-7D, FIGS. 8A-8D). Cluster 1 included proteins of the Golgi apparatus and vesicle transport, and showed an enrichment peak at 30 minutes and 1 hour with decreasing enrichment after the 2 hour time point (FIGS. 7A, 8A-8B). Cluster 2 contained endosomal proteins initially enriched at the 2 hour time point and slightly decreased at the 6 hour time point (FIG. 7C). At 2 hours, proteins were associated with “trans-Golgi network Vesicle Budding” and “Cargo recognition for clathrin mediated endocytosis”, indicative of STING exit from the Golgi through the TGN and trafficking to endosomes (FIG. 8C). In addition, at 2 hours, hits were associated with the endosome and ESCRT machinery and at 6 hours with the autolysosome and more weakly with ESCRT (FIGS. 8C-8D). Finally, cluster 3 included proteins of the ER that were enriched at time 0 (not stimulated) and decreased at all other time points (FIG. 7D).

Out of the ˜2000 proteins identified, statistical analysis led to filtering the dataset down to a network of 132 proteins that were differentially biotinylated between time-points. The inferred localization and trafficking pattern of this network of proteins was consistent with the analyses performed on the full dataset (FIGS. 1D-1E, FIGS. 7B-7D, FIGS. 8A-8D), with ER proteins enriched at time 0, Golgi proteins at 30 minutes peaking at 1 hour, and endosome proteins peaking at 2 hours and still highly abundant at 6 hours. A signaling hub was identified comprising TBK1, AZI2, TNFAIP3 and IKBKG that was mostly enriched at two hours. Components of the ESCRT machinery were also abundant at the 2 hour time-point. NCOA4 and TFRC, involved in ferritin turnover through selective autophagy and highly enriched in lysosomes, were labeled at 6 hours, along with the endosomal marker RAB5B22. Finally, two selective autophagy receptors were found, NBR1 enriched at 2 hours in the filtered dataset, and p62 enriched at 2 and 6 hours in the full dataset (FIG. 1E, FIG. 7E), indicative of a possible crosstalk between autophagy and STING trafficking to the endosome.

This dataset provides a time-resolved map of potential STING interactors and the basis to identify the hierarchy of events involved in STING trafficking and degradation.

### A Genome-Wide CRISPR Screen Identifies the HGS and VPS37A ESCRT Subunits as Required for STING Degradation

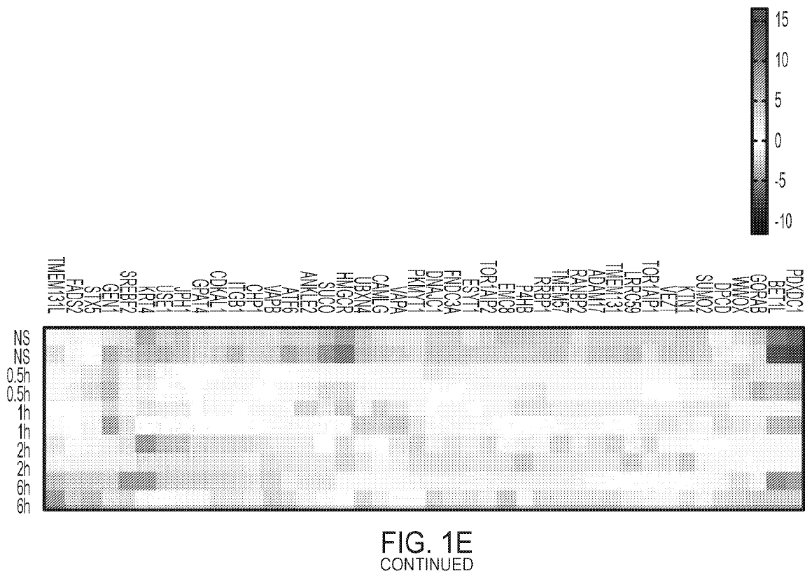

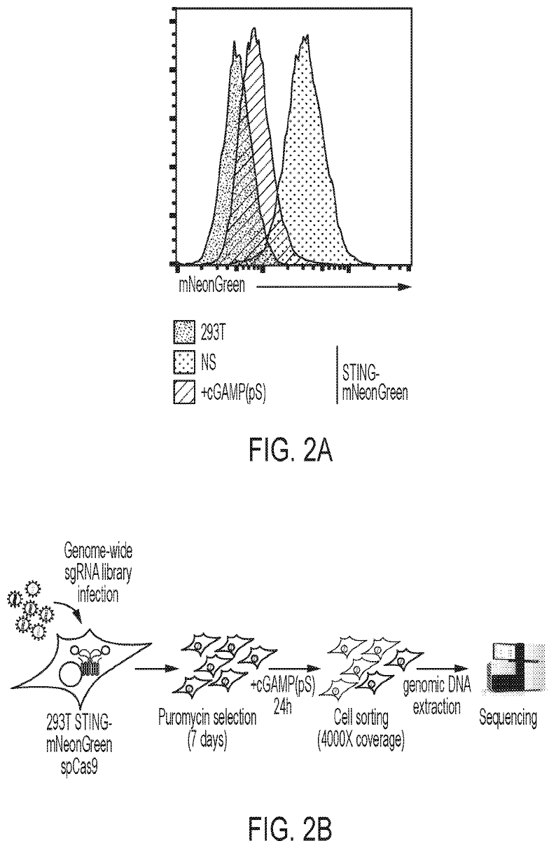

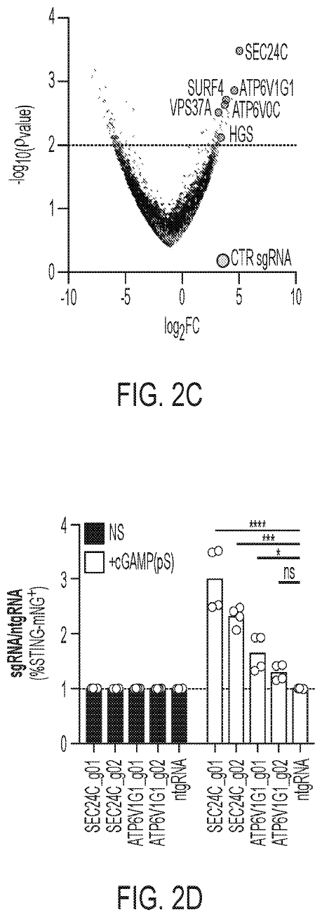

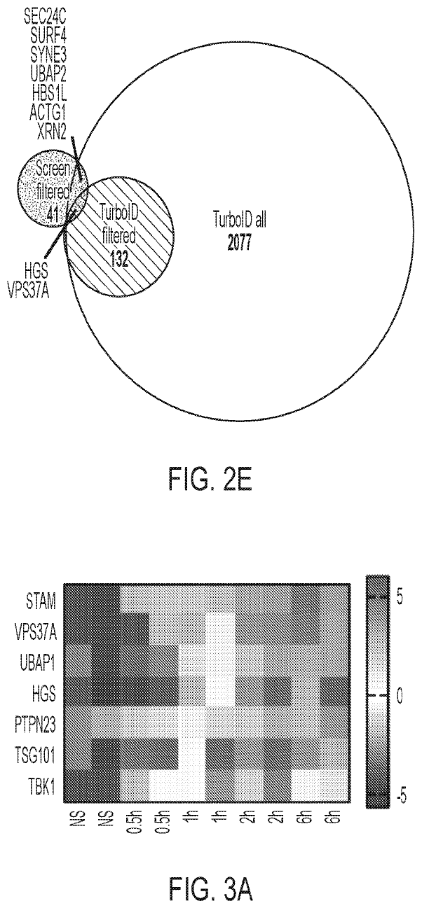

To separately identify proteins required for STING trafficking and degradation, a genome wide CRISPR screen was performed based on activity of a STING protein reporter that undergoes degradation. A system was optimized to follow STING degradation with flow cytometry by fusing STING to the bright green fluorescent protein mNeonGreen (mNG) under control of a weak promoter. Stimulation of 293T cells stably expressing the reporter with the stable cGAMP analog 2′3′-cGAMP(pS)2 led to a decrease in STING-mNG signal (FIG. 2A), thus confirming functionality of the reporter cell line.

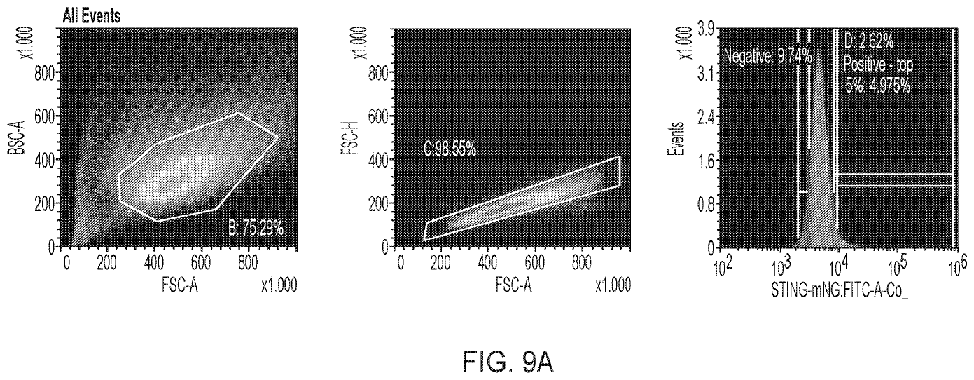

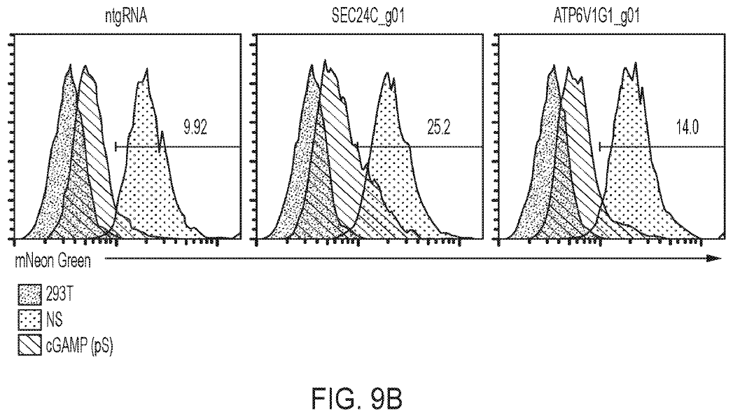

To facilitate the CRISPR screen, a cell line stably expressing spCas9 and STING-mNG (FIG. 2B) that was transduced with the pooled genome-wide human targeting sgRNA library Brunello (Doench et al., 2016) was used. One-week post selection for transduced cells, the cells were stimulated with cGAMP(pS)2, FACS-sorted mNG positive and negative cells (FIG. 9A) and genomic DNA was extracted to measure sgRNA abundance in the sorted populations by next-generation sequencing (FIG. 2B). When filtered, 41 positive regulators of degradation were found. The top scoring genes required for STING degradation were SEC24C and ATP6V1G1 (FIG. 2C). SEC24C was already described to regulate STING exit from the ER (Gui et al., 2019) and ATP6V1G1 is a lysosomal V-ATPase subunit required for lysosomal acidification and that was previously shown to be involved in STING degradation (Gonugunta et al., 2017). Individual sgRNA knock-outs for SEC24C and ATP6V1G1 recapitulated the results of the screen (FIG. 2D, FIG. 9B), adding confidence to the validity of this approach.

To determine which screen hits likely interact with STING, significant hits from the CRISPR screen were intersected with the full dataset of STING-TurboID-labelled proteins (FIG. 2E). In addition to SEC24C, SURF4 was found, which was recently implicated in STING trafficking (Deng et al., 2020), as well as SYNE3 which connects the cytoskeleton to the nucleus, the actin gene ACTG1, two genes involved in RNA processing, XRN2 and HBS1L, and the gene of unknown function UBAP2.

When intersected with the filtered 132 proteins from the TurboID dataset, only 2 genes were identified, HGS and VPS37A, which are components of the ESCRT machinery (FIG. 2E). Overall, the intersection of the proteomics dataset with the genetic screen nominates candidate genes that potentially interact with STING and are required in regulation of its trafficking. Attention was focused on the two components of the ESCRT machinery identified, HGS and VPS37A.

### An ESCRT Complex Containing HGS and VPS37A Regulates STING Degradation, Signaling Shutdown and Autophagy Resolution

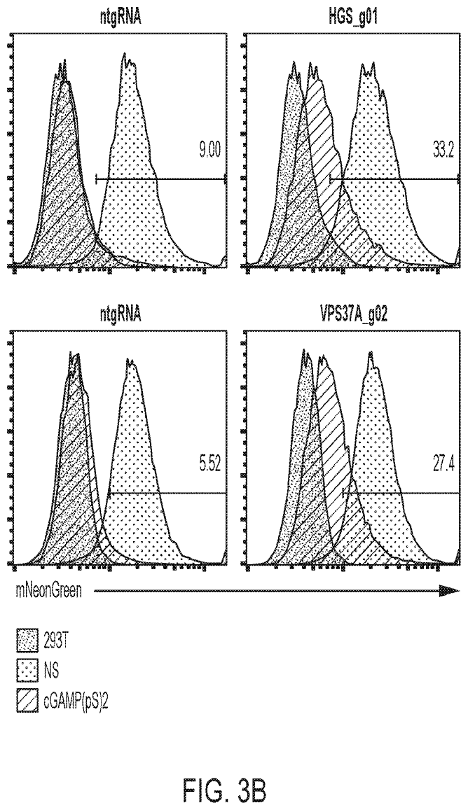

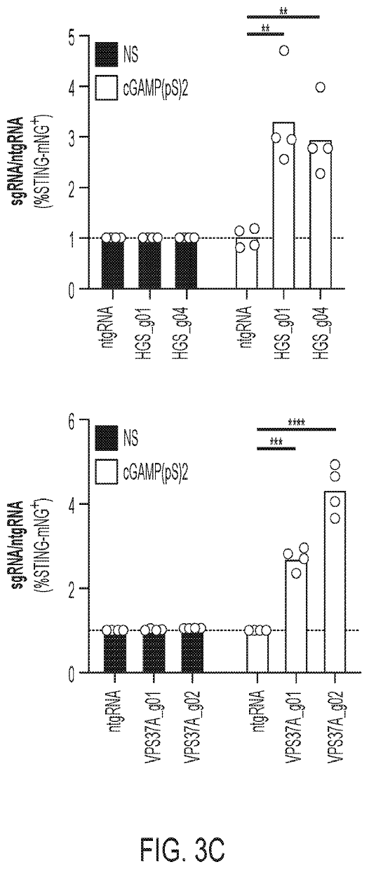

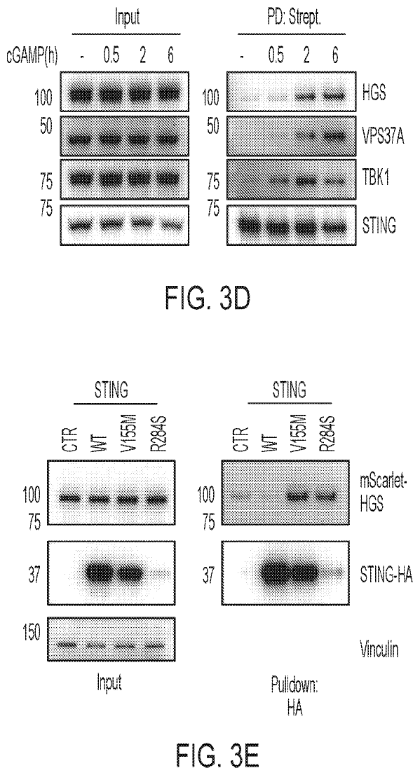

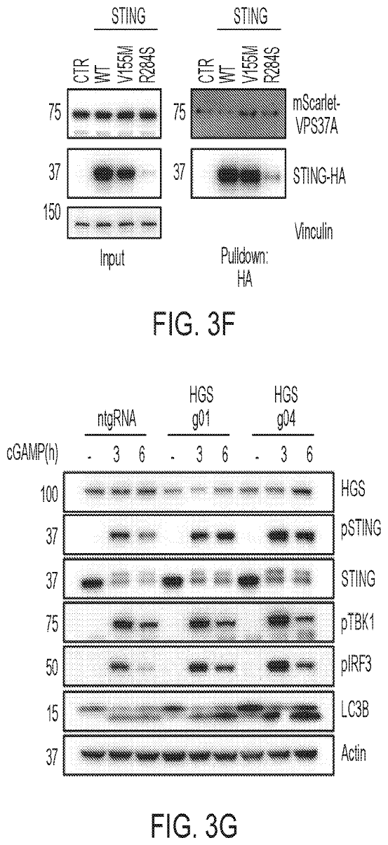

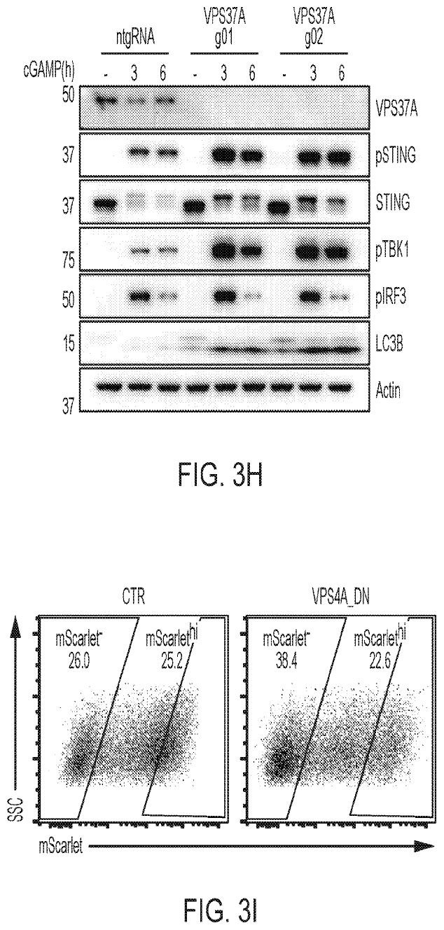

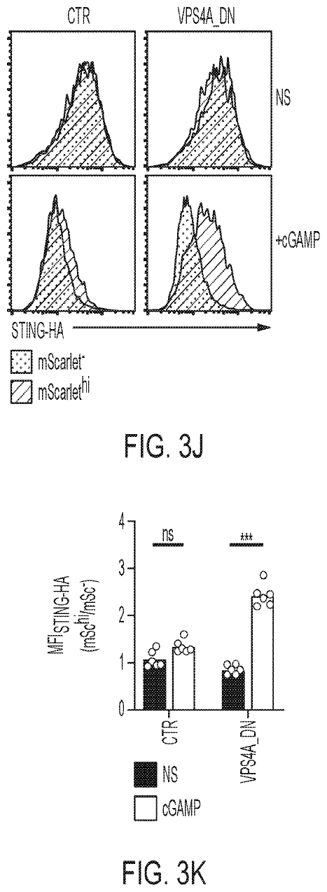

The ESCRT machinery is involved in many cellular processes that entail inverse membrane involution and formation of vesicles. Related to intracellular protein trafficking, ESCRT has been characterized to be required for intraluminal vesicles (ILVs) formation at late endosomes and more recently for resolution of particular forms of autophagy (Victri et al., 2020a). In addition to HGS (ESCRT-0) and VPS37A (ESCRT-I), the proteomics dataset identified other components of the ESCRT machinery: STAM (ESCRT-0), two subunits of the ESCRT-I heterotetrameric complex, TSG101 and UBAP1, and the Bro1 domain protein PTPN23 which has been shown to bridge ESCRT-I to ESCRT-III (Christ et al., 2017) (FIG. 3A). Interestingly, UBAP1 has been shown to be preferentially assembled in an endosome specific ESCRT complex containing VPS37A, but not its homologs VPS37B, C or D (Wunderley et al., 2014).

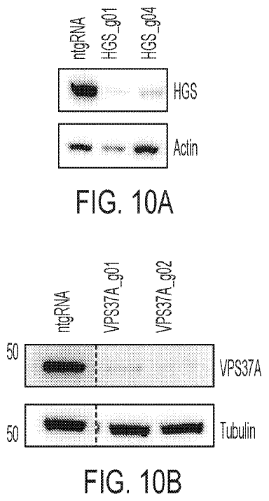

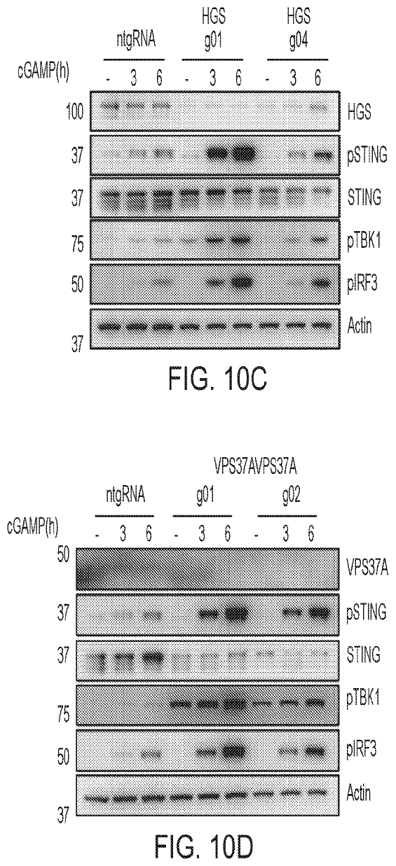

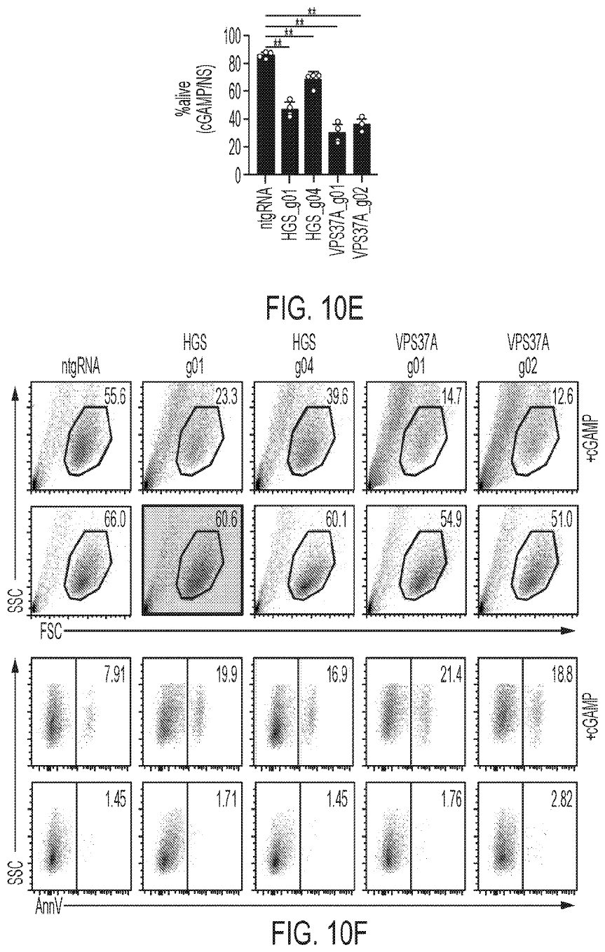

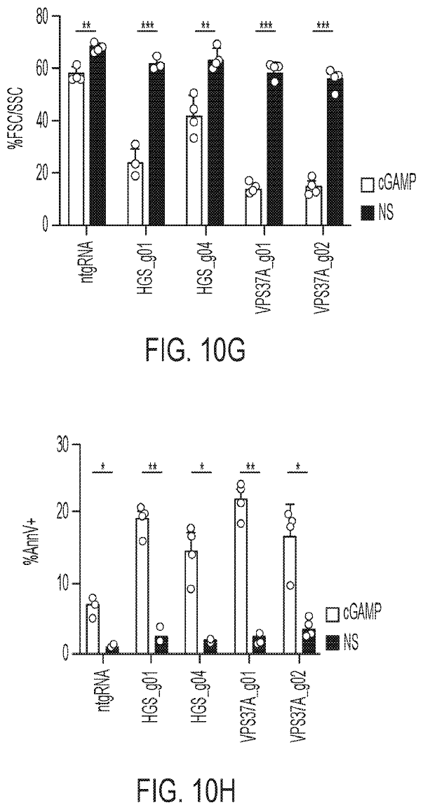

Attention was focused on HGS and VPS37A because they were identified at the intersection of the proteomics and genetics data. When knocked out with two independent sgRNAs per gene, KO STING-mNG reporter cells showed a reduction in STING degradation, confirming the screen results (FIGS. 3B-3C, FIGS. 10A-10B). STING-TurboID mediated labeling of HGS and VPS37A started at 2 hours post cGAMP stimulation and was stable up to 6 hours, confirming the proteomics data (FIG. 3D). Since proximity ligation identifies interacting and non-interacting proteins in the proximity of the bait, co-immunoprecipitations were also performed (co-IP) with STING. 293 Ts stably expressing a control vector, HA-tagged wild-type (WT) STING, or the constitutive active mutants STING V155M and STING R284S found in SAVI patients, were generated (Jeremiah et al., 2014; Konno et al., 2018; Liu et al., 2014). HGS and VPS37A co-immunoprecipitated with the STING constitutive active mutants V155M and R284S, but not with WT STING (FIGS. 3E-3F). This confirmed a physical interaction between activated STING and the ESCRT subunits HGS and VPS37A.

Since STING functions are dependent on its intracellular trafficking and degradation, it was asked if HGS and VPS37A KO would impact STING signaling. CRISPR KO human primary fibroblasts (BJ1) and monocytic U937 cell lines, with two independent sgRNAs per gene, were generated. KO of HGS or VPS37A increased STING signaling in the two cell types for both genes (FIGS. 3G-3H, FIGS. 10C-D). Impaired STING degradation and failure to shut-down STING signaling leads to cell death. U937 have been shown to be susceptible to STING dependent cell death31, 32. Consistent with exacerbated STING signaling, HGS and VPS37A KO U937 were subject to increased cell death in response to cGAMP and showed higher levels of Annexin V staining in the cells that remained alive 24 h post-treatment (FIGS. 10E-10H). In fibroblasts, KO of either gene also reduced rates of STING degradation, confirming the screen results (FIGS. 3G-3H). Since resolution of membrane involution by ESCRT is dependent on VPS4A/B, if STING degradation was dependent on these proteins was tested. The VPS4A E228Q dominant negative (DN) mutant fused to mScarlet was overexpressed in a STING-HA reporter cell line. When stimulated with cGAMP, there were comparable levels of STING degradation between non transfected cells and cells transfected with a control plasmid (FIGS. 3H-3J), while cells expressing the VPS4A DN showed a marked reduction in STING degradation (FIGS. 3H-3J), consistent with an ESCRT requirement for this process.

Taken together, these data suggest that in addition to being required for STING degradation, ESCRT is also responsible for STING signaling shutdown

### ESCRT Links STING Degradation and Autophagy Resolution at the Endosome

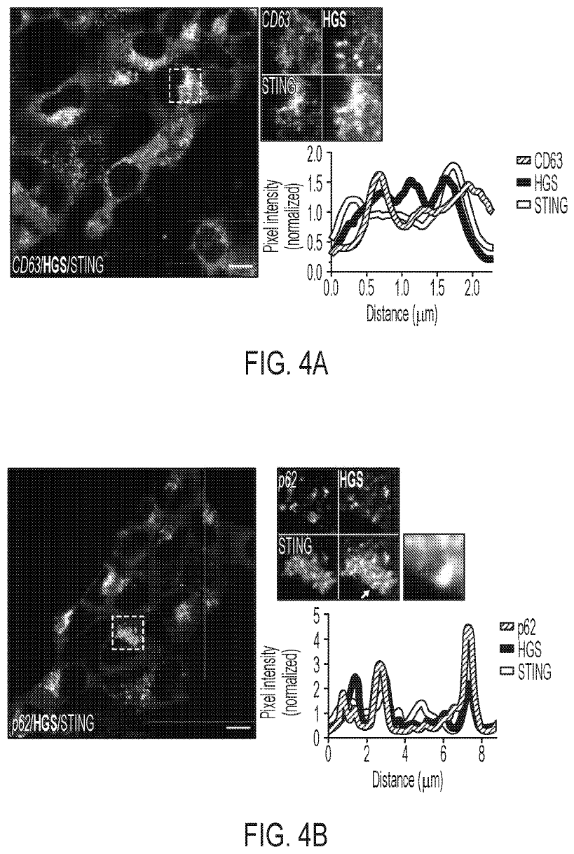

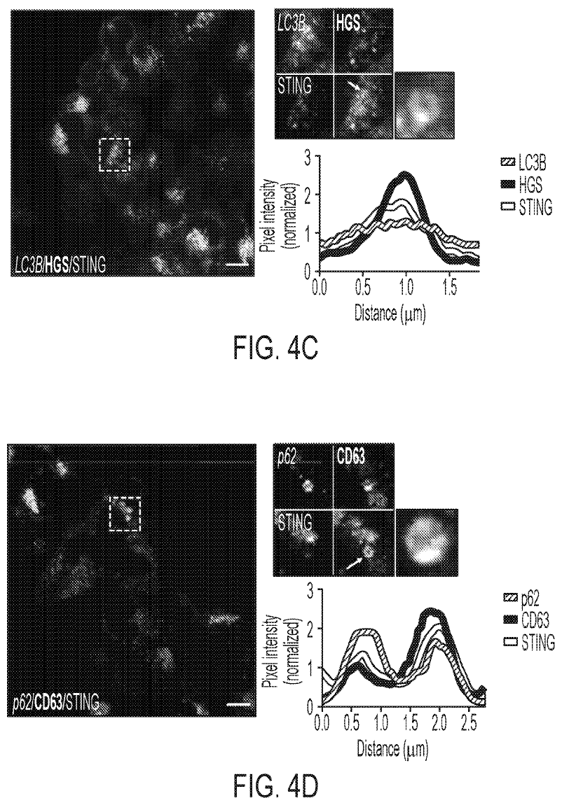

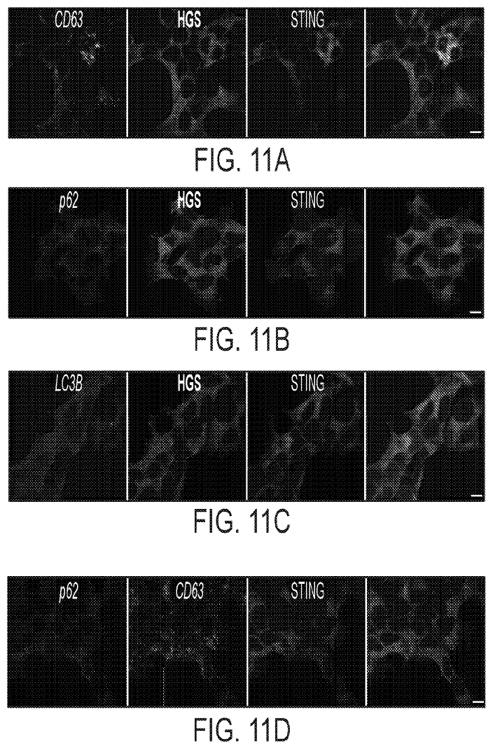

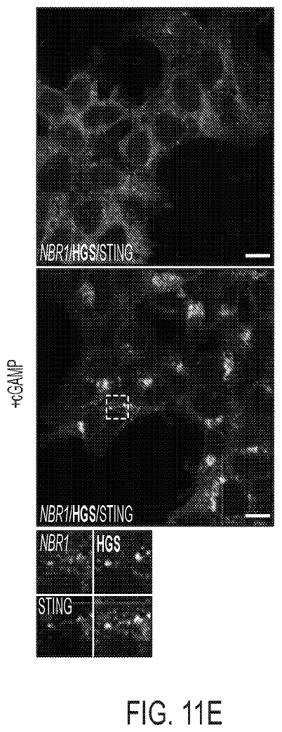

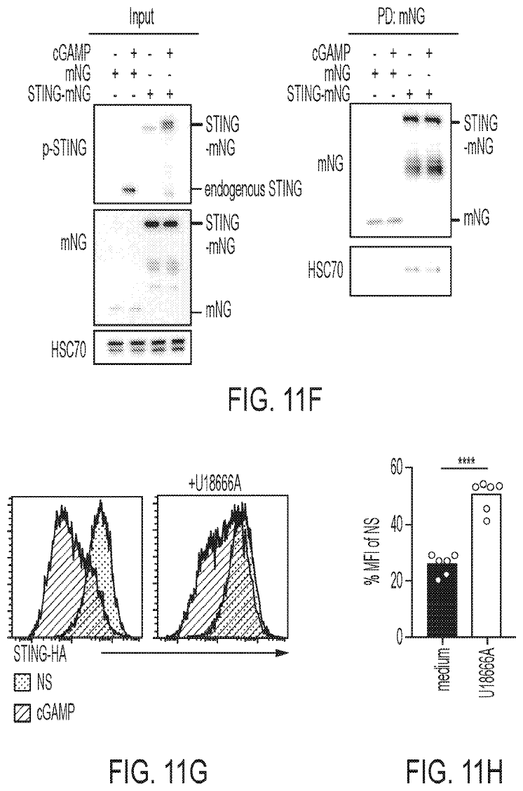

Interestingly, HGS and VPS37A KO in fibroblasts not only blocked STING degradation but also increased lipidated LC3B levels, an autophagy marker that accumulates in cells with defective resolution of autophagy (FIGS. 3G-3H). This finding led to the thought that perhaps if the endosome and the autophagy degradative pathways, previously proposed to be distinct and to proceed respectively from the TGN or from the ERGIC, were instead part of one coordinated shutdown mechanism. The ESCRT-I component VPS37A, which acts downstream of the ESCRT-0 subunit HGS, has recently been shown to be required for phagophore closure in macroautophagy (Takahashi et al., 2019). To visualize these pathways, cells were stained with antibodies to the ESCRT-0 subunit HGS, which initiates the ESCRT cascade and acts upstream of VPS37A, the late endosomal marker CD63 and the autophagy markers p62, identified in our proteomics, or LC3B. Upon cGAMP stimulation, HGS formed distinct foci in the cell that co-localized with STING and were in close proximity to CD63, indicating that STING and ESCRT co-localize at the late endosome (FIG. 4A, FIG. 11A). Co-localization of STING and the autophagy receptor p62 was then examined. Similar to HGS, p62 formed distinct foci in the cell upon cGAMP stimulation that in some cases looked like circular structures resembling autophagosomes (FIG. 4B, FIG. 11B). p62 foci colocalized with HGS and STING, suggesting that ESCRT could be involved in resolution of STING induced autophagy. Interestingly, when clear circular p62 structures could be identified, it was noticed that HGS and STING formed a bright signal at one distinct focus on these structures (FIG. 4B), reminiscent of forms of organelle autophagy such as mitophagy (FIG. 4B) (Zhen et al., 2020). Similar circular structures with a bright spot of STING and HGS colocalization were identified when stained for LC3B (FIG. 4C, FIG. 11C).

The co-localization of STING and ESCRT with the late endosomal marker CD63 and the autophagy markers p62 and LC3B prompted further investigation to determine if STING could be degraded through endosomal microautophagy, a process that involves selective autophagy of late endosomal cargo. Endosomal microautophagy requires acidification of lysosomes for selective degradation of cargo, shows co-localization of selective autophagy receptors with late endosomal markers, and requires the ESCRT subunits VPS4A/B for resolution (Mejlvang et al., 2018; Tekirdag and Cuervo, 2018). STING degradation was already shown to be dependent on lysosomal acidification, and the screen confirmed this result (FIG. 2D, FIG. 9A) (Gonugunta et al., 2017). To evaluate co-localization of selective autophagy receptors with late endosomes, STING expressing cells were stained for p62 and CD63 (FIG. 4D, FIG. 11D). Co-localization of p62 with CD63 was observed, and again p62 circular structures with a bright focus of STING that also contained CD63 were noted (FIG. 4D). The selective autophagy receptor NBR1, identified by STING-TurboID mass spectrometry (FIG. 1E), also co-localized with HGS and STING upon cGAMP treatment (FIG. 11E). NBR1 has been shown to be involved in endosomal microautophagy in yeast and to facilitate selective autophagy of cargo in complex with p62 (Liu et al., 2015b; Stolz et al., 2014). Moreover, proteins that can be degraded by endosomal microautophagy also present a KFERQ (SEQ ID NO: 7) motif required for association with the chaperone HSC70, which STING has been predicted to contain (Kirchner et al., 2019). Association of cargo with HSC70 has been shown to be a distinctive feature of proteins degraded through endosomal microautophagy (Sahu et al., 2011). STING association with HSC70 was then tested for by Co-IP, and indeed STING association with HSC70 was confirmed regardless of cGAMP stimulation, suggesting constitutive association of these two proteins (FIG. 11F).

The co-localization of STING and ESCRT in proximity of the late endosomal marker CD63 and the autophagy markers p62 and LC3B prompted the thought that if resolution of STING-dependent autophagy could happen at the endosome in the process of amphisome formation. Amphisomes are hybrid degradative organelles resulting from the fusion of autophagosomes and endosomes. Therefore, STING expressing cells were stained for p62 and CD63 (FIG. 4D, FIG. 11D) and indeed observed co-localization of the two markers when cells were treated with cGAMP. p62 circular structures with a bright focus of STING that also contained CD63 was again observed, indicating that STING acts as an organizing hub for ESCRT recruitment and regulates endosome-autophagosome fusion events (FIG. 4D). Consistent with this model, mass spectrometry showed enrichment of Myosin VI upon STING activation, which marks autophagosomes for fusion with endosomes (FIG. 1E).

To test if STING degradation was VPS4A/B dependent, the VPS4A E228Q dominant negative (DN) mutant fused to mScarlet was overexpressed in a STING-HA reporter cell line (VPS4A DN was used in lieu of genetic deletion to block both VPS4A and VPS4B since they have redundant functions). When stimulated with cGAMP, there were comparable levels of STING degradation between non transfected cells and cells transfected with a control plasmid (FIGS. 4E-4G), while cells expressing the VPS4A DN showed a marked reduction in STING degradation (FIGS. 4E-4G), consistent with an ESCRT requirement for this process. Finally, resolution of endosomal microautophagy has also been shown to be sensitive to the NPC1 inhibitor U18666A, which interferes with endosomal dynamics through cholesterol biosynthesis modulation (Mejlvang et al., 2018). Pre-treatment with U18666A led to a marked reduction in STING degradation (FIGS. 11G-11H).

Taken together, these data suggest that activated STING is sorted to endosomes where its degradation is coordinated with autophagy resolution through ESCRT mediated phagophore sealing on this organelle.

### STING Ubiquitination Creates a Platform at the Endosome for Autophagy Resolution and STING Degradation

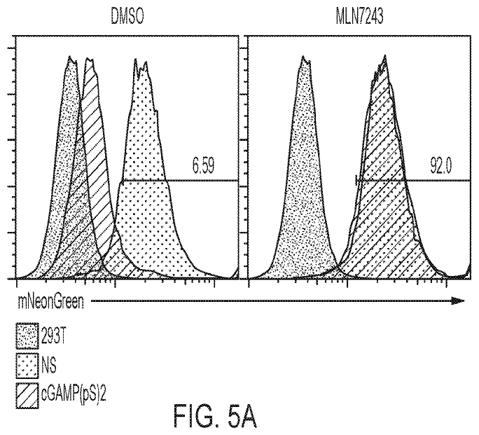

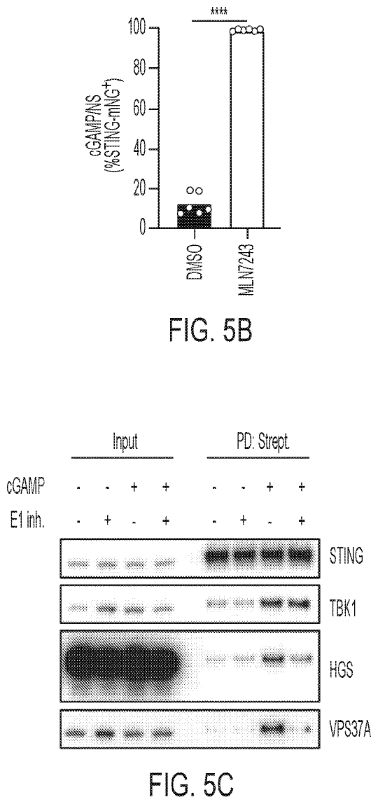

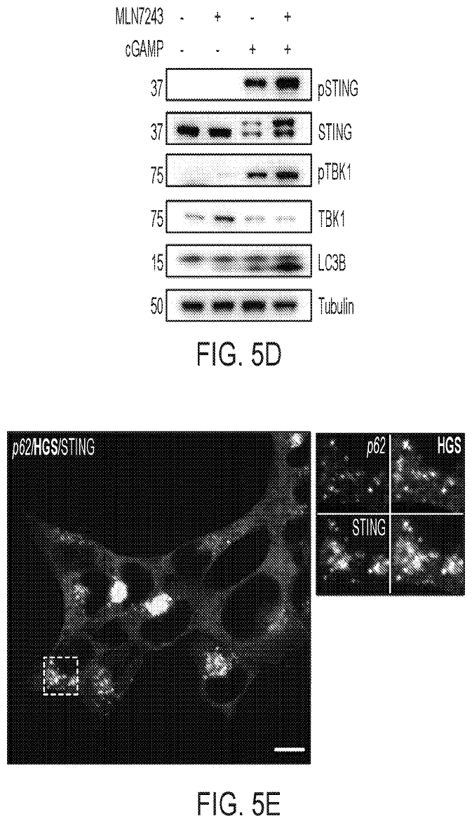

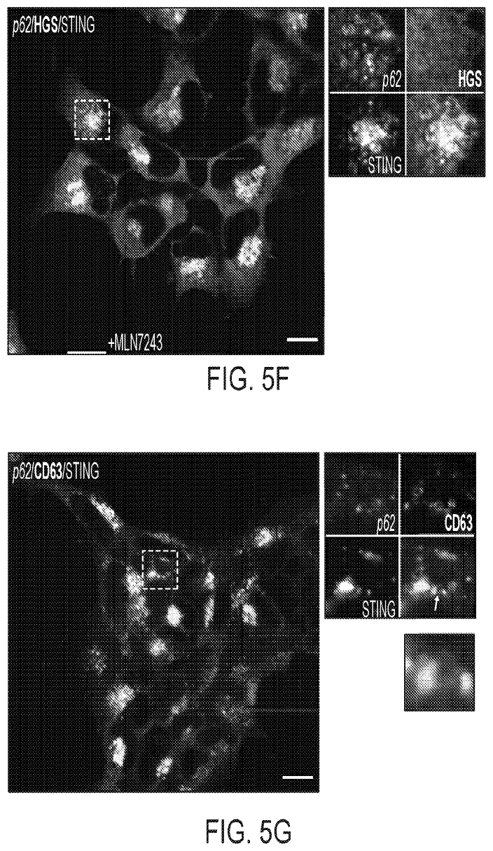

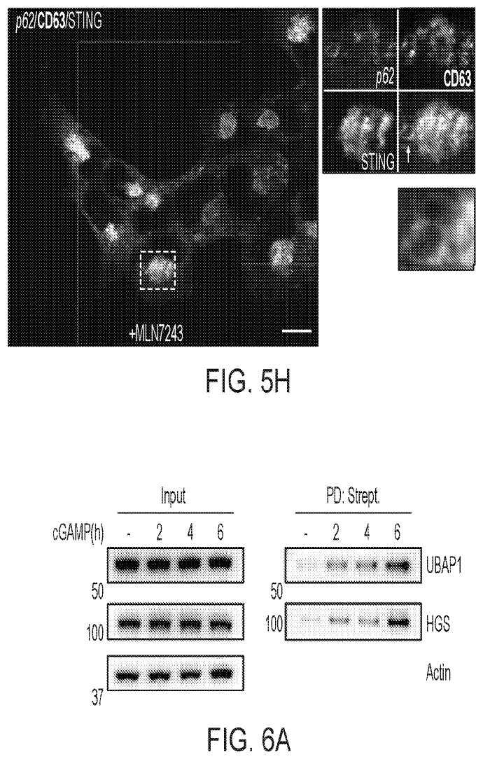

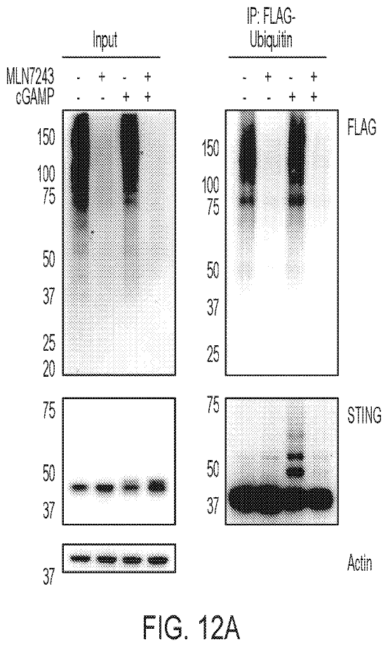

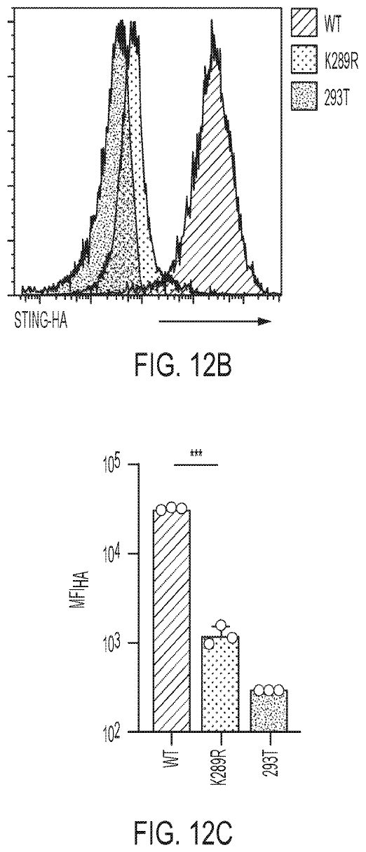

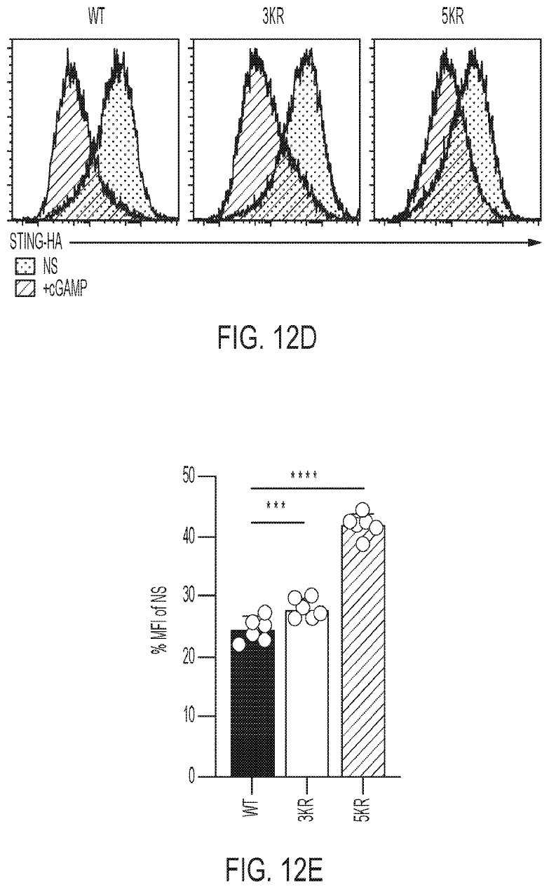

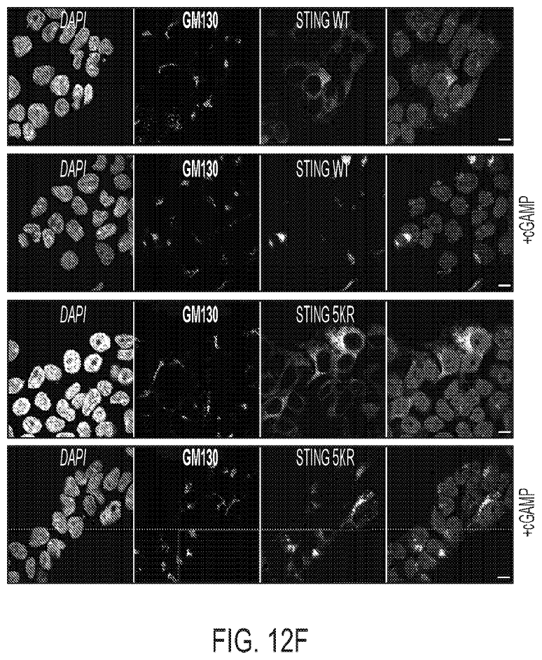

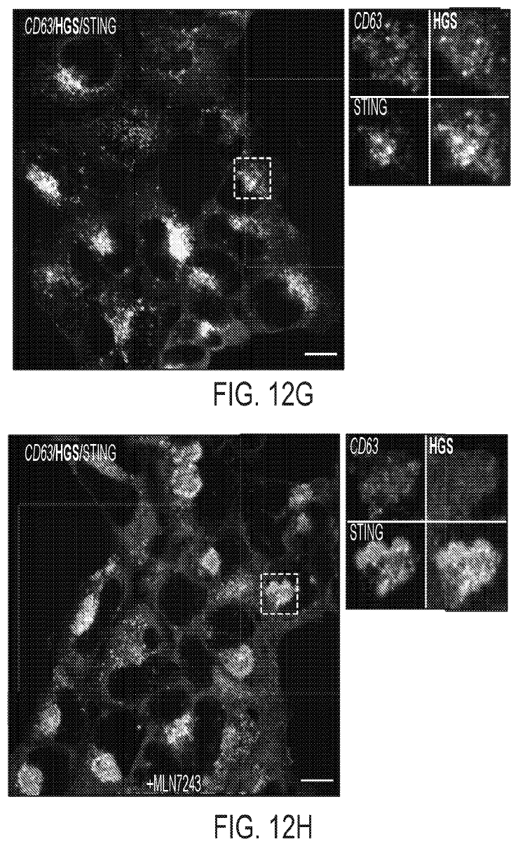

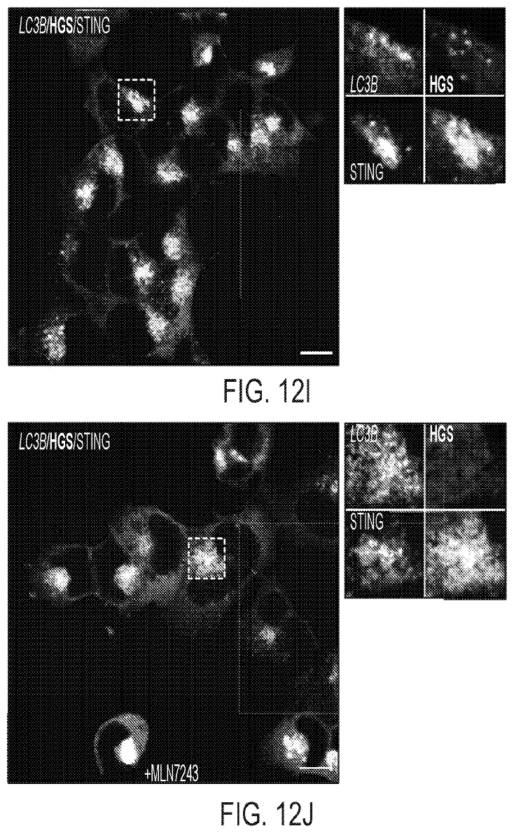

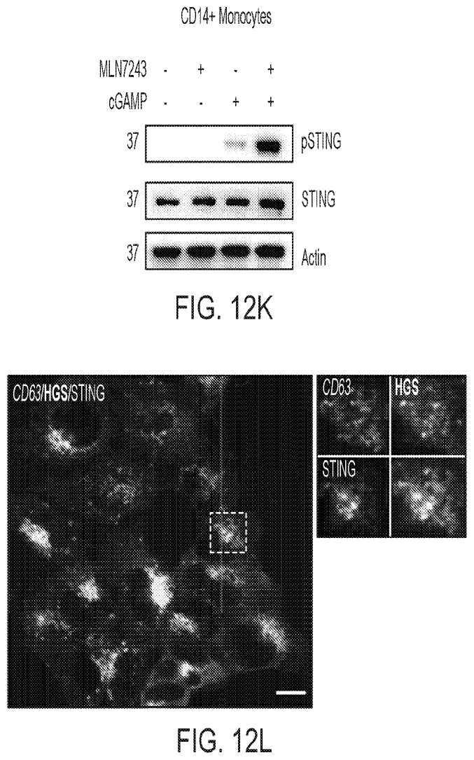

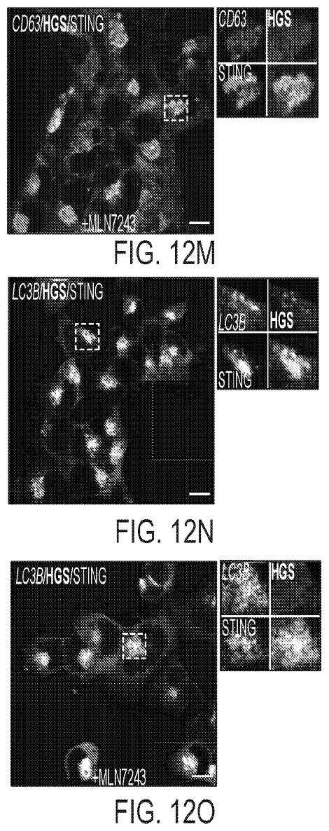

ESCRT-0 and ESCRT-I associate with ubiquitinated cargo at the endosome for formation of ILVs. HGS and VPS37A both contain a ubiquitin binding motif (Vietri et al., 2020a). It was asked if STING was ubiquitinated upon cGAMP stimulation and if ubiquitination could drive its association with ESCRT. To block ubiquitination of STING, the Ubiquitin Activating enzyme 1 (UBA1) inhibitor MLN7243 was used (Hyer et al., 2018). MLN7243 blocked STING ubiquitination upon cGAMP stimulation (FIG. 12A). When the STING-mNG reporter cell line was stimulated with cGAMP, there was a complete block of degradation of STING in presence of the inhibitor (FIGS. 5A-5B). Ubiquitination can drive protein degradation through the proteasome or through intracellular protein trafficking to MVBs. To discriminate between these two pathways, STING-mNG expressing cells were treated with two proteasome inhibitors, Bortezomib and MG-132, the UBA1 inhibitor MLN7243 and the lysosomal V-ATPase inhibitor Bafilomycin A1. Both proteasome inhibitors only partially impacted STING degradation, consistent with previous reports while this process was completely blocked by both MLN7243 and Bafilomycin A1, suggesting that sorting to MVBs and acidification of the lysosome are central to STING degradation (FIGS. 12L-12M). Therefore, it was hypothesized that MLN7243 blocked STING degradation by inhibiting its ubiquitination, consequently preventing its interaction with HGS and VPS37A. To test this hypothesis, STING-TurboID expressing cells were treated with MLN7243. Treatment with the drug reduced the biotin labeling of HGS and VPS37A in the STING-TurboID cell line, while labeling of TBK1 was unaffected (FIG. 5C). These results suggest that STING ubiquitination upon activation drives its association with ESCRT and regulates its degradation.

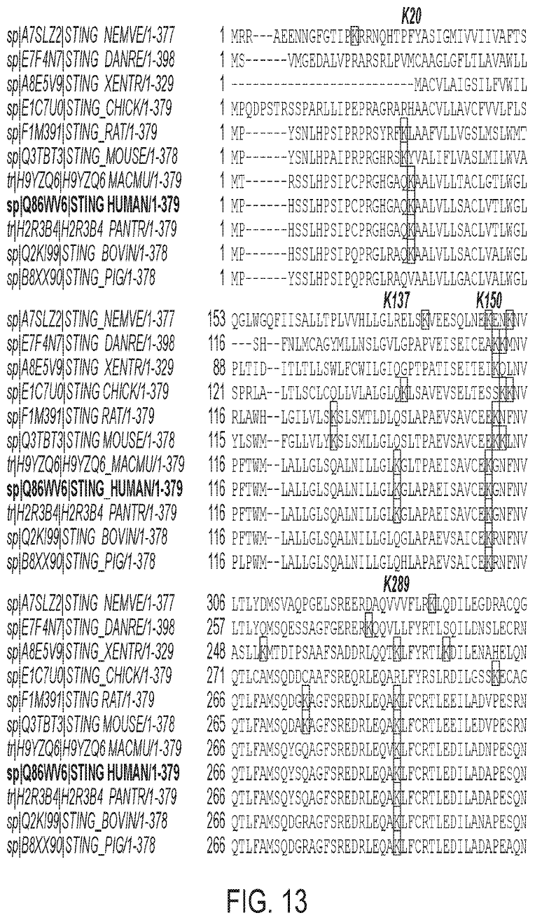

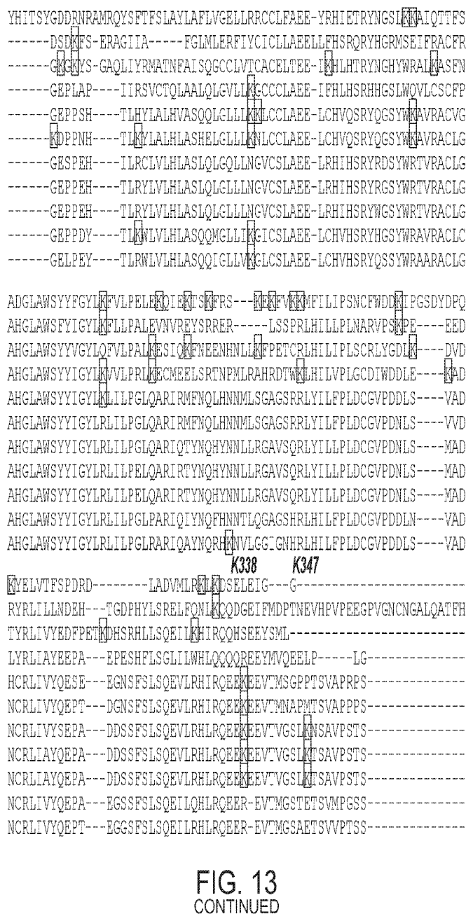

It was predicted that ubiquitination at multiple lysine residues would drive STING degradation, since HGS contains 3 ubiquitin binding sites (Vietri et al., 2020a). When lysine conservation was examined in human STING, K20, K150, K236, K289, K338 and K370 were identified as highly conserved, among 9 lysine residues (FIG. 13). K289 was excluded from experiments, since STING K289R is unstable and degraded at steady state (FIGS. 12B-12C), as previously reported (Ni et al., 2017). 293 Ts stably expressing a STING 3KR (K20R/K150R/K236R) and a STING 5KR mutant (STING K20R/K150R/K236R/K338R/K370R) fused to HA were then generated. When stimulated with cGAMP, STING 3KR showed a modest decrease in degradation, while STING 5KR almost completely abrogated STING degradation (FIGS. 12D-12E). STING 5KR was not sensitive to MLN7243 (FIG. 12F). It was also tested whether STING 5KR could still exit the ER since, for example, K224 is critical for this process (Ni et al., 2017). When stimulated with cGAMP, STING 5KR cells showed STING co-localization with the Golgi marker GM130, suggesting that the defect in STING 5KR degradation was not due to defects in ER exit (FIG. 12G). These data suggest that STING K338/K370 are at least partially driving STING degradation through ubiquitination.

Since ubiquitination is driving STING association with ESCRT, it was asked if the UBA1 inhibitor MLN7243 would recapitulate HGS and VPS37A KO effects on STING signaling, degradation and autophagy resolution. Indeed, treatment with MLN7243 abrogated STING degradation in fibroblasts, increased phospho-STING signaling and also induced accumulation of lipidated LC3B (FIG. 5D).

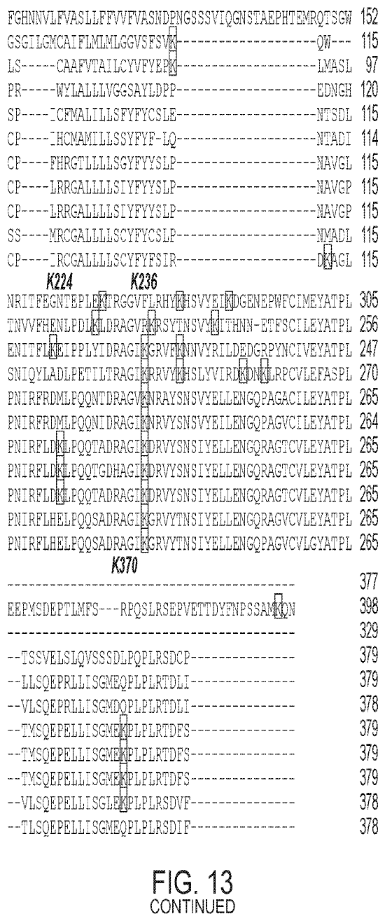

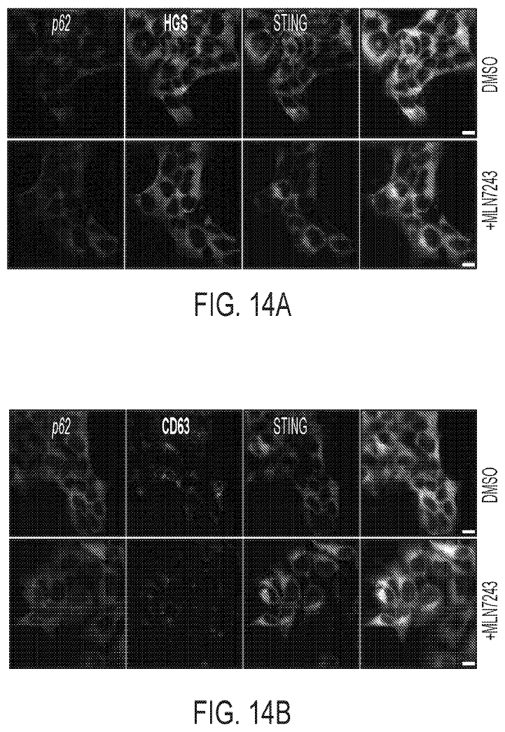

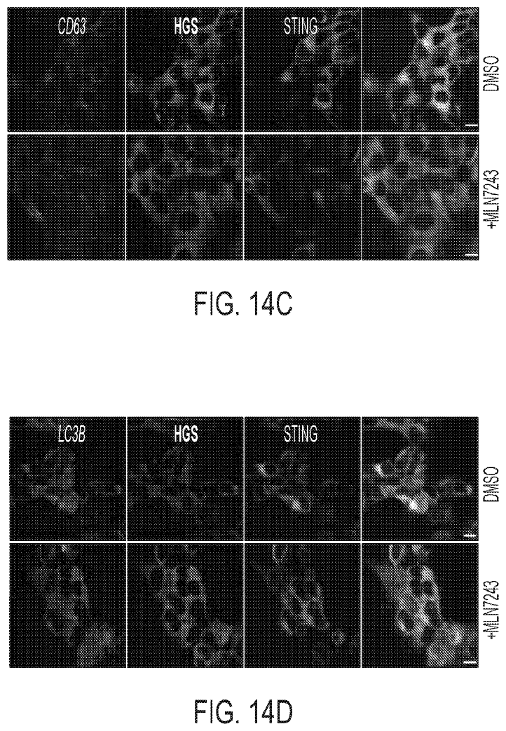

Next, the impact of UBA1 inhibition on STING subcellular localization and its co-localization with the late endosome marker CD63, autophagy markers p62 and LC3B and ESCRT was identified. When treated with MLN7243, HGS foci co-localizing with STING were completely lost (FIGS. 5E-5F, FIG. 14A) and STING showed a different intracellular distribution pattern lacking dispersion in perinuclear vesicles (FIGS. 5E-5H, FIGS. 12H-12K, FIGS. 14A-14D). HGS also failed to co-localize with p62 in presence of MLN7243 suggesting that ubiquitinated STING acts as an organizing center for ESCRT recruitment to autophagosomes (FIGS. 5E-5F, FIG. 14A). The same was true for co-localization with LC3B or CD63 and HGS (FIGS. 12H-12K, FIGS. 14C-14D). Moreover, it was noticed that the distribution of p62 changed from one spot co-localizing with STING at one edge of CD63⁺ vesicles to an accumulation of p62⁺ ring-like structures, indicative of failure of phagophore closure and autophagy resolution (FIGS. 5G-5H, FIG. 14B). In presence of MLN7243, p62⁺ vesicles in the process of fusing with CD63⁺ vesicles were identified, (FIG. 5H) suggesting that STING-induced ESCRT-dependent resolution of autophagy through autophagosome-endosome fusion was blocked.

Taken altogether, analogous to phosphorylation that creates a platform at the TGN for type I IFN signaling, ubiquitinated STING traffics to and decorates the endosome creating an organizing platform that coordinates STING degradation and autophagy resolution through ESCRT. ESCRT drives phagophore sealing and fusion with the endosome and consequent autophagy of endosomal content. Since VPS37A has been shown to be required for phagophore closure, and ubiquitinated STING assembles a specific VPS37A containing ESCRT complex, VPS37A acts as a bridge between endosomal sorting of STING and phagophore closure on this organelle.

Mutations in UBA1 have been recently identified in patients, leading to an autoinflammatory disease named Vacuoles E1 enzyme X-linked Autoinflammatory Somatic (VEXAS) syndrome (Beck et al., 2020). VEXAS syndrome patients express a de novo hypomorphic UBA1 isoform in the myeloid compartment. Since these patients show type I IFN and proinflammatory cytokine production at steady state, it was wondered if UBA1 inhibition in CD14+ monocytes could lead to an increase in STING signaling. After cGAMP stimulation, CD14+ monocytes from healthy donors showed a strong increase in phospho-STING levels when UBA1 was inhibited by MLN7243 treatment (FIG. 12K). Since VEXAS is an autoinflammatory disease with late onset (median age at onset-64), these results might suggest that the inflammation in these patients could be driven by increased STING signaling due to defective degradation triggered by an increased presence of cytosolic DNA associated with cellular senescence (Dou et al., 2017; Glück et al., 2017; Yang et al., 2017).

### A Targeted CRISPR Screen Identifies UBE2N as a Regulator of STING Degradation

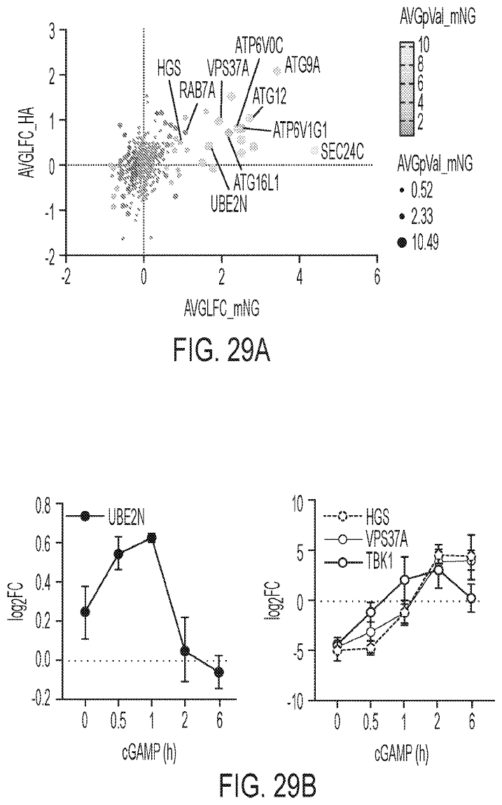

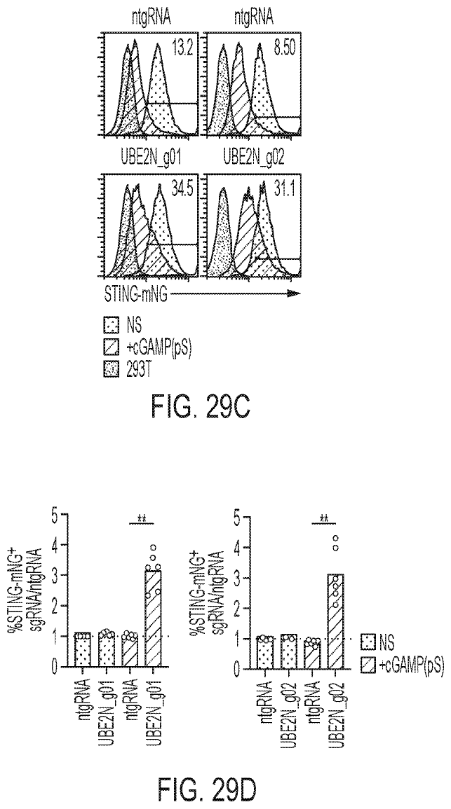

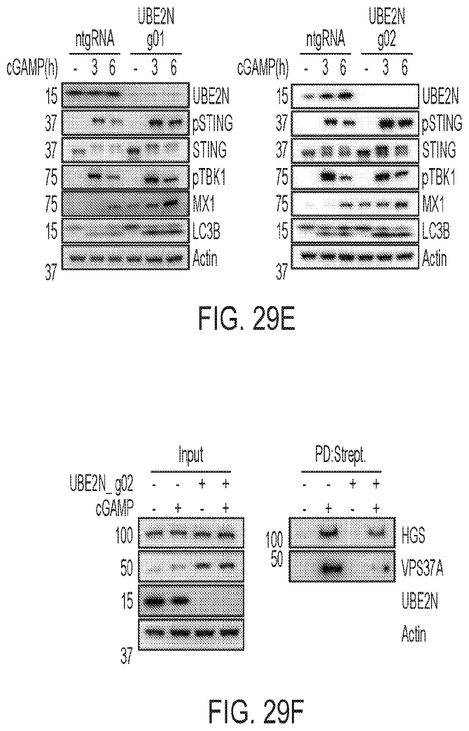

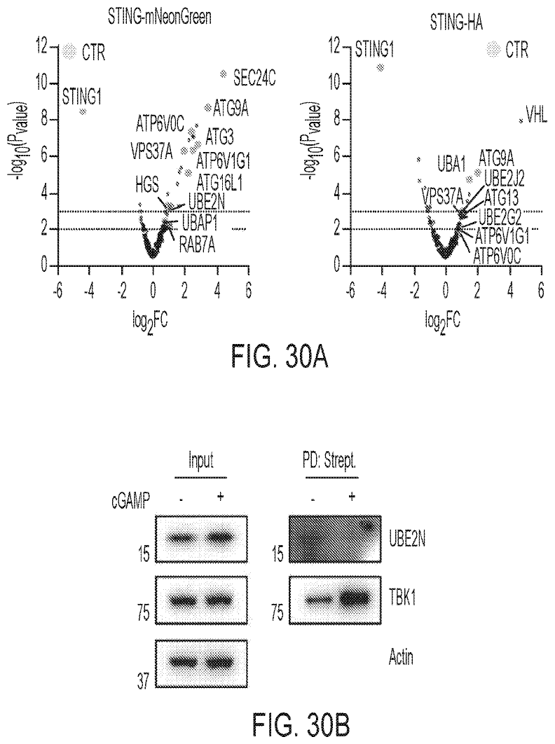

To specifically identify ubiquitin related genes involved in STING degradation, a targeted CRISPR screen was performed in both the STING-mNG and STING-HA reporter cell lines. The targeted library contained guides targeting 669 E3 and adaptors (compiled from Medvar B et al and Li W et al), 40 E2 from Interpro, 7 E1, 28 Autophagy core proteins and 10 positive controls from the genome-wide CRISPR screen. While the STING-mNeonGreen performed better than the STING-HA screen, both recovered positive controls as required for STING degradation (SEC24C, ATP6V1G1, ATP6V0C, HGS, VPS37A, UBAP1) and STING as the top depleted gene (FIG. 30A). To increase confidence in identifying relevant genes, both screens were correlated based on average fold change (FIG. 29A). Positive controls correlated strongly in both screens. Interestingly, genes involved in autophagy as required for STING degradation were clearly identified, with ATG9A, ATG12 and ATG16L1 highly enriched in both screens, suggesting that autophagy plays a role in STING degradation, contrary to previous reports. When looking at ubiquitin related genes, attention was focused on the E2 conjugating enzyme UBE2N. UBE2N was present in the TurboID dataset and showed a peak enrichment at 1 h post cGAMP stimulation that rapidly dropped at the 2 hour time-point, preceding association of STING with ESCRT (FIG. 29B). STING-TurboID labeling of UBE2N was confirmed via western blot (FIG. 30B). Based on proteomics (FIG. 1D, FIGS. 8A-8D), these results suggest that UBE2N is in proximity of STING at the Golgi (1 h time-point) and is rapidly dissociating from STING post-Golgi.

UBE2N is responsible for K63 polyubiquitination of proteins that prompts them for degradation through the endolysosomal compartment and has been shown to play a regulatory role in multiple innate immune sensing pathways. Consistent with a possible role for UBE2N in STING biology, STING has been shown in multiple studies to be K63 polyubiquitinated upon activation. To validate the results of the screens, UBE2N was knocked out with two individual sgRNAs in 293T STING-mNG and stimulated the cells with cGAMP. UBE2N KO cells showed reduced STING degradation (FIGS. 29C-29D). Then, UBE2N was knocked out in fibroblasts to study the impact on STING signaling. Consistent with ESCRT KO, UBE2N led to decreased STING degradation, increased STING signaling and accumulation of lipidated LC3B (FIG. 29E). To test if the UBE2N KO effect on STING degradation and signaling was due to impaired STING association with ESCRT, the STING TurboID cell line was used, in which UBE2N was knocked out. KO of UBE2N led to a modest reduction of association with HGS upon cGAMP stimulation, while leading to a striking reduction of association with VPS37A (FIG. 29F). These results suggest that UBE2N activity regulates STING degradation through its association with ESCRT, impacting VPS37A recruitment to STING.

### A Patient Mutation in the ESCRT-I Subunit UBAP1 Induces a Constitutive STING-Dependent Type I IFN Response

Next, proteins in the dataset were investigated to determine whether proteins in the dataset have established roles in human disease, potentially suggesting altered STING signaling as part of the underlying pathophysiology. Mutations in the ESCRT-I subunits VPS37A and UBAP1 have been shown to induce the neurodegenerative disease Hereditary Spastic Paraplegia (HSP) (Farazi Fard et al., 2019; Gu et al., 2020; Lin et al., 2019; Zivony-Elboum et al., 2012). Interestingly, a subset of patients with mutations in the Aicardi-Goutiére Syndrome genes ADAR1, IFIH1 and RNASEH2B, that present with constitutive induction of type I IFN, have been shown to develop HSP (Crow et al., 2014). Since UBAP1 is an endosome specific ESCRT subunit that does not play a role in cytokinetic abscission, it was asked if disease causing mutations in this gene could lead to dysfunctional STING degradation and exacerbate STING dependent signaling. Consistent with the mass spectrometry (FIG. 1E, FIG. 3A), it was found that STING-TurboID labeled UBAP1 (FIG. 7A). Pathogenic UBAP1 mutations in patients derive from frameshifts leading to truncated UBAP1 mutants containing only the N-terminal UBAP1-MVB 12-associated (UMA) domain, responsible for interaction with ESCRT, and lacking the Solenoid of Overlapping Ubiquitin-Associated Domains (SOUBA), responsible for association with ubiquitinated cargo. These truncated mutants are dominant negatives for ESCRT function (Agromayor et al., 2012). To mimic a pathogenic variant found in patients, a stop codon was introduced in place of residue G98 in UBAP1 (UBAP1DN) (FIG. 7B) (Farazi Fard et al., 2019) and fused it to mScarlet. When expressed in the reporter STING-HA cell line, UBAP1DN blocked STING degradation (FIGS. 7C-7E) consistent with the findings that a specific VPS37A/UBAP1 ESCRT complex regulates STING trafficking and degradation.

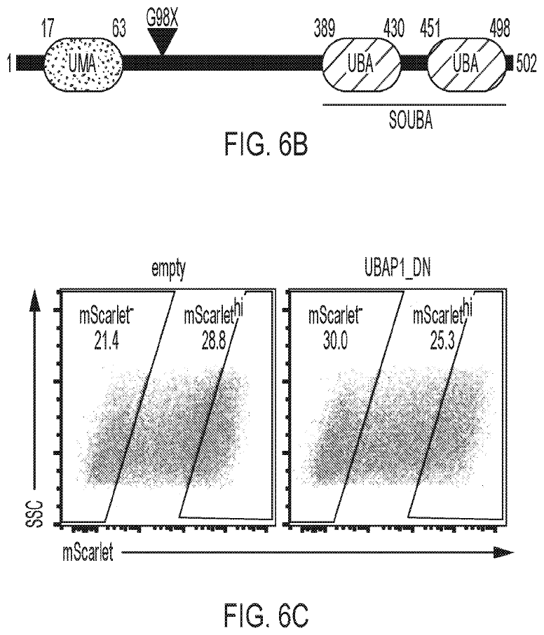

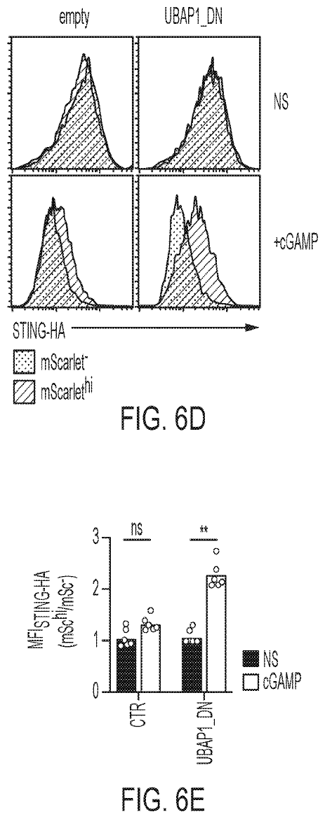

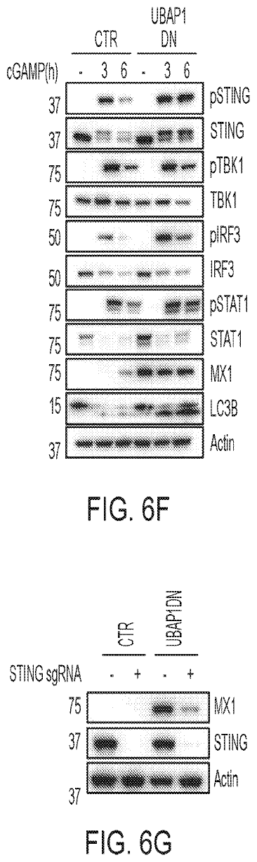

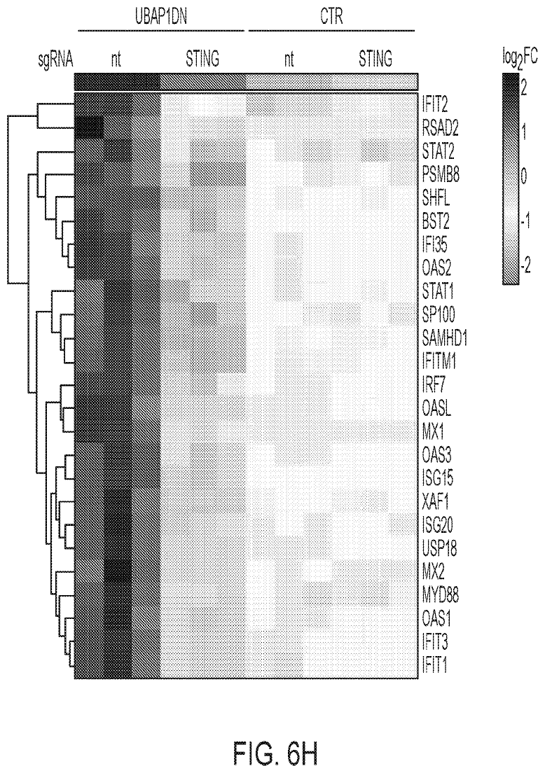

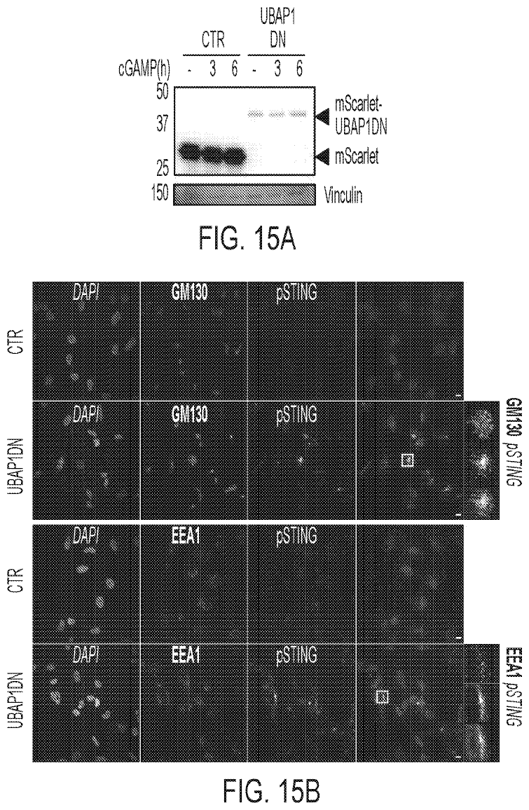

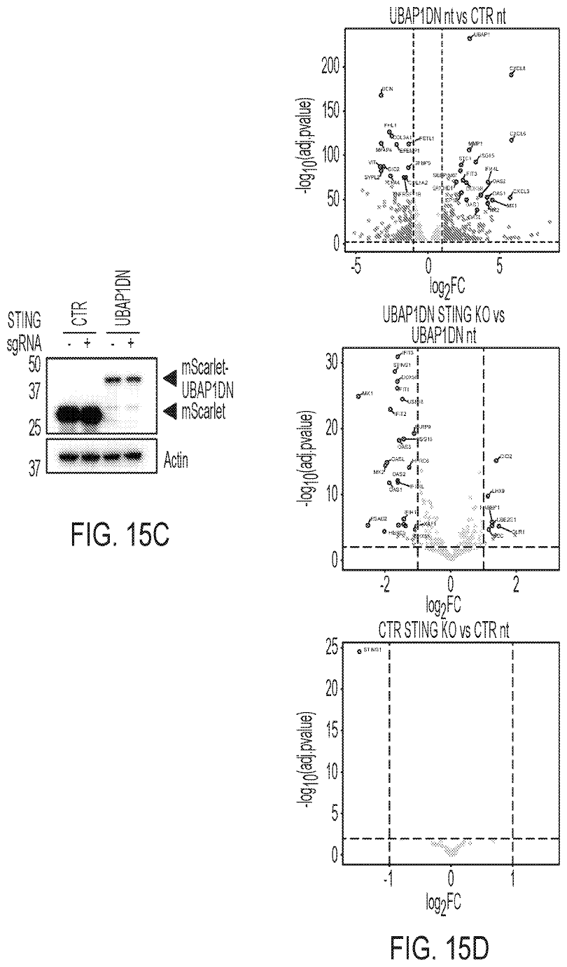

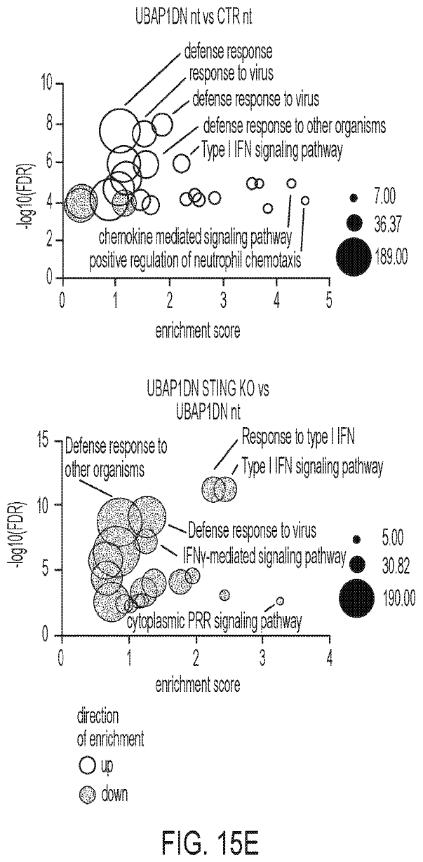

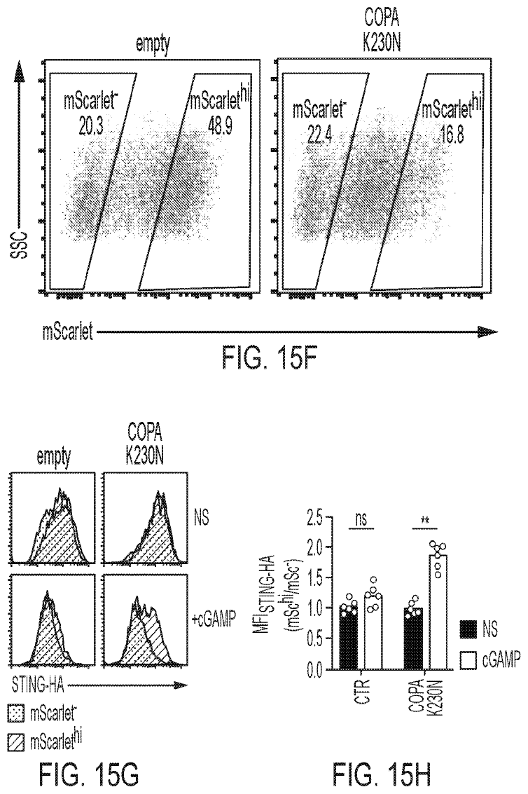

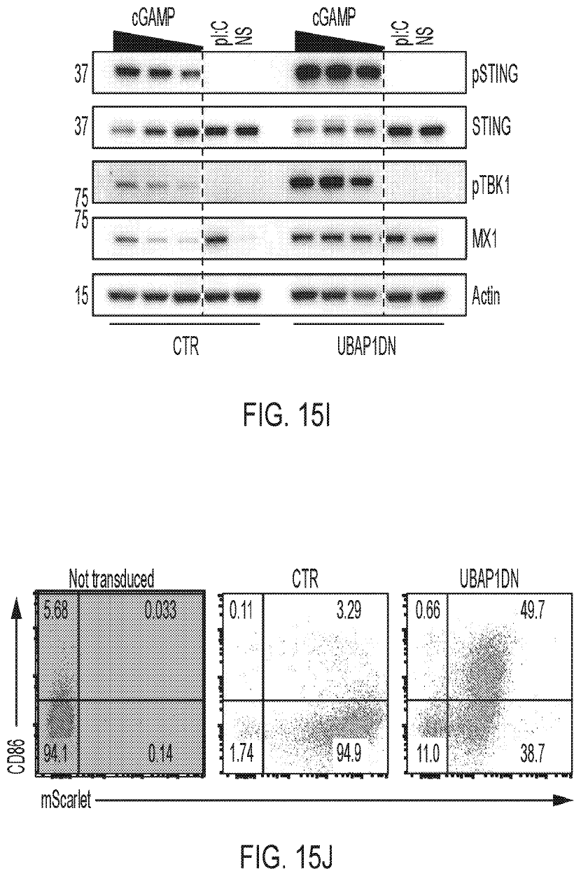

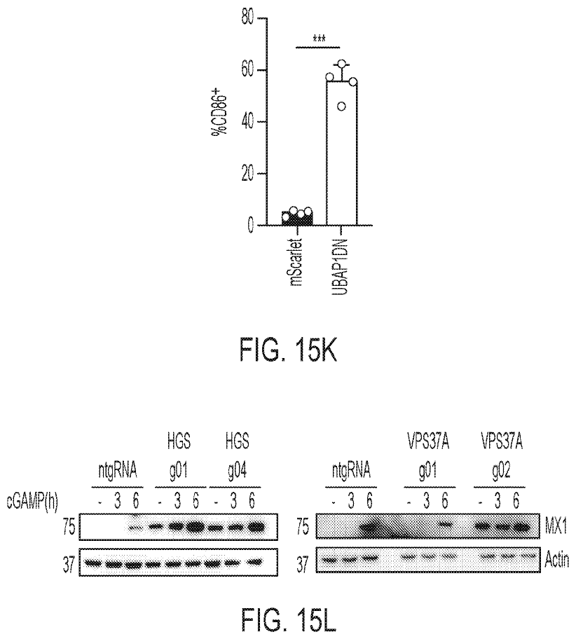

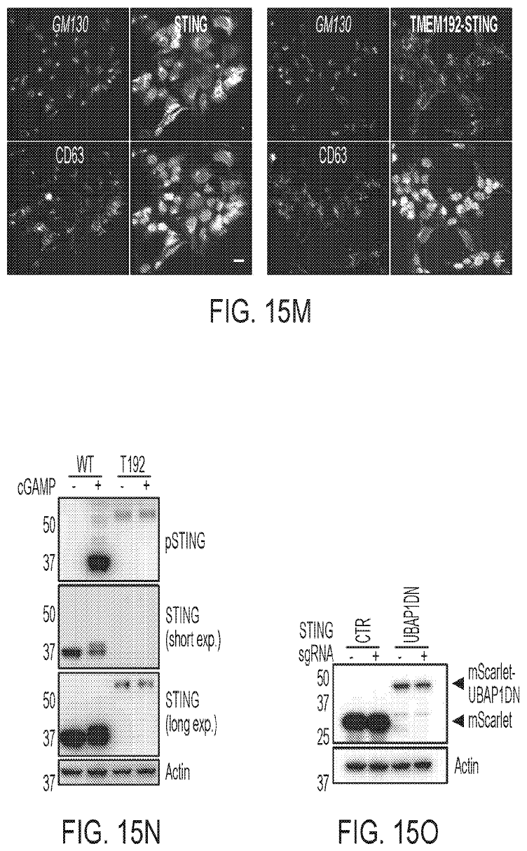

Stable UBAP1DN expressing fibroblasts were generated and stimulated them with cGAMP. UBAP1DN expressing fibroblasts showed a marked increase in STING signaling, reduction in STING degradation and lack of autophagy resolution shown by accumulation of lipidated LC3B (FIG. 7F, FIG. 15A), similar to HGS and VPS37A KO (FIGS. 3G-3H). Expression of the UBAP1DN mutant in primary human Monocyte Derived Dendritic Cells (MDDCs) also led to increased STING signaling (FIG. 15I). Interestingly, it was noticed that UBAP1DN expressing fibroblasts induced the interferon stimulated gene MX1 at steady state, in absence of cGAMP stimulation. (FIG. 6F). The MX1 upregulation at steady state was also present in UBAP1DN expressing MDDCs, which are terminally differentiated cells and do not cycle, in addition to spontaneous DC maturation, shown by CD86 upregulation (FIG. 15I-15K). This suggested that perturbation of ESCRT function by the expression of the HSP UBAP1 mutant, which blocks STING degradation, leads to a constitutive type I IFN response. Consistent with this hypothesis, KO of HGS and VPS37A in fibroblasts also led to MX1 upregulation at steady state (FIG. 15L).

Mutations in genes regulating STING trafficking, specifically COPA, have been recently shown to induce STING dependent spontaneous type I IFN production due to accumulation of STING at the Golgi (Deng et al., 2020; Lepelley et al., 2020; Mukai et al., 2020; Steiner et al., 2020; Uematsu et al., 2020). Similar to COPA, it was reasoned that activation of constitutive type I IFN in UBAP1DN expressing cells could be the result of impaired homeostatic STING trafficking, specifically post-Golgi, leading to intracellular accumulation of activated STING. Consistent with this hypothesis, UBAP1DN expressing fibroblast showed accumulation of phospho-STING that co-localized partially with the Golgi marker GM130 and the early endosome marker EEA1 (FIG. 15B). To test if accumulation of STING in the endolysosomal compartment is sufficient to spontaneously activate the sensor, the four transmembrane domains of STING were substituted with the four transmembrane domains of TMEM192 by fusing the STING C-terminal (aa139-379) to TMEM192 (TMEM192-STING). TMEM192 has been characterized as a membrane-resident protein of the late endosomal and lysosomal compartment. Therefore, it was hypothesized that the TMEM192-STING construct would be constantly trafficked to and accumulated post-Golgi, mimicking the UBAP1 mutant dependent block of STING trafficking. Expression of the TMEM192-STING construct led to dispersion of intracellular STING staining that partially co-localized with CD63 (FIG. 15M). Compared to WT full length STING, TMEM192-STING led to spontaneous signaling activation, as shown by phospho-STING, even in absence of ligand (FIG. 15N).

To test if induction of ISGs in UBAP1DN expressing fibroblasts was due to accumulation of activated STING, CRISPR KO was used. When STING was deleted, UBAP1DN expressing fibroblasts showed a drastic reduction of MX1 induction (FIG. 6G, FIG. 15C). To confirm these results, RNAseq was performed. UBAP1DN expressing fibroblasts transduced with a non-targeting sgRNA showed an increase in ISG expression compared to control fibroblasts (FIG. 6H, FIG. 15D). GO analysis of differentially expressed genes identified genes involved in the “response to virus” or “type I IFN signaling” among the most upregulated in UBAP1DN expressing cells (FIG. 15D). STING KO in UBAP1DN expressing cells led to a specific reduction in the expression of ISGs compared to UBAP1DN expressing fibroblasts transduced with a non-targeting sgRNA, including genes involved in the type I IFN pathway as the most downregulated (FIG. 6H, FIGS. 15D-15E). Overall, these data suggest that STING is subject to a homeostatic degradative flux and perturbation of ESCRT leads to post-Golgi accumulation of STING which is sufficient for activation of the sensor.

cGAS has been shown to induce tonic ISG transcription at steady-state in vitro and in vivo, regardless of presence of exogenous DNA, suggesting that cGAS produces a low level of cGAMP in cells to prime this response. To test if cGAS was required for priming STING trafficking at steady state and contributed to the phenotype shown by pathogenic UBAP1 expressing cells, cGAS was knocked out in UBAP1DN expressing fibroblasts. cGAS KO reduced induction of the ISG MX1, suggesting that cGAMP production at steady state is responsible for priming the constitutive flux of STING trafficking (FIG. 15O). This is consistent with the phenotype shown in cells with COPA deficiency that activate a spontaneous type I IFN response that is both cGAS and STING dependent56.

Taken together, these data suggest that cells expressing a pathogenic mutant of UBAP1 have a heightened response to cGAMP through impaired ESCRT mediated STING degradation and autophagy resolution. Additionally, mutant UBAP1 leads to intracellular accumulation of post-Golgi activated STING with consequent STING-dependent constitutive type I IFN activation.

Patients with COPA mutations show accumulation of STING at the Golgi at steady state with consequent constitutive STING activation (Lepelley et al., 2020). While it has been hypothesized that COP-I acts in recycling STING from the Golgi to the ER at steady state, COP-I has been also shown to be required for proper early endosome function and autophagy maturation from the endosome (Popoff et al., 2011; Razi et al., 2009). Loss of COP-I leads to defects in endosomal trafficking and inhibition of autophagosome maturation at the endosome with consequent accumulation of ubiquitinated proteins, including p62 (Razi et al., 2009). COP-I acts on STING sorting with the adaptor SURF4 (Deng et al., 2020), identified in both the proteomics and CRISPR screen as required for STING degradation (FIG. 2C, FIG. 2E). This would suggest that COPA and SURF4 are not promoting STING recycling from the Golgi to the ER, but are actually involved in STING degradation through sorting of STING into endosomes. Indeed, when COPA K230N was expressed in the reporter STING-HA cell line, STING degradation was reduced (FIG. 15F-15H).

Therefore, it is proposed that cGAS primes constant trafficking of STING between the ER and the endosome for degradation at steady state, and functionality of an endosome specific VPS37A/UBAP1 containing ESCRT complex prevents STING accumulation with consequent spontaneous activation of downstream signaling. Mutations in genes regulating STING trafficking could represent a general disease sensitizing mechanism leading to either a lowered threshold for STING activation or directly inducing STING dependent responses via disruption of the homeostatic STING degradative flux.

### Discussion

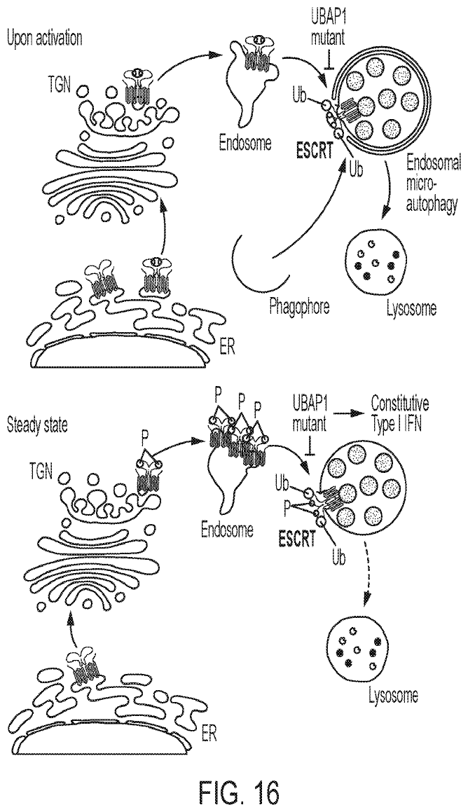

While some of the factors regulating STING exit from the ER have been characterized, the signals and genes regulating STING post-Golgi trafficking remained to be identified. To address this gap and unbiasedly identify genes involved in STING trafficking, a time-resolved map of STING neighboring proteins was generated at different intracellular compartments post cGAMP activation and a genome-wide CRISPR screen for regulators of STING degradation was carried out, providing a basis for the studies presented herein as well as resource for the field. By focusing on mechanisms of STING post-Golgi trafficking, ubiquitin was identified as the post-Golgi signal regulating STING degradation and signaling shutdown via association with an endosome-specific VPS37A/UBAP1-containing ESCRT complex. ESCRT association to ubiquitinated STING induces coordinated degradation of the sensor and resolution of autophagy in the endosomal compartment. A targeted CRISPR screen identified UBE2N as a driver of STING association with VPS37A, therefore regulating STING degradation and signaling shutdown. Similar to the signaling platform created at the TGN by phosphorylated STING for activation of IRF3 and induction of type I IFN, ubiquitinated STING in the endosomal compartment creates a platform for ESCRT recruitment driving both STING degradation and autophagy resolution (FIG. 16). Therefore, it is shown that endosomal trafficking and autophagy resolution are both part of the same ESCRT coordinated signaling shutdown mechanism.

It is becoming increasingly clear that defects in genes regulating STING trafficking can lead to human disease. Mutations in COPA lead to STING accumulation in the Golgi with spontaneous activation of the sensor. Similarly, mutations in STIM1 lead to spontaneous type I IFN activation in patients' peripheral blood mononuclear cells by releasing an ER retention signal for STING. Moreover, NPC1 deficiency has also been linked to constitutive STING activation through blockage of SREBP induced STING flux to the lysosome. It is shown that expression of a UBAP1 mutant found in patients with hereditary spastic paraplegia leads to post-Golgi accumulation of activated STING with consequent constitutive induction of type I IFN. Based on all this evidence, proposed herein is an updated model of STING trafficking in which tonic cGAMP production primes a basal flux of STING trafficking from the ER to the lysosome with consequent constant degradation. Inactivating mutations in genes controlling STING trafficking and degradation represent a generalized mechanism for inducing constitutive STING-dependent responses that could lead to disease. A similar mechanism blocking steady-state TLR7 activation has been characterized, by which UNC93B1-Syntenin1 mediated trafficking of the receptor to MVBs is required for reduction of TLR7 intracellular levels and blunting of signaling at steady state. STING is highly conserved and it is shown that accumulation of STING in the endosomal compartment is sufficient to induce STING responses. It is tempting to speculate that an ancestral function of STING in anti-pathogen defense could be sensing of perturbation of intracellular trafficking pathways due to pathogen invasion. Interestingly, STING has been shown to be activated by HSV, influenza and HIV-1 viral entry independently of nucleic acids.

Activation of exacerbated type I IFN responses has been shown to drive neuronal cell death in both cell intrinsic and extrinsic manners. Post-Golgi accumulation and constitutive type I IFN activation in cells expressing the UBAP1 mutant responsible for the development of spastic paraplegia opens the possibility that STING is involved in neurodegeneration in these patients. In addition to UBAP1, mutations in the ESCRT subunit VPS37A and the vesicular trafficking regulator AP4M1, identified in the proteomics, have also lead to HSP and been identified in patients with spastic paraplegia (Abou Jamra et al., 2011; Zivony-Elboum et al., 2012). Moreover, mutations in the ATPase domain of the ESCRT subunit VPS4A lead to a multisystem disease with abnormal neurodevelopment. As for UBAP1, it is plausible that mutations in these genes could lead to spontaneous STING dependent type I IFN activation. How defects in genes regulating STING trafficking might lead to neurodegeneration remain to be explored. Based on our model, mechanisms for disease development could include lowering the threshold with consequent increase in the magnitude of STING responses, or inducing STING-dependent type I IFN production at steady state by impairment of the homeostatic STING flux.

### Methods

### Human Cell Lines

293T (CRL-3216), hTert-BJ1 (BJ-5ta-CRL-4001) and U-937 (CRL-1593.2) were from ATCC. 293T were cultured in DMEM (Corning) supplemented with 10% FBS (VWR), 1× GlutaMax (Thermo Fisher) and 1× Penicillin/Streptomycin (Corning). hTert-BJ1 were cultured in a 4:1 mixture of DMEM (Corning): Medium 199 (Lonza) supplemented with 10% FBS (VWR), 1× GlutaMax (Thermo Fisher) and 1× Penicillin/Streptomycin (Corning). U-937 were cultured in RPMI (Thermo Fisher) supplemented with 10% FBS (VWR), 1× GlutaMax (Thermo Fisher) and 1× Penicillin/Streptomycin (Corning).

### Primary Human Cells

CD14+ monocytes were isolated from peripheral adult human blood as previously described 1. CD14+ monocytes were cultured in RPMI (Gibco) supplemented with 10% FBS (VWR), 1× GlutaMax (Thermo Fisher), 50 μg/mL Gentamicin (Thermo Fisher) and 1× Penicillin/Streptomycin (Corning). Human Monocyte Derived Dendritic Cells were differentiated as described in Gentili et al., 20192. Medium was replaced 1 days after transfection and cells were selected with 2 μg/mL Puromycin (Invivogen). Cells were replated at 0.15×10\hat{ }6c/w in a 96 well plate 5 days post differentiation, rested for 1 hour in the incubator and then stimulated with direct addition of cGAMP in the medium or with pI:C complexed with Lipofectamine.

### Constructs

The plasmids psPAX2 (#12260) and pCMV-VSV-G (#8454) were from Addgene. pSIV3+ was previously described. pTRIP-hPGK-Blast-2A was cloned from pTRIP-CMV-STING-GFP (kind gift of Nicolas Manel) by Gibson assembly of PCR amplified hPGK from pCW57-MCS1-2A-MCS2 (Addgene #71782) and a gBlock (IDT) for Blasticidin resistance. pTRIP-UbC-Blast-2A-STING-mNconGreen was cloned from Gibson assembly of PCR amplified UbC promoter from FUGW, PCR amplified Blast, PCR amplified STING and a gBlock for humanized mNconGreen described in (Tanida-Miyake et al., 2018). pTRIP-hPGK-STING-TurboID was cloned by Gibson assembly of PCR amplified TurboID from V5-TurboID-NES_pCDNA3 (Addgene #107169) and STING from pTRIP-CMV-STING-GFP. pTRIP-hPGK-Blast-2A-STING-HA/STING V155M-HA/STING R284S-HA/were cloned by Gibson assembly from PCR amplification or PCR mutagenesis. pXPR101-Hygro was cloned by Gibson assembly of a gBlock for Hygromycin resistance into pXPR101 (kind gift of Broad GPP). CROPseq-guide-Puro was a kind gift of Paul Blaincy. pXPR_BRD023 (lentiCRISPR v2) was a kind gift of the Broad GPP platform. sgRNAs were cloned by gateway cloning into the respective vectors of annealed primers. pTRIP-SFFV-mNeonGreen and pTRIP-SFFV-Blast-2A-STING-mNeonGreen were obtained by Gibson assembly. pTRIP-SFFV-Hygro-2A-mScarlet, pTRIP-SFFV-Hygro-2A-mScarlet-HGS and pTRIP-SFFV-Hygro-2A-mScarlet-VPS37A were obtained by Gibson assembly of PCR amplified Hygro from pXPR101-Hygro, PCR amplified HGS or VPS37A from U937 cDNA and PCR amplified mScarlet from pmScarlet_C1 (Addgene #85042) into pTRIP-SFFV-EGFP-NLS (Addgene #86677). pTRIP-SFFV-Hygro-2A-mScarlet-VPS4A E228Q was obtained by Gibson assembly of PCR amplified VPS4A E228Q from pEGFP-VPS4-E228Q (Addgene #80351). pTRIP-SFFV-Hygro-2A-mScarlet-UBAP1DN and pTRIP-SFFV-Puro-2A-mScarlet-UBAP1DN (truncated mutant at residue 97-mutation G98X) were obtained by Gibson assembly of a gBlock for UBAP1DN. pTRIP-hPGK-Hygro-2A-FLAG-Ubiquitin was obtained by Gibson assembly of PCR amplified Hygro from pXPR101-Hygro and a gBlock for FLAG-Ubiquitin. pTRIP-hPGK-Blast-2A-TMEM192-STING-HA was obtained by TMEM192 PCR amplification from 293T cDNA, PCR amplification of STING (aa139-379) and Gibson assembly.

### Production of Lentivirus and Lentiviral Transductions

Lentiviruses were produced as described in (Gentili et al., 2019). Briefly, 0.8 million/well 293T in a 6 well plate were transfected with 1 μg psPAX, 0.4 μg pCMV-VSV-G and 1.6 μg of viral genomic DNA with TransIT-293 (Mirus) and left O/N. To generate SIV-VLPs, cells were transfected with 2.6 μg pSIV3+ and 0.4 μg pCMV-VSV-G. Medium was then changed to 3 mL of fresh medium corresponding to the cell line to be transduced. Supernatants were harvested 30-34 hours after medium changed and filtered at 0.45 μm. 0.5 million 293T, hTert-BJ1 or U937 were infected with 2 mL of fresh virus in presence of 8 μg/mL Protamine (Millipore Sigma). To generate transduced MDDCs, 2×10\hat{ }6 freshly isolated CD14+ monocytes were transduced in a 6 well plate with 1 mL of lentivirus and 1 mL of SIV-VLPs with 8 μg/mL protamine.

### STING-TurboID

Generation of cells and stimulation. 293T cells were transduced with pTRIP-hPGK-Blast-2A-STING-TurboID and selected with 15 μg/mL Blasticidin (Invivogen) for one week. To test the construct via pull-down, 0.8 million cells were seeded in a 6 well plate. The following day, cells were permeabilized with 300 μl/well of cGAMP permeabilization buffer [50 mM HEPES (Corning), 100 mM KCl (Thermo Fisher), 3 mM MgCl₂ (Thermo Fisher), 0.1 mM DTT (Thermo fisher), 85 mM Sucrose (Thermo Fisher), 0.5 mM ATP (Cayman chemicals), 0.1 mM GTP (Cayman Chemicals), 0.2% BSA (Seracare), 0.001% Digitonin (Promega)| containing 1 μg/mL 2′3′-cGAMP (Invivogen) or water for 10 minutes at 37° C., washed with 3 mL of warm medium and then medium was replaced. For mass-spec, 6 million cells were seeded in a 10 cm dish for each condition. Cells were stimulated with 2.8 mL of cGAMP permeabilization buffer containing 6 μg total of cGAMP per dish. Cells were left stimulating for the desired times and 500 μM biotin (Cayman chemicals) was added in each well 30 minutes prior to harvest. Cells were harvested by trypsinization, washed 3 times in cold PBS and pellets were frozen until processing. For experiments in 6 well plates, 3 wells per condition were harvested. For experiments in 10 cm dishes, one dish per condition was harvested.

Pull-down. Cells were lysed on ice in 550 μL (6 well plates) or 1 mL (10 cm dishes) of RIPA buffer (Boston Bioproducts) in presence of complete, Mini, EDTA-free Protease Inhibitor Cocktail (Millipore Sigma) and PhosSTOP (Millipore Sigma) for 10 minutes. Lysates were cleared by centrifugation at 16000 g for 10 minutes at 4° C. 10% of the lysed cells was set aside as input. set aside as input. Pull-down and washes were performed as in (Branon et al., 2018). Briefly, lysates were mixed with Pierce Streptavidin Magnetic Beads (Thermo Fisher) at a ratio of 100 μl beads/4 million cells. Lysates were incubated with beads with constant rotation for one hour at room temperature and then overnight (O/N) at 4° C. Beads were then applied to a magnet and subjected to the following washes: 2 times with 1 mL of RIPA, 1 time with 1M 1 mL of KCl (Thermo Fisher), 1 time with 1 mL of 0.1M Na₂CO₃ (VWR), 1 time with 1 mL of freshly prepared 2M Urea (VWR) resuspended in 10 mM Tris-HCl pH 8.0 (Thermo Fisher) and 2 times with RIPA. Proteins were eluted from beads by adding 150 μl (6 well plates) or 500 μl (10 cm dish) of non-reducing Laemmli (Boston bioproducts) containing 20 mM DTT (Thermo Fisher) and 2 mM biotin (Cayman chemicals) and boiled for 20 minutes. Input was diluted with 2× sample buffer (Sigma). For mass spectrometry, beads were processed as follows.

Sample processing for mass spectrometry. Co-IP was performed using 2.2 mg of HEK293T cells expressing hPGK-Blasticidin-P2A-STING TurboID, 200 μL of Streptavidin beads, in duplicates, at 5 time points: not-stimulated, 30 minutes, 1, 2 and 6 hours.

Samples were received in duplicates, each in 1 mL RIPA lysis buffer. Beads were washed with 50 mM Tris HCL (200 μL, pH 7.5, 2×) and transferred to fresh 1.5 mL Eppendorf tubes. Beads were further washed with 2 M Urea/50 mM Tris HCL (200 μL, pH 7.5, 2×). Proteins were digested with trypsin (5 μg/mL, 80 μL) in 2 M urea/50 mM Tris HCL/1 mM dithiothreitol (DTT)) at 25° C. for 1 hr). Following a brief centrifugation step using a table-top centrifuge (5-10 seconds), supernatants were transferred to clean 1.5 mL Eppendorf tubes. Beads were washed once with 2 M urea/50 mM Tris HCL (60 μL, pH 7.5, 2×) and supernatants were combined with respective supernatants from the first centrifugation step. Combined supernatants were centrifuged at 5000 g for 30 s to pellet remaining beads and the supernatants were transferred to clean 1.5 mL Eppendorf tubes.

Samples were reduced with DTT (4 mM) using a shaker (1000 rpm) for 30 minutes at 25° C.) and alkylated with iodoacetamide (IAA, 10 mM) for 45 minutes at 25° C. in the dark. Proteins were digested overnight with trypsin (0.5 μg in trypsin buffer) at 25° C. using a shaker (700 rpm). Samples were acidified with formic acid (FA, 1%, 200 μL pH<3) and peptides were desalted using C18 stage tips (2 punches) following standard protocol (Mertins et al., 2018). Briefly, stage tips were activated with 50% ACN, 0.1% FA (50 μL, 1500 rcf) and conditioned with 0.1% FA (50 μL, 1500 rcf, 2×). Samples (350 μL) were loaded on the tips and spun at 1500 rcf until all volume flowed through completely without drying the stage tips. Samples were washed with 0.1% FA (50 μL, 2×, 1500 rcf), eluted with 50% ACN/0.1% FA (50 μL, 1500 rcf) and lyophilized. Peptides were subsequently reconstituted in fresh HEPES (50 mM, 95.3 μL, pH 7.5) for TMT labeling. Samples were labeled with TMT as follows: Non-Stimulated (126, 129N), 0.5 hr (127N, 129C), 1 h (127C, 130N), 2 h (128N, 130C), 6 hr (128C, 131). TMT labeling (240 μg per sample) occurred for 1 hr at room temperature with shaking (800 rpm), following standard protocol. Samples were quenched with 5% hydroxylamine (8 μL, 20° C., 700 rpm), all channels were combined in one vial and lyophilized. The combined samples were reconstituted in 0.5% acetic acid (100 μL) and fractionated following standard protocol (Rappsilber et al., 2007). Briefly, 3 SCX discs (polytetrafluoroethylene (PTFE) material) were placed in 200 μL pipette tips followed by 2×C18 discs on top. Tips were conditioned with methanol (100 μL, 3500 g, 1 minute), followed by 0.5% acetic acid/80% ACN (100 μL, 3500 g, 1 minute) and 0.5% acetic acid (100 μL, 3500 g, 1 min). Tips were equilibrated with 0.5% acetic acid (100 μL, 3500 g, 1 minute), 500 mM NH₄AcO/20% ACN (100 μL, 3500 g, 1 min) and 0.5% acetic acid (100 μL, 3500 g, 1 minute) prior to sample loading (100 μL, 3500 g). The sample was washed twice with 0.5% acetic acid (100 μL, 3500 g, 1 minute, 2×), followed by 0.5% acetic acid/80% ACN (100 μL, 3500 g, 1 minute). A stepwise elution occurred using 50 mM NH₄AcO/20% ACN (pH 5.15, 50 μL, 3500 g, 1 minute, fraction 1), 50 mM NH₄HCO₃/20% ACN (pH 8.25, 50 μL, 3500 g, 1 minute, fraction 2) and 0.1% NH₄OH/20% ACN (pH 10.3, 50 μL, 3500 g, 1 minute, fraction 3). Acetic acid (0.5%, 200 μL) was added to each eluate to reduce ACN concentration to <5%. Fractions were subsequently desalted using 2 punch C18 stage tips following the protocol described above and eluted with 80% ACN/0.5% acetic acid (60 μL, 1500 rcf). Samples were lyophilized and re-suspended in 3% ACN/0.1% FA (10 μL) for nanoLC-MS/MS analysis.

MS Analysis. Fractionated samples were analyzed on an Orbitrap Q-Exactive HF Plus MS (Thermo Fisher Scientific) equipped with a nanoflow ionization source and coupled to a nanoflow Proxcon EASY-nLC 1000 UHPLC system (Thermo Fisher Scientific). Acquisition occurred in positive ion mode. Samples were injected on an in-house packed column (22 cm×75 μm diameter C18 silica picofrit capillary column) heated at 50° C. The mobile phase flow rate was 250 nL/minutes of 3% ACN/1% FA (solvent A) and 90% ACN/0.1% FA (solvent B). Peptides were separated using the following LC gradient: 0-6% B in 1 minute, 6-30% B in 85 minutes, 30-60% B in 9 minutes, 60-90% B in 1 minute, stay at 90% B for 5 minutes, 90-50% B in 1 minute, and stay at 50% B for 5 minutes. Data was acquired in centroid mode for both MS1 and MS2 scans. Samples were analyzed in data dependent analysis (DDA) mode using a Top-12 method. Ion source parameters were: spray voltage 2 kV, source temperature 250° C. Full MS scans were acquired in the m/z range 200-2000, with an AGC target 3e6, maximum IT 10 ms and resolution 70,000 (at m/z 200). MS/MS parameters were as follows: AGC target 1e5, maximum IT 50 ms, loop count 10, isolation window 1.6 m/z, isolation offset 0.3 m/z, NCE 31, resolution 17,500 (at m/z 200) and fixed first mass 100 m/z; unassigned and singly charged ions were excluded from MS/MS.

Proteomic Data Analysis. Raw MS data were analyzed using Spectrum Mill Proteomics Workbench (prerelease version B.06.01.202, Agilent Technologies). A trypsin-specific enzyme search was performed against 2017 uniprot human fasta file (UniProt.human.20171228.RISnrNF.553smORFs.264contams) containing 65095 entries. Peptide and fragment tolerances were at 20 ppm, minimum matched peak intensity 40% and peptide false discovery rates (FDR) were calculated to be <1% using the target-decoy approach (Elias and Gygi, 2007). Fixed modifications were carbamidomethylation, TMT 10 (N-term, K) and variable modifications were Acetyl (ProN-term), Oxidized methionine (M), Pyroglutamic acid (N-termQ) and Deamidation (N). Spectra with a score<4 were filtered out. Peptides were validated using the following parameters: for charge states 2-4, a FDR of 1.2 was applied to each run and for charge state 5, a FDR of 0.6 was applied across all runs. Results were further validated at the protein level and proteins with a score of 20 or higher were accepted as valid. Reporter ion correction factors, specific to the TMT batch, were applied. A protein/peptide summary was generated using the median across all TMT channels as the denominator. Shared peptides were assigned to the protein with the highest score (SGT).

Bioinformatics Analysis. Calculated ratios at the protein level were imported into Protigy v0.8.X.X for normalization and features selection (github.com/broadinstitute/protigy). To account for variability between samples, log ratios were normalized by centering using the sample Median and scaled using the sample Median Absolute Deviation (Median MAD). A moderated F-test (Ritchie et al., 2015) was performed to identify proteins whose expression changed significantly across time points. Temporal profiles of significant proteins (FDR p-value<0.05) were z-scored and further subjected to fuzzy c-means clustering implemented in the e1071 R package. The number of clusters was set to three upon visual inspection of temporal profiles. The optimal fuzzification parameter m was determined as described in (Schwämmle and Jensen, 2010). Gene Ontology (GO) overrepresentation analysis of proteins in the resulting clusters was performed with the gProfiler R-package (Raudvere et al., 2019).

Network representation and analysis. The filtered dataset on adj p.value<0.07 was uploaded on STRING to generate a network. The network was then imported in Cytoscape (v3.8.1) and analyzed with stringApp (v1.6.0). Subcellular compartments were assigned by filtration in stringApp. Maps representing enrichments at different time-points were plotted by using the Hierarchical Clustering function in clusterMaker.

GO and Reactome enrichments. GO and Reactome enrichments present in FIGS. 9A-9B were calculated using the Functional Enrichment Analysis with values/ranks in STRING (Szklarczyk et al., 2019).

### Genome-Wide CRISPR Screen

Generation of cells and screen. 293T were transduced with pTRIP-UbC-Blast-2A-STING-mNeonGreen(mNG) and selected with 15 μg/mL of Blasticidin (Invivogen) for one week. mNG^{hi} cells were sorted on a Sony SH-800. Cells were then transduced with pXPR 101-Hygro to introduce spCas9, and kept under selection with 320 μg/mL Hygromicin for the time of culturing. Cells were then transduced with the human targeting genome-wide sgRNA library Brunello (Doench et al., 2016) at MOI 0.3 at a 1000× coverage (80 million cells). The library in lentiviral form was obtained from the Broad GPP. Cells were then selected in 2 μg/mL Puromycin and passaged maintaining 1000× representation of the library for one week. The day prior to the stimulation, cells were plated at 20 million/T225. The cells were then stimulated by adding 40 ml of fresh medium containing 1 μg/mL of 2′3′-cGAM(PS)2 (Invivogen) for 24 hours. Cells were then lifted, resuspended in MACS buffer (0.5% BSA, 2 mM EDTA in PBS) and sorted on two Sony SH-800 at a 4000× coverage (320 million total cells sorted). Cells were then pelleted, washed with PBS, and pellets were frozen until DNA extraction. DNA was extracted with DNeasy Blood & Tissue Kit (Qiagen) following manufacturer's recommendations.

Sequencing and screen analysis. Extracted DNA was submitted to the Broad Genetic Perturbation Platform (GPP) for Next Generation Sequencing. After deconvolution, reads per barcode were analyzed with the GPP Pooled Screen Analysis Tool using the Hypergeometric method (portals.broadinstitute.org/gpp/public/analysis-tools/crispr-gene-scoring).

Screen validation. 293T STING-mNG spCas9 were transduced with SEC24C or ATP6V1G1 sgRNAs cloned in CROPseq-Guide-Puro and selected with 2 μg/mL Puromycin (Invivogen) for one week. 0.016 million cells/well were plated in a 96 well plate the day prior to the stimulation, and then stimulated with 100 μL of fresh medium containing 2′3′-cGAM(PS)2 (Invivogen) for 24 hours. For HGS and VPS37A, 293T STING-mNG cells were transduced with pXPR023 (lentiCRISPR v2) expressing sgRNAs for each of the genes and selected on Puromycin for one week. 0.125 million cells/well were plated in a 24 well plate the day prior to stimulation, and then stimulated with 500 μL of fresh medium containing 2′3′-cGAM(PS)2 (Invivogen) for 24 hours.

### Ubiquitin Targeted CRISPR Screen

The library contained guides targeting 669 E3 and adaptors (compiled from Medvar et al., 201613 and Li et al., 200814), 40 E2 from Interpro, 7 E1, 28 Autophagy core proteins and 10 positive controls from the genome-wide CRISPR screen and was synthetized and cloned by the Broad GPP. Cells were generated as for the genome wide CRISPR screen. STING-mNG cells were sorted without fixation while STING-HA cells were sorted after fixation and staining as described in the flow cytometry paragraph. Both cell lines were sorted in 4 bins (top 5%, second top 5%, bottom 5%, second bottom 5%) at 4000× coverage. For STING-mNG DNA was extracted as for the genome-wide CRISPR screen. For STING-HA DNA was extracted with Quick-DNA FFPE kit (Zymo). Sequencing was performed as described in Fulco et al., 201615. Analysis was performed as for the genome-wide CRISPR screen.

### Co-Immunoprecipitation in 293 Ts

293T were transduced with either pTRIP-hPGK-Blast-2A, pTRIP-hPGK-Blast-2A-STING-HA, pTRIP-hPGK-Blast-2A-STING V155M-HA or pTRIP-hPGK-Blast-2A-STING R284S-HA and selected with 15 μg/mL Blasticidin for one week. Cells were then plated at 0.8 million cells/well in a 6 well plate and transfected with either pTRIP-SFFV-Hygro-2A-mScarlet-HGS or pTRIP-SFFV-Hygro-2A-mScarlet-VPS37A with TransIT-293 (Mirus) (3 μg DNA/well). 24 hours post-transfection, 3 wells per condition were harvested via trypsinization. Cells were washed with PBS and lysed 550 μL of Co-IP buffer (20 mM Tris-HCl PH 7.5, 150 mM NaCl, 0.5% NP-40 on ice for 30 minutes and cleared by centrifugation at 16000 g for 20 minutes). 10% of the lysate was saved as input. The lysates were then incubated with Pierce Anti-HA Magnetic Beads (Thermo Fisher) at a concentration of 100 μL beads/4 million cells O/N at 4° C. Beads were washed 5 times with Co-IP buffer and proteins were eluted by adding 150 μL of non-reducing Laemmli (Boston bioproducts) containing 20 mM DTT (Thermo Fisher) and boiled for 20 minutes. Input was diluted with 2× sample buffer (Sigma).

### Co-Immunoprecipitation in U937

U937 were transduced with either pTRIP-SFFV-mNeonGreen or pTRIP-SFFV-Blast-2A-STING-mNeonGreen. For immunoprecipitation, 5 million cells/well of a 6-well plate were seeded for each condition and stimulated with medium containing 5 μg/mL Digitonin (Promega) with or without 20 μg/mL cGAMP for 4 hours. Cells were washed with ice-cold PBS once and lysed in 500 μL Co-IP buffer (20 mM Tris-HCl pH 7.5, 150 mM NaCl, 1 mM EDTA, 0.2% NP-40) with rotation at 4° C. for 10 minutes and cleared by centrifugation at 20,000 g for 10 minutes. 10% of the lysate was saved as input. The lysates were then incubated with 30 L/sample Co-IP buffer pre-washed mNeongreen-trap Magnetic Beads (Chromotek) for 1 hour at 4° C. Beads were washed 3 times with Co-IP buffer and proteins were eluted by adding 40 μL of 2× reducing Laemmli (Sigma) and boiled for 5 minutes. Input was diluted with 2× sample buffer (Sigma).

### Flag-Ubiquitin Immunoprecipitation

293T cells were transduced with pTRIP-hPGK-Blast-2A-STING-HA and pTRIP-hPGK-Hygro-2A-FLAG-Ubiquitin and selected with 15 μL Blasticidin (Invivogen) and 320 μg/mL Hygromycin (Invivogen) for one week. 1 million cells/well were plated in a 6 well plate the day prior to the stimulation. Cells were then stimulated with 300 μL cGAMP permeabilization buffer containing 1 μg/mL cGAMP for 10 minutes, washed with 3 mL of warm medium, and the medium was replaced. 3 wells per condition were harvested 2 hours post stimulation, washed with PBS and lysed with 550 μL of RIPA buffer for 10 minutes on ice. Lysates were cleared at 16000 g for 10 minutes at 4° C. 10% of the lysate was saved as input. The remaining lysates were incubated with 150 μL Pierce Anti-DYKDDDDK Magnetic beads (Thermo Fisher) O/N at 4° C. with constant rotation. Beads were washed 3 times with a buffer containing 10 mM Tris-HCl pH7.5 (Thermo Fisher), 2 mM EDTA (Thermo Fisher), 1% Nonidet-P40 Substitute (Roche) and 50 mM NaCl (Thermo Fisher), and 2 times with RIPA buffer. Proteins were eluted by adding 150 μL of non-reducing Laemmli (Boston bioproducts) containing 20 mM DTT (Thermo Fisher) and boiled for 20 minutes. Input was diluted with 2× sample buffer (Sigma).

U937 and hTert-BJ1 Stimulation for Western Blotting

To obtain KO U937 and hTert-BJ1 for HGS and VPS37A, cells were transduced with pXPR023 expressing the corresponding guides and selected with 2 μg/mL Puromycin for one week. In regard to HGS, U937 were transduced with HGS_g01 and HGS_g02, while hTert-BJ1 were transduced with HGS_g01 and HGS_g04 (more efficient than HGS_g02). To stimulate U937, 0.2 million cells/well were seeded in a 96 well plate U bottom in 100 μL and stimulated by adding 100 μL of fresh medium containing cGAMP to a final concentration of 20 μg/mL for 6 hours. Two wells per condition were harvested 6 hours post-stimulation, washed with PBS and pellets were frozen. For hTert-BJ1, 0.25 million cells/well were seeded in a 6 well plate the day before stimulation. Cells were then stimulated with 300 μL of cGAMP permeabilization buffer containing cGAMP at 0.5 μg/mL for 10 minutes, washed with 3 mL of warm medium, and then medium was replaced. Cells were harvested at the indicated time-points post stimulation, washed with PBS, and pellets were frozen.

### U937 Stimulation for Cell Death

0.2×10\hat{ }6 cells per well were plated in a 96 well plate (U bottom) and stimulated with direct addition of 20 μg/mL cGAMP in the medium. Cells were stimulated for 24 hours. Half the cells were then recovered and used for Cell Titer Glo assay (Promega). Half the cells were stained with Annexin V Apoptosis Detection Kit (Biolegend) following manufacturer's instructions.

### Dominant Negative Transfections for STING Degradation

293T cells were transduced with pTRIP-hPGK-Blast-2A-STING-HA and selected with 15 μg/mL Blasticidin (Invivogen) for one week. 0.08 million cells/well were seeded in a 24 well plate and transfected with TransIT-293 (Mirus) with 0.5 μg/well of either pTRIP-SFFV-Hygro-2A-mScarlet, pTRIP-SFFV-Hygro-2A-mScarlet-COPA K230N, pTRIP-SFFV-Hygro-2A-mScarlet-VPS4A E228Q or pTRIP-SFFV-Hygro-2A-mScarlet-UBAP1DN and medium was replaced after O/N incubation. Cells were stimulated 40 hours post-transfection with 200 μL/well of cGAMP permeabilization buffer containing 2 μg/mL 2′3′-cGAMP for 10 minutes. Cells were then washed with 2 mL of medium and medium was replaced. Cells were lifted and stained as indicated in the Flow Cytometry paragraph.

Treatments with Drugs

MLN7243 (Selleckchem) was used at 0.5 μM in all experiments. All cell lines were pre-treated for one hour before cGAMP stimulation. 293T STING-mNeonGreen were plated the day before stimulation in a 24 well plate at 0.2 million cells/well and were stimulated by adding 4 μg/mL 2′3′-cGAM(PS) in the medium for 6 hours. hTert-BJ1 were seeded the day before stimulation in a 6 well plate at 0.25 million cells/wells. Cells were stimulated with 300 μL cGAMP permeabilization buffer containing 0.5 μg/mL 2′3′-cGAMP for 10 minutes, washed with 3 mL of medium, and medium was then replaced. MLN7243 was added again after medium replacement. Cells were stimulated for the indicated times. Bortezomib final concentration was 1 μM, MG-132 2 μM, BafA1 100 nM.

### Flow Cytometry

For flow cytometry analysis of 293T STING-mNG, cells were lifted with TrypLE (Thermo Fisher), washed in medium and resuspended in FACS buffer (1% BSA, 1 mM EDTA, 0.01% NaN₃ in PBS). For experiments involving intracellular staining of HA, 293T expressing HA-tagged WT or STING mutants, cells were lifted with TrypLE (Thermo Fisher), washed with PBS and stained using BD Cytofix/Cytoperm (BD Biosciences). Cells were fixed in Cytofix for 1 hours, washed twice with Cytoperm, and stained with Alexa Fluor 647 anti-HA.11 Epitope Tag Antibody (BioLegend) for one hour. Cells were then washed twice with Cytoperm and resuspended in FACS buffer for flow cytometry. Acquisition was performed on a Cytoflex S or Cytoflex LX (Beckman Coulter). Data was analyzed with FlowJo (BD).

### Immunofluorescence

293T STING-TurboID were seeded directly on coverslips. 293T STING-HA were seeded on Fibronectin bovine plasma (Sigma-stock: 100×) coated coverslips. Cells were seeded the day before stimulation at 0.1 million cells/well density in 24 well plates. Cells were stimulated with cGAMP permeabilization buffer containing 1 μg/mL cGAMP for 10 minutes, washed with warm medium, and incubated for the indicated times. hTert-BJ1 mScarlet or mScarlet-UBAP1DN were seeded Fibronectin bovine plasma coated coverslips at 0.05 million cells/well density in a 24 well plate and fixed 6 hours post seeding. Cells were then fixed with 2% Paraformaldehyde (Electron Microscopy Sciences) in PHEM buffer (Electron Microscopy Sciences) for 30 minutes at 37° C., washed three times with PBS and quenched with freshly prepared 0.1M Glycine for 10 minutes. Coverslips were then permeabilized and blocked with 10% goat serum (Thermo Fisher) in PBS, 0.5% BSA (Seracare), 0.05% Saponin from Quillaja bark (Sigma) for 30 minutes. Coverslips were then stained with primary antibodies for 1-2 hours at room temperature in PBS, 0.5% BSA (Seracare), 0.05% Saponin from Quillaja bark (Sigma) with 10% goat serum, washed 5 times, and then stained with secondary antibodies in PBS, 0.5% BSA (Seracare), 0.05% Saponin from Quillaja bark (Sigma) for 1-2 hours. Coverslips were then washed 5 times, mounted with Fuoromont-G, with DAPI (Thermo Fisher) and dried at 37° C. for one hour. Images were acquired on an Olympus IX83 using an Olympus PlanApo N 60×1.42NA oil immersion objective controlled by Fluoview software. Images were analyzed in FiJi. TMEM192-STING images in FIGS. 15A, 15C-15E, and 15I-15O were acquired on a Ti-2 Eclipse inverted epifluorescence microscope (Nikon) using a 20×0.75 NA CFI Plan Apo λ objective (Nikon).

### Western Blotting

Samples for pull-downs or immunoprecipitations were treated as described in the corresponding paragraphs. For experiments involving seeding of cells in 6 well plates or 24 well plates (293 Ts and hTert-BJ1), one well per condition was harvested, lysed in RIPA buffer (Boston Bioproducts) containing complete, Mini, EDTA-free Protease Inhibitor Cocktail (Millipore Sigma) and PhosSTOP (Millipore Sigma) for 10 minutes on ice. Lysates were cleared by centrifugation at 16000 g for 10 minutes at 4° C. and Laemmli 6×, Sample buffer, SDS, and reducing agents (Boston Bioproducts) were added prior to loading. Samples were run on NuPAGE 4 to 12%, Bis-Tris Gels (Thermo Fisher) and transferred on nitrocellulose membrane with an iBlot2 (Thermo Fisher). Membranes were blocked in 5% non-fat milk in TBS Tween. Antibodies against phospho-proteins were incubated in 5% BSA TBS tween. ECL signal was recorded on a ChemiDoc Biorad Imager. Data was analyzed with ImageLab (Biorad).

### RNAseq

RNA Isolation. 0.25 million cells/well in a 6 well plate were plated the day before harvesting in triplicates. Cells were left in culture for 24 hours before harvesting. RNA was isolated using the AllPrep DNA/RNA Mini Kit (Qiagen #80204). Following total RNA extraction, mRNA was purified using Dynabeads® Oligo(dT)25 (ThermoFisher #61005). Generation of RNA-seq libraries. Bulk RNA-seq libraries were assembled from purified mRNA using the Smart-Seq3 workflow (Hagemann-Jensen et al., 2020). The reactions were scaled 8-fold per sample, using 15 ng input mRNA. First-strand synthesis and template switch reactions were performed by combining RT mix 1 and RT mix 2 and incubated in the thermocycler conditions noted in table First-strand synthesis. cDNA amplification was performed using 20 μL of the first-strand synthesis reaction as input in the cDNA PCR mix and thermocycled as noted in table cDNA amplification. Amplified cDNA was cleaned up using 0.8× concentration of Ampure XP beads (Beckman Coulter #A63881). Following clean up, cDNA samples underwent tagmentation and subsequent final library amplification. Briefly, 4 ul of tagmentation mix was combined with 400 pg of cDNA sample diluted in 4 μL of H₂O. The tagmentation reaction was incubated at 55° C. for 10 minutes, with reaction being stopped with addition of 2 μL 0.2% SDS. Tagmented samples were then added to the final library mix and amplified in the conditions noted in table Final library amplification. Final libraries were cleaned using 0.8× concentration of Ampure XP beads (Beckman Coulter #A63881) and quantified using the Agilent Bioanalyzer High Sensitivity DNA (Agilent #5067-4626) system.

RNA-Seq Analysis. Libraries were sequenced on an Illumina NextSeq 500 with paired-end reads at a depth of 10-15 million reads per sample. Reads were extracted and demultiplexed using bc12fastq2 (v. 2.20.0) and sequencing quality was assessed with FastQC (v. 0.11.9), after which adapter and quality filtering was performed with Cutadapt (v. 3.1) (Martin, 2011). Reads were mapped with Salmon (v. 1.4.0) (Patro et al., 2017) using a whole genome decoy-aware transcriptome index built from GENCODE GRCh38 release 36, and mapping rates for all samples were between 90-93%. Differential expression analysis was performed in R with DESeq2 using a standard workflow. The criterion for identifying DEGs was padj≤0.01. Volcano plots were plotted with VolcaNoseR (Goedhart and Luijsterburg, 2020). GO enrichment plots were calculated using the Functional Enrichment Analysis in STRING.

List of sgRNAs

| sgRNA name | sgRNA sequence | Vector | SEQ ID NO: |
|---|---|---|---|
| ntgRNA | GTATTACTGATATTGGTGGG | CROPseq-guide-Puro or pXPR BRD023 | 8 |
| SEC24C_g01 | GAAGATGACAGGAACAACCG | CROPseq-guide-Puro | 9 |
| SEC24C_g02 | AAGAGCCCAGGGATAGCTCA | CROPseq-guide-Puro | 10 |
| ATP6V1G1_g01 | TGAACAGTACCGCCTGCAGA | pXPR_BRD023 | 11 |
| ATP6V1G1_g02 | TGCCACGGGATCCCAATGCC | pXPR_BRD023 | 12 |
| HGS_g01 | CTTGGGGTACGAAGTGTACG | pXPR_BRD023 | 13 |
| HGS_g02 | GCACGCAGCCCCCACCAACG | pXPR_BRD023 | 14 |
| HGS_g04 | CAGCCCCTCATAGTACACTG | pXPR_BRD023 | 15 |
| VPS37A_g01 | CATAAGGAGACATCCCACTT | pXPR_BRD023 | 16 |
| VPS37A_g02 | GGTGGATAAACACTGATCAC | pXPR_BRD023 | 17 |
| STING_g01 | CATTACAACAACCTGCTACG | pXPR_BRD023 | 18 |
| cGAS_g01 | ATCCCTCCGTACGAGAATGG | pXPR_BRD023 | 19 |
| UBE2N_g01 | TGGTTCTGCCAGCAAACGCT | pXPR_BRD023 | 20 |
| UBE2N_g02 | CTGTTGCCTTCATAGATAAG | pXPR_BRD023 | 21 |

### List of Antibodies

| Target | Type | Application | Source | Reference |
|---|---|---|---|---|
| STING (D2P2F) | Rbt monoclonal | WB and IF | CST | 13647S |
| STING | Ms monoclonal | WB | R&D | MAB7169 |
| pSTING (Ser366) (D7C3S) | Rbt monoclonal | WB | CST | 19781S |
| pSTING (Ser366) (D8K6H) | Rbt monoclonal | IF | CST | 40818S |
| TBK1 (E8I3G) | Rbt monoclonal | WB | CST | 38066S |
| TBK1 (E9H5S) | Ms monoclonal | WB | CST | 51872S |
| pTBK1 (Ser172) (D52C2) | Rbt monoclonal | WB | CST | 5483S |
| IRF3 (D6I4C) | Rbt monoclonal | WB | CST | 11904S |
| pIRF3 (Ser386) (E7J8G) | Rbt monoclonal | WB | CST | 37829S |
| STAT1 | Rbt monoclonal | WB | CST | 9172S |
| cGAS (D1D3G) | Rbt monoclonal | WB | CST | 15102S |
| pSTAT1 (Tyr 701) (58D6) | Rbt monoclonal | WB | CST | 9167S |
| MX1 (D3W7I) | Rbt monoclonal | WB | CST | 37849S |
| HRS (D7T5N) | Rbt monoclonal | WB and IF | CST | 15087S |
| LC3B | Rbt monoclonal | WB and IF | CST | 2775S |
| p62 (D5L7G) | Rbt monoclonal | IF | CST | 88588S |
| Ubc13 | Rbt monoclonal | WB | CST | 4919 |
| CD63 (EPR22458-28 0) | Rbt monoclonal | IF | Abcam | ab252919 |
| CD63 (MX-49.129.5) | Ms monoclonal | IF | Santa Cruz | sc-5275 |
| VPS37A (G-3) | Ms monoclonal | WB | Santa Cruz | sc-376978 |
| NBR1 (4BR) | Ms monoclonal | IF | Santa Cruz | sc-130380 |
| UBAP1 | Rbt polyclonal | WB | Proteintech | 12385-1-AP |
| RFP (6G6) | Ms monoclonal | WB | Chromotek | 6g6-100 |
| GM130 (35/GM130) | Ms monoclonal | IF | BD Biosciences | 610823 |
| EEA1 (14/EEA1) | Ms monoclonal | IF | BD Biosciences | 610457 |
| β-actin (AC-15) | Ms monoclonal | WB | Abcam | ab6276 |
| α-Tubulin (DM1A) | Ms monoclonal | WB | Thermo Fisher | 14-4502-82 |
| Vinculin | Rbt monoclonal | WB | CST | 4650S |
| HRP Anti-beta Actin | Ms monoclonal | WB | Abcam | ab49900 |
| antibody [AC-15] | | | | |
| HRP-anti β-actin | Ms Monoclonal - | WB | Abcam | ab20272 |
| (mAbcam 8226) | HRP conjugate | | | |
| HRP-Streptavidin | NA | WB | BioLegend | 405210 |
| HA | Rat monoclonal | IF | Millipore Sigma | 11867423001 |
| DYKDDDDK Tag (D6W5B) - FLAG | Rabbit | WB | CST | 14793S |
| | monoclonal | | | |
| Cy5-Streptavidin | NA | IF | Biolegend | 405209 |
| Alexa Fluor 647 anti-HA.11 | Biolegend | FC | Biolegend | 682404 |
| Alexa 488 anti-mouse IgG (H + L) | Gt polyclonal | IF | Thermo Fisher | A-11029 |
| Alexa 555 F(ab′)2 anti-Rabbit IgG | Gt polyclonal | IF | Thermo Fisher | A-21430 |
| Alexa 647 anti-rat IgG (H + L) | Gt polyclonal | IF | Thermo Fisher | A-21247 |
| Peroxidase AffiniPure Goat | Gt polyclonal | WB | Jackson | 111-035-144 |
| Anti-Rabbit IgG (H + L) | | | Immunoresearch | |
| Peroxidase AffiniPure Goat | Gt polyclonal | WB | Jackson | 111-035-146 |
| Anti-Mouse IgG (H + L) | | | Immunoresearch | |

Rbt: rabbit; Ms: mouse; Gt: goat.
WB: western blot; IF: immunofluorescence; FC: flow cytometry.

### RNA Sequencing Reagents and Conditions

RT mix 1

| Reagent | Reaction conc. | | μL required |
|---|---|---|---|
| PEG 8000 (50% solution) | 5% | | 3.2 |
| Triton X-100 (10% solution) | 0.10% | | 0.24 |
| RNase Inhibitor (40 μ/μL) | 0.5 | μ/μL | 0.32 |
| Smartseq3_OligodT30VN(100 μM) | 0.5 | μM | 0.16 |
| dNTPs (10 mM/each) | 0.5 | mM/each | 1.6 |

| | |
|---|---|
| mRNA template | μL for 15 ng |
| Nuclease Free Water | Up to 24 μL |
| Total μL | 24 |

RT mix 2

| Reagent | Reaction conc. | | μL required |
|---|---|---|---|
| Tris-HCl pH 8.5 (1M) | 25 | mM | 0.8 |
| NaCl (1M) | 30 | mM | 0.96 |
| MgCl2 (100 mM) | 2.5 | mM | 0.8 |
| GTP (100 mM) | 1 | mM | 0.32 |
| DTT (100 mM) | 8 | mM | 2.56 |
| RNase Inhibitor (40 u/μL) | 0.5 | u/μL | 0.4 |
| TSO (100 μM) | 2 | μM | 0.64 |
| Maxima H-minus RT enzyme (200 U/μL) | 2 | u/μL | 0.32 |

| | |
|---|---|
| Nuclease Free Water | 1.2 |
| Total μL | 8 |

First-strand synthesis

| | | |
|---|---|---|
| Temperature | Time | Cycles |

| | | | |
|---|---|---|---|
| 42° C. | 90 | min | 1x |
| 50° C. | 2 | min | 10x |
| 42° C. | 2 | min | |
| 85° C. | 5 | min | 1x |

cDNA PCR mix

| Reagent | Reaction conc. | | μL per. reaction |
|---|---|---|---|
| Kapa HiFi HotStart buffer (5X) | 1X | | 10 |
| dNTPs (10 mM/each) | 0.3 | mM/each | 1.5 |
| MgCl2 (100 mM) | 0.5 | mM | 0.25 |
| Fwd Primer (100 μM) AAO270 | 0.5 | μM | 0.25 |
| Rev Primer (10 μM) AAO271 | 0.1 | μM | 0.5 |
| First-strand synthesis reaction | | | 20 |
| Polymerase (1 U/μL) | 0.02 | U/μL | 1 |

| | |
|---|---|
| Nuclease Free Water | 16.5 |
| Total μL | 30 |

cDNA amplification

| | | |
|---|---|---|
| Temperature | Time | Cycles |

| | | | |
|---|---|---|---|
| 98° C. | 3 | min | 1x |
| 98° C. | 20 | sec | 25x |
| 65° C. | 30 | sec | |
| 72° C. | 6 | min | |
| 72° C. | 5 | min | 1x |

| | |
|---|---|
| 4° C. | Hold |

Tagmentation mix

| Reagent | Reaction conc. | μL per. reaction |
|---|---|---|
| Tagmentation buffer (4x) | 1X | 2 |
| (Tris-HCl pH 7.5 - 40 mM, | | |
| MgCl2 - 20 mM, DMF- 20%) | | |
| Amplicon Tagmentation | | 0.32 |
| Mix (Tn5) | | |
| UltraPure water | | 1.68 |
| Total | | 4 |

Final library amplification mix

| Reagent | Reaction conc. | | μL per. reaction |
|---|---|---|---|
| Phusion HF buffer (5X) | 1X | | 5.6 |
| dNTPs (10 mM/each) | 0.2 | mM/each | 0.6 |
| Phusion HF (2 U/μL) | 0.01 | U/μL | 0.16 |
| Nextera Index Primers (0.5 μM) | 0.1 | μM | 6 |

| | |
|---|---|
| H₂O | 5.64 |
| Tagmented cDNA | 10 |
| Total μL | 28 |

Final library amplification

| | | |
|---|---|---|
| Temperature | Time | Cycles |

| | | | |
|---|---|---|---|
| 72° C. | 3 | min | 1x |
| 98° C. | 3 | min | 1x |
| 98° C. | 10 | sec | 12x |
| 55° C. | 30 | sec | |
| 72° C. | 30 | sec | |
| 72° C. | 5 | min | 1x |

| | |
|---|---|
| 4° C. | Hold |

### Example 2: Oligomerization Drives Concentration Dependent STING Activation

STING is the sensor of cyclic di-nucleotides of bacterial origins (Burdette et al. 2011) and 2′3′-cGAMP produced inside mammalian cells by dsDNA sensor cGAS (Sun et al. 2013; Wu et al. 2013; Ablasser et al. 2013). As a transmembrane protein, STING localizes on the ER surface as a homodimer form. Upon binding with agonists, the STING dimer goes through a 180-degree rotation to its activated conformation and translocates from ER through Golgi apparatus to the endosome and degrades at the lysosome (Shang et al. 2019). Upon STING trafficking out of ER, it activates TBK1-IRF3 pathway for IFN production (Tanaka and Chen 2012) and induces autophagy (Gui et al. 2019). Localization of STING is crucial for STING activity. The blockade of STING ER exit by BFA or bacteria derived protein VirA inhibits STING activity (Dobbs et al. 2015) while the blockade of STING degradation at the endolysosome further boost its activity (Gonugunta et al. 2017). In vitro biochemistry and structure analysis provide invaluable information about details about the STING conformation switch upon ligand binding, oligomerization interface and demonstrate that TBK1 needs to trans-phosphorylation STING (Shang et al. 2019; Zhang et al. 2019; Zhao et al. 2019).

While there is no difference of STING TBK1 binding with/without cGAMP stimulation through Co-IP and STING can be activated by TBK1 upon cGAMP stimulation in vitro (Zhang et al. 2019; Zhao et al. 2019), these did not reflect STING behaviors inside the intact cells. STING without agonists or blockade of STING trafficking out of ER upon stimulation failed to recruit TBK1 and blocked STING activation, suggesting a unique localization dependent STING activity controlling mechanism in the intact living cells (Dobbs et al. 2015; Zhang et al. 2019; Zhao et al. 2019). Moreover, while agonist binding is required for oligomerization, agonist-independent STING activation has also been observed in different settings. While STING overexpression is sufficient to drive its activation (Ishikawa and Barber 2008; Sun et al. 2009; Zhong et al. 2008), mislocalization of STING protein has also been shown to activate the TBK1-IRF3 pathway without cGAMP in COPA mutant patient cells (Deng et al. 2020; Lepelley et al. 2020; Mukai et al. 2021), further challenging the classic model that agonist driven oligomerization process is essential for the actual TBK1 activation step.

At the same time, given the importance of STING translocation, the driving force of STING translocation is still highly debatable. Agonist binding or mutants in the connector helix induce STING dimer rotation, triggering STING trafficking and STING oligomerization (Shang et al. 2019). Given that palmitoylation inhibition at TGN impairs STING oligomerization dramatically without influencing STING ER translocation (Haag et al. 2018), it is unclear if oligomerization drives translocation or translocation drives oligomerization. Moreover, SAVI mutants outside the STING connector helix can also translocate and induce STING activation, further complicating the interpretation but also providing vital information for discovering the actual driving force of STING translocation.

For a long time, agonist independent STING activation and SAVI mutants outside connector helix were “outliers” of the classic STING activation process. As a result of the information provided by these outlier phenomena, three questions were sought to be answered: 1. How does localization regulate STING activity?, 2. Which factor controls STING translocation?, and 3. What genes control STING activity and the mechanism of the regulation?

### Results

### STING Density Control its Trans-Phosphorylation but not Autophagy Induction

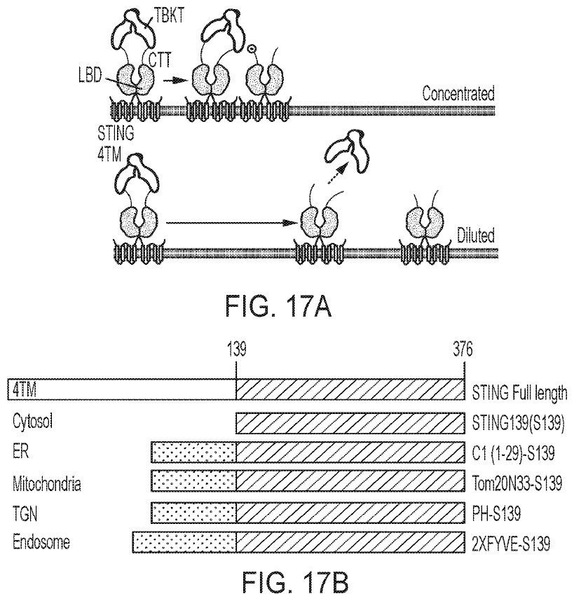

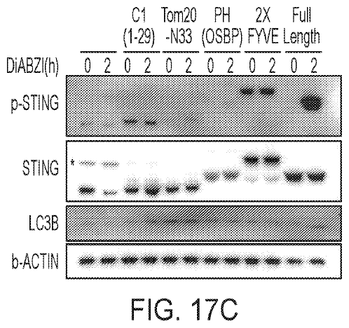

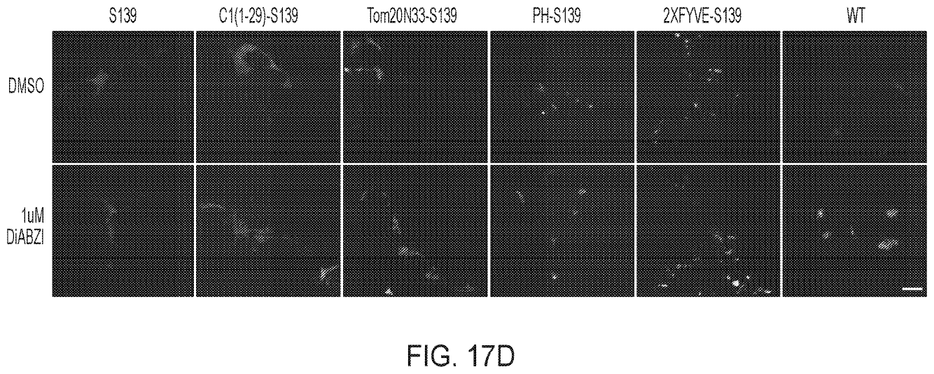

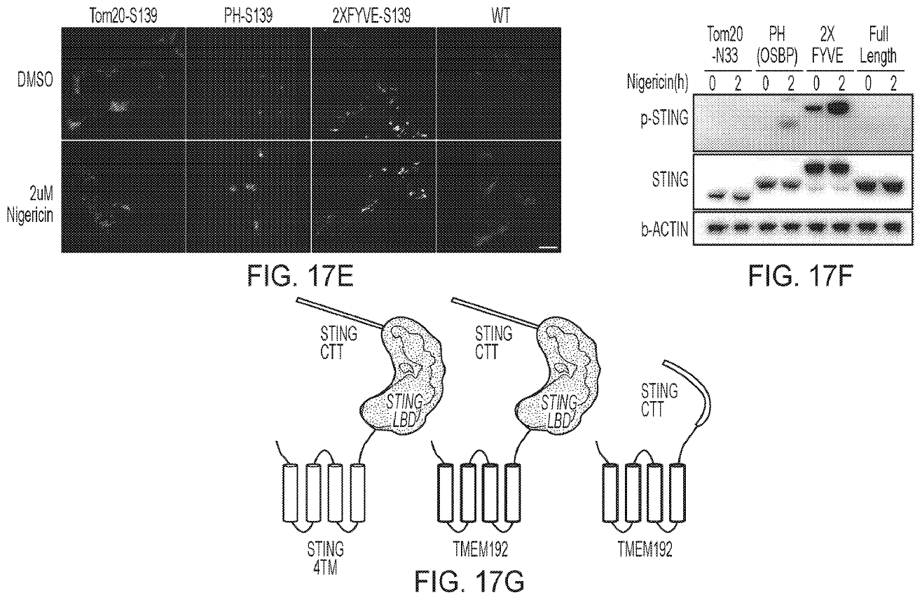

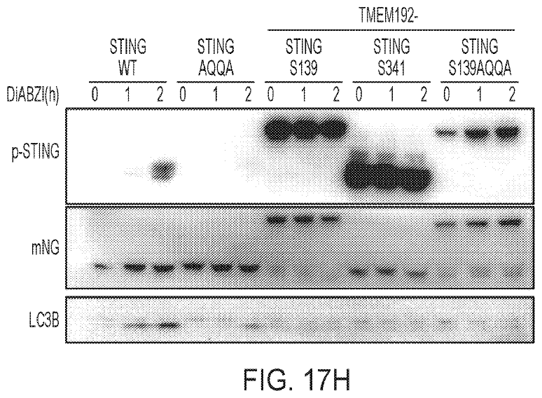

STING consists of N terminal 4 transmembrane domains and a C-terminal LBD (ligand binding domain). STING activation depends on its conserved PLPLRT/SD TBK1 binding motif and STING phosphorylation site in the C terminal tail region (CTT) (Zhang et al. 2019; Zhao et al. 2019). The structure of the STING-TBK1 complex provides clear evidence of a “trans-phosphorylation model”, that TBK1 needs to phosphorylate neighboring STING rather than the “anchoring” STING it binds due to close distance of TBK1 binding site and phosphorylation site on STING. TBK1 binds with STING directly on its CTT site. However, the affinity is ˜100 μM, suggesting a very unstable binding (Zhao et al. 2019). Combined with the fact that STING can only be phosphorylated after translocation from ER, it was hypothesized that STING activation depends on its concentration on the membrane (FIG. 17A). On organelles with small surface area, STING is more concentrated and the temporal bound TBK1 on one STING molecule is able to reach the neighboring STING to phosphorylate it before falling off. On the organelles with a big surface area, TBK1 falls off the anchoring STING before it reaches the neighboring STING, and thus is unable to activate STING signaling. Following this prediction, concentrated STING on organelles with small surface area will induce STING activation, while sparsely distributed STING on organelles will not be able to induce its downstream signaling.

The dynamic nature of the trafficking process upon STING activation makes it hard to precisely perturb STING trafficking in the living cells before. In order to test this, the STING transmembrane domain was truncated and human STING LBD (139-376) was fused with different organelle targeting sequences on its N terminal (FIG. 17B). Following the STING trafficking route, STING was fixed at each compartment it travelled through to have a better understanding of localization effects. C1 (1-29) was chosen as the ER localization domain, PI4P binding PH domain as TGN localization (Chen and Chen 2018) domain and PI3P binding FYVE domain as endosome localization domain (partially in TGN) (Lobingier et al. 2017). Additionally, Tom20N33 (Kanaji et al. 2000) was also chosen as a mitochondrial outer membrane localization domain to target STING to mitochondria that WT STING does not traffic. WT full length STING translocates out of ER membrane upon simulation while a dramatic difference of localization in all the STING fusion proteins was not observed (FIG. 17D). As expected, a strong STING phosphorylation was observed in 2XFYVE STING fusion while other fusion STING gave either no or little phosphorylation (FIG. 17C), which is consistent with knowledge that STING is activated when trafficking to TGN, endosome site, suggesting that STING density is indeed an important factor for its activation. Surprisingly, the STING phosphorylation level in these STING fusion proteins seemed independent of STING agonists (FIG. 17C), suggesting that localization change was sufficient to induce signaling potentially through density change. In order to explore if concentrating STING was sufficient to induce its activation, the known phenomenon that Nigericin promotes TGN dispersion (Chen and Chen 2018) and phosphoinositide concentration on the dispersed TGN was used. Consistently, PH-S139 localization change was observed to dispersed vesicles after nigericin stimulation (FIG. 17E). As predicted, STING activation was observed in PH-S139 and 2XFYVE-S139 but not with mitochondria localized TomS33-S139 or WT STING (FIG. 17F), which further supports the hypothesis that concentration change of STING LBD is sufficient to induce its phosphorylation. Other than STING-TBK1-IRF3 mediated IFN production, STING is also known to induce autophagy. However, how STING induced autophagy is initiated is still debatable. Interestingly, while 2XFYVE induces comparable phospho-STING signal as WT STING stimulated with agonist, only WT STING activation induced LC3 lipidation indicates that localized STING LBD on the TGN-endosome is not sufficient to induce autophagy. Given there is no transmembrane domain in 2XFYVE-S139, it was wondered whether the trafficking route of 2XFYVE-S139 could be the reason since it does not need Sec24C (Gui et al. 2019) to exit ER to TGN-endosome due to lack of transmembrane domain. Thus, S139 was fused with endolysosome localized TMEM192 (Abu-Remailch et al. 2017) which also has 4 transmembrane domains and similar topology as the STING 4TM region (FIG. 17G). Interestingly, the endolysosome localized TMEM192-S139 (FIG. 17H) induced much stronger pSTING signaling than WT STING with agonist, while TMEM192-S139 almost induces no LC3 lipidation (FIG. 17I). Most surprisingly, fuse STING CTT with TMEM192 (TMEM192-S341) is able to induce as strong pSTING signal as TMEM192-S139 indicate that the CTT motif concentration is the key factor for STING activation, this is further supported by the STING phosphorylation of TMEM192-S139AQQA (A277QQ273A) mutants which is deficient of STING oligomerization (FIG. 17I).

### Oligomerization Control STING Trafficking

While STING density change is driven by its translocation, it is unclear what is the driven force of the translocation process. Ligand binding or SAVI mutants at the connector helix induces STING dimer rotation and oligomerization (Shang et al. 2019). STING oligomerization is well conserved even to STING analogue in bacteria (Morehouse et al. 2020). STING oligomerization was explored to determine whether it was the cause of STING translocation.

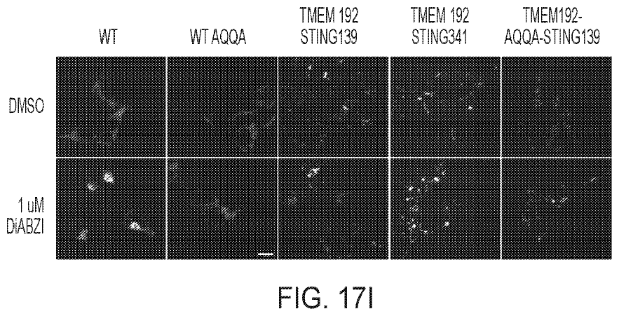

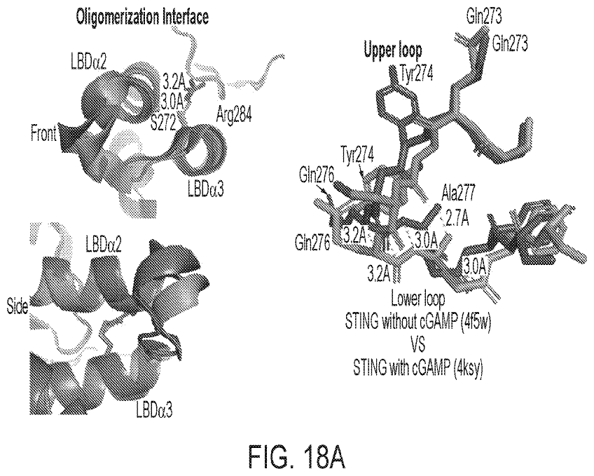

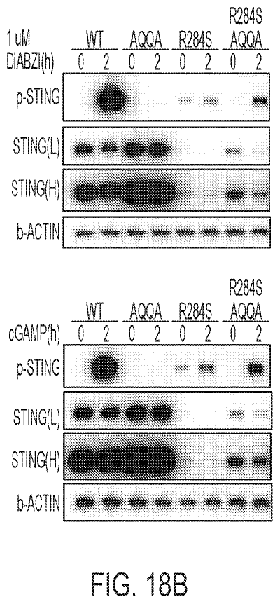

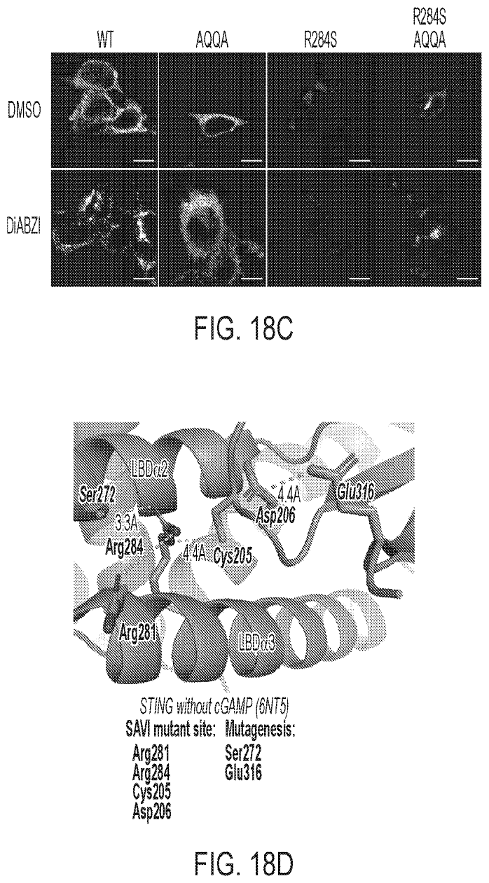

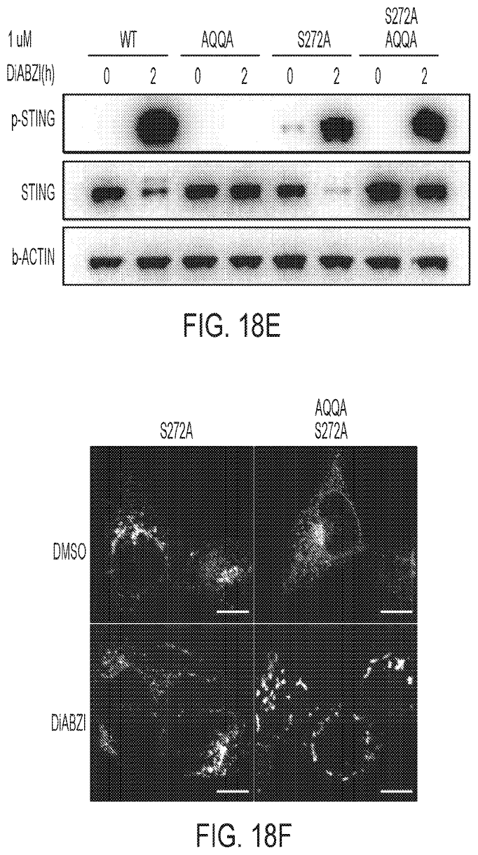

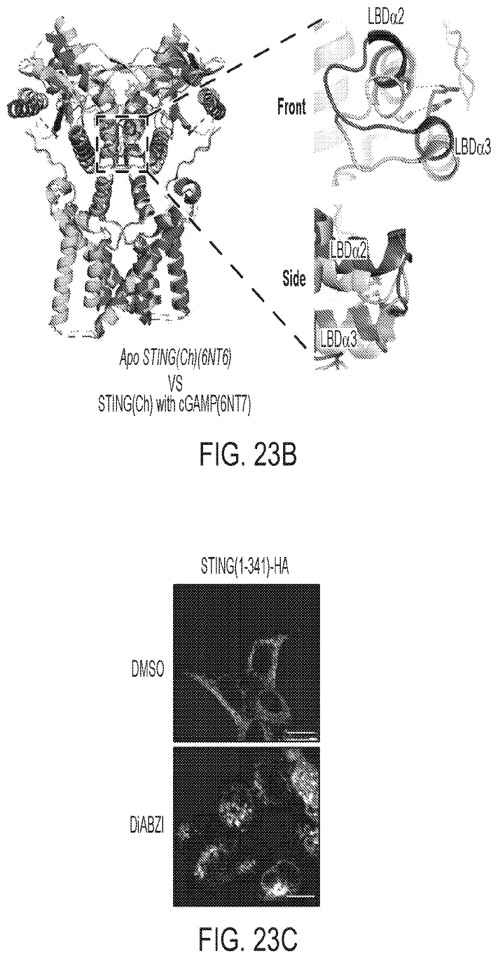

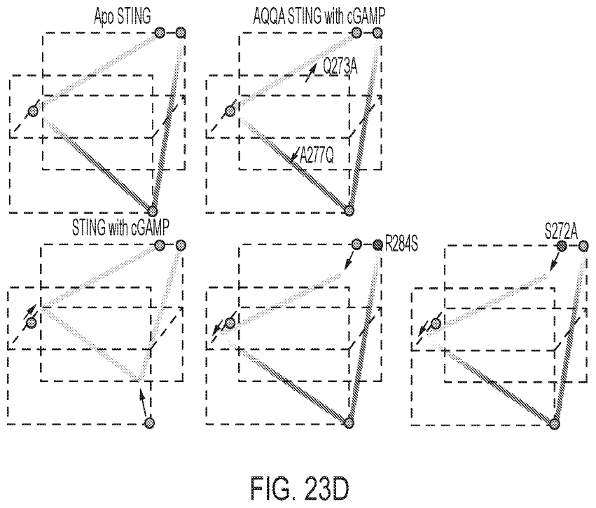

The cryo-EM chicken STING tetramer structure illustrates the packing pattern of the STING dimer and provides important insights into unresolved tetramer interface through modeling with STING dimer structure. Modeling using the chicken STING inactive dimer and active dimer illustrate the importance of movement of the oligomerization motif between LBDα2 and LBDα3 for mediating the oligomerization process (Shang et al. 2019). While the details of the interface are still not clear, a similar trend of cGAMP binding induced oligomerization motif movement in human STING (FIG. 23A and FIG. 18A) and chicken STING (FIG. 23B) was observed. Upon cGAMP binding, the distance between the end of LBDα2 and LBDα3 shortened, which induced backward movement of the lower loop of the oligomerization motif and relaxation of the oligomerization motif (FIG. 23B and (Shang et al. 2019)). It was hypothesized that the backward movement of lower loop and the relaxation of the oligomerization motif made the dimer able to oligomerize.

Mutations that push the lower loop further outward or induce upper further inward should theoretically tense the oligomerization motif and block oligomerization even with agonist binding. As expected, mutating Ala277 in the inhibitory loop to Glu together with Glu273 in the oligomerization motif to alanine (AQQA) inhibited STING activation and also its oligomerization. Interestingly, the AQQA mutant was also incapable of trafficking out of ER (FIG. 18C), suggesting that oligomerization is required for STING translocation (FIG. 23D) (Shang et al. 2019).

Next, oligomerization was explored to determine whether it was sufficient to induce STING translocation starting from understanding SAVI mutants outside the connector helix. SAVI mutants outside the connector helix like Arginine284 mutants theoretically should not induce dimer rotation as connector helix mutants but have been reported to oligomerize. While the mechanism of R284 SAVI mutant is still debated, paper reports CTT release in R284 mutant is the cause of the oligomerization and translocation (Ergun et al. 2019). STING (1-341) (CTT deletion mutant) did not show automatic translocation like STING R284S and only translocated upon agonist stimulation (FIG. 23C), this is further supported by the evidence that ancestral STING without CTT also localizes at ER without agonists, suggesting a different mechanism for R284S to be constitutively active.

Given the importance of the oligomerization motif for mediating STING oligomerization, perturbing the oligomerization motifs was explored to determine whether they were the reason for R284S's constitutive activity (FIG. 18A). When comparing STING apo and cGAMP bound, the proximity of Ser272 and Arg284 in both states of STING was observed, suggesting the Ser-Arg interaction inhibits the flexibility of oligomerization motif. Thus, Arg284 mutation would release the upper loop of the oligomerization motif to relax the oligomerization interface similar to agonist binding induced lower motif backward and oligomerization motif relaxation (FIG. 23D). To test this hypothesis, STING was constructed with both SAVI R284S mutant and AQQA mutant. If the function of R284S induces upper loop outward and oligomerization loop relaxation, the R284S mutant should be neutralized by the AQQA mutant given that AQQA tenses the oligomerization more and becomes a mutual intragenic suppressor mutation. Consistent with this prediction, while AQQA STING was constitutively inactive and R284S was constitutively active, R284S AQQA STING did not have automatic activation and could be activated by STING agonists. This was confirmed by STING phosphorylation (FIG. 18B) and localization change (FIG. 18C). Given the importance of the Ser272-Arg284 lock, mutating Ser272 would also likely destroy the lock and induce constitutive activation like R284 mutants. As expected, the Ser272A mutant indeed induced automatic activation and was also an intragenic suppressor to AQQA mutant as R284S (FIGS. 18E-18F). Surprisingly, amino acids that were predicted to stabilize Arg284 were also shown to be SAVI causing sites (R281, C205, D206) (FIG. 18D). Mutagenesis of mouse STING (Burdette et al. 2011) identified S271 (human S272) and E315 (human E316) mutants could also cause STING constitutive activation. Results showed that E316 was able to stabilize D206 position through repulsive force (FIG. 18D), which further emphasized the importance of the S272-R284 lock for mediating STING autoinhibition without agonists.

While STING oligomerization is required for its dramatic translocation, blockade of STING retrograde trafficking in COPA mutation patients induced STING accumulation at TGN, indicating STING going through a low level of constitutive trafficking out of ER. Upon agonist binding, STING binds to its trafficking adaptor Sec24C stronger (Gui et al. 2019), inducing a dramatic trafficking out of ER and induce the downstream signaling. The enhanced Sec24C binding is likely to come from the increased avidity of STING oligomer.

### Genome-Wide CRISPR Screening Systematically Identify STING Activity Regulators of Different Biological Processes

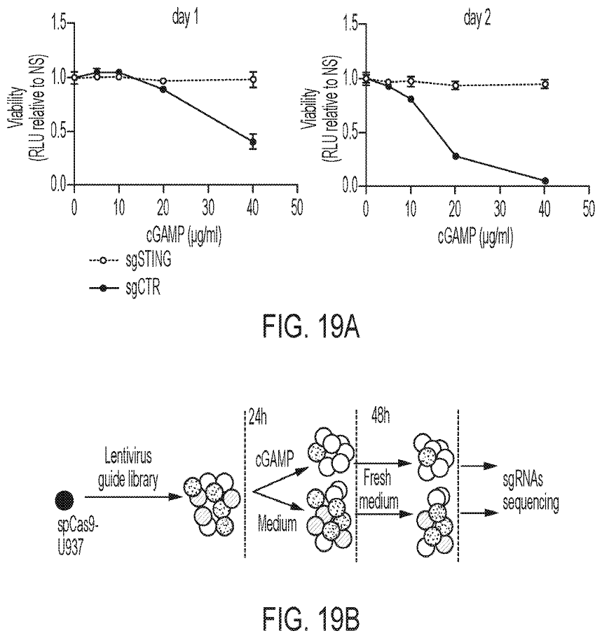

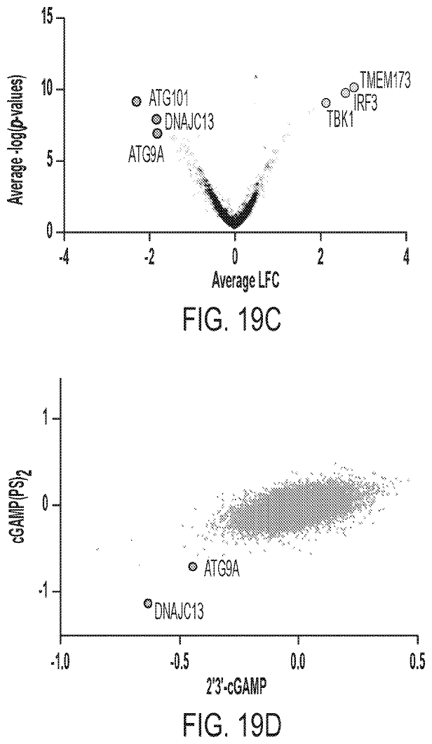

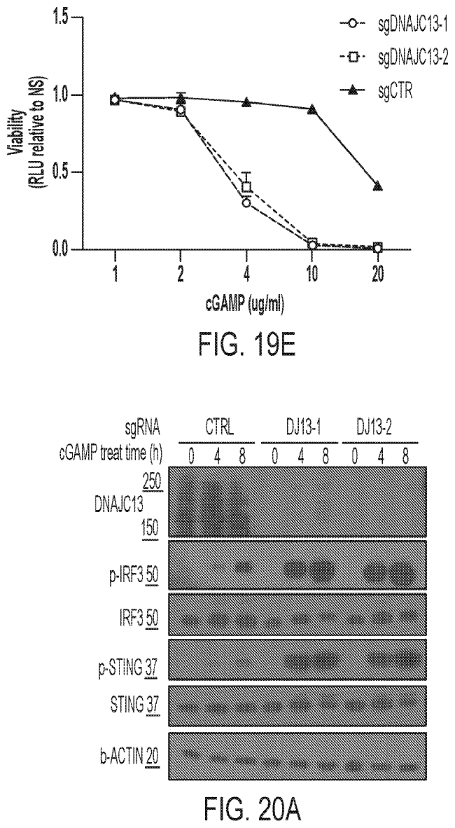

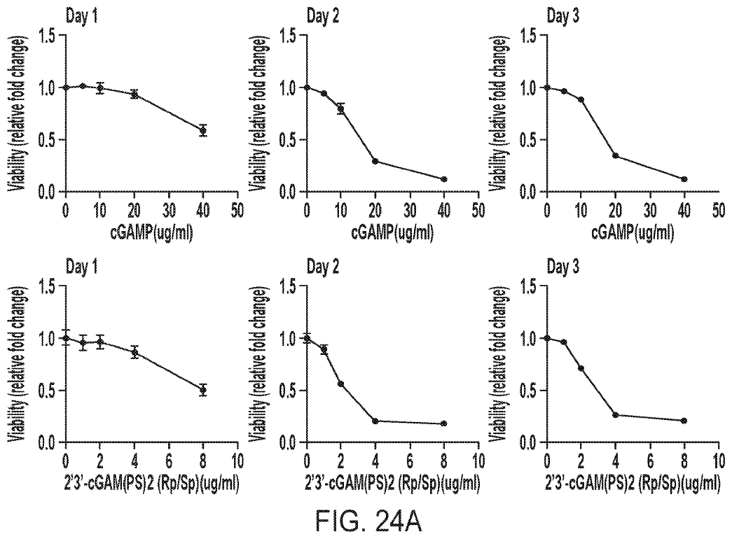

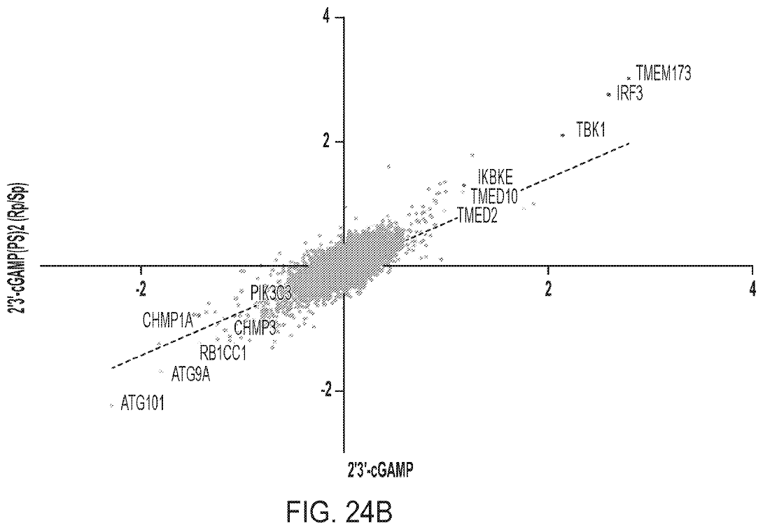

Starting from the principle for controlling STING signaling and trafficking, the genetic network regulating STING activity through these processes was explored. It was found that monocytic cell line U937 dies upon STING activation (FIG. 19A and FIG. 24A), making STING-induced death a great reporter for STING activity. Cas9-expressing U937 was transduced with genome-wide sgRNA library, cells were treated with either sublethal dose or lethal dose (EC75) of cGAMP or cGAM (PS)2 for 24 hours, and harvested with untreated group 48 h after for deep sequencing of sgRNA and comparing the frequency of sgRNA in different groups (FIG. 19B). STING (TMEM173) was the strongest death protection hit in the screening together with known downstream effector of TBK1, IRF3 as the top hits (FIG. 19c). STING trafficking essential genes TMED2, TMED10 were also top hits in the death protection arm (Sun et al. 2018). For the cell death enhancing arm (FIG. 24B), consistent with previous literature, macroautophagy related genes are known STING negative regulators (Saitoh et al. 2009) and top cell death enhancing hits (FIG. 19B and FIG. 24B), although the actual functional connection between macroautophagy and STING is still debated (Gui et al. 2019; Fischer et al. 2020).

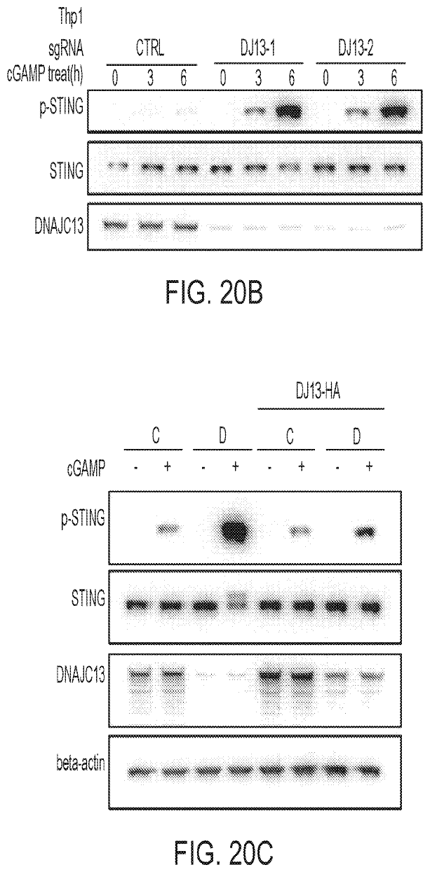

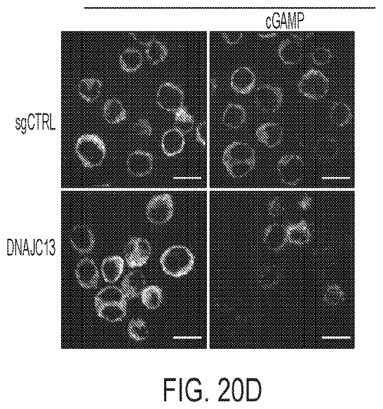

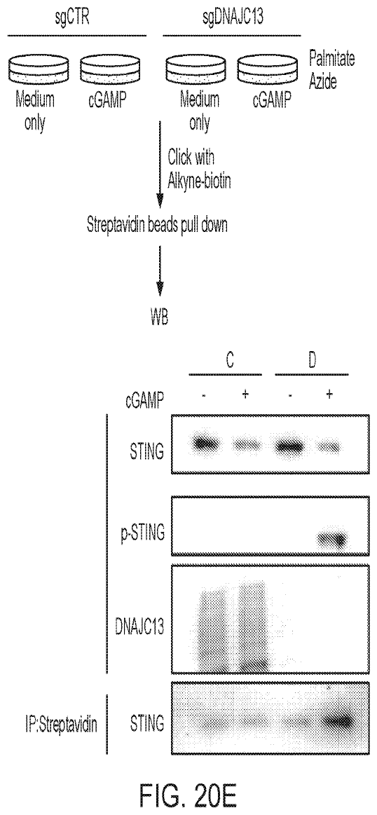

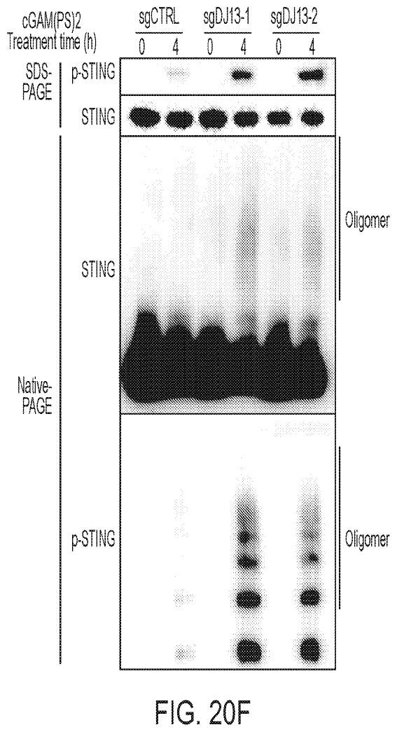

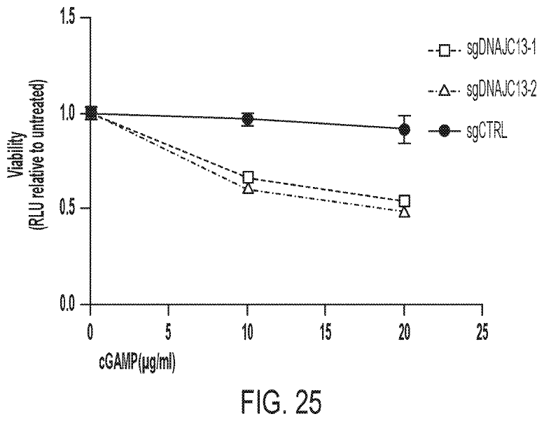

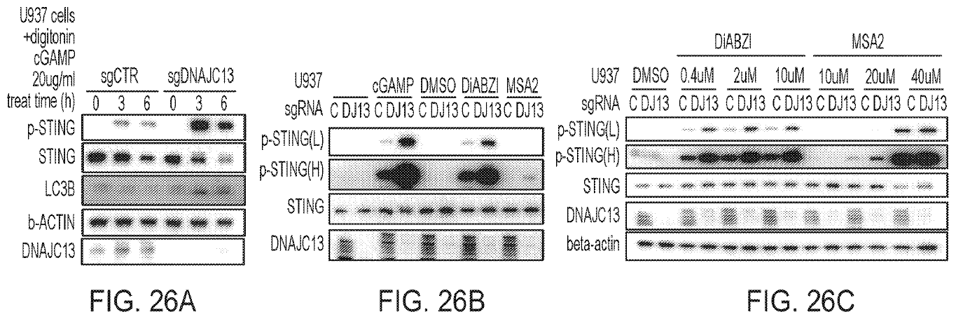

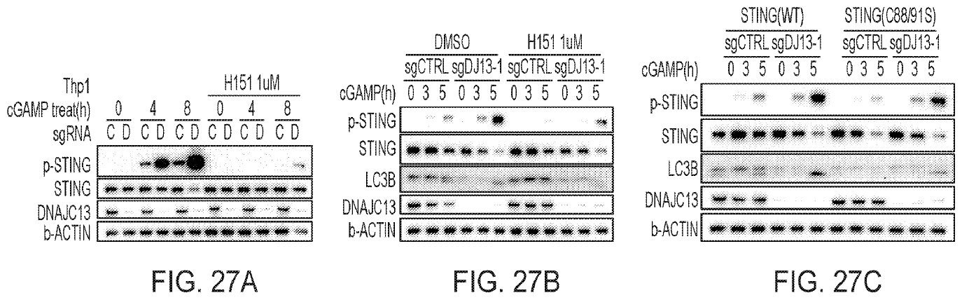

Interestingly, DNAJC13 showed up as one of the strongest hits in the cell death enhancing arm in both lethal dose and sublethal dose (FIG. 19D), suggesting it is a strong negative regulator of STING activity. Consistently, sgDNAJC13 showed enhanced cell death upon cGAMP treatment in both U937 (FIG. 19E) and Thp1 (FIG. 25). Further experiments using digitonin to permeabilize cells for cGAMP delivery and non-CDN STING agonists (Pan et al. 2020; Ramanjulu et al. 2018) further support that DNAJC13's function on STING activity is independent of cGAMP transport or metabolism on STING (FIG. 26). DNAJC13's exact function in inhibiting STING activity was then explored. Surprisingly, while most of the STING negative regulators inhibit sting activity by promoting sting degradation (Gonugunta et al. 2017), DNAJC13 seems to do the opposite. While DNAJC13 KO boosts phospho-STING and phos-IRF3 dramatically, it also dramatically enhances the degradation of STING in U937 cells (FIG. 20A and FIG. 26A) as well as in Thp1 cells (FIG. 20B and FIG. 27A), which is also cross-validated from previous independent STING activity screening, suggesting DNAJC13 inhibits STING function by limiting its translocation out of ER. Rescue of sgDNAJC13 by guide-resistant DNAJC13 further proves that the effect of sgDNAJC13 came from the absence of DNAJC13 protein itself (FIG. 20C). In imaging experiments, sgDNAJC13 cells showed much more dramatic translocation and degradation upon cGAMP stimulation than sgCTRL U937 (FIG. 20D). STING is known to be palmitoylated at TGN at its C88/C91 sites (Mukai et al. 2016), the palmitoylation level can serve as a way to quantify the amount of STING that have traffic through TGN. Using a click chemistry based palmitoylation assay, much higher levels of palmitoylation of STING were observed in sgDNAJC13 cells with cGAMP stimulation compared to sgCTRL cells (FIG. 20E), which is consistent with the imaging data that STING traffics more out of the ER for activation in sgDNAJC13 KO cells. STING palmitoylation mutants dramatically reduced the STING phosphorylation level without changing the dramatic difference between sgCTRL cells and sgDNAJC13 cells indicating the DNAJC13 effect on STING is independent of palmitoylation, this is further supported by using STING palmitoylation inhibitor H151 (Haag et al. 2018) in 293T and Thp1 cells (FIGS. 27A-27C).

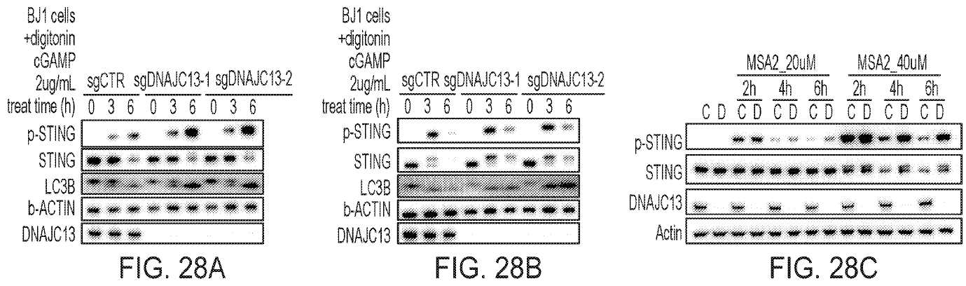

DNAJC13 is a member of the HSP40 family (J domain protein) which is known to resolve protein aggregate and oligomerization (Wentink et al. 2020). From the discussion above, oligomerization is a conserved property of STING and plays an important role in its trafficking. It was hypothesized that DNAJC13 influences STING trafficking and activity through influencing its oligomerization state. Through native gel, sgDNAJC13 U937 cells were observed to form more STING oligomers than sgCTRL cells upon STING agonist stimulation (FIG. 20F), further supporting the hypothesis. Interestingly, a unique role of DNAJC13 in STING degradation by regulating STING oligomerization was also observed. Starting from the interesting heterogeneity of DNAJC13 related phenotype in different cell types. While sgDNAJC13 boosted STING activity in BJ1 cells using low level of cGAMP stimulation, STING degradation slowed down upon high level agonist activity in sgDNAJC13 BJ1 cells, and stimulated a stronger autophagy response (FIGS. 28A-28C). STING degradation can happen cither through sorting of ubiquitinated STING into MVB or through endosomal microautophagy induced by STING on the surface of the endosome. Cytosolic protein aggregate degradation was shown to be insensitive to be degraded through proteasome machinery and highly dependent on autophagy mediated degradation (Lu et al. 2017; Li et al. 2019). It was questioned whether the aggregation of endosomal protein also highly depended on autophagy for its degradation rather than ESCRT sorting machinery. Following this hypothesis, sgDNAJC13 KO cells should have impaired ability to disassemble STING aggregate and have accumulation of STING oligomer. When sgCTRL and sgDNAJC13 BJ1 were treated with high dose of cGAMP, while sgCTRL and sgDNAJC13 form similar level of oligomer at 2 hour, there was a significant reduction of STING oligomer in sgCTRL cells while the oligomer level increased despite the significant degradation of total STING in sgDNAJC13 KO cells (FIG. 20F).

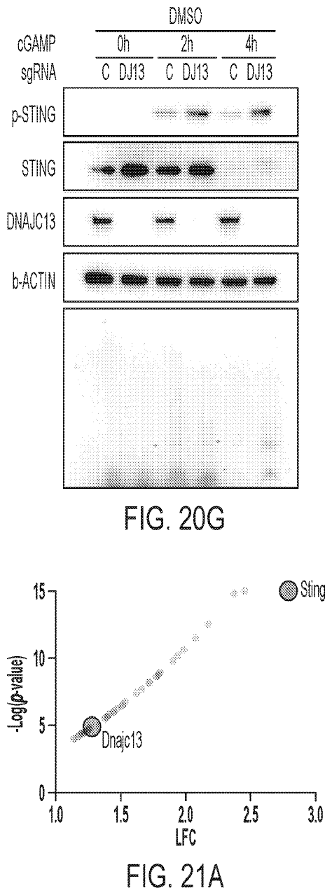

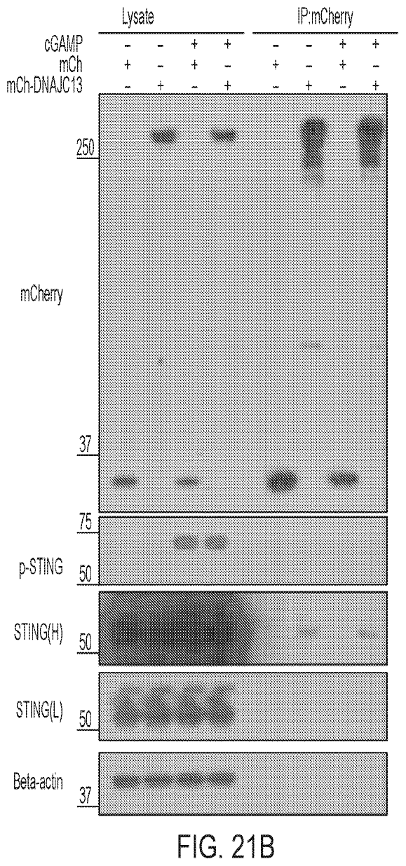

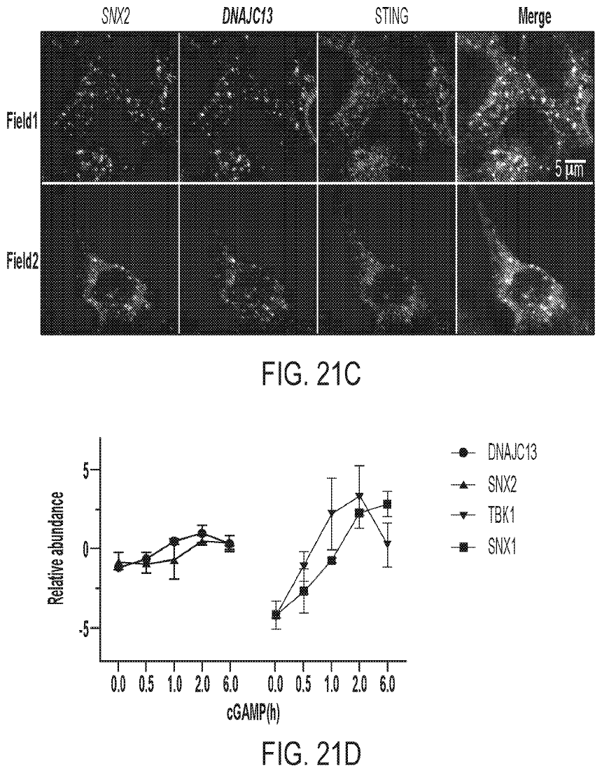

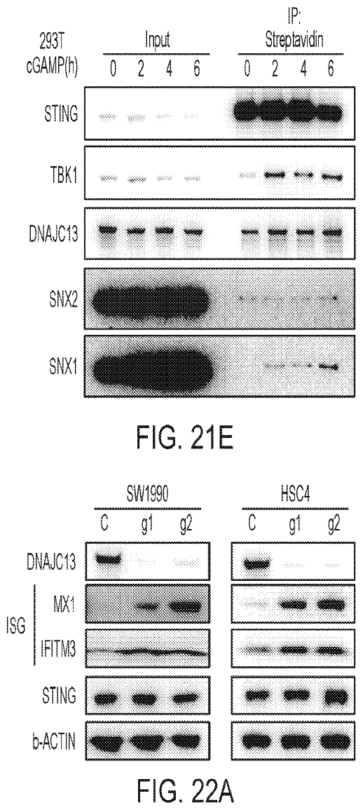

From a published IP-MS data for STING interaction proteins (Lee et al. 2013), it was discovered that DNAJC13 is a STING interaction protein suggesting DNAJC13 regulates STING activity through biochemical binding (FIG. 21A). Interestingly, there is no difference between STING and DNAJC13 binding with or without cGAMP stimulation (FIG. 21B). DNAJC13 localized on endosome through its PI3P binding motif on N terminal and STING is known to localize on ER without stimulation. It was wondered whether the constant interaction observed through Co-IP was also true in the live cells. Through imaging, it was surprisingly found that although STING was mainly sparsely localized, it indeed showed local concentration and colocalization with endosomal localized DNAJC13, together with SNX2 (Dong et al. 2016), an ER-endosomal contact site marker (FIG. 21C). Then, STING endosomes trafficking and co-localization with DNAJC13 and SNX2 through the ER-endosome interaction was explored. Taking advantage of the proximity of the biotinylation labelling enzyme TurboID, the STING proximity protein was identified at different time points during the activation process. As expected, with cGAMP stimulation, a dramatic increase of labelling of endosomal protein SNX1, and SNX2 was observed with or without cGAMP. Consistent with previous data of Co-IP and imaging, DNAJC13 also was constantly labeled as SNX2 (FIGS. 21D-21E), further proving that DNAJC13 constantly interacts with STING.

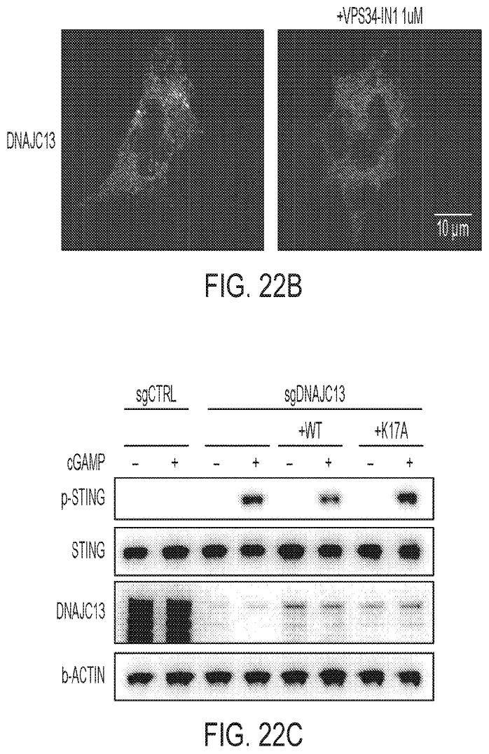

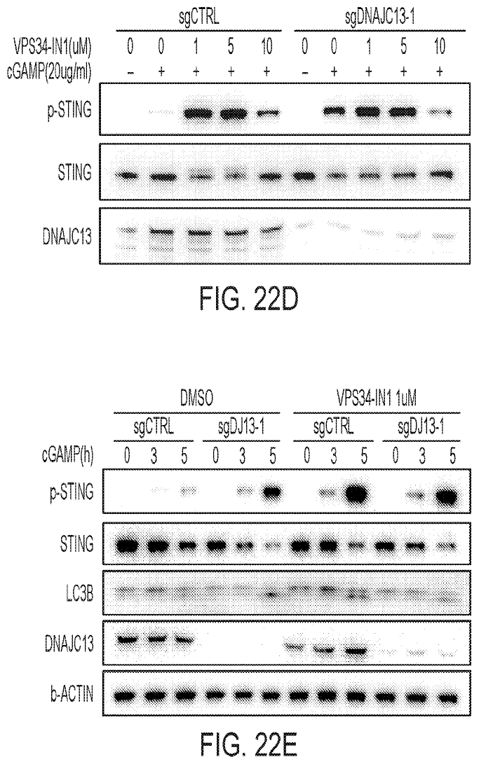

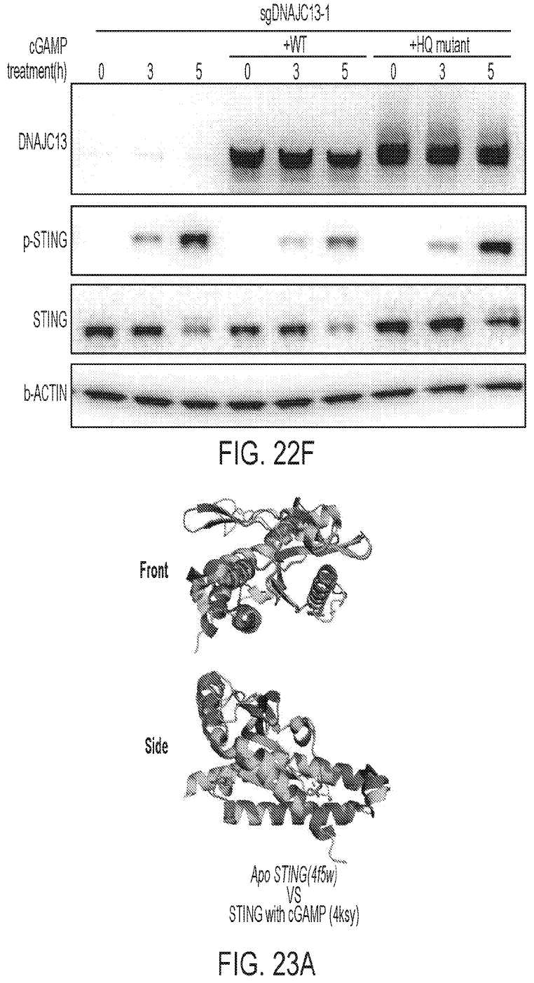

A large portion of cancer cells have a tonic level of STING activation due to reasons like DNA damage. Amplifying the tonic STING signal can be beneficial for tumor immunotherapy (Liu et al. 2019). Given the strong negative regulation effect of DNAJC13, DNAJC13 perturbation was explored to determine whether it could amplify this tonic STING activation in these cells. Indeed, there was a dramatic elevation of ISG overexpression (MX1, IFITM3) in these cells with DNAJC13 KO (FIG. 22A). However, given the limited understanding of DNAJC13's structure, known, potentially targetable DNAJC13 biochemical properties that are required for its function were explored.

While a lot of HSP40 proteins execute the function in the cytosol (Wentink et al. 2020), DNAJC13 has a conserved PI3P binding domain, it was wondered whether the endosome localization is important for its function in inhibiting STING activity. Interestingly, while the WT guide1-resistant DNAJC13 (glrDNAJC13 WT) rescued the sgDNAJC13 effect, PI3P binding site mutant glrDNAJC13 K17A was unstable (Xhabija and Vacratsis 2015) and failed to rescue the sgDNAJC13 KO effect (FIG. 22C). As a result, chemical ways to perturb DNAJC13 localization were tested. VPS34-IN1 is a highly specific inhibitor targeting VPS34 and inhibits its PI3P synthesis. It has been shown to disrupt PI3P binding dependent endosomal localization (Dong et al. 2016). Indeed, upon VPS34-IN1 treatment, clear diffusion of DNAJC13 from the endosome was observed (FIG. 22B). Clearly, with low level of VPS34-IN1 treatment (1 μM), there was strong boost of STING phosphorylation and enhancement of STING degradation in sgCTRL cells as strong as sgDNAJC13 cells upon cGAMP stimulation (FIGS. 22D-22E). Interestingly, STING induced autophagy was greatly enhanced by VPS34-IN1 which was consistent with the fact that STING induced autophagy does not require VPS34 activity which is usually essential for macroautophagy (FIG. 22E) (Fischer et al. 2020; Gui et al. 2019). Thus, VPS34 can be a valuable target to boost STING activity given VPS34-IN1 is a well characterized inhibitor (Bago et al. 2014). Contrary to reports saying STING trafficking and function can be inhibited by PI3P synthesis blockade through VPS34-IN1, results showed a non-specific effect from high doses of VPS34-IN1, since VPS34-IN1 achieved high level inhibition of the target at 1 μM. The results suggest that J domain activity was required for DNAJC13's inhibition effect on STING. HPD sites were well conserved in J domain proteins that were responsible for downstream disassembling activity together with HSP70 (Tsai and Douglas 1996). The H to Q mutant in the J domain lost the ability to disassemble oligomers. In DNAJC13, while WT DNAJC13 was able to inhibit STING activity in sgDNAJC13 BJ1 cells, the HQ mutant DNAJC13 failed to inhibit STING activity like WT, suggesting the oligomer disassembly effect is required for DNAJC13's inhibition on STING (FIG. 22F).

### Discussion

Presented herein is a STING density dependent activation model that explains the importance of localization in STING activation in the living cells, and also explains why STING mislocalization (in COPA patients) and STING overexpression trigger agonist-independent STING activation. Consistently, the blockade of STING palmitoylation, which is believed to concentrate STING on TGN, inhibits STING activity upon cGAMP binding (Haag et al. 2018) or in COPA mutation (Mukai et al. 2021), further supporting that concentration is the key for STING activity. The model presented herein takes advantage of structural analysis to show how ligand binding or mutation on different sites of STING induce the same oligomerization property change to induce STING trafficking and activation, providing mechanistic insights of STING auto-inhibition through Ser272-Arg284 lock which further explain the molecular details of constitutive activation properties of SAVI mutants.

Moreover, through genome-wide screening, J domain protein DNAJC13 was identified as a negative regulator for STING oligomerization to inhibit STING activation. DNAJC13's function depends on its J domain activity and endosome localization through ER-endosome contact sites. Furthermore, DNAJC13's role in helping resolve STING oligomers to promote its degradation was also found. Combined with DNAJC13's role in inhibiting STING trafficking, DNAJC13 inhibits STING translocation and speeds up STING degradation through disassembling STING oligomers, preventing its accumulation in TGN and endosome to control STING activation threshold.

While DNAJC13 has been shown to inhibit STING activity in different cells, the effect is much stronger in U937 and Thp1 compared to BJ1 and 293T. It is unclear if this difference comes from difference in DNAJC13 or STING protein amount or cell structural difference like ER-endosome contacts level. Clearly, the DNAJC13 protein serves as a way to control STING sensitivity in different types and the physiological relevance still needs to be further explored.

### REFERENCES

Ishikawa, H., Ma, Z. & Barber, G. N. STING regulates intracellular DNA-mediated, type I interferon-dependent innate immunity. Nature 461, 788-792 (2009).
Ablasser, A. et al. cGAS produces a 2′-5′-linked cyclic dinucleotide second messenger that activates STING. Nature 498, 380-384 (2013).
Gao, P. et al. Cyclic [G(2′,5′)pA(3′,5′)p] Is the Metazoan Second Messenger Produced by DNA-Activated Cyclic GMP-AMP Synthase. Cell 16, 1-14 (2013).
Wu, J. et al. Cyclic GMP-AMP is an endogenous second messenger in innate immune signaling by cytosolic DNA. Science 339, 826-830 (2013).
Sun, L., Wu, J., Du, F., Chen, X. & Chen, Z. J. Cyclic GMP-AMP synthase is a cytosolic DNA sensor that activates the type I interferon pathway. Science 339, 786-791 (2013).
Burdette, D. L. et al. STING is a direct innate immune sensor of cyclic di-GMP. Nature 478, 515-518 (2011).
Woodward, J. J., Iavarone, A. T. & Portnoy, D. a. c-di-AMP secreted by intracellular Listeria monocytogenes activates a host type I interferon response. Science 328, 1703-1705 (2010).
Gui, X. et al. Autophagy induction via STING trafficking is a primordial function of the cGAS pathway. Nature (2019).
Kranzusch, P. J. et al. Ancient Origin of cGAS-STING Reveals Mechanism of Universal 2′,3′ cGAMP Signaling. Mol. Cell 1-13 (2015).
Morehouse, B. R. et al. STING cyclic dinucleotide sensing originated in bacteria. Nature 586, 429-433 (2020).
Motwani, M., Pesiridis, S. & Fitzgerald, K. A. DNA sensing by the cGAS-STING pathway in health and disease. Nat. Rev. Genet. 20, 657-674 (2019).
Ishikawa, H. & Barber, G. N. STING is an endoplasmic reticulum adaptor that facilitates innate immune signalling. Nature 455, 674-678 (2008).
Mukai, K. et al. Activation of STING requires palmitoylation at the Golgi. Nat. Commun. 7, 11932 (2016).
Liu, S. et al. Phosphorylation of innate immune adaptor proteins MAVS, STING, and TRIF induces IRF3 activation. Science 347, aaa2630-aaa2630 (2015).
Fischer, T. D., Wang, C., Padman, B. S., Lazarou, M. & Youle, R. J. STING induces LC3B lipidation onto single-membrane vesicles via the V-ATPase and ATG16L1-WD40 domain. J. Cell Biol. 219, (2020).
Srikanth, S. et al. The Ca 2+ sensor STIM1 regulates the type I interferon response by retaining the signaling adaptor STING at the endoplasmic reticulum. Nat. Immunol. 20, 152-162 (2019).
Zhang, B.-C. et al. STEEP mediates STING ER exit and activation of signaling. Nat. Immunol. 21, 868-879 (2020).
Pokatayev, V. et al. Homeostatic regulation of STING protein at the resting state by stabilizer TOLLIP. Nat. Immunol. 21, 158-167 (2020).
Gonugunta, V. K. et al. Trafficking-Mediated STING Degradation Requires Sorting to Acidified Endolysosomes and Can Be Targeted to Enhance Anti-tumor Response. 21, 3234-3242 (2017).
Ablasser, A. & Hur, S. Regulation of cGAS- and RLR-mediated immunity to nucleic acids. Nat. Immunol. 21, 17-29 (2020).
Branon, T. C. et al. Efficient proximity labeling in living cells and organisms with TurboID. Nat. Biotechnol. 36, 880-898 (2018).
Goodwin, J. M. et al. Autophagy-Independent Lysosomal Targeting Regulated by ULK1/2-FIP200 and ATG9. Cell Rep. 20, 2341-2356 (2017).
Doench, J. G. et al. Optimized sgRNA design to maximize activity and minimize off-target effects of CRISPR-Cas9. Nat. Biotechnol. 34, 184-191 (2016).
Deng, Z. et al. A defect in COPI-mediated transport of STING causes immune dysregulation in COPA syndrome. J. Exp. Med. 217, 1-37 (2020).
Vietri, M., Radulovic, M. & Stenmark, H. The many functions of ESCRTs. Nat. Rev. Mol. Cell Biol. 21, 25-42 (2020).
Christ, L., Raiborg, C., Wenzel, E. M., Campsteijn, C. & Stenmark, H. Cellular Functions and Molecular Mechanisms of the ESCRT Membrane-Scission Machinery. Trends Biochem. Sci. 42, 42-56 (2017).
Wunderley, L., Brownhill, K., Stefani, F., Tabernero, L. & Woodman, P. The molecular basis for selective assembly of the UBAP1-containing endosome-specific ESCRT-I complex. J. Cell Sci. 127, 663-672 (2014).
Jeremiah, N. et al. Inherited STING-activating mutation underlies a familial inflammatory syndrome with lupus-like manifestations. J. Clin. Invest. 124, 5516-5520 (2014).
Konno, H. et al. Pro-inflammation Associated with a Gain-of-Function Mutation (R284S) in the Innate Immune Sensor STING. Cell Rep. 23, 1112-1123 (2018).
Liu, Y. et al. Activated STING in a Vascular and Pulmonary Syndrome. N. Engl. J. Med. 1-12 (2014).
Ritchie, C., Cordova, A. F., Hess, G. Bassik, M. C. & Li, L. SLC19A1 Is an Importer of the Immunotransmitter cGAMP. Molecular Cell vol. 75 372-381.e5 (2019).
Gulen, M. F. et al. Signalling strength determines proapoptotic functions of STING. Nat. Commun. 8, 427 (2017).
Gatica, D., Lahiri, V. & Klionsky, D. J. Cargo recognition and degradation by selective autophagy. Nat. Cell Biol. 20, 233-242 (2018).
Takahashi, Y. et al. VPS37A directs ESCRT recruitment for phagophore closure. J. Cell Biol. 218, jcb.201902170 (2019).
Zhen, Y. et al. ESCRT-mediated phagophore sealing during mitophagy. Autophagy 16, 826-841 (2020).
Corona, A. K. & Jackson, W. T. Finding the Middle Ground for Autophagic Fusion Requirements. Trends Cell Biol. 28, 869-881 (2018).
Tumbarello, D. A. et al. Autophagy receptors link myosin VI to autophagosomes to mediate Tom1-dependent autophagosome maturation and fusion with the lysosome. Nat14, 1024-1035 (2012).
Hyer, M. L. et al. A small-molecule inhibitor of the ubiquitin activating enzyme for cancer treatment. Nat. Med. 24, 186-193 (2018).
Prabakaran, T. et al. Attenuation of cGAS-STING signaling is mediated by a p62/SQSTM1-dependent autophagy pathway activated by TBK1. EMBO J. 37, (2018).
Beck, D. B. et al. Somatic Mutations in UBA1 and Severe Adult-Onset Autoinflammatory Disease. N. Engl. J. Med. 1-11 (2020).
Dou, Z. et al. Cytoplasmic chromatin triggers inflammation in senescence and cancer. Nature (2017) doi: 10.1038/nature24050.
Glück, S. et al. Innate immune sensing of cytosolic chromatin fragments through cGAS promotes senescence. Nat. Cell Biol. 19, (2017).
Yang, H., Wang, H., Ren, J., Chen, Q. & Chen, Z. J. cGAS is essential for cellular senescence. Proceedings of the National Academy of Sciences 201705499 (2017).
Medvar, B., Raghuram, V., Pisitkun, T., Sarkar, A. & Knepper, M. A. Comprehensive database of human E3 ubiquitin ligases: application to aquaporin-2 regulation. Physiol. Genomics 48, 502-512 (2016).
Li, W. et al. Genome-Wide and Functional Annotation of Human E3 Ubiquitin Ligases Identifies MULAN, a Mitochondrial E3 that Regulates the Organelle's Dynamics and Signaling. PLOS ONE vol. 3 e1487 (2008).
Wu, X. & Karin, M. Emerging roles of Lys63-linked polyubiquitylation in immune responses. Immunol. Rev. 266, 161-174 (2015).
Ni, G., Konno, H. & Barber, G. N. Ubiquitination of STING at lysine 224 controls IRF3 activation. Sci Immunol 2, (2017).
Takahashi, M. et al. The tumor suppressor kinase DAPK3 drives tumor-intrinsic immunity through the STING-IFN-pathway. Nat. Immunol. (2021) doi: 10.1038/s41590-021-00896-3.
Farazi Fard, M. A. et al. Truncating Mutations in UBAP1 Cause Hereditary Spastic Paraplegia. Am. J. Hum. Genet. 104, 767-773 (2019).
Gu, S. et al. Truncating variants in UBAP1 associated with childhood-onset nonsyndromic hereditary spastic paraplegia. Hum. Mutat. 41, 632-640 (2020).
Lin, X. et al. Stop-gain mutations in UBAP1 cause pure autosomal-dominant spastic paraplegia. Brain 142, 2238-2252 (2019).
Zivony-Elboum, Y. et al. A founder mutation in Vps37A causes autosomal recessive complex hereditary spastic paraparesis. J. Med. Genet. 49, 462-472 (2012).
Crow, Y. J. et al. Mutations in ADAR1, IFIH1, and RNASEH2B presenting as spastic paraplegia. Neuropediatrics 45, 386-393 (2014).
Agromayor, M. et al. The UBAP1 subunit of ESCRT-I interacts with ubiquitin via a SOUBA domain. Structure 20, 414-428 (2012).
Stefani, F. et al. UBAP1 is a component of an endosome-specific ESCRT-I complex that is essential for MVB sorting. Curr. Biol. 21, 1245-1250 (2011).
Lepelley, A. et al. Mutations in COPA lead to abnormal trafficking of STING to the Golgi and interferon signaling. J. Exp. Med. 217, (2020).
Mukai, K. et al. Homeostatic regulation of STING by Golgi-to-ER membrane traffic. (2020).
Steiner, A. et al. Activation of STING due to COPI-deficiency. bioRxiv 2020.07.09.194399 (2020).
Uematsu, R. & Shum, A. K. Homeostatic regulation of STING by Golgi-to-ER membrane traffic. bioRxiv Cell Biology (2020) doi: 10.1101/2020.05.20.107664.
Schröder, B., Wrocklage, C., Hasilik, A. & Saftig, P. Molecular characterisation of ‘transmembrane protein 192’ (TMEM192), a novel protein of the lysosomal membrane391, 695-704 (2010).
Schoggins, J. W. et al. Pan-viral specificity of IFN-induced genes reveals new roles for cGAS in innate immunity. Nature 505, 691-695 (2014).
Gentili, M. et al. The N-Terminal Domain of cGAS Determines Preferential Association with Centromeric DNA and Innate Immune Activation in the Nucleus. Cell Rep. 26, 2377-2393.e13 (2019).
Chu, T.-T. et al. Tonic prime-boost of STING signalling mediates Niemann-Pick disease type C. Nature (2021) doi: 10.1038/s41586-021-03762-2.
Majer, O., Liu, B., Kreuk, L. S. M., Krogan, N. & Barton, G. M. UNC93B1 recruits syntenin-1 to dampen TLR7 signalling and prevent autoimmunity. Nature 575, 366-370 (2019).
Holm, C. K. et al. Virus-cell fusion as a trigger of innate immunity dependent on the adaptor STING. Nat. Immunol. 13, 737-743 (2012).
Holm, C. K. et al. Influenza A virus targets a cGAS-independent STING pathway that controls enveloped RNA viruses. Nat. Commun. 7, 10680 (2016).
Decalf, J. et al. Sensing of HIV-1 entry triggers a type I interferon response in human primary macrophages. J. Virol. 91, JVI.00147-17 (2017).
Yu, C.-H. et al. TDP-43 Triggers Mitochondrial DNA Release via mPTP to Activate cGAS/STING in ALS. Cell 183, 636-649.e18 (2020).
Thomas, C. A. et al. Modeling of TREX1-dependent autoimmune disease using human stem cells highlights L1 accumulation as a source of neuroinflammation. Cell Stem Cell 21, 319-331.e8 (2017).
Abou Jamra, R. et al. Adaptor protein complex 4 deficiency causes severe autosomal-recessive intellectual disability, progressive spastic paraplegia, shy character, and short stature. Am. J. Hum. Genet. 88, 788-795 (2011).
Rodger, C. et al. De Novo VPS4A Mutations Cause Multisystem Disease with Abnormal Neurodevelopment. 1-20 (2020).
Gentili, M. et al. Baruch, K., Deczkowska, A., David, E., Castellano, J. M., Miller, O., Kertser, A., Berkutzki, T., Barnett-Itzhaki, Z., Bezalel, D., Wyss-Coray, T., et al. (2014). Aging-induced type I interferon response at the choroid plexus negatively affects brain function. Science 346, 89-93.
Transmission of innate immune signaling by packaging of cGAMP in viral particles. Science 349, 1232-1236 (2015).
Gentili, M. et al. The N-Terminal Domain of cGAS Determines Preferential Association with Centromeric DNA and Innate Immune Activation in the Nucleus. Cell Rep. 26, 2377-2393.e13 (2019).
Tanida-Miyake, E., Koike, M., Uchiyama, Y. & Tanida, I. Optimization of mNeonGreen for Homo sapiens increases its fluorescent intensity in mammalian cells. PLOS One 13, e0191108 (2018).
Branon, T. C. et al. Efficient proximity labeling in living cells and organisms with TurboID. Nat. Biotechnol. 36, 880-898 (2018).
Mertins, P. et al. Reproducible workflow for multiplexed deep-scale proteome and phosphoproteome analysis of tumor tissues by liquid chromatography-mass spectrometry. Nat. Protoc. 13, 1632-1661 (2018).
Rappsilber, J., Mann, M. & Ishihama, Y. Protocol for micro-purification, enrichment, pre-fractionation and storage of peptides for proteomics using StageTips. Nat. Protoc. 2, 1896-1906 (2007).
Elias, J. E. & Gygi, S. P. Target-decoy search strategy for increased confidence in large-scale protein identifications by mass spectrometry. Nat. Methods 4, 207-214 (2007).
Ritchie, M. E. et al. limma powers differential expression analyses for RNA-sequencing and microarray studies. Nucleic Acids Res. 43, e47 (2015).
Schwämmle, V. & Jensen, O. N. A simple and fast method to determine the parameters for fuzzy c-means cluster analysis. Bioinformatics 26, 2841-2848 (2010).
Raudvere, U. et al. g:Profiler: a web server for functional enrichment analysis and conversions of gene lists (2019 update). Nucleic Acids Res. 47, W191-W198 (2019).
Szklarczyk, D. et al. STRING v11: protein-protein association networks with increased coverage, supporting functional discovery in genome-wide experimental datasets. Nucleic Acids Res. 47, D607-D613 (2019).
Doench, J. G. et al. Optimized sgRNA design to maximize activity and minimize off-target effects of CRISPR-Cas9. Nat. Biotechnol. 34, 184-191 (2016).
Medvar, B., Raghuram, V., Pisitkun, T., Sarkar, A. & Knepper, M. A. Comprehensive database of human E3 ubiquitin ligases: application to aquaporin-2 regulation. Physiol. Genomics 48, 502-512 (2016).
Li, W. et al. Genome-Wide and Functional Annotation of Human E3 Ubiquitin Ligases Identifies MULAN, a Mitochondrial E3 that Regulates the Organelle's Dynamics and Signaling. PLOS ONE vol. 3 c1487 (2008).
Fulco, C. P. et al. Systematic mapping of functional enhancer-promoter connections with CRISPR interference. Science 354, 769-773 (2016).
Hagemann-Jensen, M. et al. Gonugunta, V. K., Sakai, T., Pokatayev, V., Yang, K., Wu, J., Dobbs, N., and Yan, N. (2017). Trafficking-Mediated STING Degradation Requires Sorting to Acidified Endolysosomes and Can Be Targeted to Enhance Anti-tumor Response. Cell Rep. 21, 3234-3242.
Single-cell RNA counting at allele and isoform resolution using Smart-seq3. Nat. Biotechnol. 38, 708-714 (2020).
MartinLiu, X. M., Sun, L. L., Hu, W., Ding, Y. H., Dong, M. Q., and Du, L. L. (2015b). ESCRTs Cooperate with a Selective Autophagy Receptor to Mediate Vacuolar Targeting of Soluble Cargos. Mol. Cell 59, 1035-1042.
, M. Cutadapt removes adapter sequences from high-throughput sequencing reads. EMBnet.journal 17, 10-12 (2011).
Mejlvang, J., Olsvik, H., Svenning, S., Bruun, J. A., Abudu, Y. P., Larsen, K. B., Brech, A., Hansen, T. E., Brenne, H., Hansen, T., et al. (2018). Starvation induces rapid degradation of selective autophagy receptors by endosomal microautophagy. J. Cell Biol. 217, 3640-3655.
Morehouse, B. R., Govande, A. A., Millman, A., Keszei, A. F. A., Lowey, B., Ofir, G., Shao, S., Sorek, R., and Kranzusch, P. J. (2020). STING cyclic dinucleotide sensing originated in bacteria. Nature 586, 429-433.
Mukai, K., Ogawa, E., Uematsu, R., Kuchitsu, Y., Uemura, T., Waguri, S., Suzuki, T., Dohmae, N., Arai, H., ShHoum, A. K., et al. (2020). Homeostatic regulation of STING by Golgi-to-ER membrane traffic.
Ni, G., Konno, H., and Barber, G. N. (2017). Ubiquitination of STING at lysine 224 controls IRF3 activation. Science Immunology 2, 1-13.
Patro, R., Duggal, G., Love, M. I., Irizarry, R. A. & Kingsford, C. Salmon provides fast and bias-aware quantification of transcript expression. Nat. Methods 14, 417-419 (2017).
Goedhart, J. & Luijsterburg, M. S. VolcaNoseR is a web app for creating, exploring, labeling and sharing volcano plots. Sci. Rep. 10, 20560 Popoff, V., Adolf, F., Brügge, B., and Wieland, F. (2011). COPI budding within the Golgi stack. Cold Spring Harb. Perspect. Biol. 3, 1-19.
Razi, M., Chan, E. Y. W., and Tooze, S. A. (2009). Early endosomes and endosomal coatomer are required for Autophagy. J. Cell Biol. 185, 305-321.
Sahu, R., Kaushik, S., Clement, C. C., Cannizzo, E. S., Scharf, B., Follenzi, A., Potolicchio, I., Nieves, E., Cuervo, A. M., and Santambrogio, L. (2011). Microautophagy of Cytosolic Proteins by Late Endosomes. Dev. Cell 20, 131-139.
Saitoh, T., Fujita, N., Hayashi, T., Takahara, K., Satoh, T., Lee, H., Matsunaga, K., Kageyama, S., Omori, H., Noda, T., et al. (2009). Atg9a controls dsDNA-driven dynamic translocation of STING and the innate immune response. Proc. Natl. Acad. Sci. U.S.A 106, 20842-20846.
Steiner, A., Schaale, K. H., Prigione, I., De Nardo, D., Dagley, L., Yu, C.-H., Laohamonthonkul, P., Harapas, C., Gantier, M., Gattorno, M., et al. (2020). Activation of STING due to COPI-deficiency. bioRxiv 2020.07.09.194399.
Tekirdag, K., and Cuervo, A. M. (2018). Chaperone-mediated autophagy and endosomal microautophagy: Joint by a chaperone. J. Biol. Chem. 293, 5414-5424.
Zhen, Y., Spangenberg, H., Munson, M. J., Brech, A., Schink, K. O., Tan, K. W., Sørensen, V., Wenzel, E. M., Radulovic, M., Engedal, N., et al. ESCRT-mediated phagophore sealing during mitophagy. Autophagy 16, 826-841. (2020).

### OTHER EMBODIMENTS

In addition to the embodiments expressly described herein, it is to be understood that all of the features disclosed in this disclosure may be combined in any combination (e.g., permutation, combination). Each element disclosed in the disclosure may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, and can make various changes and modifications of the invention to adapt it to various usages and conditions. Thus, other embodiments are also within the claims.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following embodiments.

What is claimed is:

1. A method of increasing STING activation by administering an effective amount of an agent that reduces or inhibits DNAJC13 activity, thereby increasing a STING-dependent innate immune pathway.

2. The method of claim 1, wherein the STING-dependent innate immune pathway comprises type I interferon induction.

3. The method of claim 1, wherein the STING-dependent innate immune pathway comprises autophagy.

4. The method of claim 1, wherein the agent is a small molecule inhibitor of DNAJC13.

5. The method of claim 1, wherein the agent is a nucleic acid molecule capable of reducing expression of DNAJC13.

6. The method of claim 5, wherein the nucleic acid molecule is a short interfering RNA (siRNA), a short hairpin RNA (shRNA), or an antisense oligonucleotide (ASO).

7. The method of claim 5, wherein the nucleic acid molecule is capable of hybridizing to messenger RNA (mRNA) encoding the negative regulator of STING.

8. The method of claim 5, wherein the agent is a recombinant protein that inhibits DNAJC13.

9. The method of claim 5, wherein the agent is an antibody or antibody binding fragment that specifically binds to DNAJC13.

10. A method of augmenting a host's STING-dependent innate immunity, comprising administering an effect amount of an agent that reduces or inhibits DNAJC13 activity.

11. The method of claim 1, wherein the STING-dependent innate immunity comprises type I interferon induction.

12. The method of claim 1, wherein the STING-dependent innate immunity comprises autophagy.

13. The method of claim 1, wherein the agent is a small molecule inhibitor of DNAJC13.

14. The method of claim 1, wherein the agent is a nucleic acid molecule capable of reducing expression of DNAJC13.

15. The method of claim 5, wherein the nucleic acid molecule is a short interfering RNA (siRNA), a short hairpin RNA (shRNA), or an antisense oligonucleotide (ASO).

16. The method of claim 5, wherein the nucleic acid molecule is capable of hybridizing to messenger RNA (mRNA) encoding the negative regulator of STING.

17. The method of claim 5, wherein the agent is a recombinant protein that inhibits DNAJC13.

18. The method of claim 5, wherein the agent is an antibody or antibody binding fragment that specifically binds to DNAJC13.

19. A method of increasing STING activation by administering an effect amount of an agent that reduces or inhibits ESCRT activity, thereby increasing a STING-dependent innate immune pathway.

20. The method of claim 1, wherein increasing STING activation results from increasing a STING-dependent innate immune pathway.

21. The method of claim 1, wherein the STING-dependent innate immune pathway comprises autophagy of type I interferon induction.

22. The method of claim 1, wherein the agent is a small molecule inhibitor of ESCRT.

23. The method of claim 1, wherein the agent is a nucleic acid molecule capable of reducing expression of ESCRT.

24. The method of claim 5, wherein the nucleic acid molecule is a short interfering RNA (siRNA), a short hairpin RNA (shRNA), or an antisense oligonucleotide (ASO).

25. The method of claim 5, wherein the nucleic acid molecule is capable of hybridizing to messenger RNA (mRNA) encoding the negative regulator of STING.

26. The method of claim 5, wherein the agent is a recombinant protein that inhibits DNAJC13.

27. The method of claim 5, wherein the agent is an antibody or antibody binding fragment that specifically binds to ESCRT.

28. A method of augmenting a host's STING-dependent innate immunity, comprising administering an effective amount of an agent that reduces or inhibits ESCRT activity.

29. The method of claim 1, wherein the STING-dependent innate immunity comprises type I interferon induction.

30. The method of claim 1, wherein the STING-dependent innate immunity comprises autophagy.

31. The method of claim 1, wherein the agent is a small molecule inhibitor of ESCRT.

32. The method of claim 1, wherein the agent is a nucleic acid molecule capable of reducing expression of ESCRT.

33. The method of claim 5, wherein the nucleic acid molecule is a short interfering RNA (siRNA), a short hairpin RNA (shRNA), or an antisense oligonucleotide (ASO).

34. The method of claim 5, wherein the nucleic acid molecule is capable of hybridizing to messenger RNA (mRNA) encoding the negative regulator of STING.

35. The method of claim 5, wherein the agent is a recombinant protein that inhibits ESCRT.

36. The method of claim 5, wherein the agent is an antibody or antibody binding fragment that specifically binds ESCRT.

37. A method for modulating the activity of a Stimulator of Interferon Genes (STING) protein, comprising administering an effective amount of an agent that results in an increase in STING activity.

38. The method of claim 37, wherein the STING activity is STING signaling.

39. The method of claim 37 or 38, wherein administration of the agent results in a decrease in the intracellular degradation of STING.

40. The method of claim 39, wherein intracellular degradation of STING is decreased by up to 10%, up to 20%, up to 30%, up to 40%, up to 50%, up to 60%, up to 70%, up to 80%, up to 90%, up to 99%, or up to 100%.

41. The method of any one of claims 37-40, wherein the administration of the agent results in an increase in type I interferon (IFN) expression.

42. The method of any one of claims 37-41, wherein the administration of the agent results in an increase in production of one or more pro-inflammatory cytokines.

43. The method of any one of claims 37-42, wherein the administration of the agent enhances an immune response against a disease in a subject.

44. The method of claim 43, wherein the immune response is an innate immune response.

45. The method of claim 43, wherein the disease is a cancer.

46. The method of claim 45, wherein the cancer is selected from: colorectal cancer, lung cancer, breast cancer, pancreatic cancer, prostate cancer, bladder cancer, kidney cancer, thyroid cancer, uterine cancer, cervical cancer, ovarian cancer, testicular cancer, esophageal cancer, stomach cancer, liver cancer, brain cancer, peritoneal cancer, lymphoma, leukemia, multiple myeloma, neuroblastoma, osteosarcoma, and soft tissue sarcoma.

47. The method of any one of claims 43-46, wherein the subject is a human patient.

48. The method of any one of claims 37-47, wherein the agent reduces the activity of a negative regulator of STING.

49. The method of claim 48, wherein the negative regulator is a component of the ESCRT pathway.

50. The method of claim 48, wherein the negative regulator is hepatocyte growth factor-regulated tyrosine kinase substrate (HGS) or vacuolar protein sorting-associated protein 37 (VPS37A).

51. The method of claim 48, wherein the negative regulator of STING is a protein containing a J-domain.

52. The method of claim 51, wherein the protein containing a J-domain facilitates the palmitoylation of STING.

53. The method of claim 51 or claim 52, wherein the negative regulator of STING is DnaJ homolog subfamily C member 13 (DNAJC13).

54. The method of any one of claims 49-53, wherein the agent is a small molecular inhibitor of the negative regulator of STING.

55. The method of any one of claims 49-53, wherein the agent is a nucleic acid capable of reducing expression of the negative regulator of STING.

56. The method of claim 55, wherein the nucleic acid is a short interfering RNA (siRNA), a short hairpin RNA (shRNA), or an antisense oligonucleotide (ASO).

57. The method of claim 56, wherein the nucleic acid is capable of hybridizing to messenger RNA (mRNA) encoding the negative regulator of STING.

58. The method of any one of claims 55-57, wherein the nucleic acid reduces expression of the negative regulator of sting by up to 10%, up to 20%, up to 30%, up to 40%, up to 50%, up to 60%, up to 70%, up to 80%, up to 90%, up to 99%, or up to 100%.

59. The method of claim any one of claims 47-57, wherein the agent is a recombinant protein having STING activity or a nucleic acid that encodes a recombinant protein having STING activity.

60. The method of claim 59, wherein the nucleic acid is mRNA.

61. The method of claim 59 or claim 60, wherein the recombinant protein comprises recombinant STING.

62. The method of claim 61, wherein the recombinant protein is a fusion protein comprising STING and one or more additional proteins or fragments thereof.

63. The method of claim 62, wherein the recombinant protein is a fusion protein comprising STING and one or more FYVE domains.

64. The method of claim 63, wherein the recombinant protein is a fusion protein comprising STING and two FYVE domains.

65. The method of claim 63 or claim 64, wherein the one or more FYVE domains are fused to the N-terminus of STING.

66. The method of claim 65, wherein the recombinant protein does not comprise the endogenous transmembrane domain of STING.

67. The method of any one of claims 59-66, wherein the recombinant protein comprises constitutively active STING.

68. A method for treating a disease in a subject, comprising administering to the subject an effective amount of an agent that results in an increase in STING activity.

69. The method of claim 68, wherein the STING activity is STING signaling.

70. The method of claim 68 or 69, wherein administration of the agent results in a decrease in the intracellular degradation of STING in the subject.

71. The method of claim 70, wherein intracellular degradation of STING is decreased by up to 10%, up to 20%, up to 30%, up to 40%, up to 50%, up to 60%, up to 70%, up to 80%, up to 90%, up to 99%, or up to 100%.

72. The method of any one of claims 68-71, wherein the administration of the agent results in an increase in type I interferon (IFN) expression in the subject.

73. The method of any one of claims 68-72, wherein the administration of the agent results in an increase in production of one or more pro-inflammatory cytokines in the subject.

74. The method of any one of claims 68-73, wherein the administration of the agent enhances an immune response against the disease in a subject.

75. The method of claim 74, wherein the immune response is an innate immune response.

76. The method of any one of claims 68-75, wherein the disease is a cancer.

77. The method of claim 76, wherein the cancer is selected from: colorectal cancer, lung cancer, breast cancer, pancreatic cancer, prostate cancer, bladder cancer, kidney cancer, thyroid cancer, uterine cancer, cervical cancer, ovarian cancer, testicular cancer, esophageal cancer, stomach cancer, liver cancer, brain cancer, peritoneal cancer, lymphoma, leukemia, multiple myeloma, neuroblastoma, osteosarcoma, and soft tissue sarcoma.

78. The method of any one of claims 68-77, wherein the subject is a human patient.

79. The method of any one of claims 68-78, wherein the agent reduces the activity of a negative regulator of STING in the subject.

80. The method of claim 79, wherein the negative regulator is a component of the ESCRT pathway.

81. The method of claim 80, wherein the negative regulator is hepatocyte growth factor-regulated tyrosine kinase substrate (HGS) or vacuolar protein sorting-associated protein 37 (VPS37A).

82. The method of claim 79, wherein the negative regulator of STING is a protein containing a J-domain.

83. The method of claim 81, wherein the protein containing a J-domain facilitates the palmitoylation of STING.

84. The method of claim 81 or claim 83, wherein the negative regulator of STING is DnaJ homolog subfamily C member 13 (DNAJC13).

85. The method of any one of claims 79-84, wherein the agent is a small molecular inhibitor of the negative regulator of STING.

86. The method of any one of claims 79-84, wherein the agent is a nucleic acid capable of reducing expression of the negative regulator of STING.

87. The method of claim 86, wherein the nucleic acid is a short interfering RNA (siRNA), a short hairpin RNA (shRNA), or an antisense oligonucleotide (ASO).

88. The method of claim 87, wherein the nucleic acid is capable of hybridizing to messenger RNA (mRNA) encoding the negative regulator of STING.

89. The method of any one of claims 86-88, wherein the nucleic acid reduces expression of the negative regulator of sting by up to 10%, up to 20%, up to 30%, up to 40%, up to 50%, up to 60%, up to 70%, up to 80%, up to 90%, up to 99%, or up to 100%.

90. The method of claim any one of claims 68-78, wherein the agent is a recombinant protein having STING activity or a nucleic acid that encodes a recombinant protein having STING activity.

91. The method of claim 90, wherein the nucleic acid is mRNA.

92. The method of claim 90 or claim 91, wherein the recombinant protein comprises recombinant STING.

93. The method of claim 92, wherein the recombinant protein is a fusion protein comprising STING and one or more additional proteins or fragments thereof.

94. The method of claim 93, wherein the recombinant protein is a fusion protein comprising STING and one or more FYVE domains.

95. The method of claim 94, wherein the recombinant protein is a fusion protein comprising STING and two FYVE domains.

96. The method of claim 94 or claim 95, wherein the one or more FYVE domains are fused to the N-terminus of STING.

97. The method of claim 96, wherein the recombinant protein does not comprise the endogenous transmembrane domain of STING.

98. The method of any one of claims 90-97, wherein the recombinant protein comprises constitutively active STING.

99. A method for enhancing the efficacy of a therapy for a disease, comprising administering to a subject in need thereof the therapy and an effective amount of an agent for modulating the STING pathway.

100. The method of claim 99, wherein administration of the therapy and the agent enhances an immune response to the disease in the subject, compared to administration of the therapy alone.

101. The method of claim 99 or claim 100, wherein the disease is a cancer.

102. The method of claim 101, wherein the cancer is selected from: colorectal cancer, lung cancer, breast cancer, pancreatic cancer, prostate cancer, bladder cancer, kidney cancer, thyroid cancer, uterine cancer, cervical cancer, ovarian cancer, testicular cancer, esophageal cancer, stomach cancer, liver cancer, brain cancer, peritoneal cancer, lymphoma, leukemia, multiple myeloma, neuroblastoma, osteosarcoma, and soft tissue sarcoma.

103. The method of claim 101 or claim 102, wherein the therapy is a cancer immunotherapy and/or a cancer radiotherapy.

104. The method of any one of claims 99-103, wherein administration of the agent is intravenous, intramuscular, intradermal, oral, sublingual, or topical.

105. The method of any one of claims 99-104, wherein administration of the agent is systemic or local.

106. The method of any one of claims 99-105, wherein administration of the agent is simultaneous with the therapy.

107. The method of any one of claims 99-106, wherein administration of the agent is sequential with the therapy.

108. The method of any one of claims 99-107, wherein the agent reduces the activity of a negative regulator of STING in the subject.

109. The method of claim 108, wherein the negative regulator is a component of the ESCRT pathway.

110. The method of claim 109, wherein the negative regulator is hepatocyte growth factor-regulated tyrosine kinase substrate (HGS) or vacuolar protein sorting-associated protein 37 (VPS37A).

111. The method of claim 109, wherein the negative regulator of STING is a protein containing a J-domain.

112. The method of claim 111, wherein the protein containing a J-domain facilitates the palmitoylation of STING.

113. The method of claim 111 or claim 112, wherein the negative regulator of STING is DnaJ homolog subfamily C member 13 (DNAJC13).

114. The method of any one of claims 99-113, wherein the agent is a small molecular inhibitor of the negative regulator of STING.

115. The method of any one of claims 99-113, wherein the agent is a nucleic acid capable of reducing expression of the negative regulator of STING.

116. The method of claim 115, wherein the nucleic acid is a short interfering RNA (siRNA), a short hairpin RNA (shRNA), or an antisense oligonucleotide (ASO).

117. The method of claim 116, wherein the nucleic acid is capable of hybridizing to messenger RNA (mRNA) encoding the negative regulator of STING.

118. The method of any one of claims 115-117, wherein the nucleic acid reduces expression of the negative regulator of sting by up to 10%, up to 20%, up to 30%, up to 40%, up to 50%, up to 60%, up to 70%, up to 80%, up to 90%, up to 99%, or up to 100%.

119. The method of claim any one of claims 99-107, wherein the agent is a recombinant protein having STING activity or a nucleic acid that encodes a recombinant protein having STING activity.

120. The method of claim 119, wherein the nucleic acid is mRNA.

121. The method of claim 119 or claim 120, wherein the recombinant protein comprises recombinant STING.

122. The method of claim 121, wherein the recombinant protein is a fusion protein comprising STING and one or more additional proteins or fragments thereof.

123. The method of claim 123, wherein the recombinant protein is a fusion protein comprising STING and one or more FYVE domains.

124. The method of claim 123, wherein the recombinant protein is a fusion protein comprising STING and two FYVE domains.

125. The method of claim 123 or claim 124, wherein the one or more FYVE domains are fused to the N-terminus of STING.

126. The method of claim 125, wherein the recombinant protein does not comprise the endogenous transmembrane domain of STING.

127. The method of any one of claims 119-126, wherein the recombinant protein comprises constitutively active STING.

128. A method for screening for agents that modulate the activity of a Stimulator of Interferon Genes (STING) protein, comprising:
a) contacting a first population of cells expressing sting with an agent;
b) measuring the level of STING activity in the first population of cells; and
c) comparing the level of STING activity to the level of STING activity in a second population of cells;
wherein the second population of cells are not contacted with the agent; and
wherein a deviation in the level of STING activity in the first population of cells from that of the second population of cells is indicative that the agent is a modulator of STING.

129. The method of claim 128, further comprising a step of comparing the level of STING activity in the first population of cells to the level of STING activity in a third population of cells in which expression of a negative regulator of STING has been ablated,
wherein a lack of deviation in the level of STING activity in the first population of cells from that of the third population of cells is indicative that the agent inhibits the activity of the negative regulator.

130. The method of claim 128 or claim 129, wherein first, second, and/or third population of cells express STING endogenously or heterologously.

131. The method of any one of claims 128-130, wherein the agent is a small molecule, a nucleic acid, or a polypeptide.

132. The method of claim 131, wherein the nucleic acid is a short interfering RNA (siRNA), a short hairpin RNA (shRNA), or an antisense oligonucleotide (ASO).

133. The method of any one of claims 129-132, wherein expression of the negative regulator of STING has been ablated by treating the third population of cells with a short interfering RNA (siRNA), a short hairpin RNA (shRNA), or an antisense oligonucleotide (ASO) that reduces expression of the negative regulator.

134. The method of any one of claims 129-133, wherein the negative regulator is a component of the ESCRT pathway.

135. The method of claim 134, wherein the negative regulator is hepatocyte growth factor-regulated tyrosine kinase substrate (HGS) or vacuolar protein sorting-associated protein 37 (VPS37A).

136. The method of any one of claims 129-133, wherein the negative regulator of STING is a protein containing a J-domain.

137. The method any one of claims 129-133, wherein the negative regulator facilitates palmitoylation of STING.

138. The method of claim 136 or claim 137, wherein the negative regulator of STING is DnaJ homolog subfamily C member 13 (DNAJC13).

139. A pharmaceutical composition comprising an agent that results in an increase in STING activity when administered to a subject and a pharmaceutically acceptable excipient.

140. The composition of claim 139, wherein the agent reduces the activity of a negative regulator of STING.

141. The composition of claim 140, wherein the negative regulator is a component of the ESCRT pathway.

142. The composition of claim 141, wherein the negative regulator is hepatocyte growth factor-regulated tyrosine kinase substrate (HGS) or vacuolar protein sorting-associated protein 37 (VPS37A).

143. The composition of claim 140, wherein the negative regulator of STING is a protein containing a J-domain.

144. The composition of claim 143, wherein the protein containing a J-domain facilitates the palmitoylation of STING.

145. The composition of claim 143 or claim 144, wherein the negative regulator of STING is DnaJ homolog subfamily C member 13 (DNAJC13).

146. The composition of any one of claims 139-145, wherein the agent is a small molecular inhibitor of the negative regulator of STING.

147. The composition of any one of claims 139-145, wherein the agent is a nucleic acid capable of reducing expression of the negative regulator of STING.

148. The composition of claim 147, wherein the nucleic acid is a short interfering RNA (siRNA), a short hairpin RNA (shRNA), or an antisense oligonucleotide (ASO).

149. The composition of claim 148, wherein the nucleic acid is capable of hybridizing to messenger RNA (mRNA) encoding the negative regulator of STING.

150. The composition of claim 139, wherein the agent is a recombinant protein having STING activity or a nucleic acid that encodes a recombinant protein having STING activity.

151. The composition of claim 150, wherein the nucleic acid is mRNA.

152. The composition of claim 150 or claim 151, wherein the recombinant protein comprises recombinant STING.

153. The composition of claim 152, wherein the recombinant protein is a fusion protein comprising STING and one or more additional proteins or fragments thereof.

154. The composition of claim 153, wherein the recombinant protein is a fusion protein comprising STING and one or more FYVE domains.

155. The composition of claim 154, wherein the recombinant protein is a fusion protein comprising STING and two FYVE domains.

156. The composition of claim 154 or claim 155, wherein the one or more FYVE domains are fused to the N-terminus of STING.

157. The composition of claim 156, wherein the recombinant protein does not comprise the endogenous transmembrane domain of STING.

158. The composition of any one of claims 150-157, wherein the recombinant protein comprises constitutively active STING.


SEQUENCE LISTING (32 sequences)

SEQ ID NO: 1 - DNA, 2893 bp, Homo sapiens
gcgccagctcgtagcaggggagcgcccgcggcgtcgggtttgggctggaggtcgccatggggcgaggcagcggcaccttcgagcgtctcctagacaaggcgaccagccagctcctgttggagacagattgggagtccattttgcagatctgcgacctgatccgccaaggggacacacaagcaaaatatgctgtgaattccatcaagaagaaagtcaacgacaagaacccacacgtcgccttgtatgccctggaggtcatggaatctgtggtaaagaactgtggccagacagttcatgatgaggtggccaacaagcagaccatggaggagctgaaggacctgctgaagagacaagtggaggtaaacgtccgtaacaagatcctgtacctgatccaggcctgggcgcatgccttccggaacgagcccaagtacaaggtggtccaggacacctaccagatcatgaaggtggaggggcacgtctttccagaattcaaagagagcgatgccatgtttgctgccgagagagccccagactgggtggacgctgaggaatgccaccgctgcagggtgcagttcggggtgatgacccgtaagcaccactgccgggcgtgtgggcagatattctgtggaaagtgttcttccaagtactccaccatccccaagtttggcatcgagaaggaggtgcgcgtgtgtgagccctgctacgagcagctgaacaggaaagcggagggaaaggccacttccaccactgagctgccccccgagtacctgaccagccccctgtctcagcagtcccagctgccccccaagagggacgagacggccctgcaggaggaggaggagctgcagctggccctggcgctgtcacagtcagaggcggaggagaaggagaggctgagacagaagtccacgtacacttcgtaccccaaggcggagcccatgccctcggcctcctcagcgccccccgccagcagcctgtactcttcacctgtgaactcgtcggcgcctctggctgaggacatcgaccctgagctcgcacggtatctcaaccggaactactgggagaagaagcaggaggaggctcgcaagagccccacgccatctgcgcccgtgcccctgacggagccggctgcacagcctggggaagggcacgcagcccccaccaacgtggtggagaaccccctcccggagacagactctcagcccattcctccctctggtggcccctttagtgagccacagttccacaatggcgagtctgaggagagccacgagcagttcctgaaggcgctgcagaacgccgtcaccaccttcgtgaaccgcatgaagagtaaccacatgcggggccgcagcatcaccaatgactcggccgtgctctcactcttccagtccatcaacggcatgcacccgcagctgctggagctgctcaaccagctggacgagcgcaggctgtactatgaggggctgcaggacaagctggcacagatccgcgatgcccggggggcgctgagtgccctgcgcgaagagcaccgggagaagcttcgccgggcagccgaggaggcagagcgccagcgccagatccagctggcccagaagctggagataatgcggcagaagaagcaggagtacctggaggtgcagaggcagctggccatccagcgcctgcaggagcaggagaaggagcggcagatgcggctggagcagcagaagcagacggtccagatgcgcgcgcagatgcccgccttccccctgccctacgcccagctccaggccatgcccgcagccggaggtgtgctctaccagccctcgggaccagccagcttccccagcaccttcagccctgccggctcggtggagggctccccaatgcacggcgtgtacatgagccagccggcccctgccgctggcccctaccccagcatgcccagcactgcggctgatcccagcatggtgagtgcctacatgtacccagcaggggccactggggcgcaggcggccccccaggcccaggccggacccaccgccagccccgcttactcatcctaccagcctactcccacagcgggctaccagaacgtggcctcccaggccccacagagcctcccggccatctctcagcctccgcagtccagcaccatgggctacatggggagccagtcagtctccatgggctaccagccttacaacatgcagaatctcatgaccaccctcccaagccaggatgcgtctctgccaccccagcagccctacatcgcggggcagcagcccatgtaccagcagatggcaccctctggcggtcccccccagcagcagccccccgtggcccagcaaccgcaggcacaggggccgccggcacagggcagcgaggcccagctcatttcattcgactgacccaggccatgctcacgtccggagtaacactacatacagttcacctgaaacgcctcgtctctaactgccgtcgtcctgcctccctgtcctctactgccggtagtgtcccttctctgcgagtgagggggggccttcaccccaagcccacctcccttgtcctcagcctactgcagtccctgagttagtctctgctttctttccccagggctgggccatggggagggaaggactttctcccaggggaagcccccagccctgtgggtcatggtctgtgagaggtggcaggaatggggaccctcaccccccaagcagcctgtgccctctggccgcactgtgagctggctgtggtgtctgggtgtggcctggggctccctctgcaggggcctctctcggcagccacagccaagggtggaggcttcaggtctccagcttctctgcttctcagctgccatctccagtgccccagaatggtacagcgataataaaatgtatttcagaaagca

SEQ ID NO: 2 - protein, 777 aa, Homo sapiens
MGRGSGTFERLLDKATSQLLLETDWESILQICDLIRQGDTQAKYAVNSIKKKVNDKNPHVALYALEVMESVVKNCGQTVHDEVANKQTMEELKDLLKRQVEVNVRNKILYLIQAWAHAFRNEPKYKVVQDTYQIMKVEGHVFPEFKESDAMFAAERAPDWVDAEECHRCRVQFGVMTRKHHCRACGQIFCGKCSSKYSTIPKFGIEKEVRVCEPCYEQLNRKAEGKATSTTELPPEYLTSPLSQQSQLPPKRDETALQEEEELQLALALSQSEAEEKERLRQKSTYTSYPKAEPMPSASSAPPASSLYSSPVNSSAPLAEDIDPELARYLNRNYWEKKQEEARKSPTPSAPVPLTEPAAQPGEGHAAPTNVVENPLPETDSQPIPPSGGPFSEPQFHNGESEESHEQFLKALQNAVTTFVNRMKSNHMRGRSITNDSAVLSLFQSINGMHPQLLELLNQLDERRLYYEGLQDKLAQIRDARGALSALREEHREKLRRAAEEAERQRQIQLAQKLEIMRQKKQEYLEVQRQLAIQRLQEQEKERQMRLEQQKQTVQMRAQMPAFPLPYAQLQAMPAAGGVLYQPSGPASFPSTFSPAGSVEGSPMHGVYMSQPAPAAGPYPSMPSTAADPSMVSAYMYPAGATGAQAAPQAQAGPTASPAYSSYQPTPTAGYQNVASQAPQSLPAISQPPQSSTMGYMGSQSVSMGYQPYNMQNLMTTLPSQDASLPPQQPYIAGQQPMYQQMAPSGGPPQQQPPVAQQPQAQGPPAQGSEAQLISFD

SEQ ID NO: 3 - DNA, 4519 bp, Homo sapiens
gctgggcggccaggctccctggctggccggtttgggcgtctgggccgtgaaggtgggacctcctgttccgggccgcaagtttccctctccagccgcccgccgttcgtagcatgtcccccagaactcggggagcgcaggcaggacaggcttagagaagacgcggtccccagcgcttgggccacggacgtcccaccccgctcctctgtcgctggagaaccgccgggccgagccactgggagaagcaggccagagccttccagggcctccggcccgtggacccgaggaggatgagctggctttttcccctgaccaagagcgcctcctcctccgcggctgggtcccccggtggcctcaccagcctccagcagcagaagcagcgcctgatcgagtccctccggaactcacactccagtatagccgaaatacagaaagatgtggaatacagattgccattcaccataaacaacctgacaattaacattaatatattgcttcctccacagtttcctcaggaaaaaccagtgatcagtgtttatccaccaatacgacatcacttaatggataaacaaggagtgtatgttacctctccattagtaaacaattttacaatgcactcagatcttggaaaaattattcagagtctgttggatgagttttggaagaatcctccagttttagctcctacttcaacagcatttccttatctatacagtaacccaagtgggatgtctccttatgcttctcagggttttccatttcttcctccatatcctccacaagaagcaaacaggagtatcacttctttatctgttgctgacactgtttcttcttcaacaacaagtcataccacagccaagcctgccgctccttcatttggtgtcctttcaaatctgccattacccattcccacagtggatgcttcaataccgacaagccaaaatggttttgggtacaagatgccagatgtccctgatgcatttccagaactctcagaactaagtgtgtcacaactcacagatatgaatgaacaagaggaggtattactagaacagtttctgactttgcctcaactaaaacaaattattaccgacaaagatgacttagtaaaaagtattgaggaactagcaagaaaaaatctccttttggagcccagcttggaagccaaaagacaaactgttttagataagtatgaattacttacacagatgaagtccactttcgaaaagaagatgcaaaggcagcatgaacttagtgagagctgtagtgcaagtgcccttcaggcaagattgaaagtagctgcacatgaagctgaggaagaatctgataatattgcagaagacttcttggagggaaagatggaaatagatgattttctcagtagcttcatggaaaagagaacaatttgccactgtagaagagccaaggaagagaaacttcagcaggcgatagcaatgcacagccaatttcatgctccactatagattttcctggaaacatgaactgccaagagaggaatgggacacaaaaccaaacactgttttatatttatggtttgcaaactggcatttcatcagtggctaaattcacagatatcctatatagattgtatacagaactgagactgattttgtaccgattagaatgattgctatgatctttgagaaatttttctgcactatttgcactgaaatgtttatttattgttgataaattgtatcatatttaagttccactgctgttcctcttaccttgattaaatgcctatgcatgtacttttagctagtttttaatattttataaaacttcatttaaatttgtatttttaacttgaagttccatttctttatcaaggatggtatttagatttttttcctcttaaccttttttcaaaaactattttcaactgtgaggaaacccttatttttctttctttgtggataaaactttcaaaagcaatttaagatattcatagtgttaggaaacaccaaacctgcctatgtgccatctcacaaaagaaacttttaatacctacaataaatcaaaagaataaaccagctgttcttatatattgtttcatttttaaaactaaagatgcatttaagaagcaatacaagtaaatattttacctaataggaaaaaaaaaagttgcctttcatttaaaccattccaacagaaattcttatgctaatttaaaacatatatatatctggtaggtttgtggttggataggttttctaaattcctaatgttaaaaacaatctttatgttaatatacactaaatctatacacaaaaaaagtcagtgaacttttctgacctttactgtgagttaccttttcctaagaggaaagctatagtaataagtaaaatttaatttttaggcaatcctgatttttaatgaatttaattgagtgttcttgtatactacattgagcagtttgcttctataccgtgtcacaaaattcatgtatttcttgagaagccctaaaagctcataaaggaaaatgccgtgaactatgtagctcaggcttggtaaggtgccatctaaattacaaaacaaactaatgcataattttgcttaaatttcatcccagtatgattgtcttcccaacaccagcatatagtatagattgtctgtcttttttatattttttagttcttcctgtacatgtttttggcaataaagttataggaagaacaaaattattttgttagaattaaaacatgcttaatatttagtctgtttgtggagggcaggtattcacgtggactgagatacaatgttggatacagaaaataactttcattgtcttcctgacactgtgctaaggacatgctgttaaagcttcaaagtgaccagatgaggaaggaataattaattattactcctgatttgtagataactgaggtaagagtgtttcaaatttatgatagtcttttgggtattcagaaacctttccttatactgcactggccaccagagcttaattttcccagcagttacagcaatgggagatagaacagtctcaatcttttgccaaccatcaggttcctagaaaccaggtaggtgtatcccataacaagggaggagcataccacagcccctcatttgattaattcatttgatctatctatgttattaagtacctactaggaataaggcattgtggaaatactatacaaagataaacattgtttagatgcttatctactttccttttcaccagaaaaacagaaaaaaaagaaacattttcttacagagtaaaaatgttctacataatcacatgagtagttcatctcagtgttttttattctttaaagttgaactatcccagtttcattctataccattcattggataaccttgttacaacccagtcatgaaacagagcagtgtgatcagttatctgcatttaacaaatagacaaatcagtttacataaaggttatgtatgtcacccacgatgaaaagaatctgcatttgaatatgcccgtatgaatgtgggttctgtttttgcaacagagattaagtgaccattttttctaattttatggctatatattttcttcataaaaattggtcacatcggagaagcagtgccacaggaaaaatgaaatgcatgtgaaagtttgtattctgattttacaagatgagatagaaatcagaattaaagaggaatacttaggagttactaggctaatcagtgtacgaatttgtcataggtagagatttaaaggttaatatcttaaaatagaagaaaattctaaatcaatcaatcagtgagatataaactaaacagacccacttcaaagttgaaagaaatttctaggcataaattgagactaggaaatttatatcagaatagagggtgcttgacacatatatatgcttaaattgaaggacagctcagattcatttttaggagaagaaagtaaactaatgtgctcttaaagaataaaaatttattctatggtttctgtctctgatcatcaccttccattctataaaaagctcagttactgatttgctgggtcatggtcaaaattcttacctatttatttcatatcaactttaaaaaataaattacttgcattctatatattactaattgggaagtaatatgcctcaaatcagttttatactggattattccctatgctttaaaccactgctctcaataaaacacttcctgattaatgtttgattattagatattttagtcttgttggggatattttagtcttgttgggttagccatgctctgaagaatctgtgaaagtacagtaaagttttaataagcaataaatgtaaccttttatataaatctcagtgctaggttaacttctaataagcagacgaacatgttacataaattataatgtctgtcttgtaaaaaagttgaggggactaaaagtttatgactctgatatggaagttgtcatattaaaaaactacattttaaaacatcaaatatttatactatttgcttttcaaataaaagcatagtgctgtttggcata

SEQ ID NO: 4 - protein, 397 aa, Homo sapiens
MSWLFPLTKSASSSAAGSPGGLTSLQQQKQRLIESLRNSHSSIAEIQKDVEYRLPFTINNLTININILLPPQFPQEKPVISVYPPIRHHLMDKQGVYVTSPLVNNFTMHSDLGKIIQSLLDEFWKNPPVLAPTSTAFPYLYSNPSGMSPYASQGFPFLPPYPPQEANRSITSLSVADTVSSSTTSHTTAKPAAPSFGVLSNLPLPIPTVDASIPTSQNGFGYKMPDVPDAFPELSELSVSQLTDMNEQEEVLLEQFLTLPQLKQIITDKDDLVKSIEELARKNLLLEPSLEAKRQTVLDKYELLTQMKSTFEKKMQRQHELSESCSASALQARLKVAAHEAEEESDNIAEDFLEGKMEIDDFLSSFMEKRTICHCRRAKEEKLQQAIAMHSQFHAPL

SEQ ID NO: 5 - DNA, 7769 bp, Homo sapiens
agagcgcgctcgctcaaagcctgagcgaagatggcggcctccagagtgaaactctgagaggcagagggaggaggcggagggggcggggaggcagcgccgcggcggcaccaggaacccgcggcagcggagctgcagggccccggcgggggaaaggggaagtggtgagggtgaggaggccggagggcgcctcatcccactctggaagctgagccggcggcgggaggagctagggctggagcctctccagcctcccgcgaaggtttgagcacaaaatgaacataattagggaaaataaggatctggcatgtttctacacaacaaaacattcatggagggggaagtataagcgtgtcttttcagttggaactcatgcgattactacatataatcccaataccttagaagtaacaaatcagtggccttatggagacatttgcagcatcagccctgttggaaaaggacaaggaacggagttcaacctcacatttcgtaaaggcagtggaaaaaagtcagaaactttaaaattttctacagagcacagaacagaacttcttacagaagcattgagatttagaactgatttttcagagggaaaaatcacaggaaggagatacaactgctataagcatcactggagtgactcaagaaaacctgtaattttggaagtaactccaggaggctttgaccaaattaatcctgcaaccaacagagtactctgttcctatgactatagaaatattgaaggatttgtagatctctcagattatcaaggaggattttgtatactttatggaggatttagtagattgcatttatttgcgtcagagcaaagagaagagattattaaaagtgcaatagaccatgctggtaactacataggtatttcattgcggatcaggaaagagcctttagaattcgagcaatatttgaatcttcgctttggaaaatacagcactgatgaatccatcacatctttagcagagtttgtagtccaaaaaatatcacctagacattcggagcctgttaaaagagttctagcacttacagaaacatgtttagtagaacgtgatccggcaacctataatattgcaacattgaagcctttaggagaagtatttgcgttggtctgtgactcagaaaatccacaactttttaccattgaatttataaaagggcaagtacggaaatattcttcaacagagagagattccttattagcaagtttgctggatggagtaagagcctctggtaatagagatgtttgtgtaaaaatgacaccaacccataaaggtcagcgatgggggttactcagcatgcctgttgatgaggaagtagagagccttcacctcaggttcttagctacgcctccaaatggcaactttgcagatgctgtattcaggttcaatgctaatatttcatacagtggagtcctacatgcagtaacacaagatggtctcttctcagaaaacaaagaaaaactgatcaataatgccataacagcattactgtcccaagaaggggatgtcgttgcttcaaatgcggaacttgagagtcagttccaggctgtgaggaggcttgtggcatccaaagctggtttcctggctttcactcagcttccaaagtttcgcgagcgtctaggggtgaaggtagtaaaagcactcaaaagaagcaacaacggaataatccatgcagcagttgatatgctttgtgcccttatgtgtcccatgcatgatgactatgacttaagacaagaacagttgaacaaagcttctcttctctcgtcaaagaagtttctggaaaacttactggagaaatttaattcccatgtggatcatgggactggtgccctagttattagttcgctcttggacttccttacctttgccctctgtgctccatatagtgagacaactgaagggcagcagtttgatatgctcttggagatggtagcatccaatggaagaaccctttttaaactttttcagcatccttccatggcaataataaaaggagctgggttggttatgaaggcaataatagaggaaggtgataaagaaattgctacaaaaatgcaggagcttgccctaagtgaaggtgccttacctcgacacttgcatactgcgatgtttacaataagctcagatcaaaggatgcttacaaatagacagctaagtagacatttagtgggactctggacagctgataatgcaactgcaacaaacttgttgaaacgcattttgccgccaggcttgctggcatacttggaaagctcagatctcgtacctgagaaggatgctgatcggatgcatgttagagacaatgtgaaaatagcaatggatcagtatggaaaatttaataaagttccagagtggcaaagactagctggaaaagctgctaaagaagttgaaaaatttgccaaagaaaaagtggatcttgtattgatgcactggagggataggatgggcattgctcaaaaagaggacaaaaacaatatgaatataaatcagaagccagtggttcttcgaaagagaagacaaagaataaaaatagaagcaaattgggatctcttctattataggtttggtcaagaccatgccaggtcaaaccttatttggaatttcaaaacacgagaagaactgaaagatactcttgaatctgaaatgagagcatttaatattgacagagaacttggaagtgcaaatgtgatctcctggaaccaccatgagtttgaggttaaatatgagtgcctggcagaggaaattaaaataggagactattacctgagattactattggaggaagatgagaatgaagaaagtggatcaattaagagatcgtatgaatttttcaatgagctttatcatcgcttcttgctcaccccaaaagtaaacatgaagtgtttatgtttacaagcccttgctattgtttatggcagatgtcacgaagaaataggaccttttacagataccagatatatcattggaatgttagagaggtgcacagataaacttgaacgagataggttgattctcttccttaacaagttgatccttaataagaaaaatgttaaggatctcatggattcaaatggaataagaatccttgtggacttgcttacccttgcacatctccatgtaagccgagctacagtaccactgcaaagcaatgtaattgaagctgctccagatatgaaaagagagagtgaaaaggaatggtattttggcaacgcagacaaagaaaggagtggcccgtatggatttcatgagatgcaagaattgtggaccaaaggaatgttaaatgcaaaaaccagatgctgggctcaaggcatggatggatggcgaccacttcagtccataccccagcttaagtggtgtctcttagccagtggacaggctgtcctgaatgaaactgaccttgctacccttatattgaacatgttgatcacaatgtgtggatattttccaagcagggatcaagacaatgccatcattcggcctctacccaaagtgaaaagactgctgtcagatagcacttgccttccccatattattcagctactgctgacctttgaccctatccttgttgagaaggttgctattttgttataccatatcatgcaagataacccacagttaccccgcctttatctgagtggagtatttttctttatcatgatgtacacaggttccaatgtgcttcctgttgctcgatttttgaaatacacacataccaaacaggctttcaagtcagaagagacaaaaggacaagatatttttcagagaagtatacttgggcacattctacctgaagcaatggtttgttacttagaaaattatgaacctgaaaagttttctgagatttttctaggagaatttgatactccagaagcaatctggagcagtgaaatgaggcgcctgatgatagagaagattgctgcccatctcgcggatttcacacctcgtcttcagagtaacacaagagcactttatcagtattgccccattcctataatcaactatccacaactcgaaaatgaactattttgtaatatttattacctcaaacaactgtgtgatacactccggtttccagattggccaattaaagacccggttaagcttctaaaagatacccttgatgcctggaagaaagaagtagaaaagaagccacctatgatgtcaatagatgatgcttatgaagtgcttaatctgcctcaaggacagggaccgcatgatgagagcaagattaggaaagcttacttcagacttgcacaaaagtaccaccctgataagaatccagaagggagggacatgtttgaaaaagtaaataaagcatatgaatttttatgtaccaaatcagcaaaaatagtggatgggccagatccagagaatataattttaattctaaaaacacagagcatcctcttcaaccgtcataaagaagatttacagccttataaatatgcaggataccccatgcttattcggactataacaatggaaacttcagatgacctccttttctcaaaagaatcaccattgttgcctgcggctacagagctagctttccatactgtcaactgttcagccctcaatgctgaagagctcagaagagagaatggactagaggtgttacaagaggcatttagtcgctgtgtggctgtcttgactcgtgctagtaaaccaagtgacatgtcagtacaggtgtgtggatacataagtaaatgctacagtgtggctgctcagtttgaggaatgccgagagaagatcacggaaatgcctagcatcatcaaggatctctgtcgggtactatattttggcaagagtattccccgcgtagctgctcttggggtagaatgtgtcagttcttttgctgtggatttctggctacagacacacctatttcaggctggaattttgtggtatctccttggttttctgtttaattatgactacacactagaagagagtggcattcagaaaagtgaagaaacaaaccagcaggaggtagcaaacagccttgccaaactgagtgtccatgctctgagtcgccttggagggtatttggctgaagaacaagcaactccagaaaatccaaccataaggaaaagcttagctggcatgctgacaccctatgttgctagaaaacttgctgtggctagtgtgactgagattttgaagatgcttaacagcaacacagaaagtccatatttgatatggaacaattctacaagagcagaattacttgaatttcttgaatcccaacaagaaaacatgattaaaaaaggtgattgtgacaaaacttatggatcagaatttgtctacagtgatcatgccaaagaacttattgtaggggagatttttgttagggtgtataatgaagttcctactttccaactggaggttccaaaagcatttgctgcaagtctcttggattatataggctcgcaggcccaatacttgcacacattcatggccatcacacacgcggcaaaagtggagtcagagcaacatggagatcgcttaccgagagtagaaatggctttggaggctctgagaaatgtcataaaatacaatccaggttctgagagtgaatgcattgggcactttaagttgatattttctcttctccgagttcatggagctggtcaagtgcagcagttggctttagaggttgtgaatatagtgacatctaaccaagactgtgtcaacaatattgctgaatcaatggttttgtccagtttattggctcttctacattcattgccatcaagtcgtcagcttgttctggaaactctttatgctttgacatcgagtacaaaaataatcaaagaagcaatggcaaagggtgctttgatctatttactggatatgttctgcaattcaacacatccacaggttcgagcccaaacagcagaactttttgccaaaatgacagcagataaactgataggtccaaaggttcgaattacgttaatgaaatttctaccaagcgttttcatggatgctatgagagacaatcctgaagctgctgtacatatttttgaaggaactcatgaaaatcctgagttaatttggaatgataattccagagataaagtgtccacaacagttagggaaatgatgctagagcactttaaaaatcagcaggacaaccctgaggcaaactggaagttgcctgaagattttgctgtggtgtttggagaagcagagggtgaacttgctgttggaggagtcttcttgaggatctttattgcacaaccagcctgggttctaagaaagcctagagaatttcttattgccctgttagaaaaattaactgagctcctagagaagaacaatcctcatggagaaactctggaaaccttgacaatggcaacagtgtgtctcttcagcgcacaacctcagctggcagatcaggtcccgccattgggccatcttcccaaagttatccaggcaatgaatcataggaacaatgccattcctaagagtgccattcgggttatccatgccttgtctgaaaatgagctgtgtgttcgagccatggcatctttagagaccattggcccactgatgaatggaatgaaaaagcgagcagatactgttggtctagcctgtgaagcaattaatcgaatgtttcagaaggagcagagtgaattagtagcacaagccctgaaagcagatttggttccatacctcttaaaattactcgaaggcattggccttgaaaacctggacagcccagcagccactaaggctcagattgttaaagctctcaaggcaatgactcgaagtttgcagtatggagaacaggtgaatgaaatcctgtgccgttcttcagtctggagtgccttcaaagatcagaaacatgatttgttcatttctgagtcacaaacagcaggatacctcacaggacctggagttgctggctaccttaccgcaggtacatctacatcagtcatgtctaacctgccacctcctgtagaccatgaggcaggcgaccttggctatcagacttgaaatattcacgagagacaataaacgctgaaaggccagtgccaagtccacattcctccagctgatacgttgaagcaaactcttactgcctttctcctggtttcatgacagtgttattcctttttctataaatatatttttaggaaaaaaagtcagtgatcctaattgtatcacattataagaaagcactctgtggatcaacataagtgggtacacaagaattttttttttcttggtgtatgtaagcacatttgttcctttatatctgtttacaaaactgtgaatcaaaaagacaaaactttcttcctagtttttgtaattttttttttgaactagcatgactgtagggttgagctacagtcaacaaaaattgggctaagtcacttttccccaggaaagaatatttccctctcctgcatcaagtctgcgtggccatcctccccccaccatccaagactattaggttttgtccctgcacccttcactggcatcctcaatcattaaccttctgaaagctcacagtacacattagtatgtataactggctttaccaaattgaatgaaaaggagcttgtgcaaaaaaatttaaaaatggatgtcaagatgttatgtaaaagatgagtgtaattgtgaaatgttctatacactatcaaatatataaagctttctatattgaatgtacattatacagatcattcatatgtgtacataaaattttaaaaataaagggaattgactgctttgttaatgaga

SEQ ID NO: 6 - protein, 2248 aa, Homo sapiens
MNIIRENKDLACFYTTKHSWRGKYKRVFSVGTHAITTYNPNTLEVTNQWPYGDICSISPVGKGQGTEFNLTFRKGSGKKSETLKFSTEHRTELLTEALRFRTDFSEGKITGRRYNCYKHHWSDSRKPVILEVTPGGFDQINPATNRVLCSYDYRNIEGFVDLSDYQGGFCILYGGFSRLHLFASEQREEIIKSAIDHAGNYIGISLRIRKEPLEFEQYLNLRFGKYSTDESITSLAEFVVQKISPRHSEPVKRVLALTETCLVERDPATYNIATLKPLGEVFALVCDSENPQLFTIEFIKGQVRKYSSTERDSLLASLLDGVRASGNRDVCVKMTPTHKGQRWGLLSMPVDEEVESLHLRFLATPPNGNFADAVFRFNANISYSGVLHAVTQDGLFSENKEKLINNAITALLSQEGDVVASNAELESQFQAVRRLVASKAGFLAFTQLPKFRERLGVKVVKALKRSNNGIIHAAVDMLCALMCPMHDDYDLRQEQLNKASLLSSKKFLENLLEKFNSHVDHGTGALVISSLLDFLTFALCAPYSETTEGQQFDMLLEMVASNGRTLFKLFQHPSMAIIKGAGLVMKAIIEEGDKEIATKMQELALSEGALPRHLHTAMFTISSDQRMLTNRQLSRHLVGLWTADNATATNLLKRILPPGLLAYLESSDLVPEKDADRMHVRDNVKIAMDQYGKFNKVPEWQRLAGKAAKEVEKFAKEKVDLVLMHWRDRMGIAQKEDKNNMNINQKPVVLRKRRQRIKIEANWDLFYYRFGQDHARSNLIWNFKTREELKDTLESEMRAFNIDRELGSANVISWNHHEFEVKYECLAEEIKIGDYYLRLLLEEDENEESGSIKRSYEFFNELYHRFLLTPKVNMKCLCLQALAIVYGRCHEEIGPFTDTRYIIGMLERCTDKLERDRLILFLNKLILNKKNVKDLMDSNGIRILVDLLTLAHLHVSRATVPLQSNVIEAAPDMKRESEKEWYFGNADKERSGPYGFHEMQELWTKGMLNAKTRCWAQGMDGWRPLQSIPQLKWCLLASGQAVLNETDLATLILNMLITMCGYFPSRDQDNAIIRPLPKVKRLLSDSTCLPHIIQLLLTFDPILVEKVAILLYHIMQDNPQLPRLYLSGVFFFIMMYTGSNVLPVARFLKYTHTKQAFKSEETKGQDIFQRSILGHILPEAMVCYLENYEPEKFSEIFLGEFDTPEAIWSSEMRRLMIEKIAAHLADFTPRLQSNTRALYQYCPIPIINYPQLENELFCNIYYLKQLCDTLRFPDWPIKDPVKLLKDTLDAWKKEVEKKPPMMSIDDAYEVLNLPQGQGPHDESKIRKAYFRLAQKYHPDKNPEGRDMFEKVNKAYEFLCTKSAKIVDGPDPENIILILKTQSILFNRHKEDLQPYKYAGYPMLIRTITMETSDDLLFSKESPLLPAATELAFHTVNCSALNAEELRRENGLEVLQEAFSRCVAVLTRASKPSDMSVQVCGYISKCYSVAAQFEECREKITEMPSIIKDLCRVLYFGKSIPRVAALGVECVSSFAVDFWLQTHLFQAGILWYLLGFLFNYDYTLEESGIQKSEETNQQEVANSLAKLSVHALSRLGGYLAEEQATPENPTIRKSLAGMLTPYVARKLAVASVTEILKMLNSNTESPYLIWNNSTRAELLEFLESQQENMIKKGDCDKTYGSEFVYSDHAKELIVGEIFVRVYNEVPTFQLEVPKAFAASLLDYIGSQAQYLHTFMAITHAAKVESEQHGDRLPRVEMALEALRNVIKYNPGSESECIGHFKLIFSLLRVHGAGQVQQLALEVVNIVTSNQDCVNNIAESMVLSSLLALLHSLPSSRQLVLETLYALTSSTKIIKEAMAKGALIYLLDMFCNSTHPQVRAQTAELFAKMTADKLIGPKVRITLMKFLPSVFMDAMRDNPEAAVHIFEGTHENPELIWNDNSRDKVSTTVREMMLEHFKNQQDNPEANWKLPEDFAVVFGEAEGELAVGGVFLRIFIAQPAWVLRKPREFLIALLEKLTELLEKNNPHGETLETLTMATVCLFSAQPQLADQVPPLGHLPKVIQAMNHRNNAIPKSAIRVIHALSENELCVRAMASLETIGPLMNGMKKRADTVGLACEAINRMFQKEQSELVAQALKADLVPYLLKLLEGIGLENLDSPAATKAQIVKALKAMTRSLQYGEQVNEILCRSSVWSAFKDQKHDLFISESQTAGYLTGPGVAGYLTAGTSTSVMSNLPPPVDHEAGDLGYQT

SEQ ID NO: 7 - skipped

SEQ ID NO: 8 - DNA, 20 bp, Artificial sequence
  Synthetic
gtattactgatattggtggg

SEQ ID NO: 9 - DNA, 20 bp, Artificial sequence
  Synthetic
gaagatgacaggaacaaccg

SEQ ID NO: 10 - DNA, 20 bp, Artificial sequence
  Synthetic
aagagcccagggatagctca

SEQ ID NO: 11 - DNA, 20 bp, Artificial sequence
  Synthetic
tgaacagtaccgcctgcaga

SEQ ID NO: 12 - DNA, 20 bp, Artificial sequence
  Synthetic
tgccacgggatcccaatgcc

SEQ ID NO: 13 - DNA, 20 bp, Artificial sequence
  Synthetic
cttggggtacgaagtgtacg

SEQ ID NO: 14 - DNA, 20 bp, Artificial sequence
  Synthetic
gcacgcagcccccaccaacg

SEQ ID NO: 15 - DNA, 20 bp, Artificial sequence
  Synthetic
cagcccctcatagtacactg

SEQ ID NO: 16 - DNA, 20 bp, Artificial sequence
  Synthetic
cataaggagacatcccactt

SEQ ID NO: 17 - DNA, 20 bp, Artificial sequence
  Synthetic
ggtggataaacactgatcac

SEQ ID NO: 18 - DNA, 20 bp, Artificial sequence
  Synthetic
cattacaacaacctgctacg

SEQ ID NO: 19 - DNA, 20 bp, Artificial sequence
  Synthetic
atccctccgtacgagaatgg

SEQ ID NO: 20 - DNA, 20 bp, Artificial sequence
  Synthetic
tggttctgccagcaaacgct

SEQ ID NO: 21 - DNA, 20 bp, Artificial Sequence
  Synthetic
ctgttgccttcatagataag

SEQ ID NO: 22 - protein, 377 aa, Nematostella vectensis
MRRAEENNGFGTIPKRRNQHTPFYASIGMIVVIIVAFTSYHITSYGDDRNRAMRQYSFTFSLAYLAFLVGELLRRCCLFAEEYRHIETRYNGSLKKAIQTTFSFGHNNVLFVASLLFFVVFVASNDPNGSSSVIQGNSTAEPHTEMRQTSGWQGLWGQFIISALLTPLVVHLLGLRELSKVEESQLNEKENKNVADGLAWSYYFGYLKFVLPELEKQIEKTSKFRSKEKFVKKMFILIPSNCFWDDKIPGSDYDPQNRITFEGNTEPLEKTRGGVFLRHYKHSVYEIKDGENEPWFCIMEYATPLLTLYDMSVAQPGELSREERDAQVVVFLRKLQDILEGDRACQGKYELVTFSPDRDLADVMLRKLKDSELEIGG

SEQ ID NO: 23 - protein, 398 aa, Unknown
  sp|E7F4N7|STING DANRE
MSVMGEDALVPRARSRLPVMCAAGLGFLTLAVAWLLDSDKFSERAGIIAFGLMLERFIYCICLLAEELLFHSRQRYHGRMSEIFRACFRGSGILGMCAIFLMLMLGGVSFSVKQWSHFNLMCAGYMLLNSLGVLGPAPVEISEICEAKKMNVAHGLAWSFYIGYLKFLLPALEVNVREYSRRERLSSPRLHILLPLNARVPSKPEEEDTNVVFHENLPDLKLDRAGVRKRSYTNSVYKITHNNETFSCILEYATPLLTLYQMSQESSAGFGERERKQQVLLFYRTLSQILDNSLECRNRYRLILLNDEHTGDPHYLSRELFQNLKQQDGEIFMDPTNEVHPVPEEGPVGNCNGALQATFHEEPMSDEPTLMFSRPQSLRSEPVETTDYFNPSSAMKQN

SEQ ID NO: 24 - protein, 298 aa, Unknown
  sp|A8E5V9|STING XENTR
MACVLAIGSILFVWILGKGKYSGAQLIYRMATNFAISQGCCLVTCACELTEEIKHLHTRYNGHYWRALKASFNLSCAAFVTAILCYVFYEPKLMASLPLTIDITLTLLSWLFCWILGIQGPTPATISEITEIKQLNVAHGLAWSYYVGYLQFVLPALKESIQKFDVDENITFLKEIPPLYIDRAGIKGRVFKNNVYRILDEDGRPYNCIVEYATPLASLLKMTDIPSAAFSADDRLQQTKLFYRTLKDILENAHELQNTYRLIVYEDFPETKDHSRHLLSQEILKHIRQQHSEEYSML

SEQ ID NO: 25 - protein, 375 aa, Unknown
  sp\E1C7U0\STING_CHICK
MPQDPSTRSSPARLLIPEPRAGRARHAACVLLAVCFVVLFLSGEPLAPIIRSVCTQLAALQLGVLLKGCCCLAEEIFHLHSRHHGSLWQVLCSCFWYLALLLVGGSAYLDPPEDNGHSPRLALTLSCLCQLLVLALGLQKLSAVEVSELTESSKKNVAHGLAWSYYIGYLKVVPRLKECMEELSRTNPMLRAHRDTWKLHILVPLGCDIWDDLEKADSNIQYLADLPETILTRAGIKRRVYKHSLYVIRDKDNKLRPCVLEFASPLQTLCAMSQDDCAAFSREQRLEQARLFYRSLRDILGSSKECAGLYRLIAYEEPAEPESHFLSGLILWHLQQQQREEYMVQEELPLGTSSVELSLQVSSSDLPQPLRSDCP

SEQ ID NO: 26 - protein, 379 aa, Rattus norvegicus
MPYSNLHPSIPRPRSYRFKLAAFVLLVGSLMSLWMTGEPPSHTLHYLALHVASQQLGLLLKKLCCLAEELCHVQSRYQGSYWKAVRACVGSPICFMALILLSFYFYCSLENTSDLRLAWHLGILVLSKSLSMTLDLQSLAPAEVSAVCEEKNFNVAHGLAWSYYIGYLKLILPGLQARIRMFNQLHNNMLSGAGSRRLYILFPLDCGVPDDLSVADPNIRFRDMLPQQNTDRAGVKNRAYSNSVYELLENGQPAGACILEYATPLQTLFAMSQDGKAGFSREDRLEQAKLFCRTLEEILADVPESRNHCRLIVYQESEEGNSFSLSQEVLRHIRQEEKEEVTMSGPPTSVAPRPSLLSQEPRLLISGMEQPLPLRTDLI

SEQ ID NO: 27 - protein, 341 aa, Mus musculus
MPYSNLHPAIPRPRGHRSKYVALIFLVASLMILWVAKDPPNHTLKYLALHLASHELGLLLKNLCCLAEELCHVQSRYQGSYWKAVRACLGCPIHCMAMILLSSYFYFLQNTADIYLSWMFGLLVLYKSLSMLLGLQSLTPAEVSAVCEEKKLNVAHGLAWSYYIGYLRLILPGLQARIRMFNQLHNNMLSGAGSRRLYILFPLDCGVPDNLSVVDPNIRFRDMLPQQNIDRAGIKNRVYSNSVYEILENGQPAGVCILEYATPLQTLFAMSQDAKAGFSREDRLEQAKLFCRTLEEILEDVPESRNNCRLIVYQEPTVLSQEPRLLISGMDQPLPLRTDLI

SEQ ID NO: 28 - protein, 389 aa, Unknown
  tr|H9YZQ6|H9YZQ6 MACMU
MTRSSLHPSIPCPRGHGAQKAALVLLTACLGTLWGLGESPEHILRCLVLHLASLQLGQLLNGVCSLAEELRHIHSRYRDSYWRTVRACLGCPFHRGTLLLLSGYFYYSLPNAVGLPFTWMLALLGLSQALNILLGLKGLTPAEISAVCEKGNFNVAHGLAWSYYIGYLRLILPGLQARIQTYNQHYNNLLRGAVSQRLYILLPLDCGVPDNLSLDCGVPDNLSMADPNIRFLDKLPQQTADRAGIKDRVYSNSIYELLENGQRAGTCVLEYATPLQTLFAMSQYGQAGFSREDRLEQVKLFCRTLEDILADNPESQNNCRLIVYSEPADDSSFSLSQEVLRHLRQEEKEEVTVGSLKNSAVPSTSTMSQEPELLISGMEKPLPLRTDFS

SEQ ID NO: 29 - protein, 379 aa, Homo sapiens
MPHSSLHPSIPCPRGHGAQKAALVLLSACLVTLWGLGEPPEHTLRYLVLHLASLQLGLLLNGVCSLAEELRHIHSRYRGSYWRTVRACLGCPLRRGALLLLSIYFYYSLPNAVGPPFTWMLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTGDHAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 30 - protein, 379 aa, Panthera pardus
MPHSSLHPSIPCPRGHGAQKAALVLLSACLVTLWGLGEPPEHTLRYLVLHLASLQLGLLLNGVCSLAEELRHIHSRYWGSYWRTVRACLGCPLRRGALLLLSIYFYYSLPNAVGPPFTWMLALLGLSQALNILLGLKGLAPAEISAVCEKGNFNVAHGLAWSYYIGYLRLILPELQARIRTYNQHYNNLLRGAVSQRLYILLPLDCGVPDNLSMADPNIRFLDKLPQQTADRAGIKDRVYSNSIYELLENGQRAGTCVLEYATPLQTLFAMSQYSQAGFSREDRLEQAKLFCRTLEDILADAPESQNNCRLIAYQEPADDSSFSLSQEVLRHLRQEEKEEVTVGSLKTSAVPSTSTMSQEPELLISGMEKPLPLRTDFS

SEQ ID NO: 31 - protein, 378 aa, Bos taurus
MPHSSLHPSIPQPRGLRAQKAALVLLSACLVALWGLGEPPDYTLKWLVLHLASQQMGLLIKGICSLAEELCHVHSRYHGSYWRAVRACLCSSMRCGALLLLSCYFYCSLPNMADLPFTWMLALLGLSQALNILLGLQGLAPAEVSAICEKRNFNVAHGLAWSYYIGYLRLILPGLPARIQIYNQFHNNTLQGAGSHRLHILFPLDCGVPDDLNVADPNIRFLHELPQQSADRAGIKGRVYTNSIYELLENGQRAGVCVLEYATPLQTLFAMSQDGRAGFSREDRLEQAKLFCRTLEDILANAPESQNNCRLIVYQEPAEGSSFSLSQEILQHLRQEEREVTMGSTETSVMPGSSVLSQEPELLISGLEKPLPLRSDVF

SEQ ID NO: 32 - protein, 385 aa, Sus scrofa
MPYSSLHPSIPQPRGLRAQVAALVLLGACLVALWGLGELPEYTLRWLVLHLASQQIGLLVKGLCSLAEELCHVHSRYQSSYWRAARACLGCPIRCGALLLLSCYFYFSIRDKAGLPLPWMLALLGLSQALNILLGLQHLAPAEVSAICEKRNFNVAHGLAWSYYIGYLRLILPGLRARIQAYNQRHKNVLGGIGNHRLHILFPLDCGVPDDLSGVPDDLSVADPNIRFLHELPQQSADRAGIKGRVYTNSIYELLENGQPAGVCVLGYATPLQTLFAMSQDGRAGFSREDRLEQAKLFCRTLEDILADAPEAQNNCRLIVYQEPTEGGSFSLSQEILRHLRQEEREVTMGSAETSVVPTSSTLSQEPELLISGMEQPLPLRSDIF